(19) United States
(12) Patent Application Publication
(10) Pub. No.: US 2024/0270695 A1
(43) Pub. Date: Aug. 15, 2024

(54) COMPOUNDS, COMPOSITIONS AND METHODS OF TREATING DISORDERS

(71) Applicant: Chengdu Anticancer Bioscience LTD, Chengdu (CN)

(72) Inventors: Dun Yang, Chengdu (CN); Jing Zhang, Chengdu (CN); Shenqiu Zhang, St. Andrews (GB); Thaddeus Allen, Daly City, CA (US); Qiong Shi, Chengdu (CN); Naganna Nimishetti, Chengdu (CN); Jian Huang, Chengdu (CN); Hongmei Li, Chengdu (CN); Chenglu Yang, Chengdu (CN)

(21) Appl. No.: 18/271,924

(22) PCT Filed: Jan. 11, 2022
(86) PCT No.: PCT/CN2022/071178

§ 371 Date: Jul. 12, 2023

(30) Foreign Application Priority Data
Jan. 12, 2021 (WO) PCT/CN2021/071225
Nov. 19, 2021 (WO) PCT/CN2021/131733

(51) Int. Cl. C07D 223/16; A61K 31/55; A61P 35/00; C07D 471/04

(52) U.S. Cl. CPC C07D 223/16; A61K 31/55; A61P 35/00; C07D 471/04

(57) ABSTRACT

The present disclose includes, among other things, compounds that treat or lessen the severity of cancer, pharmaceutical compositions and methods of making and using the same.

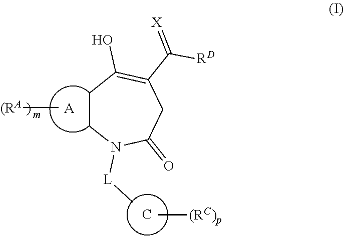

### BACKGROUND

Cancer is a term used for diseases in which abnormal cells divide without control and may invade other tissues. Cancer cells may also spread to other parts of the body through the blood and lymph systems.

There are more than 100 different types of cancer, with most cancers named for the organ or type of cell in which they start. For example, cancer that begins in the colon may be referred to as colon cancer; cancer that begins in basal cells of the skin may be referred to as basal cell carcinoma. Common types of cancer include breast cancer and lung cancer.

Cancer types can also be grouped into broader categories. The main categories of cancer include: carcinoma—cancer that begins in the skin or in tissues that line or cover internal organs; sarcoma—cancer that begins in bone, cartilage, fat, muscle, blood vessels, or other connective or supportive tissue; leukemia—cancer that starts in blood-forming tissue such as the bone marrow and causes large numbers of abnormal blood cells to be produced and enter the blood; lymphoma and myeloma-cancers that begin in the cells of the immune system; central nervous system cancers-cancers that begin in the tissues of the brain and spinal cord.

Several techniques for treating cancer are known in the art. Such techniques include chemotherapy, radiation therapy, surgery, and transplantation. Each of these techniques, however, have undesirable side effects and varying success rates. Therefore, a need exists to develop new methods for treating cancer and/or diseases associated with cellular proliferation.

### SUMMARY

The present disclosure provides for compounds of formula (I):

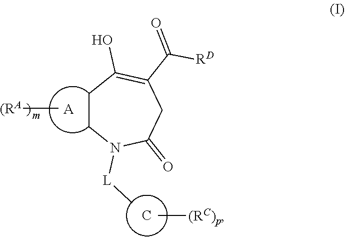

or a pharmaceutically acceptable salt thereof. Additionally, the present disclosure includes, among other things, pharmaceutical compositions, methods of using, and methods of making a compound of formula (I).

### BRIEF DESCRIPTION OF DRAWINGS

FIG. 1 depicts a graph of tumor volume of NCI-H23 mouse xenografts versus days after initiation of administration of compounds 55 and 58.

FIG. 2 depicts a graph of tumor volume of MDA-MB-231 mouse xenografts versus days after initiation of administration of compounds 55 and 58.

### DETAILED DESCRIPTION

In some embodiments, the present disclosure includes a compound of Formula (I):

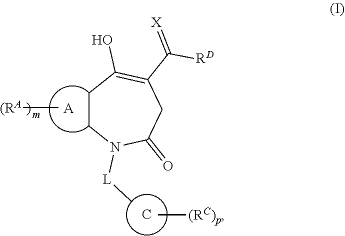

or a pharmaceutically acceptable salt thereof,
   wherein
X is O or S;
Ring A is selected from the group consisting of optionally substituted phenyl and optionally substituted 6-membered heteroaryl;
L is selected from the group consisting of a bond, optionally substituted C₁-C₃ alkylene, —C(O)—, —C(O)O—, —C(O)NH—, and —S(O)₂—;
Ring C is selected from the group consisting of optionally substituted phenyl, naphthyl, optionally substituted 3-7 membered carbocyclyl, and optionally substituted 6-10-membered heteroaryl;
each R^{A} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂; or
each R^{B} is independently selected from the group consisting of hydrogen and optionally substituted C₁-C₆ aliphatic,
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —S(O)₂R³, —N(R³)₂, —C(O)R³, —C(O)OR³, C(O)N(R³)₂, —N(H)C(O)R³, and —N(H)C(O)N(R³)₂;
   two instances of R^{C} are taken together with the atoms on which they are attached to form an optionally substituted 6-membered aryl or optionally substituted 6-membered heteroaryl;
R^{D} is selected from the group consisting of optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —(CH₂)₀₋₃OR², —SR², and —N(R²)₂;
each R¹ is independently selected from the group consisting of hydrogen, —(CH₂)₁₋₃R², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R¹ is optionally substituted with 1-6 instances of R⁴;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R² is optionally substituted with 1-6 instances of R⁴;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R³ is optionally substituted with 1-6 instances of R⁴;
each R⁴ is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OH, —O(C₁-C₃ alkyl), —O(C₁-C₃ haloalkyl), —SH, —S(C₁-C₃ alkyl), —S(C₁-C₃ haloalkyl), —C(O)OH, and —C(O)O(C₁-C₃ alkyl); m is 0, 1, 2, 3, or 4;
p is 0, 1, 2, 3, 4, or 5.

In some embodiments, present disclosure includes a compound of formula (I-a):

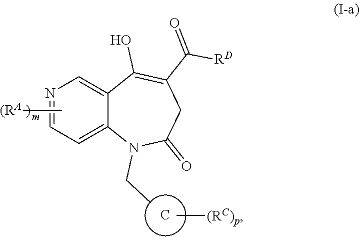

or a pharmaceutically acceptable salt thereof, wherein Ring C, R^{A}, R^{C}, R^{D}, m, and p are defined above and described in classes and subclasses herein.

In some embodiments, present disclosure includes a compound of formula (I-b):

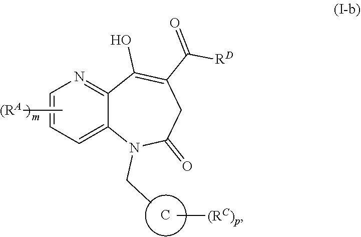

or a pharmaceutically acceptable salt thereof, wherein Ring C, R^{A}, R^{C}, R^{D}, m, and p are defined above and described in classes and subclasses herein.

In some embodiments, present disclosure includes a compound of formula (I-c):

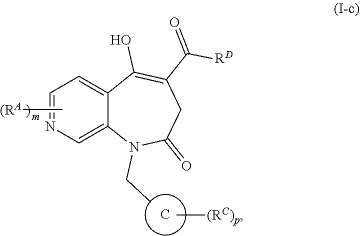

or a pharmaceutically acceptable salt thereof, wherein Ring C, R^{A}, R^{C}, R^{D}, m, and p are defined above and described in classes and subclasses herein.

In some embodiments, present disclosure includes a compound of formula (I-d):

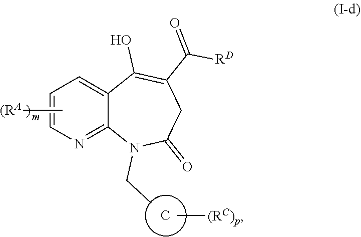

or a pharmaceutically acceptable salt thereof, wherein Ring C, R^{A}, R^{C}, R^{D}, m, and p are defined above and described in classes and subclasses herein.

In some embodiments, present disclosure includes a compound of formula (I-e):

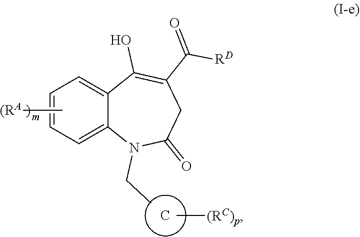

or a pharmaceutically acceptable salt thereof, wherein R^{A}, R^{C}, R^{D}, m, and p are defined above and described in classes and subclasses herein.

### Ring A

In some embodiments. Ring A is selected from the group consisting of optionally substituted phenyl and optionally substituted 6-membered heteroaryl. In some embodiments, Ring A is optionally substituted phenyl. In some embodiments. Ring A is optionally substituted heteroaryl. In some embodiments, Ring A is an optionally substituted 6-membered heteroaryl selected from pyridine, pyrazine, pyridazine, and pyrimidine. In some embodiments, Ring A is optionally substituted pyridine.

### Ring C

In some embodiments, Ring C is selected from the group consisting of optionally substituted phenyl, naphthyl, optionally substituted 3-7 membered carbocyclyl, and optionally substituted 6-10-membered heteroaryl. In some embodiments, Ring C is selected from the group consisting of optionally substituted phenyl and optionally substituted 6-membered heteroaryl. In some embodiments, Ring C is optionally substituted phenyl. In some embodiments, Ring C is optionally substituted heteroaryl. In some embodiments, Ring C is an optionally substituted 6-membered heteroaryl selected from pyridine, pyrazine, pyridazine, and pyrimidine. In some embodiments, Ring C is optionally substituted pyridine. In some embodiments, Ring C is optionally substituted phenyl or optionally substituted pyridine. In some embodiments, Ring C is optionally substituted phenyl.

### L

In some embodiments, L is selected from the group consisting of a bond, optionally substituted C₁-C₃ alkylene, —C(O)—, —C(O)O—, —C(O)NH—, and —S(O)₂—. In some embodiments, L is selected from the group consisting of a bond, optionally substituted C₁-C₃ alkylene, and —C(O)—. In some embodiments, L is a bond. In some embodiments, L is an optionally substituted C₁-C₃ alkylene chain. In some embodiments, L is an optionally substituted C₁ alkylene chain. In some embodiments, L is an optionally substituted C₂ alkylene chain. In some embodiments, L is an optionally substituted C₃ alkylene chain. In some embodiments, L is an unsubstituted C₁-C₃ alkylene chain. In some embodiments, L is —CH₂—. In some embodiments, L is —CH₂CH₂—. In some embodiments. L is —CH₂CH₂CH₂—.

In some embodiments, L and Ring C are taken together to form a group selected from

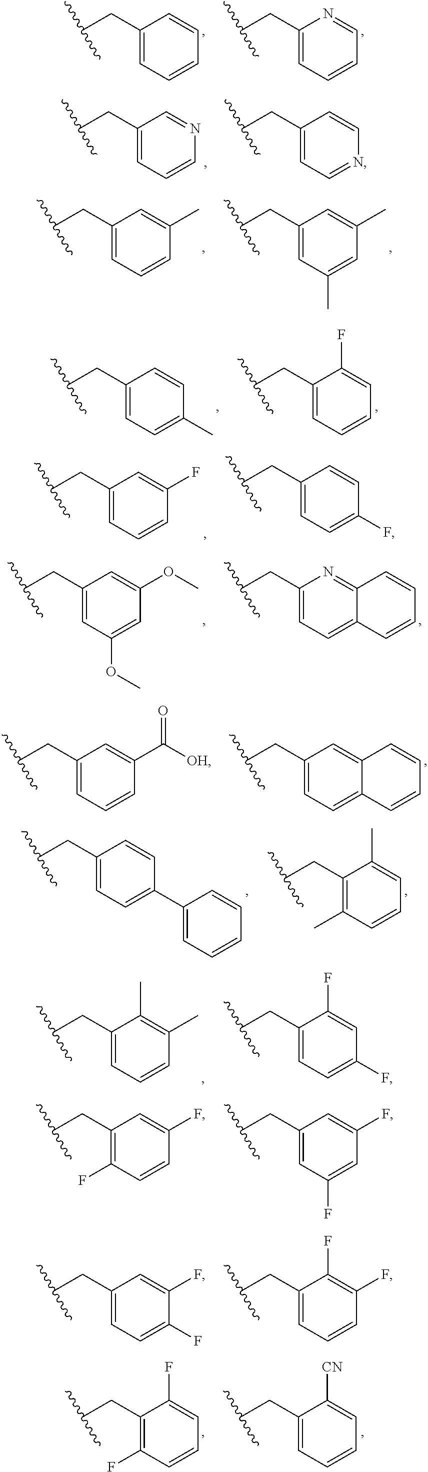

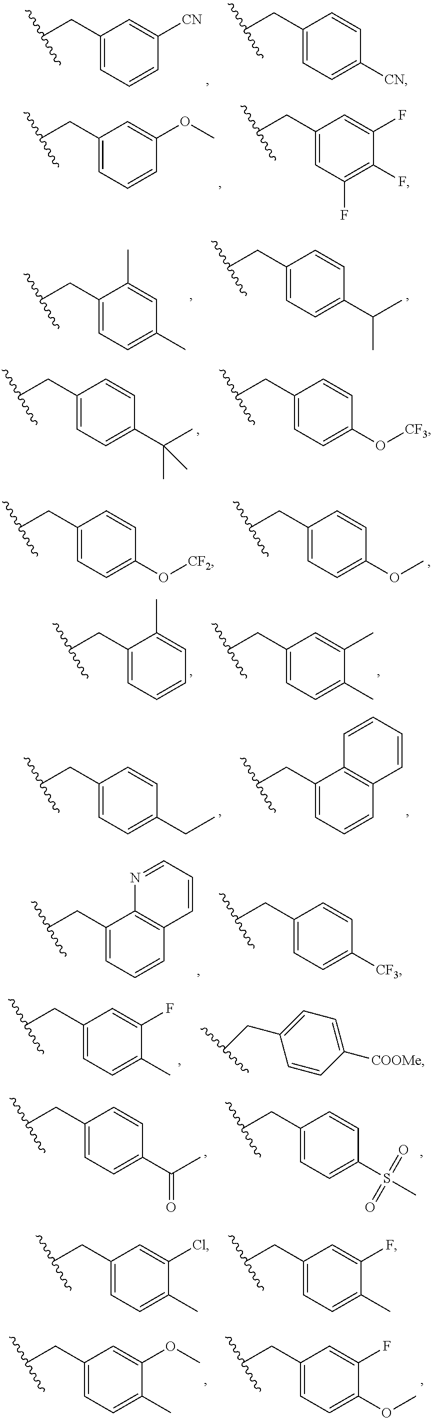

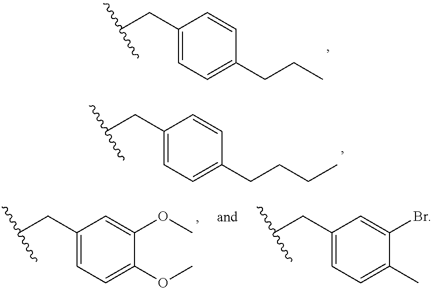

In some embodiments, L and Ring C are taken together to form a group selected from

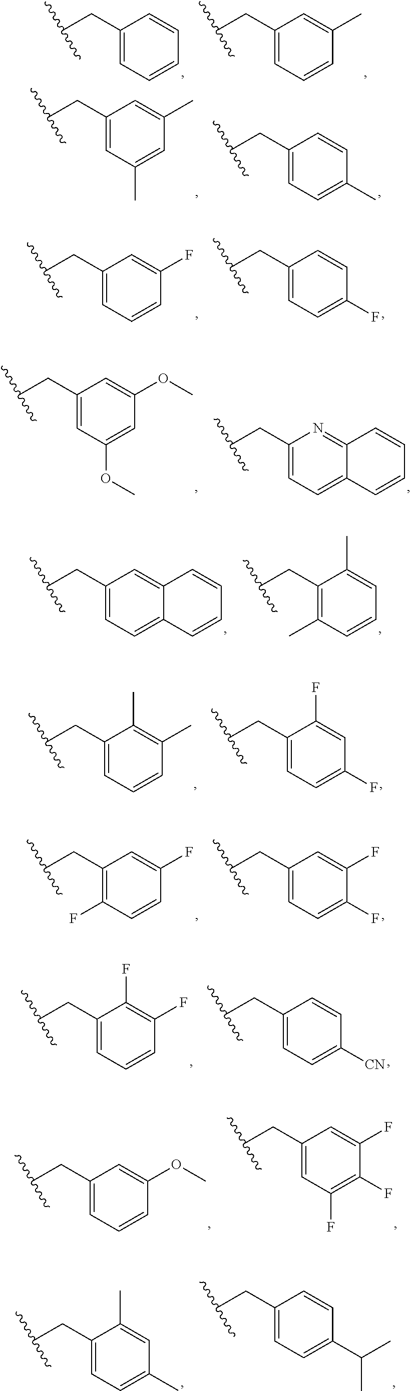

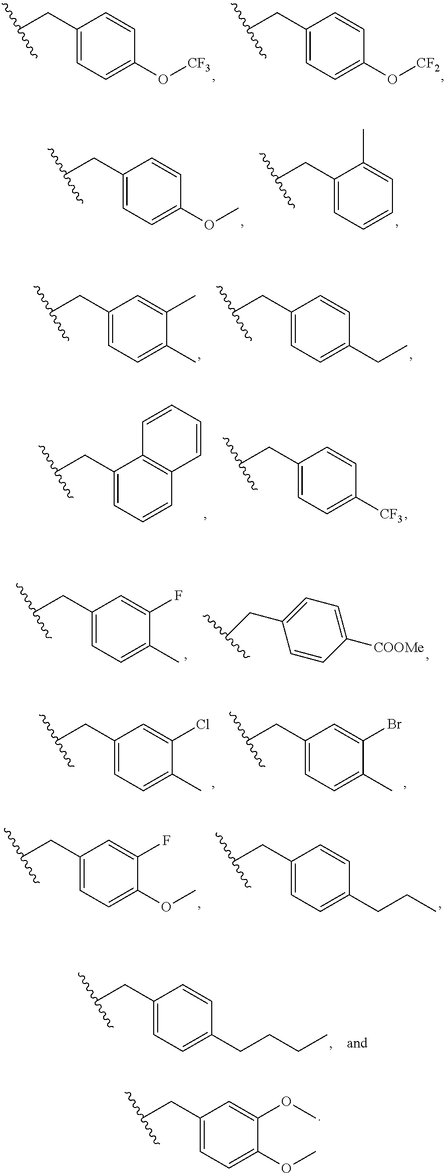

### R^{A}

In some embodiments each R^{A} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹); —N(H)C(O)R¹, and —N(H)C(O)N(R¹)₂. In some embodiments each R^{A} is independently selected from the group consisting of halogen, —CN, optionally substituted C₁-C₆ aliphatic, —OR¹, —SR¹, —N(R¹)₂, —C(O)OR¹, C(O)N(R¹)₂, —N(H)C(O)R¹, and —N(H)C(O)N(R′)₂.

### R^{C}

In some embodiments, each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —S(O)₂R³, —N(R¹)³, —C(O)R¹, —C(O)OR³, C(O)N(R³)₂, —N(H)C(O)R³, and —N(H)C(O)N(R³)₂; two instances of R^{C} are taken together with the atoms on which they are attached to form an optionally substituted 6-membered aryl or optionally substituted 6-membered heteroaryl. In some embodiments, each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —S(O)₂R³, —N(R³)₂, —C(O)OR³, C(O)N(R³)₂, —N(H)C(O)R³, and —N(H)C(O)N(R³)₂. In some embodiments, each R^{C} is independently selected from the group consisting of halogen, —CN, optionally substituted C₁-C₆ aliphatic, optionally substituted phenyl, —OR³, —S(O)₂R³, and —C(O)OR³. In some embodiments, each R^{C} is independently selected from the group consisting of halogen and optionally substituted C₁-C₆ aliphatic

In some embodiments, one instance of R^{C} is halogen and one instance is optionally substituted C₁-C₆ aliphatic.

In some embodiments, R^{C} is halogen. In some embodiments, R^{C} is fluorine. In some embodiments, R^{C} is chlorine. In some embodiments, R^{C} is bromine. In some embodiments, R^{C} is iodine

In some embodiments, R^{C} is —CN. In some embodiments, R^{C} is —S(O)₂Me.

In some embodiments, each R^{C} is independently optionally substituted C₁-C₆ aliphatic. In some embodiments, each R^{C} is independently optionally substituted C₁-C₄ aliphatic. In some embodiments, each R^{C} is independently selected from the group consisting of optionally substituted methyl, optionally substituted ethyl, optionally substituted iso-propyl, optionally substituted n-propyl, optionally substituted n-butyl and optionally substituted tert-butyl. In some embodiments, each R^{C} is independently selected from the group consisting of —CF₃ and —CHF₂.

In some embodiments, R^{C} is —OR³. In some embodiments each R^{C} is independently selected from the group consisting of —OMe, —OCF₃ and —OCHF₂.

In some embodiments, two instances of R^{C} are taken together with the atoms on which they are attached to form an optionally substituted 6-membered aryl or optionally substituted 6-membered heteroaryl. In some embodiments two instances of R^{C} are taken together with Ring C to form an optionally substituted naphthyl or optionally substituted 10-membered heteroaryl. In some embodiments two instances of R^{C} are taken together with Ring C to form an optionally substituted naphthyl, optionally substituted quinolinyl, or isoquinolinyl. In some embodiments two instances of R^{C} are taken together with Ring C to form an optionally substituted naphthyl or optionally substituted quinolinyl. In some embodiments two instances of R^{C} are taken together with Ring C to form an optionally substituted naphthyl. In some embodiments two instances of R^{C} are taken together with Ring C to form an optionally substituted quinolinyl.

### R^{D}

In some embodiments, R^{D} is independently selected from the group consisting of optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR², —SR², and —N(R²)₂. In some embodiments, R^{D} is selected from the group consisting of optionally substituted C₁-C₆ aliphatic, and —OR². In some embodiments, R^{D} is selected from the group consisting of optionally substituted methyl, optionally substituted ethyl, and —OMe. In some embodiments, R^{D} is optionally substituted methyl. In some embodiments, R^{D} is optionally substituted ethyl. In some embodiments, R^{D} is optionally substituted —OMe.

### R¹

In some embodiments, each R¹ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R¹ is independently optionally substituted C₁-C₆ aliphatic. In some embodiments, R¹ is optionally substituted methyl.

### R²

In some embodiments, each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R² is independently optionally substituted C₁-C₆ aliphatic. In some embodiments, R² is optionally substituted methyl.

### R³

In some embodiments, each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl. In some embodiments, each R³ is independently optionally substituted C₁-C₆ aliphatic. In some embodiments, each R³ is independently selected from the group consisting of hydrogen, optionally substituted methyl, —CF₃, and —CHF₂.

In some embodiments, the present disclosure includes compounds described in Tables 1-4.

TABLE 1

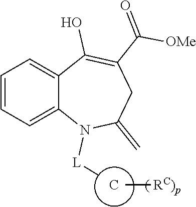

| Compound No. | -L-Ring C |
|---|---|
| 1 | 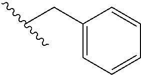 [SMILES: *Cc1ccccc1] |
| 2 | 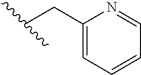 [SMILES: *Cc1ccccn1] |
| 3 | 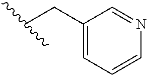 [SMILES: *Cc1cccnc1] |
| 4 | 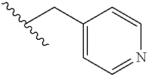 [SMILES: *Cc1ccncc1] |
| 5 | 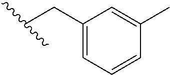 [SMILES: *Cc1cccc(C)c1] |
| 6 | 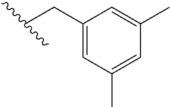 [SMILES: *Cc1cc(C)cc(C)c1] |
| 7 | 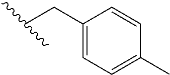 [SMILES: *Cc1ccc(C)cc1] |
| 8 | 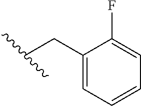 [SMILES: *Cc1ccccc1F] |
| 9 | 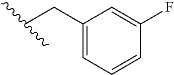 [SMILES: *Cc1cccc(F)c1] |
| 10 | 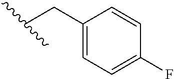 [SMILES: *Cc1ccc(F)cc1] |
| 11 | 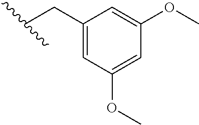 [SMILES: *Cc1cc(OC)cc(OC)c1] |
| 12 | 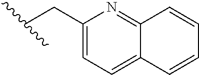 [SMILES: *Cc1ccc2ccccc2n1] |
| 13 | 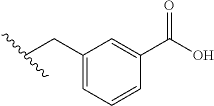 [SMILES: *Cc1cccc(C(=O)O)c1] |
| 14 | 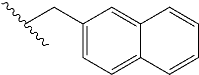 [SMILES: *Cc1ccc2ccccc2c1] |
| 15 | 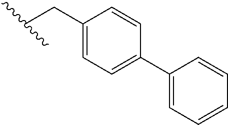 [SMILES: *Cc1ccc(-c2ccccc2)cc1] |
| 16 | 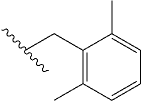 [SMILES: *Cc1c(C)cccc1C] |
| 17 | 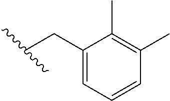 [SMILES: *Cc1cccc(C)c1C] |
| 18 | 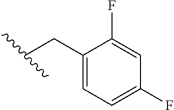 [SMILES: *Cc1ccc(F)cc1F] |
| 19 | 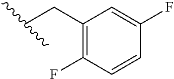 [SMILES: *Cc1cc(F)ccc1F] |
| 20 | 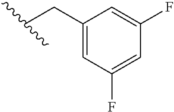 [SMILES: *Cc1cc(F)cc(F)c1] |
| 21 | 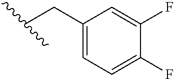 [SMILES: *Cc1ccc(F)c(F)c1] |
| 22 | 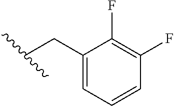 [SMILES: *Cc1cccc(F)c1F] |
| 23 | 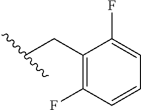 [SMILES: *Cc1c(F)cccc1F] |
| 24 | 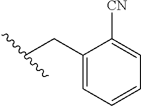 [SMILES: *Cc1ccccc1C#N] |
| 25 | 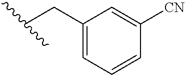 [SMILES: *Cc1cccc(C#N)c1] |
| 26 | 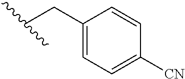 [SMILES: *Cc1ccc(C#N)cc1] |
| 27 | 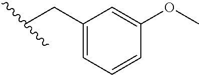 [SMILES: *Cc1cccc(OC)c1] |
| 28 | 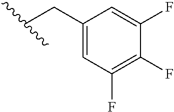 [SMILES: *Cc1cc(F)c(F)c(F)c1] |
| 29 | 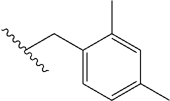 [SMILES: *Cc1ccc(C)cc1C] |
| 30 | 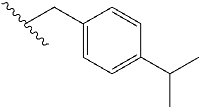 [SMILES: *Cc1ccc(C(C)C)cc1] |
| 31 | 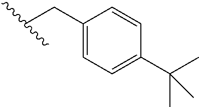 [SMILES: *Cc1ccc(C(C)(C)C)cc1] |
| 32 | 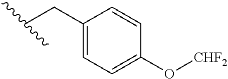 [SMILES: *Cc1ccc(OC(F)F)cc1] |
| 33 | 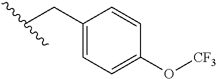 [SMILES: *Cc1ccc(OC(F)(F)F)cc1] |
| 34 | 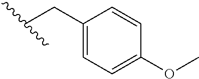 [SMILES: *Cc1ccc(OC)cc1] |
| 35 | 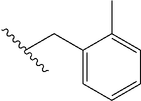 [SMILES: *Cc1ccccc1C] |
| 36 | 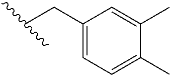 [SMILES: *Cc1ccc(C)c(C)c1] |
| 37 | 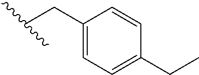 [SMILES: *Cc1ccc(CC)cc1] |
| 38 | 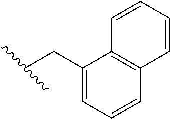 [SMILES: *Cc1cccc2ccccc12] |
| 39 | 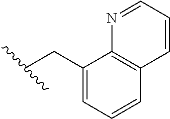 [SMILES: *Cc1cccc2cccnc12] |
| 40 | 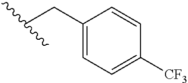 [SMILES: *Cc1ccc(C(F)(F)F)cc1] |
| 41 | 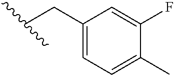 [SMILES: *Cc1ccc(C)c(F)c1] |
| 42 | 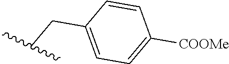 [SMILES: *Cc1ccc(C(=O)OC)cc1] |
| 43 | 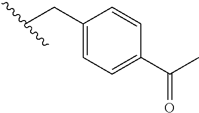 [SMILES: *Cc1ccc(C(C)=O)cc1] |
| 44 | 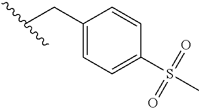 [SMILES: *Cc1ccc(S(C)(=O)=O)cc1] |
| 45 | 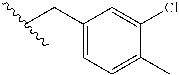 [SMILES: *Cc1ccc(C)c(Cl)c1] |
| 46 | 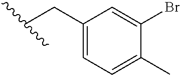 [SMILES: *Cc1ccc(C)c(Br)c1] |
| 47 | 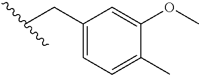 [SMILES: *Cc1ccc(C)c(OC)c1] |
| 48 | 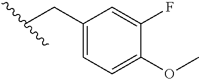 [SMILES: *Cc1ccc(OC)c(F)c1] |
| 49 | 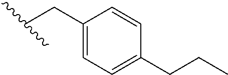 [SMILES: *Cc1ccc(CCC)cc1] |
| 50 | 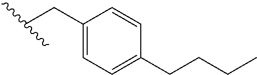 [SMILES: *Cc1ccc(CCCC)cc1] |
| 51 | 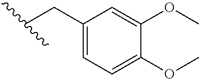 [SMILES: *Cc1ccc(OC)c(OC)c1] |

TABLE 2

| Compound No. | Structure |
|---|---|
| 52 | 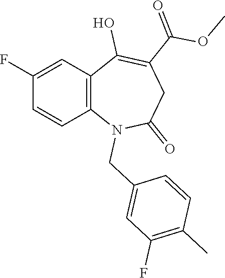 [SMILES: COC(=O)C1=C(O)c2cc(F)ccc2N(Cc2ccc(C)c(F)c2)C(=O)C1] |
| 53 | 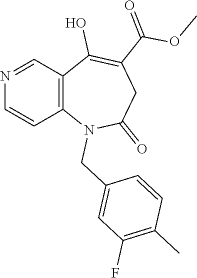 [SMILES: COC(=O)C1=C(O)c2cnccc2N(Cc2ccc(C)c(F)c2)C(=O)C1] |
| 54 | 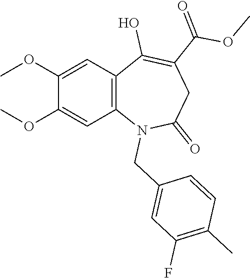 |
| 55 | 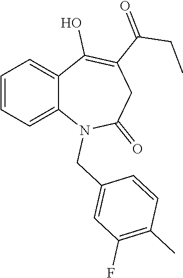 [SMILES: CCC(=O)C1=C(O)c2ccccc2N(Cc2ccc(C)c(F)c2)C(=O)C1] |
| 56 | 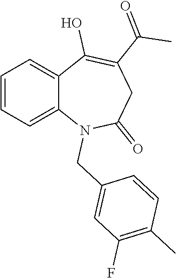 [SMILES: CC(=O)C1=C(O)c2ccccc2N(Cc2ccc(C)c(F)c2)C(=O)C1] |
| 57 | 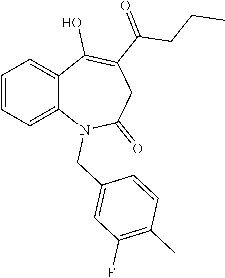 [SMILES: CCCC(=O)C1=C(O)c2ccccc2N(Cc2ccc(C)c(F)c2)C(=O)C1] |
| 58 | 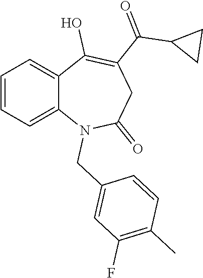 [SMILES: Cc1ccc(CN2C(=O)CC(C(=O)C3CC3)=C(O)c3ccccc32)cc1F] |
| 59 | 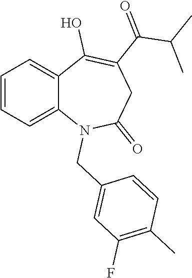 [SMILES: Cc1ccc(CN2C(=O)CC(C(=O)C(C)C)=C(O)c3ccccc32)cc1F] |
| 60 | 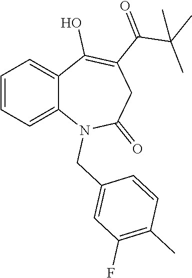 [SMILES: Cc1ccc(CN2C(=O)CC(C(=O)C(C)(C)C)=C(O)c3ccccc32)cc1F] |
| 61 | 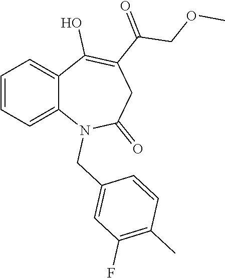 [SMILES: COCC(=O)C1=C(O)c2ccccc2N(Cc2ccc(C)c(F)c2)C(=O)C1] |
| 62 | 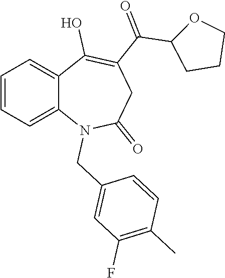 [SMILES: Cc1ccc(CN2C(=O)CC(C(=O)C3CCCO3)=C(O)c3ccccc32)cc1F] |
| 63 | 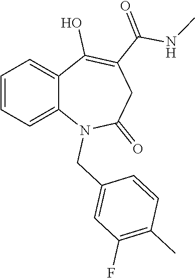 [SMILES: CNC(=O)C1=C(O)c2ccccc2N(Cc2ccc(C)c(F)c2)C(=O)C1] |
| 64 | 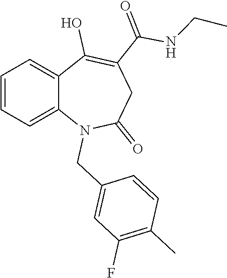 [SMILES: CCNC(=O)C1=C(O)c2ccccc2N(Cc2ccc(C)c(F)c2)C(=O)C1] |
| 65 | 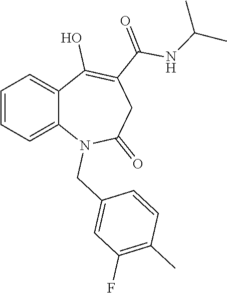 [SMILES: Cc1ccc(CN2C(=O)CC(C(=O)NC(C)C)=C(O)c3ccccc32)cc1F] |
| 66 | 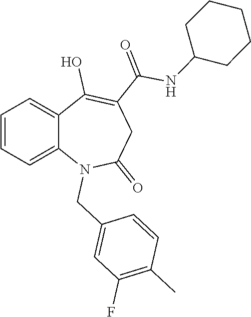 [SMILES: Cc1ccc(CN2C(=O)CC(C(=O)NC3CCCCC3)=C(O)c3ccccc32)cc1F] |
| 67 | 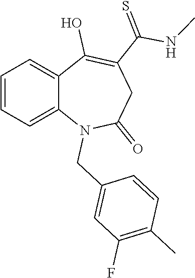 [SMILES: CNC(=S)C1=C(O)c2ccccc2N(Cc2ccc(C)c(F)c2)C(=O)C1] |
| 68 | 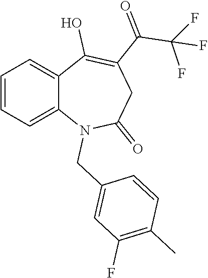 [SMILES: Cc1ccc(CN2C(=O)CC(C(=O)C(F)(F)F)=C(O)c3ccccc32)cc1F] |
| 69 | 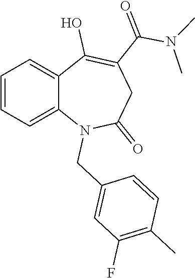 [SMILES: Cc1ccc(CN2C(=O)CC(C(=O)N(C)C)=C(O)c3ccccc32)cc1F] |

TABLE 3

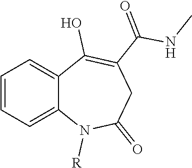

| No. | R = |
|---|---|
| 70 | 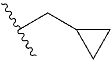 [SMILES: *CC1CC1] |
| 71 | 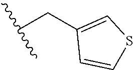 [SMILES: *Cc1ccsc1] |
| 72 | 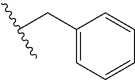 [SMILES: *Cc1ccccc1] |
| 73 | 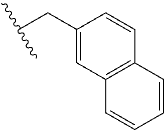 [SMILES: *Cc1ccc2ccccc2c1] |
| 74 | 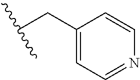 [SMILES: *Cc1ccncc1] |
| 75 | 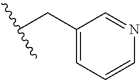 [SMILES: *Cc1cccnc1] |
| 76 | 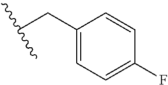 [SMILES: *Cc1ccc(F)cc1] |
| 77 | 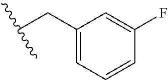 [SMILES: *Cc1cccc(F)c1] |
| 78 | 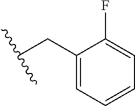 [SMILES: *Cc1ccccc1F] |
| 79 | 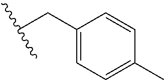 [SMILES: *Cc1ccc(C)cc1] |
| 80 | 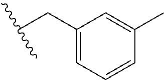 [SMILES: *Cc1cccc(C)c1] |
| 81 | 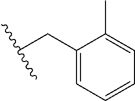 [SMILES: *Cc1ccccc1C] |
| 82 | 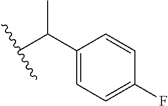 [SMILES: *C(C)c1ccc(F)cc1] |
| 83 | 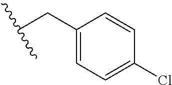 [SMILES: *Cc1ccc(Cl)cc1] |
| 84 | 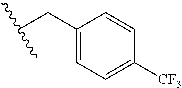 [SMILES: *Cc1ccc(C(F)(F)F)cc1] |
| 85 | 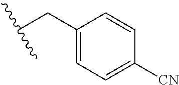 [SMILES: *Cc1ccc(C#N)cc1] |
| 86 | 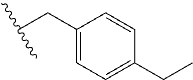 [SMILES: *Cc1ccc(CC)cc1] |
| 87 | 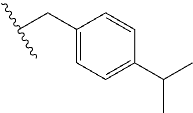 [SMILES: *Cc1ccc(C(C)C)cc1] |
| 88 | 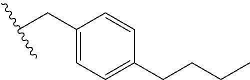 [SMILES: *Cc1ccc(CCCC)cc1] |
| 89 | 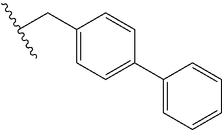 [SMILES: *Cc1ccc(-c2ccccc2)cc1] |
| 90 | 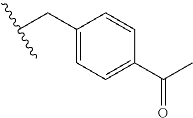 [SMILES: *Cc1ccc(C(C)=O)cc1] |
| 91 | 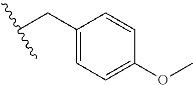 [SMILES: *Cc1ccc(OC)cc1] |
| 92 | 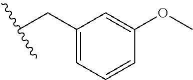 [SMILES: *Cc1cccc(OC)c1] |
| 93 | 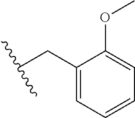 [SMILES: *Cc1ccccc1OC] |
| 94 | 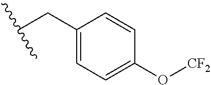 |
| 95 | 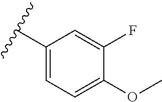 [SMILES: *c1ccc(OC)c(F)c1] |
| 96 | 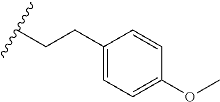 [SMILES: *CCc1ccc(OC)cc1] |
| 97 | 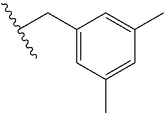 [SMILES: *Cc1cc(C)cc(C)c1] |
| 98 | 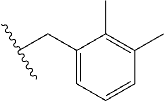 [SMILES: *Cc1cccc(C)c1C] |
| 99 | 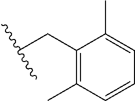 [SMILES: *Cc1c(C)cccc1C] |
| 100 | 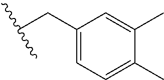 [SMILES: *Cc1ccc(C)c(C)c1] |
| 101 | 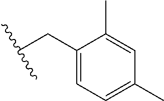 [SMILES: *Cc1ccc(C)cc1C] |
| 102 | 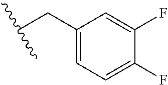 [SMILES: *Cc1ccc(F)c(F)c1] |
| 103 | 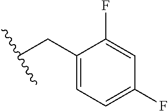 [SMILES: *Cc1ccc(F)cc1F] |
| 104 | 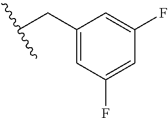 [SMILES: *Cc1cc(F)cc(F)c1] |
| 105 | 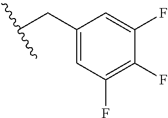 [SMILES: *Cc1cc(F)c(F)c(F)c1] |
| 106 | 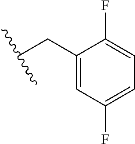 [SMILES: *Cc1cc(F)ccc1F] |
| 107 | 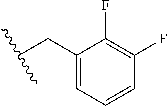 [SMILES: *Cc1cccc(F)c1F] |
| 108 | 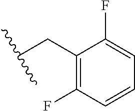 [SMILES: *Cc1c(F)cccc1F] |
| 109 | 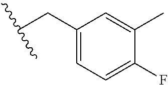 [SMILES: *Cc1ccc(F)c(C)c1] |
| 110 | 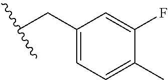 [SMILES: *Cc1ccc(C)c(F)c1] |
| 111 | 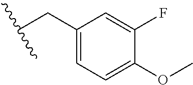 [SMILES: *Cc1ccc(OC)c(F)c1] |
| 112 | 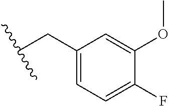 [SMILES: *Cc1ccc(F)c(OC)c1] |
| 113 | 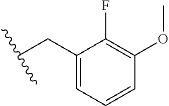 [SMILES: *Cc1cccc(OC)c1F] |
| 114 | 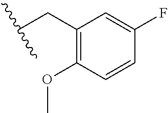 [SMILES: *Cc1cc(F)ccc1OC] |
| 115 | 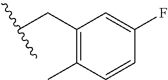 [SMILES: *Cc1cc(F)ccc1C] |
| 116 | 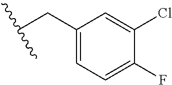 [SMILES: *Cc1ccc(F)c(Cl)c1] |
| 117 | 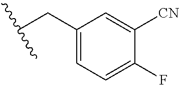 [SMILES: *Cc1ccc(F)c(C#N)c1] |
| 118 | 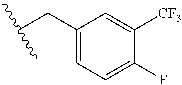 [SMILES: *Cc1ccc(F)c(C(F)(F)F)c1] |
| 119 | 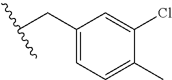 [SMILES: *Cc1ccc(C)c(Cl)c1] |
| 120 | 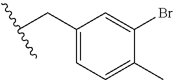 [SMILES: *Cc1ccc(C)c(Br)c1] |
| 121 | 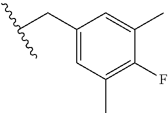 [SMILES: *Cc1cc(C)c(F)c(C)c1] |
| 122 | 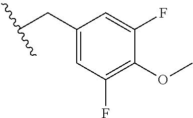 [SMILES: *Cc1cc(F)c(OC)c(F)c1] |
| 123 | 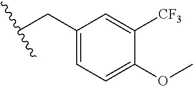 [SMILES: *Cc1ccc(OC)c(C(F)(F)F)c1] |
| 124 | 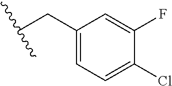 [SMILES: *Cc1ccc(Cl)c(F)c1] |
| 125 | 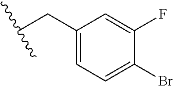 [SMILES: *Cc1ccc(Br)c(F)c1] |

TABLE 4

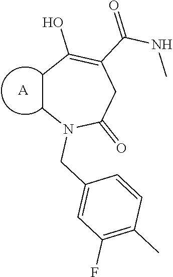

| No. | A = |
|---|---|
| 126 | 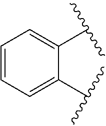 [SMILES: *c1ccccc1*] |
| 127 | 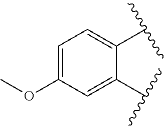 [SMILES: *c1ccc(OC)cc1*] |
| 128 | 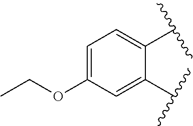 [SMILES: *c1ccc(OCC)cc1*] |
| 129 | 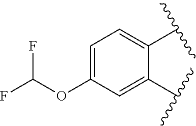 [SMILES: *c1ccc(OC(F)F)cc1*] |
| 130 | 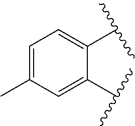 [SMILES: *c1ccc(C)cc1*] |
| 131 | 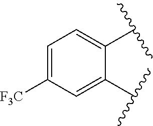 [SMILES: *c1ccc(C(F)(F)F)cc1*] |
| 132 | 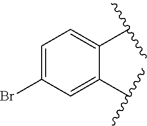 [SMILES: *c1ccc(Br)cc1*] |
| 133 | 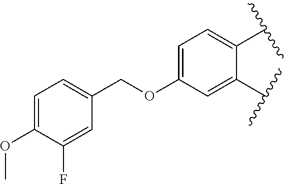 [SMILES: *c1ccc(OCc2ccc(OC)c(F)c2)cc1*] |
| 134 | 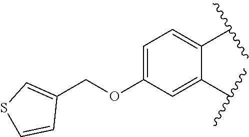 [SMILES: *c1ccc(OCc2ccsc2)cc1*] |
| 135 | 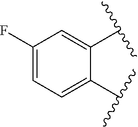 [SMILES: *c1ccc(F)cc1*] |
| 136 | 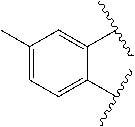 [SMILES: *c1ccc(C)cc1*] |
| 137 | 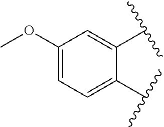 [SMILES: *c1ccc(OC)cc1*] |
| 138 | 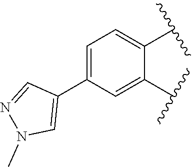 |
| 139 | 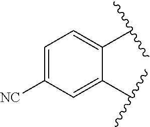 [SMILES: *c1ccc(C#N)cc1*] |
| 140 | 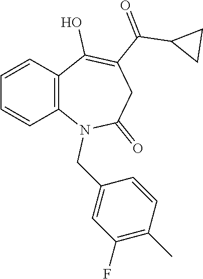 |
| 141 | 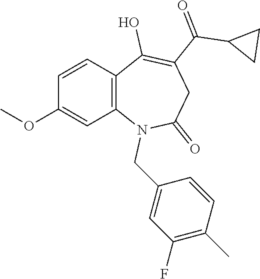 |

### Definitions

The term “aliphatic” or “aliphatic group”, as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic (also referred to herein as “carbocycle” “cycloaliphatic” or “cycloalkyl”), that has a single point of attachment to the rest of the molecule. Unless otherwise specified, aliphatic groups contain 1-6 aliphatic carbon atoms. In some embodiments, aliphatic groups contain 1-5 aliphatic carbon atoms. In other embodiments, aliphatic groups contain 1-4 aliphatic carbon atoms. In still other embodiments, aliphatic groups contain 1-3 aliphatic carbon atoms, and in yet other embodiments, aliphatic groups contain 1-2 aliphatic carbon atoms. In some embodiments, “cycloaliphatic” (or “carbocycle” or “cycloalkyl”) refers to a monocyclic C₃-C₆ hydrocarbon that is completely saturated or that contains one or more units of unsaturation, but which is not aromatic, that has a single point of attachment to the rest of the molecule. Suitable aliphatic groups include, but are not limited to, linear or branched, substituted or unsubstituted alkyl, alkenyl, alkynyl groups and hybrids thereof such as (cycloalkyl)alkyl, (cycloalkenyl)alkyl or (cycloalkyl)alkenyl.

The term “haloaliphatic” refers to an aliphatic group that is substituted with one or more halogen atoms.

The term “alkyl” refers to a straight or branched alkyl group. Exemplary alkyl groups are methyl, ethyl, propyl, isopropyl, butyl, isobutyl, and ter-butyl.

The term “haloalkyl” refers to a straight or branched alkyl group that is substituted with one or more halogen atoms.

The term “halogen” means F, Cl, Br, or I.

The term “aryl” used alone or as part of a larger moiety as in “aralkyl”, “aralkoxy”, or “aryloxyalkyl”, refers to monocyclic and bicyclic ring systems having a total of five to fourteen ring members, wherein at least one ring in the system is aromatic and wherein each ring in the system contains three to seven ring members. The term “aryl” may be used interchangeably with the term “aryl ring” In certain embodiments of the present disclosure, “aryl” refers to an aromatic ring system which includes, but not limited to, phenyl, biphenyl, naphthyl, anthracyl and the like, which may bear one or more substituents. Also included within the scope of the term “aryl”, as it is used herein, is a group in which an aromatic ring is fused to one or more non-aromatic rings, such as indanyl, phthalimidyl, naphthimidyl, phenanthridinyl, or tetrahydronaphthyl, and the like.

The terms “heteroaryl” and “heteroar-”, used alone or as part of a larger moiety, e.g., “heteroaralkyl”, or “heteroaralkoxy”, refer to groups having 5 to 10 ring atoms, preferably 5, 6, or 9 ring atoms, having 6, 10, or 14 π electrons shared in a cyclic array; and having, in addition to carbon atoms, from one to five heteroatoms. The term “heteroatom” refers to nitrogen, oxygen, or sulfur, and includes any oxidized form of nitrogen or sulfur, and any quaternized form of a basic nitrogen. Heteroaryl groups include, without limitation, thienyl, furanyl, pyrrolyl, imidazolyl, pyrazolyl, triazolyl, tetrazolyl, oxazolyl, isoxazolyl, oxadiazolyl, thiazolyl, isothiazolyl, thiadiazolyl, pyridyl, pyridazinyl, pyrimidinyl, pyrazinyl, indolizinyl, purinyl, naphthyridinyl, and pteridinyl. The terms “heteroaryl” and “heteroar-”, as used herein, also include groups in which a heteroaromatic ring is fused to one or more aryl, cycloaliphatic, or heterocyclyl rings, where the radical or point of attachment is on the heteroaromatic ring. Nonlimiting examples include indolyl, isoindolyl, benzothienyl, benzofuranyl, dibenzofuranyl, indazolyl, benzimidazolyl, benzthiazolyl, quinolyl, isoquinolyl, cinnolinyl, phthalazinyl, quinazolinyl, quinoxalinyl, 4H-quinolizinyl, carbazolyl, acridinyl, phenazinyl, phenothiazinyl, phenoxazinyl, tetrahydroquinolinyl, tetrahydroisoquinolinyl, and pyrido[2,3-b]-1,4-oxazin-3 (4H)-one. A heteroaryl group may be mono- or bicyclic. The term “heteroaryl” may be used interchangeably with the terms “heteroaryl ring”, “heteroaryl group”, or “heteroaromatic”, any of which terms include rings that are optionally substituted. The term “heteroaralkyl” refers to an alkyl group substituted by a heteroaryl, wherein the alkyl and heteroaryl portions independently are optionally substituted.

As used herein, the terms “heterocycle”, “heterocyclyl”, “heterocyclic radical”, and “heterocyclic ring” are used interchangeably and refer to a stable 5- to 7-membered monocyclic or 7-10-membered bicyclic heterocyclic moiety that is either saturated or partially unsaturated, and having, in addition to carbon atoms, one or more, preferably one to four, heteroatoms, as defined above. When used in reference to a ring atom of a heterocycle, the term “nitrogen” includes a substituted nitrogen. As an example, in a saturated or partially unsaturated ring having 0-3 heteroatoms selected from oxygen, sulfur or nitrogen, the nitrogen may be N (as m 3,4-dihydro-2H-pyrrolyl), NH (as in pyrrolidinyl), or ⁺NR (as in TV-substituted pyrrolidinyl). A heterocyclic ring can be attached to its pendant group at any heteroatom or carbon atom that results in a stable structure and any of the ring atoms can be optionally substituted. Examples of such saturated or partially unsaturated heterocyclic radicals include, without limitation, tetrahydrofuranyl, tetrahydrothiophenyl pyrrolidinyl, piperidinyl, pyrrolinyl, tetrahydroquinolinyl, tetrahydroisoquinolinyl, decahydroquinolinyl, oxazolidinyl, piperazinyl, dioxanyl, dioxolanyl, diazepinyl, oxazepinyl, thiazepinyl, morpholinyl, and quinuclidinyl. The terms “heterocycle”, “heterocyclyl”, “heterocyclyl ring”, “heterocyclic group”, “heterocyclic moiety”, and “heterocyclic radical”, are used interchangeably herein, and also include groups in which a heterocyclyl ring is fused to one or more aryl, heteroaryl, or cycloaliphatic rings, such as indolinyl, 3H-indolyl, chromanyl, phenanthridinyl, or tetrahydroquinolinyl, where the radical or point of attachment is on the heterocyclyl ring. A heterocyclyl group may be mono- or bicyclic. The term “heterocyclylalkyl” refers to an alkyl group substituted by a heterocyclyl, wherein the alkyl and heterocyclyl portions independently are optionally substituted.

As used herein, the term “partially unsaturated” refers to a ring moiety that includes at least one double or triple bond. The term “partially unsaturated” is intended to encompass rings having multiple sites of unsaturation, but is not intended to include aryl or heteroaryl moieties, as herein defined.

As described herein, compounds of the invention may contain “optionally substituted” moieties. In general, the term “substituted”, whether preceded by the term “optionally” or not, means that one or more hydrogens of the designated moiety are replaced with a suitable substituent. Unless otherwise indicated, an “optionally substituted” group may have a suitable substituent at each substitutable position of the group, and when more than one position in any given structure may be substituted with more than one substituent selected from a specified group, the substituent may be either the same or different at every position. Combinations of substituents envisioned by this invention are preferably those that result in the formation of stable or chemically feasible compounds. The term “stable”, as used herein, refers to compounds that are not substantially altered when subjected to conditions to allow for their production, detection, and, in certain embodiments, their recovery, purification, and use for one or more of the purposes disclosed herein.

Suitable monovalent substituents on a substitutable carbon atom of an “optionally substituted” group are independently halogen; —(CH₂)₀₋₄R^{∘}; —(CH₂)₀₋₄OR^{∘}; —O(CH₂)₀₋₄R^{∘}, O—(CH₂)₀₋₄C(O)OR^{∘}; —(CH₂)₀₋₄CH(OR)₂; —(CH₂)₀₋₄SR^{∘}, —(CH₂)₀₋₄Ph, which may be substituted with R^{∘}, —(CH₂)₀₋₄O(CH₂)₀₋₁Ph which may be substituted with R^{∘}, —CH═CHPh, which may be substituted with R^{∘}; —(CH₂)₀₋₄O(CH₂)₀₋₁-pyridyl which may be substituted with R^{∘}; —NO₂; —CN, —N₃; —(CH₂)₀₋₄N(R^{∘})₂; —(CH₂)₀₋₄N(R^{∘})C(O)R^{∘}, —N(R^{∘})C(S)R^{∘}, —(CH₂)_{0- 4}N(R^{∘})C(O)NR^{∘}₂; —N(R^{∘})C(S)NR^{∘}₂; —(CH₂)₀₋₄N(R^{∘})C(O)OR^{∘}; —N(R^{∘})N(R^{∘})C(O)R^{∘}; —N(R^{∘})N(R^{∘})C(O)NR^{∘}₂; —N(R^{∘})N(R^{∘})C(O)OR^{∘}; —(CH₃)₀₋₄C(O)R^{∘}; —C(S)R^{∘}; —(CH₂)₀₋₄C(O)OR^{∘}; —(CH₂)₀₋₄C(O)SR^{∘}; —(CH₂)₀₋₄C(O)OSiR^{∘}₃; —(CH₂)₀₋₄OC(O)R^{∘}; —OC(O)(CH₂)₀₋₄SR^{∘}, SC(S)SR^{∘}; —(CH₂)₀₋₄SC(O)R^{∘}; —(CH₂)₀₋₄C(O)NR^{∘}₂; —C(S)NR^{∘}₂; —C(S)SR^{∘}; —SC(S)SR^{∘}, —(CH₂)₀₋₄OC(O)NR^{∘}₂; —C(O)N(OR^{∘})R^{∘}; —C(O)C(O)R^{∘}; —C(O)CH₂C(O)R^{∘}, —C(NOR^{∘})R^{∘}; —(CH₂)₀₋₄SSR^{∘}; —(CH₂)₀₋₄S(O)₂R^{∘}; —(CH₂)₀₋₄S(O)₂OR^{∘}; —(CH₂)₀₋₄OS(O)₂R^{∘}; —S(O)NR^{∘}₂; —(CH₂)₀₋₄S(O)R^{∘}; —N(R)S(O)₂NR^{∘}₂; —N(R)S(O)₂R^{∘}; —N(OR)R^{∘}; —C(NH)NR^{∘}₂, —P(O)₂R^{∘}, —P(O)R^{∘}₂; —OP(O)R^{∘}₂; —OP(O)(OR^{∘})₂; SiR^{∘}₃, —(C₁₋₄ straight or branched alkylene)O—N(R^{∘})₂; or —(C₁₋₄ straight or branched alkylene)C(O)O—N(R^{∘})₂, wherein each R^{∘} may be substituted as defined below and is independently hydrogen, C₁₋₆ aliphatic, —CH₂Ph, —O(CH₂)₀₋₁Ph, —CH₂-(5-6 membered heteroaryl ring), or a 5-6-membered saturated, partially unsaturated, or aryl ring having 0-4 heteroatoms independently selected from nitrogen, oxygen, or sulfur, or, notwithstanding the definition above, two independent occurrences of R^{∘}, taken together with their intervening atom(s), form a 3-12-membered saturated, partially unsaturated, or aryl mono- or bicyclic ring having 0-4 heteroatoms independently selected from nitrogen, oxygen, or sulfur, which may be substituted as defined below

Suitable monovalent substituents on R^{∘} (or the ring formed by taking two independent occurrences of R^{∘} together with their intervening atoms), are independently halogen, —(CH₂)₀₋₂R^{●}, -(haloR^{●}), —(CH₂)₀₋₂OH, —(CH₂)₀₋₂OR^{●}, —(CH₂)₀₋₂CH(OR^{●})₂; —O(haloR^{●}), —CN, —N₃, —(CH₂)₂C(O)R^{●}, —(CH₂)₀₋₂C(O)OH, —(CH₂)₀₋₂C(O)OR^{●}, —(CH₂)₀₋₂SR^{●}, —(CH₂)₀₋₂SH, —(CH₂)₀₋₂NH₂, —(CH₂)₀₋₂ NHR^{●}, —(CH₂)₀₋₂NR^{●}₂, —NO₂, —SiR^{●}₃, —OSiR^{●}₃, —C(O)SR^{●}, —(C₁₋₄ straight or branched alkylene)C(O)OR^{●}, or —SSR^{●} wherein each R^{●} is unsubstituted or where preceded by “halo” is substituted only with one or more halogens, and is independently selected from C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur. Suitable divalent substituents on a saturated carbon atom of R^{∘} include ═O and ═S.

Suitable divalent substituents on a saturated carbon atom of an “optionally substituted” group include the following: ═O, ═S, ═NNR*₂, ═NNHC(O)R*, ═NNHC(O)OR*, ═NNHS(O)₂R*, ═NR*, ═NOR*, —(O)(C(R*₂))₂₋₃O—, or —S(C(R*₂))₂₋₃S—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur. Suitable divalent substituents that are bound to vicinal substitutable carbons of an “optionally substituted” group include —O(CR*₂)₂₋₃O—, wherein each independent occurrence of R* is selected from hydrogen, C₁₋₆ aliphatic which may be substituted as defined below, or an unsubstituted 5-6-membered saturated, partially unsaturated, or aryl ring having 0-4 heteroatoms independently selected from nitrogen, oxygen, or sulfur.

Suitable substituents on the aliphatic group of R* include halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Ph, —O(CH₂)₀₋₁Ph, or a 5-6-membered saturated, partially unsaturated, or aryl ring having 0-4 heteroatoms independently selected from nitrogen, oxygen, or sulfur.

Suitable substituents on a substitutable nitrogen of an “optionally substituted” group include —R^{†}, —NR^{†}₂, —C(O)R^{†}, —C(O)OR^{†}, —C(O)C(O)R^{†}, —C(O)CH₂C(O)R^{†}, —S(O)₂R^{†}, —S(O)₂NR^{†}₂, —C(S)NR^{†}₂, —C(NH)NR^{†}₂, or —N(R^{†})S(O)₂R^{†}; wherein each R^{†} is independently hydrogen, C₁₋₆ aliphatic which may be substituted as defined below, unsubstituted —OPh, or an unsubstituted 5-6-membered saturated, partially unsaturated, or aryl ring having 0-4 heteroatoms independently selected from nitrogen, oxygen, or sulfur, or, notwithstanding the definition above, two independent occurrences of R^{†}, taken together with their intervening atom(s) form an unsubstituted 3-12-membered saturated, partially unsaturated, or aryl mono- or bicyclic ring having 0-4 heteroatoms independently selected from nitrogen, oxygen, or sulfur

Suitable substituents on the aliphatic group of R^{†} are independently halogen, —R^{●}, -(haloR^{●}), —OH, —OR^{●}, —O(haloR^{●}), —CN, —C(O)OH, —C(O)OR^{●}, —NH₂, —NHR^{●}, —NR^{●}₂, or —NO₂, wherein each R^{●} is unsubstituted or where preceded by “halo” is substituted only with one or more halogens, and is independently C₁₋₄ aliphatic, —CH₂Pb, —((CH₂)₀₋₁Ph, or a 5-6-membered saturated, partially unsaturated, or aryl ring having 0-4 heteroatoms independently selected from nitrogen, oxygen, or sulfur

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge et al., describe pharmaceutically acceptable salts in detail in J. Pharmaceutical Sciences, 1977, 66, 1-19, incorporated herein by reference. Pharmaceutically acceptable salts of the compounds of this disclosure include those derived from suitable inorganic and organic acids and bases Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, pivalate, propionate, stearate, succinate, sulfate, tartrate, thiocyanate, p-toluenesulfonate, undecanoate, valerate salts, and the like.

Salts derived from appropriate bases include alkali metal, alkaline earth metal, ammonium and N(C₁₋₄alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Combinations of substituents and variables envisioned by this disclosure are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

The term “biological sample”, as used herein, includes, without limitation, cell cultures or extracts thereof, biopsied material obtained from a mammal or extracts thereof; and blood, saliva, urine, feces, semen, tears, or other body fluids or extracts thereof. Examples of such purposes include, but are not limited to, blood transfusion, organ transplantation, biological specimen storage, and biological assays.

As used herein, a “therapeutically effective amount” means an amount of a substance (e.g., a therapeutic agent, composition, and/or formulation) that elicits a desired biological response. In some embodiments, a therapeutically effective amount of a substance is an amount that is sufficient, when administered as part of a dosing regimen to a subject suffering from or susceptible to a disease, disorder, and/or condition, to treat, diagnose, prevent, and/or delay the onset of the disease, disorder, and/or condition. As will be appreciated by those of ordinary skill in this art, the effective amount of a substance may vary depending on such factors as the desired biological endpoint, the substance to be delivered, the target cell or tissue, etc. For example, the effective amount of a provided compound in a formulation to treat a disease, disorder, and/or condition is the amount that alleviates, ameliorates, relieves, inhibits, prevents, delays onset of, reduces severity of and/or reduces incidence of one or more symptoms or features of the disease, disorder, and/or condition.

### I

As used herein, the terms “treatment,” “treat,” and “treating” refer to partially or completely alleviating, inhibiting, delaying onset of, preventing, ameliorating and/or relieving a disorder or condition, or one or more symptoms of the disorder or condition, as described herein. In some embodiments, treatment may be administered after one or more symptoms have developed. In some embodiments, the term “treating” includes preventing or halting the progression of a disease or disorder. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence. Thus, in some embodiments, the term “treating” includes preventing relapse or recurrence of a disease or disorder

The term “patient”, as used herein, means an animal, preferably a mammal, and most preferably a human.

The term “pharmaceutically acceptable carrier, adjuvant, or vehicle” refers to a non-toxic carrier, adjuvant, or vehicle that does not destroy the pharmacological activity of the compound(s) with which it is formulated. Pharmaceutically acceptable carriers, adjuvants or vehicles that may be used in the compositions of the compounds disclosed herein include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zine salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

A “pharmaceutically acceptable derivative” means any non-toxic salt, ester, salt of an ester or other derivative of a compound of this disclosure that, upon administration to a recipient, is capable of providing, either directly or indirectly, a compound of this disclosure or an inhibitorily active metabolite or residue thereof.

The expression “dosage unit form” as used herein refers to a physically discrete unit of agent appropriate for the patient to be treated. It will be understood, however, that total daily usage of compounds and compositions of the present disclosure will be decided by the attending physician within the scope of sound medical judgment. Specific effective dose level for any particular patient or organism will depend upon a variety of factors including disorder being treated and severity of the disorder; activity of specific compound employed; specific composition employed; age, body weight, general health, sex and diet of the patient; time of administration, route of administration, and rate of excretion of a specific compound employed; duration of treatment; drugs used in combination or coincidental with a specific compound employed, and like factors well known in the medical arts.

### Alternative Embodiments

In an alternative embodiment, compounds described herein may also comprise one or more isotopic substitutions. For example, hydrogen may be ²H (D or deuterium) or ³H (T or tritium); carbon may be, for example, ¹³C or ¹⁴C; oxygen may be, for example, ¹⁸O; nitrogen may be, for example, ¹⁵N, and the like. In other embodiments, a particular isotope (e.g., ³H, ¹³C, ¹⁴C, ¹⁸O), or ¹⁵N) can represent at least 1%, at least 5%, at least 10%, at least 15%, at least 20%, at least 25%, at least 30%, at least 35%, at least 40%, at least 45%, at least 50%, at least 60%, at least 65%, at least 70%, at least 75%, at least 80%, at least 85%, at least 90%, at least 95%, at least 99%, or at least 99.9% of the total isotopic abundance of an element that occupies a specific site of the compound.

### Pharmaceutical Compositions

In some embodiments, the present disclosure provides a composition comprising a compound of Formula (I) and a pharmaceutically acceptable carrier, adjuvant, or vehicle. In some embodiments, the amount of compound in compositions contemplated herein is such that is effective to measurably treat a disease or disorder in a biological sample or in a patient. In certain embodiments, the amount of compound in compositions of this disclosure is such that is effective to measurably treat a disease or disorder in a biological sample or in a patient. In certain embodiments, a composition contemplated by this disclosure is formulated for administration to a patient in need of such composition. In some embodiments, a composition contemplated by this disclosure is formulated for oral administration to a patient.

In some embodiments, compositions of the present disclosure may be administered orally, parenterally, by inhalation spray, topically, rectally, nasally, buccally, vaginally or via an implanted reservoir. In some preferred embodiments, compositions are administered orally, intraperitoneally or intravenously. In some embodiments, sterile injectable forms of the compositions comprising one or more compounds of Formula (I) may be aqueous or oleaginous suspension. In some embodiments, suspensions may be formulated according to techniques known in the art using suitable dispersing or wetting agents and suspending agents. In some embodiments, sterile injectable preparation may also be a sterile injectable solution or suspension in a non-toxic parenterally acceptable diluent or solvent, for example as a solution in 1,3-butanediol. In some embodiments, among the acceptable vehicles and solvents that may be employed are water, Ringer's solution and isotonic sodium chloride solution. In some embodiments, additional examples include, but are not limited to, sterile, fixed oils are conventionally employed as a solvent or suspending medium

The term “parenteral” as used herein includes subcutaneous, intravenous, intramuscular, intra-articular, intra-synovial, intrasternal, intrathecal, intrahepatic, intralesional and intracranial injection or infusion techniques.

Pharmaceutically acceptable compositions comprising one or more compounds of Formula (I) may be orally administered in any orally acceptable dosage form including, but not limited to, capsules, tablets, aqueous suspensions or solutions. In some embodiments, carriers used include lactose and corn starch. Lubricating agents, such as magnesium stearate, are also typically added. In some embodiments, useful diluents include lactose and dried cornstarch. In some embodiments, when aqueous suspensions are required for oral use, an active ingredient is combined with emulsifying and suspending agents. In some embodiments, certain sweetening, flavoring or coloring agents may also be added.

Alternatively, pharmaceutically acceptable compositions comprising a compound of Formula (I) may be administered in the form of suppositories for rectal administration. These can be prepared by mixing the agent with a suitable non-irritating excipient that is solid at room temperature but liquid at rectal temperature and therefore will melt in the rectum to release the drug. Such materials include cocoa butter, beeswax and polyethylene glycols.

Pharmaceutically acceptable compositions comprising a compound of Formula (I) may also be administered topically, especially when the target of treatment includes areas or organs readily accessible by topical application, including diseases of the eye, the skin, or the lower intestinal tract. Suitable topical formulations are readily prepared for each of these areas or organs. In some embodiments, pharmaceutically acceptable compositions may be formulated in a suitable ointment containing the active component suspended or dissolved in one or more carriers. Carriers for topical administration of compounds of this disclosure include, but are not limited to, mineral oil, liquid petrolatum, white petrolatum, propylene glycol, polyoxyethylene, polyoxypropylene compound, emulsifying wax and water. Alternatively, provided pharmaceutically acceptable compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

Pharmaceutically acceptable compositions comprising a compound of Formula (I) may also be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

In some embodiments, an amount of a compound of the present disclosure that may be combined with the carrier materials to produce a composition in a single dosage form will vary depending upon the host treated, the particular mode of administration. Preferably, provided compositions should be formulated so that a dosage of between 0.01-100 mg/kg body weight/day of the inhibitor can be administered to a patient receiving these compositions.

### Methods of Using Compounds of the Present Disclosure

In some embodiments, the present disclosure provides a method for treating or lessening the severity of a disease or condition associated with cell proliferation in a patient comprising the step of administering to said patient a composition according to the present disclosure.

The term “disease or condition associated with cell proliferation”, as used herein means any disease or other deleterious condition in which cell proliferation is known to play a role. Accordingly, another embodiment of the present disclosure relates to treating or lessening the severity of one or more diseases in which cell proliferation is known to play a role. In some embodiments, a disease or condition associated with cell proliferation is cancer.

In some embodiments, administration of a compound of the present disclosure results in arrest of mitosis or change in DNA content.

In some embodiments, administration of a compound of the present disclosure results in arrest of mitosis. In some embodiments, mitotic arrest is defined as a 10-100% reduction in mitosis. In some embodiments, mitotic arrest is defined as a 20-100% reduction in mitosis. In some embodiments, mitotic arrest is defined as a 30-100% reduction in mitosis. In some embodiments, mitotic arrest is defined as a 40-100% reduction in mitosis. In some embodiments, mitotic arrest is defined as a 50-100% reduction in mitosis. In some embodiments, mitotic arrest is defined as a 60-100% reduction in mitosis. In some embodiments, mitotic arrest is defined as a 70-100% reduction in mitosis. In some embodiments, mitotic arrest is defined as a 80-100% reduction in mitosis. In some embodiments, mitotic arrest is defined as a 90-100% reduction in mitosis. In some embodiments, mitotic arrest is defined as a 100% reduction in mitosis.

In some embodiments, administration of a compound of the present disclosure results in change in DNA content. In some embodiments, change of DNA content is induction of polyploidy.

In some embodiments, compounds and compositions, according to a method of the present disclosure, may be administered using any amount and any route of administration effective for treating or lessening the severity of cancer. The exact amount required will vary from subject to subject, depending on the species, age, and general condition of the subject, severity of the infection, particular agent, its mode of administration, and the like. Compounds of the present disclosure are preferably formulated in dosage unit form for ease of administration and uniformity of dosage.

In some embodiments, cancer is selected from the group consisting of lung cancer and breast cancer. In some embodiments, cancer is lung cancer. In some embodiments, lung cancer is non-small cell lung cancer. In some embodiments, non-small cell lung cancer is lung adenocarcinoma. In some embodiments, cancer is breast cancer. In some embodiments, breast cancer is mammary cancer. In some embodiments, breast cancer is breast adenocarcinoma.

In some embodiments, pharmaceutically acceptable compositions of comprising compounds of the present disclosure can be administered to humans and other animals orally, rectally, parenterally, intracisternally, intravaginally, intraperitoneally, topically (as by powders, ointments, or drops), buccally, as an oral or nasal spray, or the like, depending on the severity of infection being treated. In certain embodiments, compounds of the present disclose may be administered orally or parenterally at dosage levels of about 0.01 mg/kg to about 50 mg/kg and preferably from about 1 mg/kg to about 25 mg/kg, of subject body weight per day, one or more times a day, to obtain desired therapeutic effect.

In some embodiments, one or more additional therapeutic agents, may also be administered in combination with compounds of the present disclosure. In some embodiments, a compound of the present disclosure and one or more additional therapeutic agents may be administered as part of a multiple dosage regime. In some embodiments, a compound of the present disclosure and one or more additional therapeutic agents may be administered may be administered simultaneously, sequentially or within a period of time. In some embodiments, a compound of the present disclosure and one or more additional therapeutic agents may be administered within five hours of one another. In some embodiments, a compound of the present disclosure and one or more additional therapeutic agents may be administered within 24 hours of one another. In some embodiments, a compound of the present disclosure and one or more additional therapeutic agents may be administered within one week of one another.

In some embodiments, a compound of the present disclosure and one or more additional therapeutic agents may be formulated into a single dosage form.

### Enumerated Embodiments

The present disclosure includes enumerated embodiments 1-20:
1. A compound of formula (I):

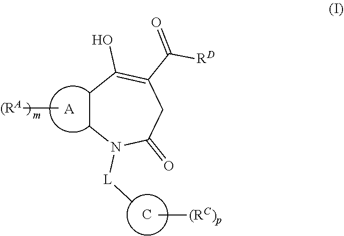

or a pharmaceutically acceptable salt thereof
wherein
Ring A is selected from the group consisting of optionally substituted phenyl and optionally substituted 6-membered heteroaryl;
L is selected from the group consisting of a bond, optionally substituted C₁-C₃ alkylene, —C(O)—, —C(O)O—, —C(O)NH—, and —S(O)₂—;
Ring C is selected from the group consisting of optionally substituted phenyl, optionally substituted 6-membered heteroaryl, optionally substituted 6-membered carbocyclyl, and optionally substituted 6-membered heterocyclyl;
each R^{A} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂; or
each R^{B} is independently selected from the group consisting of hydrogen and optionally substituted C₁-C₆ aliphatic,
each R^{C} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR³, —SR³, —N(R³)₂, —C(O)OR³, C(O)N(R³)₂, —N(H)C(O)R³, and —N(H)C(O)N(R³)₂;
   two instances of R^{C} are taken together with the atoms on which they are attached to form an optionally substituted 6-membered aryl or optionally substituted 6-membered heteroaryl;
R^{D} is selected from the group consisting of optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR², —SR², and —N(R²)₂;
each R¹ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl;
m is 0, 1, 2, 3, or 4;
p is 0, 1, 2, 3, 4, or 5.
2. The compound of embodiment 1, wherein the compound is of Formula (I-a), (I-b), (I-c), (I-d):

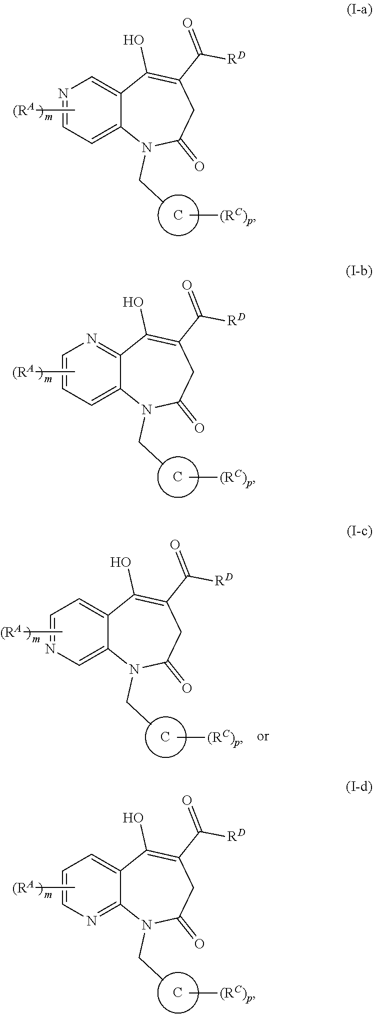

or a pharmaceutically acceptable salt thereof.
3. The compound of embodiment 1, wherein the compound is of Formula (I-e):

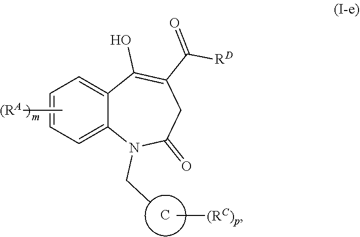

or a pharmaceutically acceptable salt thereof.
4. The compound of any of the previous embodiments, wherein Ring C is selected from optionally substituted phenyl and optionally substituted pyridinyl.
5. The compound of any of the previous embodiments, wherein Ring C is optionally substituted phenyl.
6. The compound of any of the previous embodiments, wherein Ring C is an optionally substituted pyridinyl.
7. The compound of any of the previous embodiments, wherein R^{C} is selected from halogen, optionally substituted C₁-C₆ aliphatic, —COOR³, —CN, and —OR³.
8. The compound of any of the previous embodiments, wherein p is 2.
9. The compound of embodiment 8, wherein one instance of R^{C} is halogen and the other is optionally substituted C₁-C₆ aliphatic or —OR³.
10. The compound of any of the previous embodiments, wherein R^{C} is selected from —F, -Me, and —OMe.
11. The compound of any of the previous embodiments, wherein p is 2 and one instance of R^{C} is fluoro and the other is -Me or —OMe.
12. The compound of any of the previous embodiment, wherein L and Ring C are taken together to form a group selected from

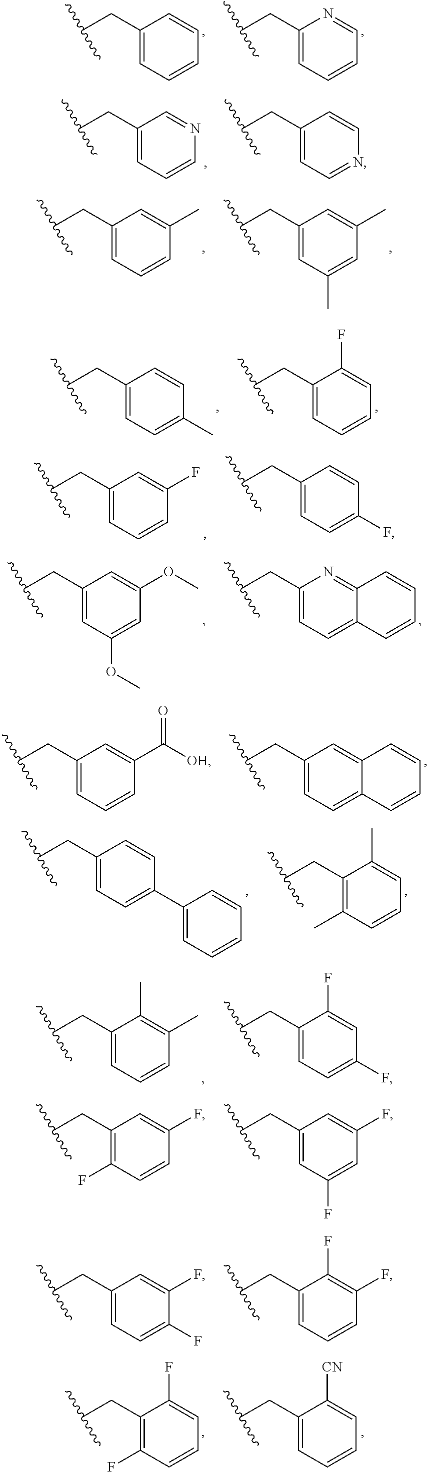

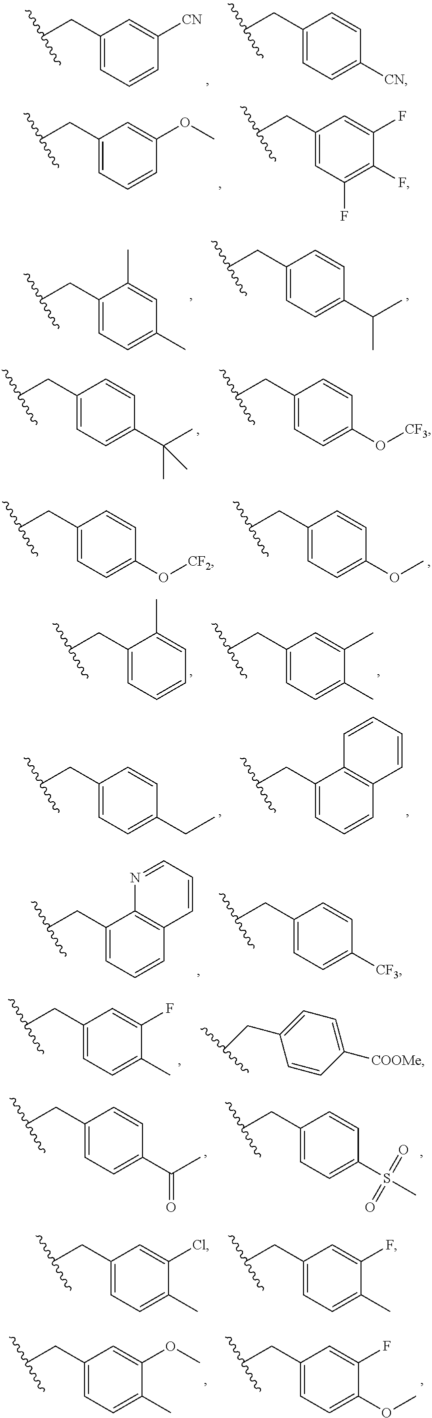

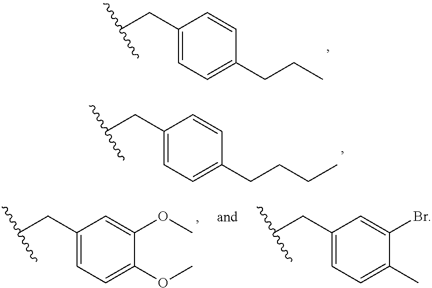

13. The compound of any of the previous embodiments, wherein R^{D} is selected from optionally substituted C₁-C₃ alphatic and OR²
14. The compound of any of the previous embodiments, wherein R² is optionally substituted C₁-C₃ aliphatic.
15. The compound of any of the previous embodiments, wherein R² is optionally substituted C₁-C₃ alkyl.
16. The compound of any of the previous embodiments, wherein R^{D} is selected from optionally substituted methyl, optionally substituted ethyl, and —OMe.
17. The compound of any of the previous embodiments, wherein m is 0.
18. The compound of any of the previous embodiments, wherein the compound is described in Tables 1-4.
19. A pharmaceutical composition comprising a compound of any of the previous embodiments and a pharmaceutically acceptable excipient.
20. A method of treat cancer comprising administering to a patient in need thereof the compound of any of embodiments 1-18 or the pharmaceutical composition of embodiment 19.

### Exemplification

### 1.1 General Methods

Unless stated otherwise, all the chemicals required for synthesis were purchased from commercially available suppliers and used without further purification. ¹H NMR spectra was determined with a Bruker Avance III-400 at 400 MHz. LC-MS analysis was performed on a platform equipped with Agilent LC-MS 1260-6110 or Agilent LC-MS 1260-6120, using a Waters X Bridge C18: 50 mm×4.6 mm×3.5 um column. Flash column chromatography was conducted with silica gel (200-300 mesh, Qingdao Haiyang Chemical Co. Ltd., China). Analytical and preparative TLC analysis were performed on GF254 silica gel plates (Yantai Jiangyou Inc., China). Unless otherwise noted, reagents and all solvents are analytically pure grade and were obtained commercially from vendors such as Chron Chemical or Energy-Chemical.

Abbreviations: TLC: Thin Layer Chromatography, EA: Ethyl Acetate, PE: Petroleum
Ether, DMF: N,N-dimethylformamide, THF: Eetrahydrofuran,
DCM: Dichloromethane, DIPEA: N,N-diisopropylethylamine,
DMAP: 4-dimethylaminopyridine, NaH: Sodium hydride

### 2. Synthesis and Analytical Data of Compounds

### 2.1 General Scheme for the Synthesis of Library Compounds

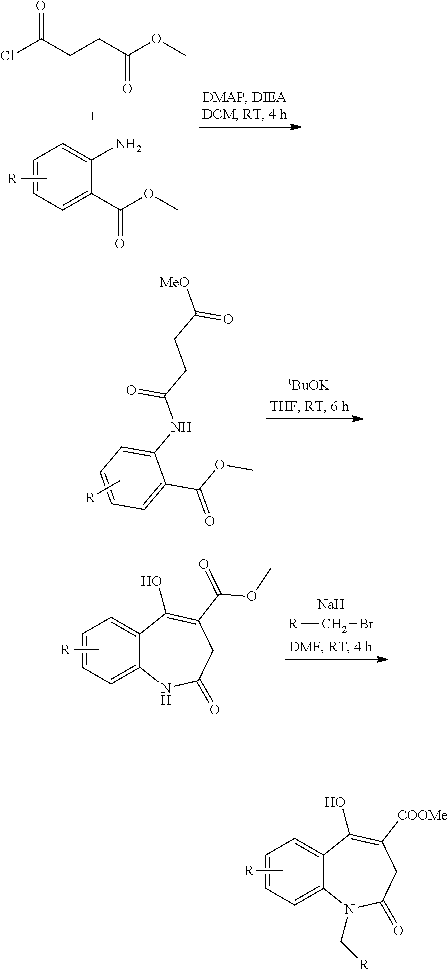

### 2.2 General Scheme for the Synthesis of Library Compounds

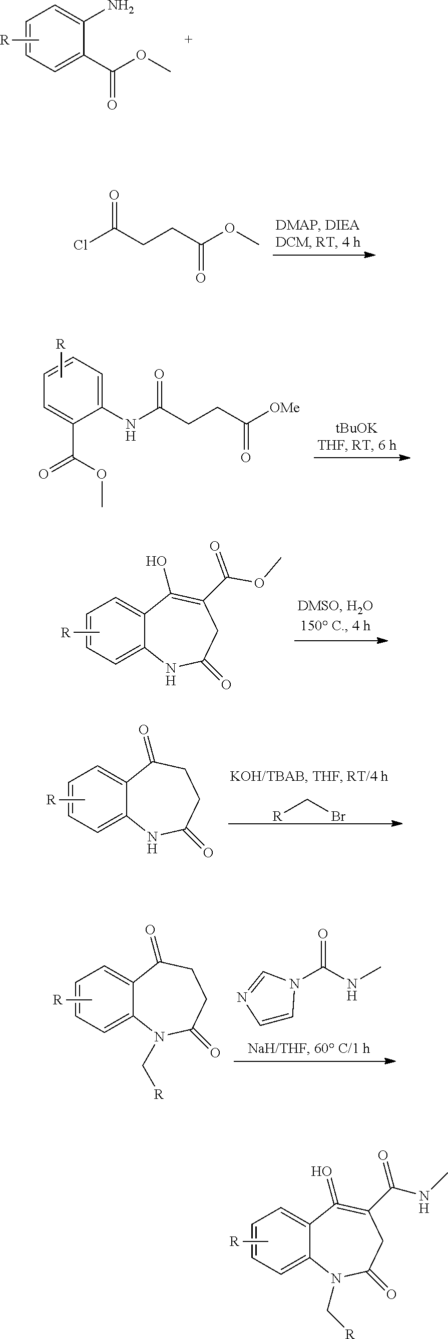

### Intermediate 3-1. Methyl 2-(3-methoxy-3-oxopropanamido) benzoate

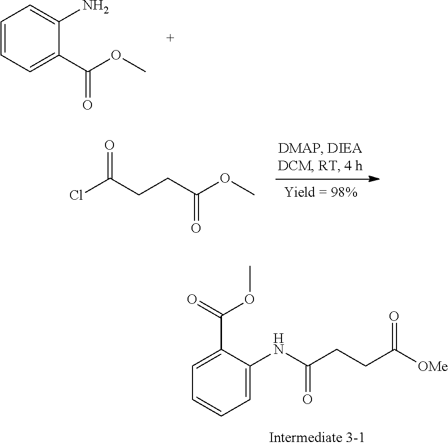

To a solution of methyl 2-aminobenzoate (1.95 mL, 15 mmol, 1 equiv) in DCM (45 mL), was added DIEA (2.73 mL, 16.5 mmol, 1.3 equiv) and DMAP (30 mg, 0.075 mmol, 0.5% equiv) under Nitrogen. To this solution was added 3-(carbomethoxy) propionyl chloride (2.5 mL, 19.5 mmol, 1.1 equiv) dropwise over a period of 10 min, and the reaction mixture was allowed to stir at rt for 4 h. The reaction progress was monitored by TLC until the complete disappearance of methyl 2-aminobenzoate. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 ml), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/8, flow rate: 20 mL/min, collections at 11-14 min elution time contained the target compound) to get 4.8 g of the light yellow oil Intermediate 1 with a yield of 98%.

¹H NMR (400 MHZ, DMSO) δ 10.65 (s, 1H), 8.30-8.18 (m, 1H), 7.89 (dd, J=7.9, 1.5 Hz, 1H), 7.65-7.51 (m, 1H), 7.25-7.08 (m, 1H), 3.85 (s, 3H), 3.60 (s, 3H), 2.69 (dd, J=9.8, 4.1 Hz, 1H), 2.62 (dd, J=9.8, 4.1 Hz, 1H). ¹³C NMR (100 MHz, DMSO) δ 172.59, 169.98, 167.56, 139.59, 133.87, 130.42, 122.99, 120.90, 117.41, 52.31, 51.35, 31.56, 28.38.

### Intermediate 3-2. Methyl 5-hydroxy-2-oxo-2,3-dihydro-1H-benzo[b]azepine-4-carboxylate

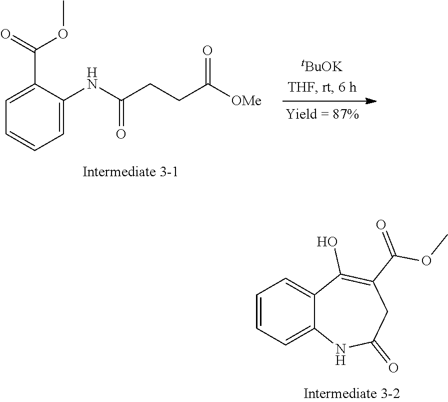 [SMILES: COC(=O)CCC(=O)Nc1ccccc1C(=O)OC ; COC(=O)C1=C(O)c2ccccc2NC(=O)C1]

To a solution of methyl 2-(3-methoxy-3-oxopropanamido)benzoate (Intermediate 1) (4.8 g, 18.7 mmol, 1 equiv) in THE (74 mL) at 5° C. was added a 1 M solution of t-BuOK in THE (55 mL, 56 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 2.8 g of the white solid Intermediate 2 with a yield of 87%. Purity=95% LCMS (m/z)=234.3 (M+H).

¹H NMR (400 MHZ, DMSO) δ 12.46 (s, 1H), 10.35 (s, 1H), 7.79 (dd, J=8.0, 1.5 Hz, 1H), 7.53 (ddd, J=8.3, 7.4, 1.6 Hz, 1H), 7.32-7.21 (m, 1H), 7.18 (dd, J=8.2, 0.8 Hz, 1H), 3.83 (s, 3H), 2.92 (s, 2H). ¹³C NMR (100 MHz, DMSO) δ 171.99, 170.80, 166.22, 137.92, 131.91, 128.15, 124.75, 123.47, 121.61, 95.82, 52.49, 30.70.

### Intermediate (3-2): Methyl 5-methoxy-2-(4-methoxy-4-oxobutanamido) benzoate

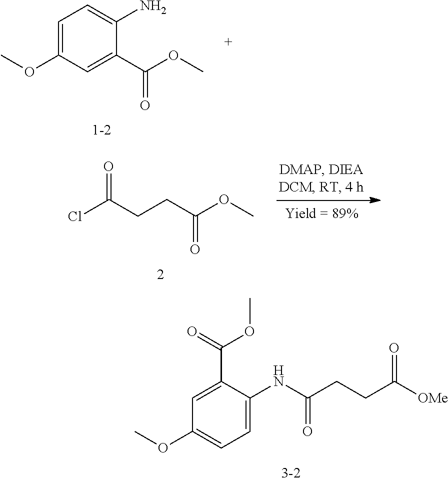

To a solution of methyl 2-amino-5-methoxybenzoate 1-2 (5430 mg, 30 mmol, 1 equiv) in DCM (90 mL), was added DIEA (5460 mg, 33 mmol, 1.3 equiv) and DMAP (60 mg, 0.15 mmol, 0.05 equiv) under nitrogen, to this solution was added 3-(carbomethoxy) propionyl chloride 2 (5000 μL, 39 mmol, 1.3 equiv) dropwise over a period of 10 min, and the reaction mixture was allowed to stir at rt for 4 h. The reaction progress was monitored by TLC until the complete disappearance of compound 1-2. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 7.9 g (purity=99%) of the light-yellow oil compound with a yield of 89%.

TLC R_{f}=0.6 (PE:EA=2:1);

LCMS (m/z)=296.40 (M+H)

### Intermediate (3-3): Methyl 4-bromo-2-(4-methoxy-4-oxobutanamido) benzoate

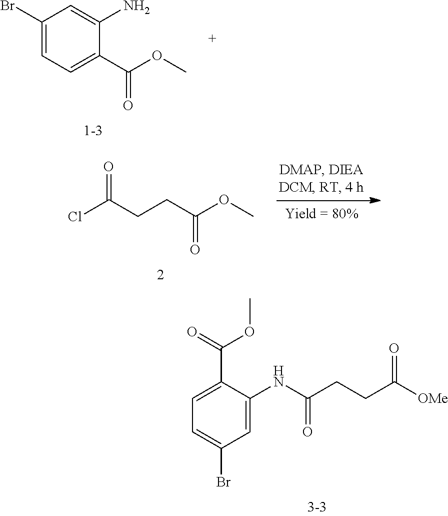

To a solution of methyl 2-amino-4-bromobenzoate 1-3 (10 g, 43.5 mmol, 1 equiv) in DCM (130 mL), was added DIEA (11.34 mL, 65.2 mmol, 1.5 equiv) and DMAP (265 mg, 2.17 mmol, 0.05 equiv) under nitrogen, to this solution was added 3-(carbomethoxy) propionyl chloride 2 (6.89 mL, 56.52 mmol, 1.3 equiv) dropwise over a period of 10 min, and the reaction mixture was allowed to stir at rt for 4 h. The reaction progress was monitored by TLC until the complete disappearance of compound 1-3. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 11-14 min elution time contained the target compound) to get 12 g (purity=95%) of the light yellow oil compound with a yield of 80%.

TLC R_{f}=0.3 (PE:EA=2:1);

LCMS (m/z)=346.20 (M+H)

### Intermediate (3-4): Methyl 4-methoxy-2-(4-methoxy-4-oxobutanamido) benzoate

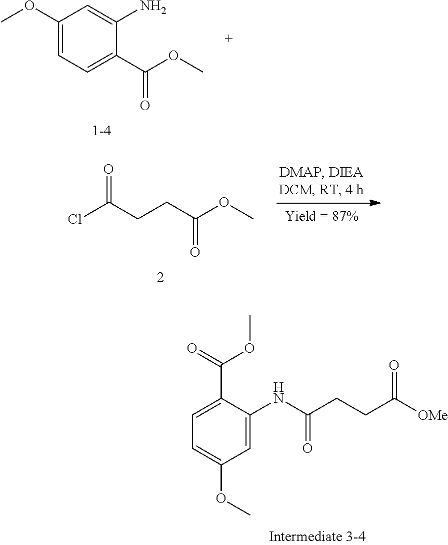

To a solution of methyl 2-amino-4-methoxybenzoate 1-4 (5000 mg, 27 mmol, 1 equiv) in DCM (80 mL), was added DIEA (5100 mg, 30 mmol, 1.3 equiv) and DMAP (50 mg, 0.135 mmol, 0.05 equiv) under nitrogen, to this solution was added 3-(carbomethoxy) propionyl chloride 2 (4280 μL, 39 mmol, 1.3 equiv) dropwise over a period of 10 min, and the reaction mixture was allowed to stir at rt for 4 h. The reaction progress was monitored by TLC until the complete disappearance of compound 1-4. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 7 g (purity=98%) of the light-yellow oil compound with a yield of 87%.

TLC R_{f}=0.6 (PE:EA=2:1);

LCMS (m/z)=296.50 (M+H)

### Intermediate (3-5): Methyl 2-(4-methoxy-4-oxobutanamido)-4 methylbenzoate

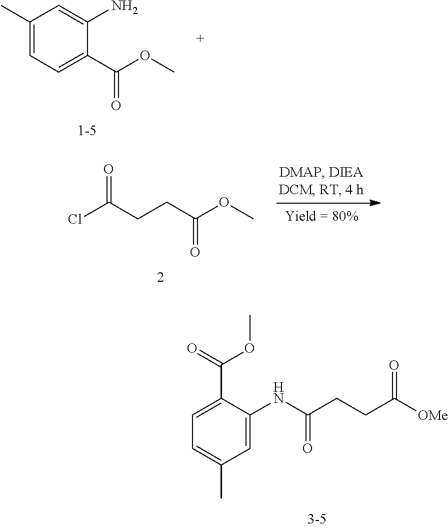

To a solution of methyl 2-amino-4-methylbenzoate 1-5 (5000 mg, 30 mmol, 1 equiv) in DCM (90 mL), was added DIEA (5460 mg, 39 mmol, 1.3 equiv) and DMAP (60 mg, 0.15 mmol, 0.05 equiv) under nitrogen, to this solution was added 3-(carbomethoxy) propionyl chloride 2 (5000 μL, 39 mmol, 1.3 equiv) dropwise over a period of 10 min, and the reaction mixture was allowed to stir at rt for 4 h. The reaction progress was monitored by TLC until the complete disappearance of compound 1-5. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 6.7 g (purity=97%) of the light-yellow oil compound with a yield of 80%.

TLC R_{f}=0.6 (PE:EA=2:1);

LCMS (m/z)=280.0 (M+H)

### Intermediate (3-6): Methyl methyl 2-(4-methoxy-4-oxobutanamido)-4-(trifluoromethyl) benzoate

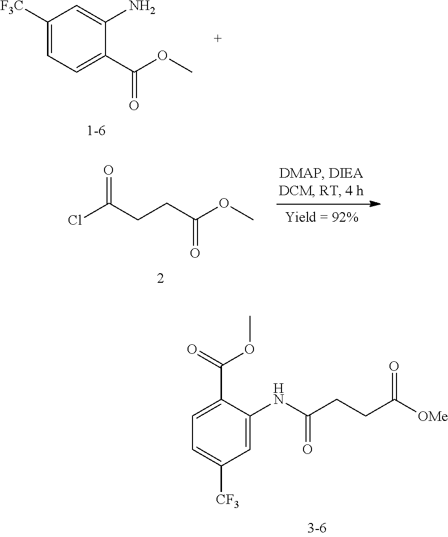

To a solution of methyl 2-amino-4-(trifluoromethyl) benzoate 1-6 (5 g, 22.8 mmol, 1 equiv) in DCM (69 mL), was added DIEA (6 mL, 34.3 mmol, 1.5 equiv) and DMAP (140 mg, 1.15 mmol, 0.05 equiv) under nitrogen, to this solution was added 3-(carbomethoxy) propionyl chloride 2 (3.6 mL, 29.8 mmol, 1.3 equiv) dropwise over a period of 10 min, and the reaction mixture was allowed to stir at rt for 4 h. The reaction progress was monitored by TLC until the complete disappearance of compound 1-6. To the reaction mixture was then added H₂O (40 mL) and the mixture was allowed to stir for 30 min. The organic solution was separated and the aqueous solution was extracted with DCM (3×50 mL). The organic solutions were combined, washed with H₂O (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 11-14 min elution time contained the target compound) to get 7 g (purity=99%) of the light yellow oil compound with a yield of 92%.

TLC R_{f}=0.5 (PE:EA=2:1);

LCMS (m/z)=334.30 (M+H)

### Intermediate (3-7): Methyl 5-fluoro-2-(4-methoxy-4-oxobutanamido)benzoate

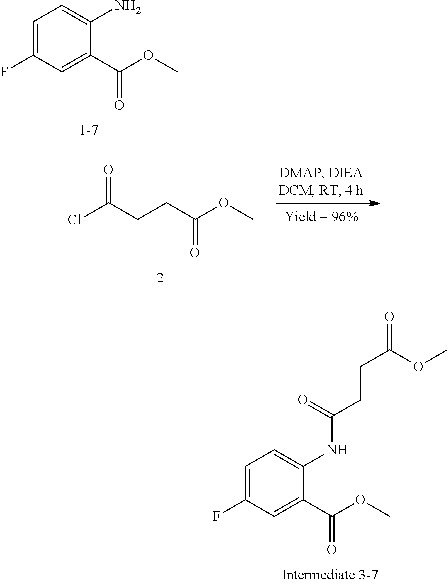

To a solution of methyl 2-amino-5-fluorobenzoate 1-7 (3 g, 17.7 mmol, 1 equiv) in DCM (53 mL), was added DIEA (4.6 mL, 26.60 mmol, 1.5 equiv) and DMAP (110 mg, 0.90 mmol, 0.05 equiv) under nitrogen, to this solution was added 3-(carbomethoxy) propionyl chloride 2 (2.82 mL, 23.06 mmol, 1.3 equiv) dropwise over a period of 10 min, and the reaction mixture was allowed to stir at rt for 4 h. The reaction progress was monitored by TLC until the complete disappearance of compound 1-7. To the reaction mixture was then added H₂O (40 mL) and the mixture was allowed to stir for 30 min. The organic solution was separated and the aqueous solution was extracted with DCM (3×50 mL). The organic solutions were combined, washed with H₂O) (2×50 mL) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively, silica gel: 200-300 mesh, eluent: EA/PE=1/2, flow rate: 40 mL/min, collections at 11-14 min elution time contained the target compound) to get 4.8 g of the light yellow oil compound with a yield of 96%. Purity=99%

TLC R_{f}=0.6 (PE:EA=2:1);

LCMS (m/z)=283.90 (M+H)

### Intermediate (3-8): methyl 2-(4-methoxy-4-oxobutanamido)-5-methylbenzoate

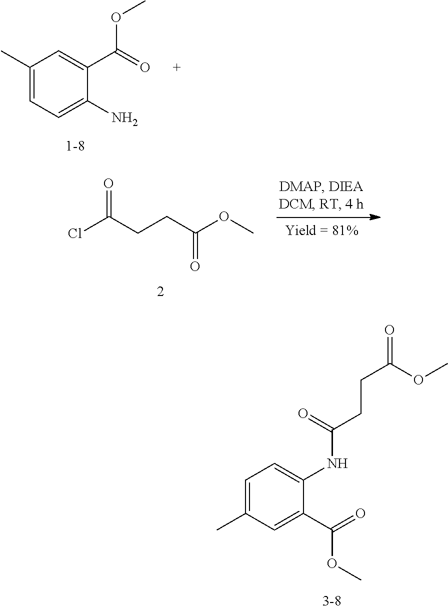

To a stirred solution of methyl 2-amino-5-methylbenzoate 1-8 (3 g, 18.16 mmol, 1 equiv) in DCM (54 mL), was added DIEA (4.74 mL, 27.24 mmol, 1.5 equiv) and DMAP (0.11 g, 0.91 mmol, 0.05 equiv) under nitrogen, to this solution was added 3-(carbomethoxy) propionyl chloride 2 (2.89 mL, 23.61 mmol, 1.3 equiv) dropwise over a period of 10 min, and the reaction mixture was allowed to stir at rt for 4 h. The reaction progress was monitored by TLC until the complete disappearance of compound 1-8. To the reaction mixture was then added H₂O (40 mL) and the mixture was allowed to stir for 30 min. The organic solution was separated, and the aqueous solution was extracted with DCM (3×50 mL). The organic solutions were combined, washed with H₂O (2×50 ml) and brine (1×100 mL), dried over anhydrous Na₂SO₄, filtered and concentrated by rotary evaporation to get the crude compound. Then, the resulting concentrate was purified on a silica column (The height and diameter of the silica gel column is 10 and 2 cm, respectively; silica gel: 200-300 mesh, eluent: EA/PE=1/2, flow rate: 40 mL/min, collections at 11-14 min elution time contained the target compound) to get 189.7 mg of the light-yellow oil compound with a yield of 81%. Purity=96%

TLC R_{f}=0.55 (PE:EA=2:1);

LCMS (m/z)=280.40 (M+H)

### Intermediate (4-1): Methyl 5-hydroxy-2-oxo-2,3-dihydro-1H-benzo[b]azepine-4-carboxylate

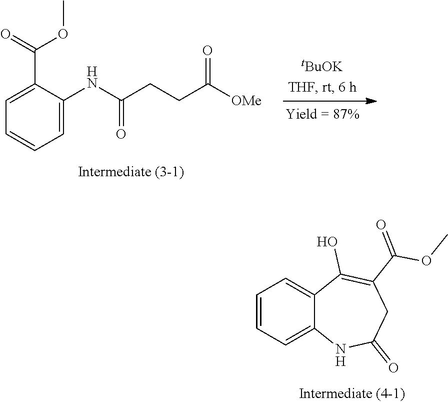 [SMILES: COC(=O)CCC(=O)Nc1ccccc1C(=O)OC ; COC(=O)C1=C(O)c2ccccc2NC(=O)C1]

To a solution of methyl 2-(3-methoxy-3-oxopropanamido)benzoate 3-1 (4.8 g, 18.7 mmol, 1 equiv) in THF (74 mL) at 5° C. was added a 1 M solution of t-BuOK in THF (SS mL, 56 mmol, 3 equiv) dropwise over 10 min while maintaining the temperature at 5° C. After 6 h, 20 mL of H₂O followed by 80 mL of 1N HCl were added to bring the solution to pH=4. The resulting mixture was allowed to stir at rt for 40 min. The organic solution was separated, and the aqueous solution was extracted with EtOAc (3×100 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 2.8 g of the white solid compound with a yield of 87%. Purity=95%

TLC R_{f}=0.3 (PE:EA=2:1);

LCMS (m/z)=234.3 (M+H)

¹H NMR (400 MHZ, DMSO) δ 12.46 (s, 1H), 10.35 (s, 1H), 7.79 (dd, J=8.0, 1.5 Hz, 1H), 7.53 (ddd, J=8.3, 7.4, 1.6 Hz, 1H), 7.32-7.21 (m, 1H), 7.18 (dd, J=8.2, 0.8 Hz, 1H), 3.83 (s, 3H), 2.92 (s, 2H). ¹³C NMR (100 MHz, DMSO) δ 171.99, 170.80, 166.22, 137.92, 131.91, 128.15, 124.75, 123.47, 121.61, 95.82, 52.49, 30.70.

### Intermediate (4-2): Methyl 5-hydroxy-7-methoxy-2-oxo-2,3-dihydro-1H-benzo[b]azepine-4-carboxylate

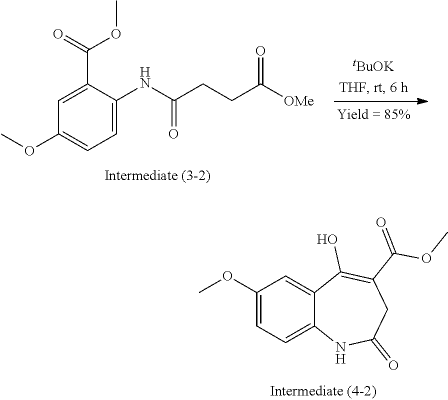

To a solution of methyl 5-methoxy-2-(4-methoxy-4-oxobutanamido) benzoate 3-2 (7900 mg, 26.77 mmol, 1 equiv) in THF (100 mL) at 5° C. was added a 1 M solution of t-BuOK in THF (80 mL, 80.33 mmol, 3 equiv) dropwise over 10 min while maintaining the temperature at 5° C. After 6 h, 114 mL of H₂O followed by 114 mL of 1N HCl were added to bring the solution to pH=4. The resulting mixture was allowed to stir at rt for 40 min. The organic solution was separated, and the aqueous solution was extracted with EtOAc (3×50 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 1 h elution time contained the target compound) to get 6000 mg of the white solid with a yield of 85%. Purity=96%

TLC R_{f}=0.3 (PE:EA=2:1);

LCMS (m/z)=264.1 (M+H)

### Intermediate (4-3): Methyl 9-bromo-5-hydroxy-2-oxo-2,3-dihydro-1H-benzo[b]azepine-4-carboxylate

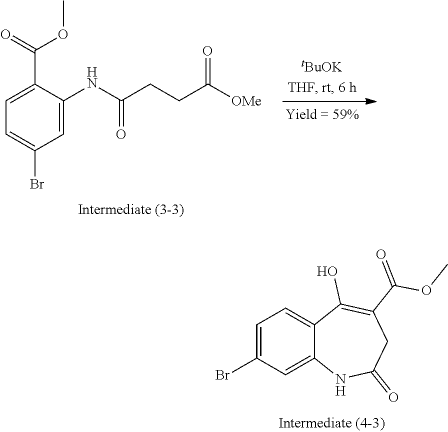 [SMILES: COC(=O)CCC(=O)Nc1cc(Br)ccc1C(=O)OC ; COC(=O)C1=C(O)c2ccc(Br)cc2NC(=O)C1]

To a solution of methyl methyl 4-bromo-2-(4-methoxy-4-oxobutanamido) benzoate 3-3 (12 g, 34.88 mmol, 1 equiv) in THF (138 mL) at 5° C. was added a 1 M solution of t-BuOK in THE (103 mL, 105 mmol, 3 equiv) dropwise over 10 min while maintaining the temperature at 5° C. After 6 h, 20 ml of H₂O followed by 120 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 6.4 g of the white solid compound with a yield of 59%. Purity=99%

TLC R_{f}=0.3 (PE:EA=2:1);
LCMS (m/z)=313.83 (M+H)

### Intermediate (4-4): Methyl 5-hydroxy-8-methoxy-2-oxo-2,3-dihydro-1H-benzo[b]azepine-4-carboxylate

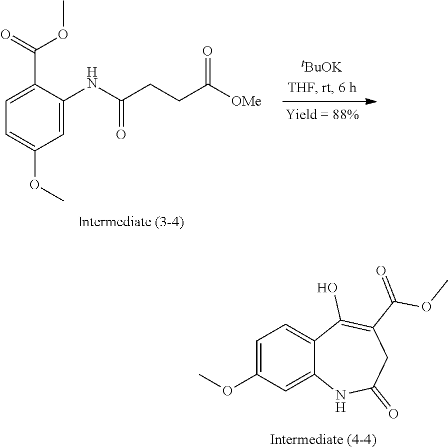

To a solution of 3-4 (7000 mg, 23.77 mmol, 1 equiv) in THF (90 mL) at 5° C. was added a 1 M solution of t-BuOK in THF (71 mL, 71 mmol, 3 equiv) dropwise over 10 min while maintaining the temperature at 5° C. After 6 b, 114 mL of H₂O followed by 114 mL of 1N HCl were added to bring the solution to pH=4. The resulting mixture was allowed to stir at rt for 40 min. The organic solution was separated and the aqueous solution was extracted with EtOAc (3×50 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 1 h elution time contained the target compound) to get 5500 mg of the white solid with a yield of 88%. Purity=91%

TLC R_{f}=0.3 (PE:EA=2:1);

LCMS (m/z)=296.30 (M+H)

### Intermediate (4-5): Methyl 5-hydroxy-8-methyl-2-oxo-2,3-dihydro-1H-benzo[b]azepine-4-carboxylate

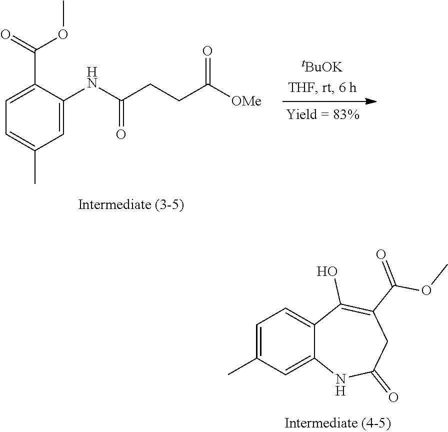

To a solution of 3-5 (6700 mg, 24 mmol, 1 equiv) in THF (96 mL) at 5° C. was added a 1 M solution of t-BuOK in THF (72 mL, 72 mmol, 3 equiv) dropwise over 10 min while maintaining the temperature at 5° C. After 6 h, 114 mL of H₂O followed by 114 ml, of 1N HCl were added to bring the solution to pH=4. The resulting mixture was allowed to stir at rt for 40 min. The organic solution was separated and the aqueous solution was extracted with EtOAc (3×50 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 50 mL/min, collections at 1b elution time contained the target compound) to get 4900 mg of the white solid with a yield of 83%. Purity=93%

TLC R_{f}=0.3 (PE:EA=2:1);

LCMS (m/z)=248.1 (M+H)

### Intermediate (4-6): Methyl-5-hydroxy-2-oxo-8-(trifluoromethyl)-2,3-dihydro-1H-benzo[b]azepine-4-carboxylate

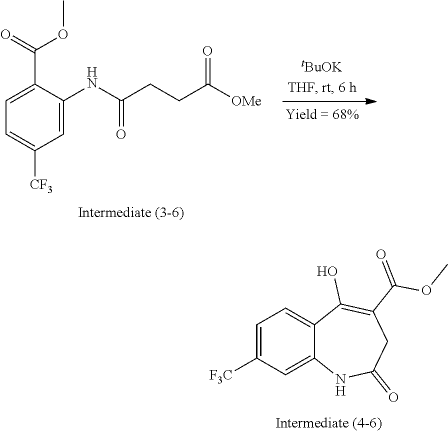 [SMILES: COC(=O)CCC(=O)Nc1cc(C(F)(F)F)ccc1C(=O)OC ; COC(=O)C1=C(O)c2ccc(C(F)(F)F)cc2NC(=O)C1]

To a solution of methyl methyl 4-bromo-2-(4-methoxy-4-oxobutanamido)benzoate 3-6 (7 g, 21.0 mmol, 1 equiv) in THF (83 mL) at 5° C. was added a 1 M solution of t-BuOK in THF (63 mL, 63.0 mmol, 3 equiv) dropwise over 10 min while maintaining the temperature at 5° C. After 6 h, 20 mL of H₂O) followed by 70 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/4, flow rate: 20 mL/min, collections at 8-9 min elution time contained the target compound) to get 4.3 g of the white solid compound with a yield of 68%. Purity=99%

TLC R_{f}=0.3 (PE:EA=2:1);

LCMS (m/z)=302.40 (M+H)

### Intermediate (4-7): Methyl 7-fluoro-3,5-dioxo-2,3,4,5-tetrahydro-1H-benzo[b]azepine-4-carboxylate

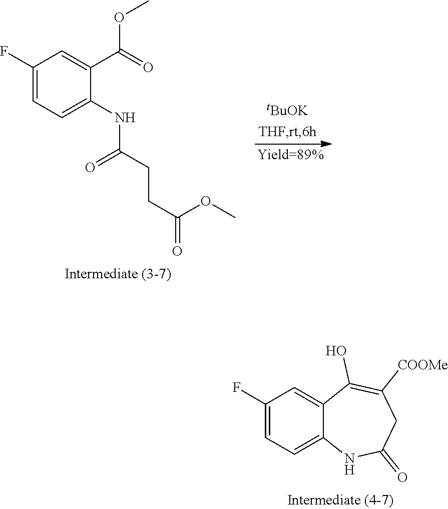 [SMILES: COC(=O)CCC(=O)Nc1ccc(F)cc1C(=O)OC ; COC(=O)C1=C(O)c2cc(F)ccc2NC(=O)C1]

To a solution of methyl 5-fluoro-2-(4-methoxy-4-oxobutanamido) benzoate 3-7 (4.8 g, 17.08 mmol, 1 equiv) in THF (72 mL) at 5° C. was added a 1 M solution of t-BuOK in THF (50 mL, 51 mmol, 3 equiv) dropwise over 10 min while maintaining the temperature at 5° C. After 6 h, 20 mL of H₂O followed by 120 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 40 mL/min, collections at 8-9 min elution time contained the target compound) to get 3.81 g of the white solid compound with a yield of 89%. Purity=99%

TLC R_{f}=0.4 (PE:EA=2:1);

LCMS (m/z)=220.40 (M+H)

### Intermediate (4-8): Methyl 5-hydroxy-7-methyl-2-oxo-2,3-dihydro-1H-benzo[b]azepine-4-carboxylate

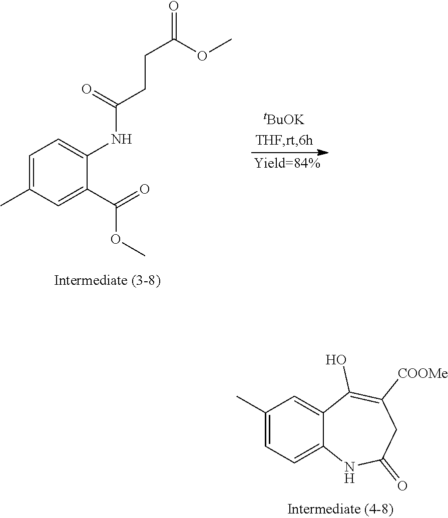

To a solution of methyl 2-(4-methoxy-4-oxobutanamido)-5-methylbenzoate 3-8 (10 g, 43.5 mmol, 1 equiv) in THE (62 mL) at 5° C. was added a 1 M solution of t-BuOK (48 mL, 50 mmol, 3 equiv) in THE, dropwise over 10 min while maintaining the temperature at 5° C. After 6 h, 20 mL of H₂O followed by 120 mL of 1N HCl were added to bring the solution to pH=4. The resulting mixture was allowed to stir at rt for 40 min. The organic solution was separated and the aqueous solution was extracted with EtOAc (3×120 mL). The organic solutions were combined, dried over anhydrous Na₂SO₄, filtered and concentrated by rotary evaporation to afford crude compound. Then, the resulting concentrate was purified on a silica column (The height and diameter of the silica gel column is 10 and 2 cm, respectively; silica gel: 200-300 mesh, eluent: EA/PE=1/2, flow rate: 40 mL/min, collections at 8-9 min elution time contained the target compound) to get 3.47 g (purity=99%) of the light yellow oil compound with a yield of 84%.

TLC R_{f}=0.3 (PE:EA=2:1);

LCMS (m/z)=247.08 (M+H)

### Intermediate (5-1): 3,4-dihydro-1H-benzo[b]azepine-2,5-dione

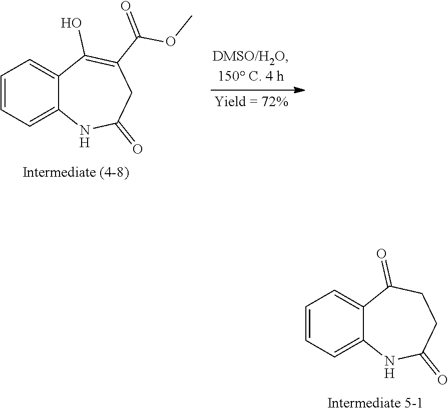 [SMILES: COC(=O)C1=C(O)c2ccccc2NC(=O)C1 ; O=C1CCC(=O)c2ccccc2N1]

A mixture of methyl 5-hydroxy-2-oxo-2,3-dihydro-1H-benzo[b]azepine-4-carboxylate 4-1 (43.4 g, 0.2 mol) in DMSO (520 mL) and H₂O (15 mL) was heated at 150° C. for 4 b. The reaction mixture was allowed to cool to rt, ice (1.0 L) was added, and the mixture was allowed to stir 12 h. To the flask was added 1N HCl (1.0 L) at 0° C. and the mixture allowed to stir for 3 h. The resulting precipitate was filtered and dried under reduced pressure to afford product 23.5 g (purity=92%) light yellow solid compound with a yield of 72%.

TLC R_{f}=0.4 (PE:EA=1:1)

LCMS (m/z)=176.30 (M+H)

¹H NMR (400 MHZ, DMSO) δ 10.11 (s, 1H), 7.90-7.75 (m, 1H), 7.54 (dd, J=10.9, 4.4 Hz, 1H), 7.22-7.11 (m, 2H), 2.97-2.85 (m, 2H), 2.67 (dd, J=7.6, 4.7 Hz, 2H). ¹³C NMR (100 MHz, DMSO) δ 199.16, 174.02, 139.72, 134.58, 130.77, 127.20, 123.83, 122.14, 38.75, 29.74.

### Intermediate (5-2): 7-methoxy-3,4-dihydro-1H-benzo[b]azepine-2,5-dione

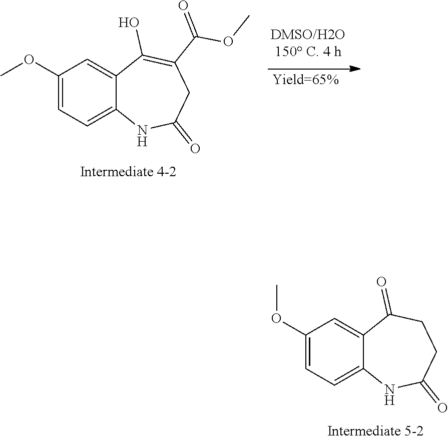

A mixture of methyl 5-hydroxy-7-methoxy-2-oxo-2,3-dihydro-1H-benzo[b]azepine-4-carboxylate 4-2 (6 g, 0.022 mol) in DMSO (63.8 mL) and H₂O (1.82 mL) was heated at 150° C. for 4 h. The reaction mixture was allowed to cool to rt, ice (0.5 L) was added, and the mixture was allowed to stir 12 h. To the flask was added 1N HCl (0.5 L) at 0° C. and the mixture allowed to stir for 3 h. The resulting precipitate was filtered and dried under reduced pressure to afford product 3 g (purity=94%) light yellow solid compound with a yield of 65%.

TLC R_{f}=0.3 (PE:EA=1:1)

LCMS (m/z)=206.20 (M+H)

### Intermediate (5-3): 8-bromo-3,4-dihydro-1H-benzo[b]azepine-2,5-dione

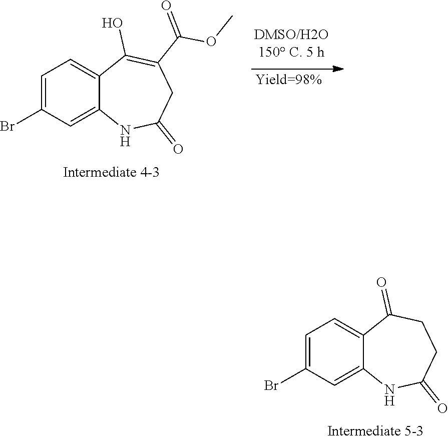 [SMILES: COC(=O)C1=C(O)c2ccc(Br)cc2NC(=O)C1 ; O=C1CCC(=O)c2ccc(Br)cc2N1]

Methyl 8-bromo-5-hydroxy-2-oxo-2,3-dihydro-1H-benzo[b]azepine-4-carboxylate 4-3 (4 g, 0.0128 mol) in DMSO (23 mL) and H₂O (0.66 mL) was heated at 150° C. for 5 h. The reaction mixture was allowed to cool to rt, ice (420 mL) was added, and the mixture was allowed to stir 12 h. To the flask was added 1N HCl (420 mL) at 0° C. and the mixture allowed to stir for 3 h. The resulting precipitate was filtered and dried under reduced pressure to afford product 3.2 g (purity=96%) light yellow solid compound with a yield of 98%.

TLC R_{f}=0.4 (PE:EA=1:2)

LCMS (m/z)=254.30 (M+H)

### Intermediate (5-4): 8-methoxy-3,4-dihydro-1H-benzo[b]azepine-2,5-dione

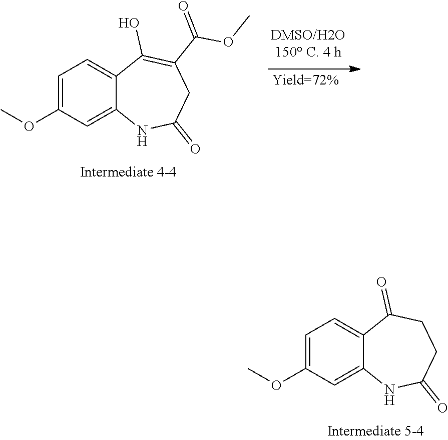

A mixture of methyl 5-hydroxy-8-methoxy-2-oxo-2,3-dihydro-1H-benzo[b]azepine-4-carboxylate 4-4 (5.5 g, 0.02 mol) in DMSO (58 mL) and H₂O (2 mL) was heated at 150° C. for 4 h. The reaction mixture was allowed to cool to rt, ice (0.5 L) was added, and the mixture was allowed to stir 12 h. To the flask was added 1N HCl (0.5 L) at 0° C. and the mixture allowed to stir for 3 h. The resulting precipitate was filtered and dried under reduced pressure to afford product 3.1 g (purity=99%) light yellow solid compound with a yield of 72%.

TLC R_{f}=0.3 (PE:EA=1:1)

LCMS (m/z)=205.70 (M+H)

### Intermediate (5-5): 8-methyl-3,4-dihydro-1H-benzo[b]azepine-2,5-dione

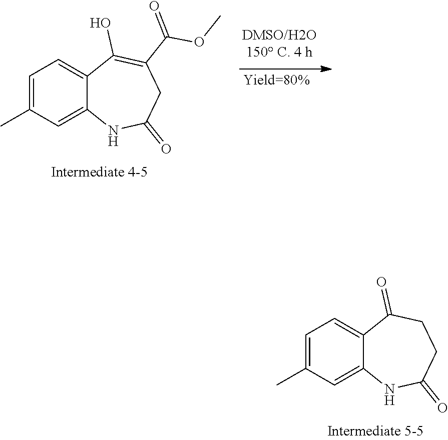

A mixture of methyl 5-hydroxy-8-methyl-2-oxo-2,3-dihydro-1H-benzo[b]azepine-4-carboxylate 4-5 (4.9 g, 0.019 mol) in DMSO (55 mL) and H₂O (2 mL) was heated at 150° C. for 4 h. The reaction mixture was allowed to cool to rt, ice (0.5 L) was added, and the mixture was allowed to stir 12 h. To the flask was added 1N HCl (0.5 L) at 0° C. and the mixture allowed to stir for 3 h. The resulting precipitate was filtered and dried under reduced pressure to afford product 2 g (purity=97%) light yellow solid compound with a yield of 80%.

TLC R_{f}=0.3 (PE:EA=1:1)

LCMS (m/z)=189.80 (M+H)

### Intermediate (5-6): 8-(trifluoromethyl)-3,4-dihydro-1H-benzo[b]azepine-2,5-dione

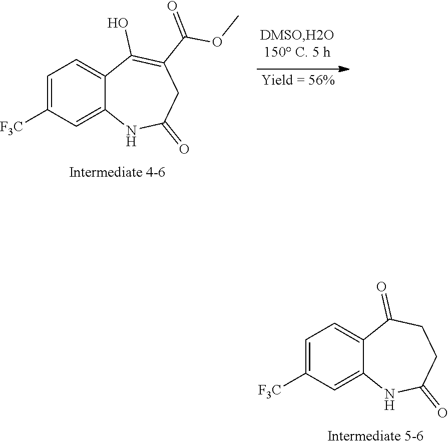 [SMILES: COC(=O)C1=C(O)c2ccc(C(F)(F)F)cc2NC(=O)C1 ; O=C1CCC(=O)c2ccc(C(F)(F)F)cc2N1]

To a solution of Methyl-5-hydroxy-2-oxo-8-(trifluoromethyl)-2,3-dihydro-1H-benzo[b]azepine-4-carboxylate 4-6 (4.3 g, 13.95 mmol) in DMSO (25 mL), was added H₂O (1 mL) under nitrogen, and the reaction mixture was allowed to stir at 150° C. for 5 h. The reaction progress was monitored by TLC until the complete disappearance of compound 4-6. The reaction mixture was allowed to cool to rt, ice (45 mL) was added, and the mixture was allowed to stir 12 h. To the flask was added 1N HCl (45 mL) at 0° C. and the mixture allowed to stir for 3 h. The resulting precipitate was filtered. Then, the crude product was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 1.9 g (purity=99%) light yellow oil compound with a yield of 56%.

TLC R_{f}=0.3 (PE:EA=1:1)

LCMS (m/z)=244.20 (M+H)

### Intermediate (5-7): 7-fluoro-3,4-dihydro-1H-benzo[b]azepine-2,5-dione

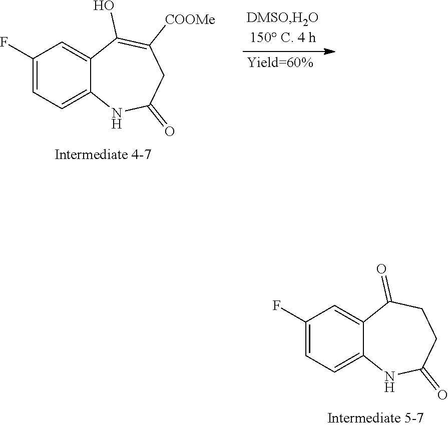 [SMILES: COC(=O)C1=C(O)c2cc(F)ccc2NC(=O)C1 ; O=C1CCC(=O)c2cc(F)ccc2N1]

A mixture of Methyl 5-hydroxy-7-methyl-2-oxo-2,3-dihydro-1H-benzo[b]azepine-4-carboxylate 4-7 (3.47 g, 14 mmol) in DMSO (25 mL) was added H₂O (1.5 mL) was heated at 150° C. for 4 h. The reaction mixture was allowed to cool to rt, ice (460 mL) was added, and the mixture was allowed to stir for 12 h. To the flask was added 1 N HCl (460 mL) at 0° C. and the mixture allowed to stir for 3 h. The resulting precipitate was filtered and dried under reduced pressure to afford product 1.76 g (purity=99%) of the light yellow oil compound with a yield of 60%.

TLC R_{f}=0.2 (EA:PE=1:1)

LCMS (m/z)=193.60 (M+H)

### Intermediate (5-8): 7-methyl-3,4-dihydro-1H-benzo[b]azepine-2,5-dione

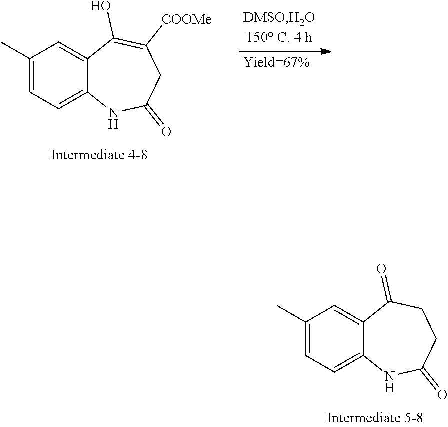 [SMILES: COC(=O)C1=C(O)c2cc(C)ccc2NC(=O)C1 ; Cc1ccc2c(c1)C(=O)CCC(=O)N2]

A mixture of Methyl 5-hydroxy-7-methyl-2-oxo-2,3-dihydro-1H-benzo[b]azepine-4-carboxylate 4-8 (3.47 g, 14 mmol) in DMSO (25 mL) was added H₂O (1.5 mL) was heated at 150° C. for 4 h. The reaction mixture was allowed to cool to rt, ice (46 mL) was added, and the mixture was allowed to stir 12 h. To the flask was added 1 N HCl (46 mL) at 0° C. and the mixture allowed to stir for 3 h. The resulting precipitate was filtered and dried under reduced pressure to afford product 1.78 g (purity=99%) of the light yellow oil compound with a yield of 67%.

TLC R_{f}=0.33 (EA:PE=1:1)

LCMS (m/z)=189.90 (M+H)

### Intermediate (6-70): 1-(3-fluoro-4-methylbenzyl)-8-hydroxy-3,4-dihydro-1H-benzo[b]azepine-2,5-dione

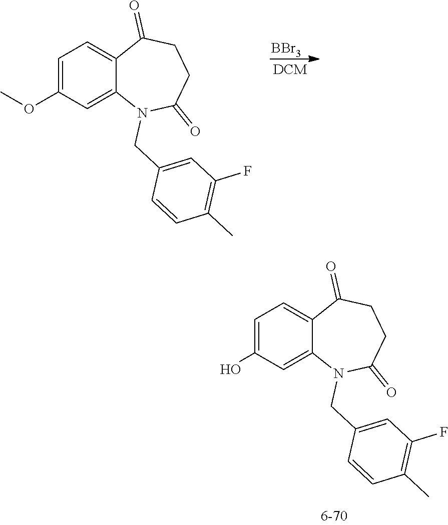 [SMILES: COc1ccc2c(c1)N(Cc1ccc(C)c(F)c1)C(=O)CCC2=O ; "BBr3" ; Cc1ccc(CN2C(=O)CCC(=O)c3ccc(O)cc32)cc1F]

A suspension of ketone (163 mg, 0.5 mmol, 1 equiv), in dry DCM (3 mL), was cooled to −78° C. then BBr3 (240 mg, 2.5 mmol, 5 equiv) was added, it was slowly warmed to ambient temperature and then stirred for 6 h. The solution was quenched with saturated NaHCO3 (5 mL) and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 100 mg light yellow solid compound with a yield of 60%.

TLC R_{f}=0.2 (DCM:MeOH=40:1)

LCMS (m/z)=314.50 (M+H)

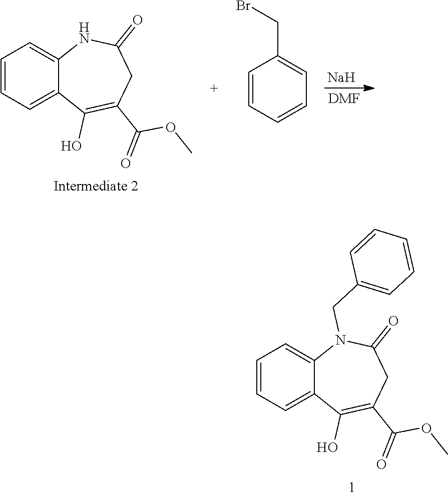

### Example 1. Methyl 1-benzyl-5-hydroxy-2-oxo-2,3-dihydro-1H-benzo[b]azepine-4-carboxylate

To a solution of methyl 5-hydroxy-2-oxo-2,3-dihydro-1H-benzo[b]azepine-4-carboxy late (Intermediate 2) (233 mg, 1 mmol, 1 equiv) in DMF (4 mL) was added NaH 60% dispersion in mineral oil (60 mg, 1.5 mmol, 3 equiv). To this solution was added (bromomethyl)benzene (1.2 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 100 mg of the white solid compound with a yield of 31%, purity=83%.

TLC R_{f}=0.5 (PE:EA=2:1)

LCMS (m/z)=324.2 (M+H).

¹H NMR (400 MHZ, DMSO) δ 12.28 (s, 1H), 7.74 (d, J=7.5 Hz, 1H), 7.58-7.43 (m, 2H), 7.37-7.12 (m, 4H), 7.02 (d, J=7.2 Hz, 2H), 5.25 (d, J=16.0 Hz, 1H), 4.98 (d, J=16.1 Hz, 1H), 3.85 (s, 3H), 3.50 (d, J=13.9 Hz, 1H), 2.66 (d, J=13.9 Hz, 1H). ¹³C NMR (100 MHZ, DMSO) δ 170.40, 170.23, 165.63, 140.71, 137.44, 131.81, 128.38, 128.16, 127.75, 126.88, 126.53, 125.06, 123.13, 97.72, 52.56, 49.97, 31.11.

### Example 2. Methyl 5-hydroxy-2-oxo-1-(pyridin-2-ylmethyl)-2,3,4,5-tetrahydro-1H-benzo[b]azepine-4-carboxylate (2)

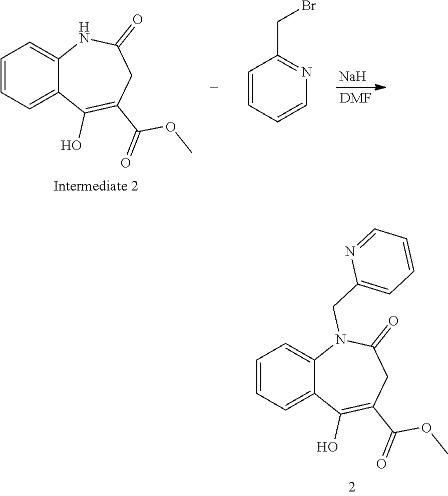

To a solution of methyl 5-hydroxy-2-oxo-2,3-dihydro-1H-benzo[b]azepine-4-carboxylate (Intermediate 2) (70 mg, 0.3 mmol, 1 equiv) in DMF (2 mL) was added NaH 60% dispersion in mineral oil (91 mg, 0.9 mmol, 3 equiv). To this solution was added 2-(bromomethyl) pyridine hydrobromide (0.36 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 ml), dried over anhydrous Na₂SO₄, and concentrated by rotary evaporation. Then, the resulting concentrate was purified on a silica column to get 72 mg of the white solid compound with a yield of 75%, purity=98%.

TLC R_{f}=0.3 (EA)

LCMS (m/z)=325.2 (M+H).

### Example 3. Methyl 5-hydroxy-2-oxo-1-(pyridin-3-ylmethyl)-2,3-dihydro-1H-benzo[b]azepine-4-carboxylate (3)

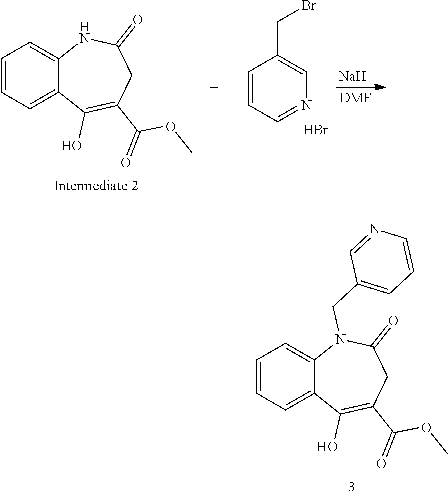

To a solution of methyl 5-hydroxy-2-oxo-2,3-dihydro-1H-benzo[b]azepine-4-carboxylate (Intermediate 2) (70 mg, 0.3 mmol, 1 equiv) in DMF (2 mL) was added NaH 60% dispersion in mineral oil (91 mg, 0.9 mmol, 3 equiv). To this solution was added 3-(bromomethyl) pyridine hydrobromide (0.36 mmol, 1.2 equiv), and the reaction mixture was allowed to stir at rt for 4 h. The reaction progress was monitored by TLC until the complete disappearance of Intermediate 2. The resulting solution was poured into water (30 ml) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 40 mg of the white solid compound with a yield of 42%, purity=92%.

TLC R_{f}=0.3 (EA)

LCMS (m/z)=325.3 (M+H).

### Example 4. Methyl 5-hydroxy-2-oxo-1-(pyridin-4-ylmethyl)-2,3-dihydro-1H-benzo[b]azepine

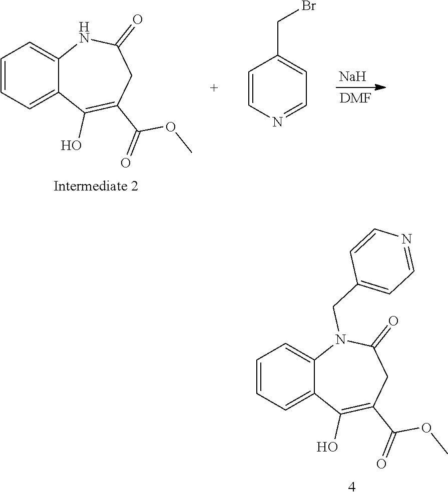

4-carboxylate (4)

To a solution of methyl 5-hydroxy-2-oxo-2,3-dihydro-1H-benzo[b]azepine-4-carboxylate (Intermediate 2) (70 mg, 0.3 mmol, 1 equiv) in DMF (2 mL) was added NaH 60% dispersion in mineral oil (91 mg, 0.9 mmol, 3 equiv). To this solution was added 4-(bromomethyl) pyridine hydrobromide (0.36 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 30 mg of the white solid compound with a yield of 31%, purity=93%.

TLC R_{f}=0.3 (EA)

LCMS (m/z)=325.3 (M+H).

### Example 5. Methyl 5-hydroxy-1-(3-methylbenzyl)-2-oxo-2,3-dihydro-1H-benzo[b]azepine-4-carboxylate (5)

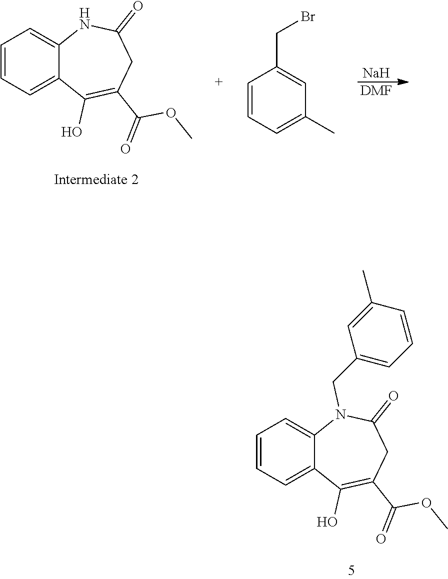

To a solution of methyl 5-hydroxy-2-oxo-2,3-dihydro-1H-benzo[b]azepine-4-carboxy late (Intermediate 2) (70 mg, 0.3 mmol, 1 equiv) in DMF (2 mL) was added NaH 60% dispersion in mineral oil (91 mg, 0.9 mmol, 3 equiv). To this solution was added 1-(bromomethyl)-3-methylbenzene (0.36 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 80 mg of the white solid compound with a yield of 79%, purity=95%.

TLC R_{f}=0.5 (PE:EA=2:1)

LCMS (m/z)=338.2 (M+H).

### Example 6. Methyl 1-(3,5-dimethylbenzyl)-5-hydroxy-2-oxo-2,3-dihydro-1H-benzo[b]azepine-4-carboxylate (6)

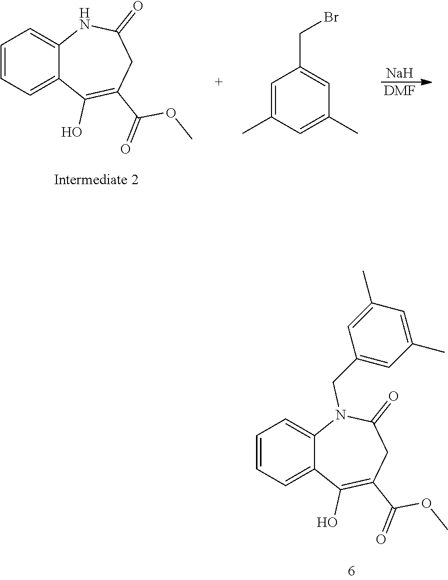

To a solution of methyl 5-hydroxy-2-oxo-2,3-dihydro-1H-benzo[b]azepine-4-carboxylate (Intermediate 2) (70 mg, 0.3 mmol, 1 equiv) in DMF (2 mL) was added NaH 60% dispersion in mineral oil (91 mg, 09 mmol, 3 equiv). To this solution was added 1-(bromomethyl)-3,5-dimethylbenzene (0.36 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 70 mg of the white sold compound with a yield of 80%, purity=95%.

TLC R_{f}=0.5 (PE:EA=2:1)

LCMS (m/z)=352.3 (M+H).

### Example 7. Methyl 5-hydroxy-1-(4-methylbenzyl)-2-oxo-2,3-dihydro-1H-benzo[b]azepine-4-carboxylate (7)

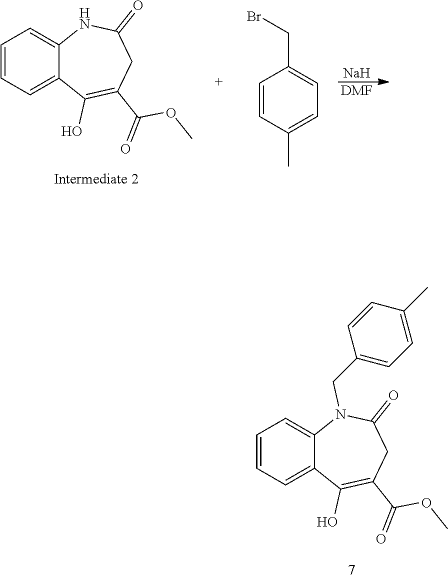

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 30 mg of the white solid compound with a yield of 90%, purity=92%.

TLC R_{f}=0.5 (PE:EA=2:1)

LCMS (m/z)=338.2 (M+H).

¹H NMR (400 MHZ, DMSO) δ 12.28 (s, 1H), 7.72 (d, J=7.8 Hz, 1H), 7.52 (d, J=3.0 Hz, 2H), 7.28 (dd, J=7.4, 3.6 Hz, 1H), 7.01 (t, J=6.5 Hz, 2H), 6.91 (d, J=7.9 Hz, 2H), 5.22 (d, J=15.8 Hz, 1H), 4.91 (d, J=15.9 Hz, 1H), 3.84 (s, 3H), 3.49 (d, J=13.8 Hz, 1H), 2.63 (d, J=13.8 Hz, 1H), 2.19 (s, 3H). ¹³C NMR (100 MHz, DMSO) δ 170.36, 170.24, 165.67, 140.67, 135.95, 134.37, 131.74, 128.94, 128.20, 127.73, 126.57, 125.00, 123.15, 97.68, 52.52, 49.60, 31.10, 20.52.

### Example 8. Methyl 1-(3-fluorobenzyl)-5-hydroxy-2-oxo-2,3-dihydro-1H-benzo[b]azepine-4-carboxylate (8)

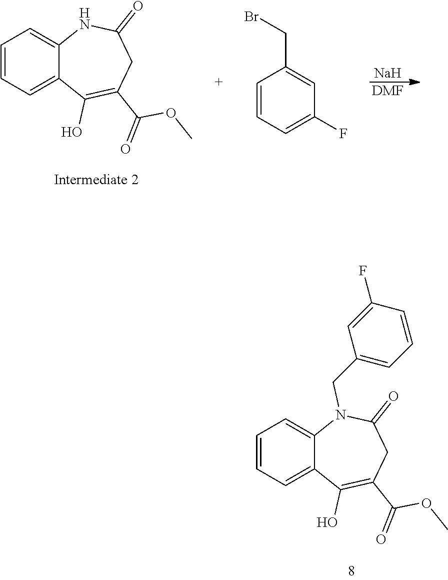

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-3-fluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 20 mg of the white solid compound with a yield of 62%, purity=95%.

TLC R_{f}=0.5 (PE:EA=2:1)

LCMS (m/z)=342.2 (M+H).

### Example 9. Methyl 1-(2-fluorobenzyl)-5-hydroxy-2-oxo-2,3-dihydro-1H-benzo[b]azepine-4-carboxylate (9)

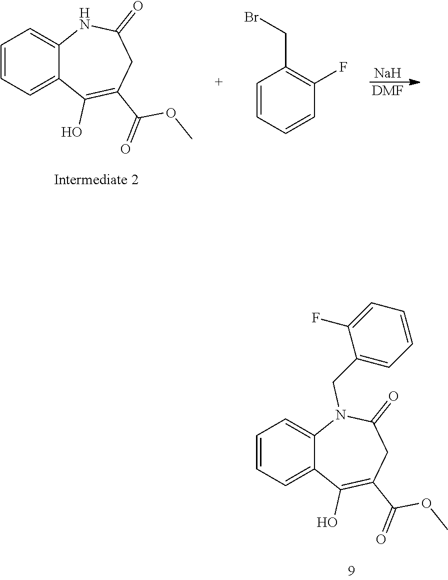

To a solution of methyl 5-hydroxy-2-oxo-2,3-dihydro-1H-benzo[b]azepine-4-carboxy late (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2-fluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 28 mg of the white solid compound with a yield of 87%, purity=90%.

TLC R_{f}=0.5 (PE:EA=2:1)

LCMS (m/z)=342.2 (M+H).

### Example 10. Methyl 1-(4-fluorobenzyl)-5-hydroxy-2-oxo-2,3-dihydro-1H-benzo[b]azepine-4-carboxylate (10)

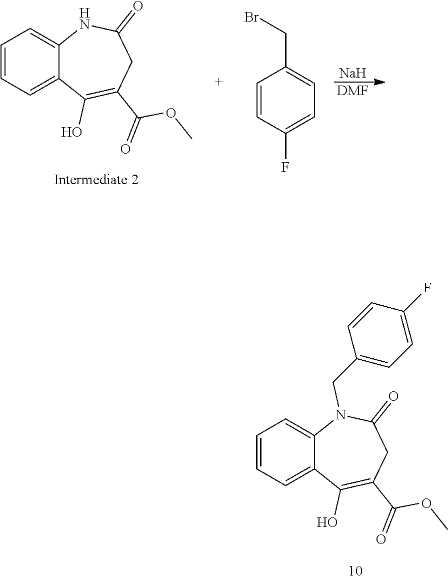

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-fluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 12 mg of the white solid compound with a yield of 37%, purity=90%.

TLC R_{f}=0.5 (PE:EA=2:1)

LCMS (m/z)=342.3 (M+H)

### Example 11. Methyl 1-(3,5-dimethoxybenzyl)-5-hydroxy-2-oxo-2,3-dihydro-1H-benzo[b]azepine-4-carboxylate (11)

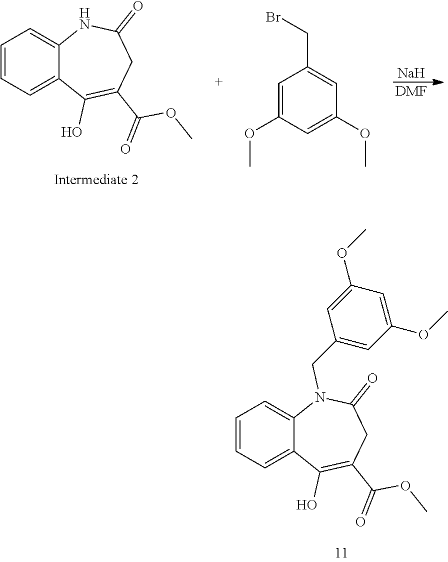

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-3,5-dimethoxybenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 17 mg of the white solid compound with a yield of 44%, purity=90%.

TLC R_{f}=0.5 (PE:EA=2:1)

LCMS (m/z)=384.2 (M+H).

### Example 12. Methyl 5-hydroxy-2-oxo-1-(quinolin-3-ylmethyl)-2,3-dihydro-1H-benzo[b]azepine-4-carboxylate (12)

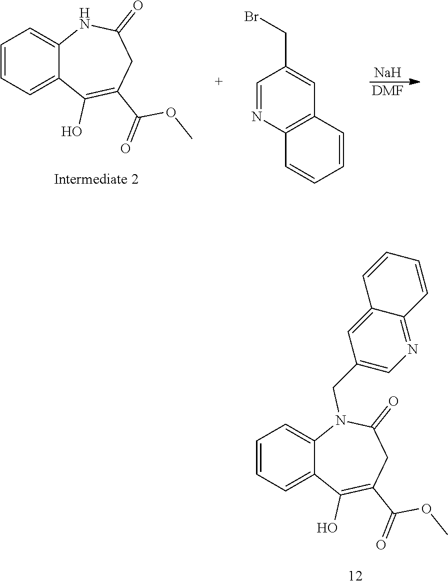

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 3-(bromomethyl) quinoline (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 37 mg of the white solid compound with a yield of 98%, purity=79%.

TLC R_{f}=0.5 (PE:EA=2:1)

LCMS (m/z)=375.40 (M+H).

### Example 13. 3-((5-hydroxy-4-(methoxycarbonyl)-2-oxo-2,3-dihydro-1H-benzo[b]azepin-1-yl) methyl) benzoic acid (13)

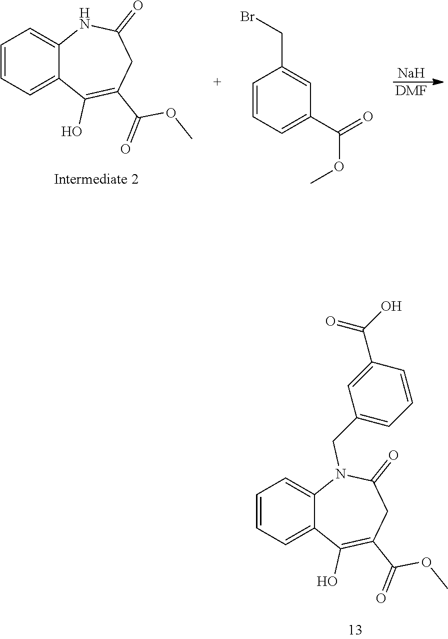

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added methyl 3-(bromomethyl) benzoate (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 38 mg of the white solid compound with a yield of 90%, purity=95%.

TLC R_{f}=0.1 (PE:EA=2:1)

LCMS (m/z)=368.40 (M+H).

### Example 14. Methyl 5-hydroxy-1-(naphthalen-2-ylmethyl)-2-oxo-2,3-dihydro-1H-benzo[b]azepine-4-carboxylate (14)

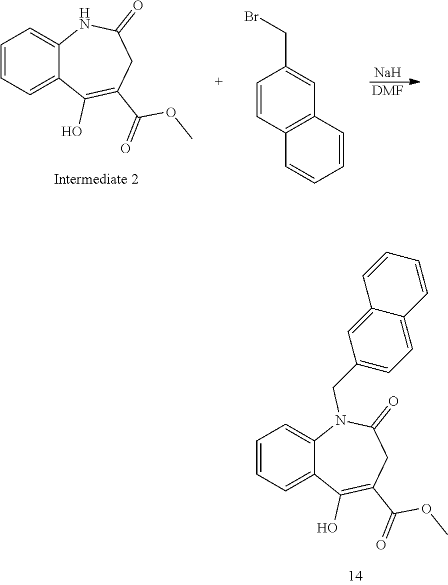

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-(bromomethyl) naphthalene (0.12 mmol, 1.2 equiv), and the reaction mixture was allowed to stir at rt for 4 h. The reaction progress was monitored by TLC until the complete disappearance of Intermediate 2. The resulting solution was poured into water (30 ml) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 43 mg of the white solid compound with a yield of 99%, purity=67%.

TLC R_{f}=0.5 (PE:EA=2:1)

LCMS (m/z)=374.60 (M+H).

### Example 15. Methyl 1-([1,1′-biphenyl]-4-ylmethyl)-5-hydroxy-2-oxo-2,3,4,5-tetrahydro-1H-benzo[b]azepine-4-carboxylate (15)

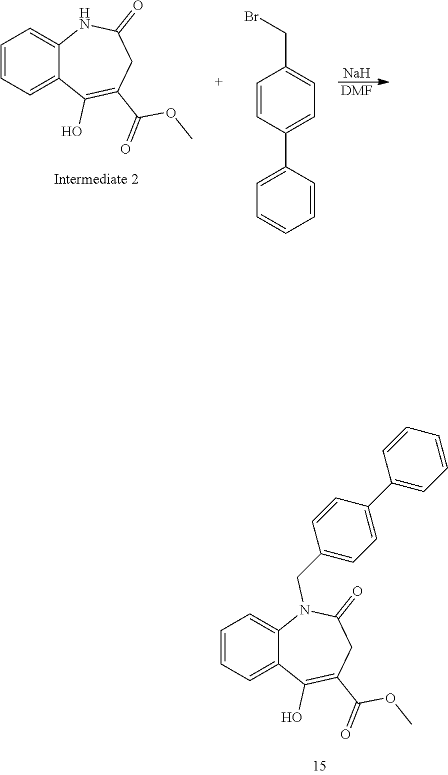

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-1, l′-biphenyl (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 44 mg of the white solid compound with a yield of 98%, purity=91%.

TLC R_{f}=0.5 (PE:EA=2:1)

LCMS (m/z)=400.2 (M+H).

### Example 16. Methyl 1-(2,6-dimethylbenzyl)-5-hydroxy-2-oxo-2,3-dihydro-1H-benzo[b]azepine-4-carboxylate (16)

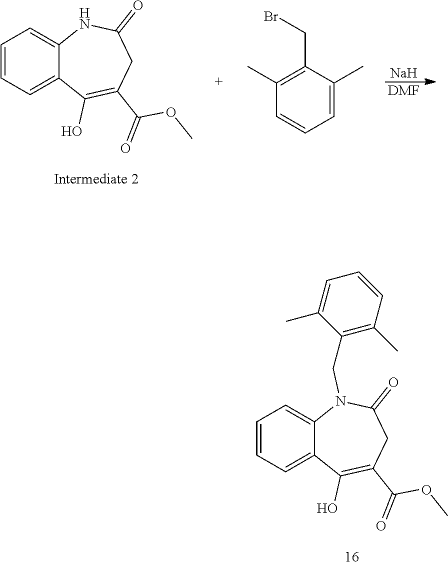

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-(bromomethyl)-1,3-dimethylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 48 mg of the white solid compound with a yield of 95%, purity=90%.

TLC R_{f}=0.5 (PE:EA=2:1)

LCMS (m/z)=352.2 (M+H).

### Example 17. Methyl 1-(2,3-dimethylbenzyl)-5-hydroxy-2-oxo-2,3-dihydro-1H-benzo[b]azepine-4-carboxylate (17)

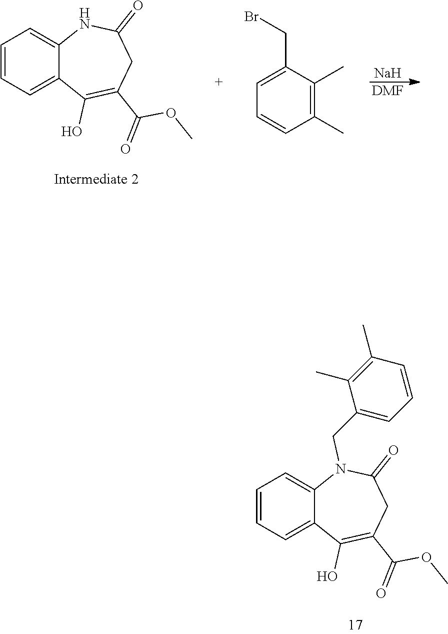

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2,3-dimethylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 39 mg of the white solid compound with a yield of 90%, purity=90%.

TLC R_{f}=0.5 (PE:EA=2:1)

LCMS (m/z)=252.3 (M+H).

### Example 18. Methyl 1-(2,4-difluorobenzyl)-5-hydroxy-2-oxo-2,3-dihydro-1H-benzo[b]azepine-4-carboxylate (18)

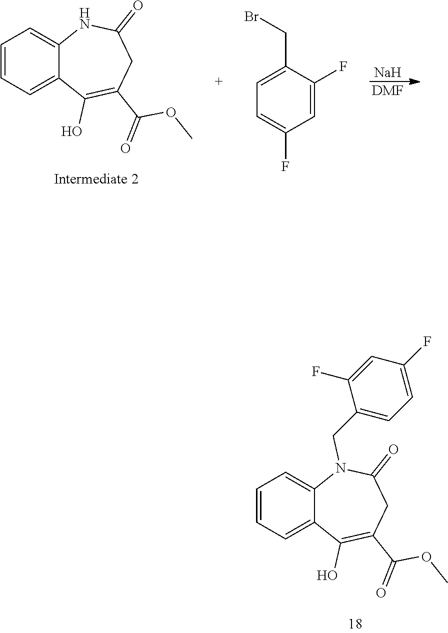

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2,4-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 42 mg of the white solid compound with a yield of 99%, purity=93%.

TLC R_{f}=0.5 (PE:EA=2:1)

LCMS (m/z)=360.2 (M+H).

### Methyl 1-(2,5-difluorobenzyl)-5-hydroxy-2-oxo-2,3-dihydro-1H-benzo[b]azepine-4-carboxylate (19)

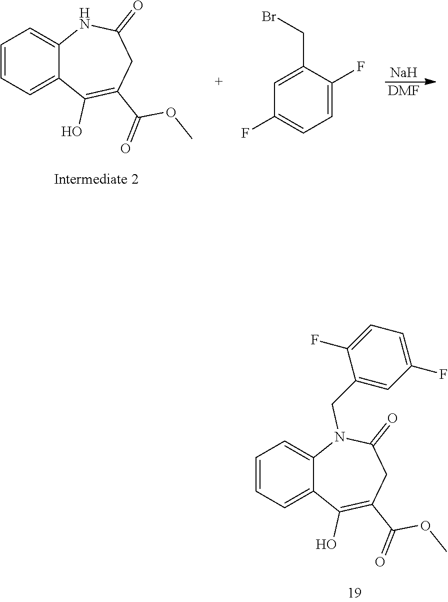

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-(bromomethyl)-1,4-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 38 mg of the white solid compound with a yield of 98%, purity=93%.

TLC R_{f}=0.5 (PE:EA=2:1)

LCMS (m/z)=360.2 (M+H).

### Example 19. Methyl 1-(3,5-difluorobenzyl)-5-hydroxy-2-oxo-2,3-dihydro-1H-benzo[b]azepine-4-carboxylate (20)

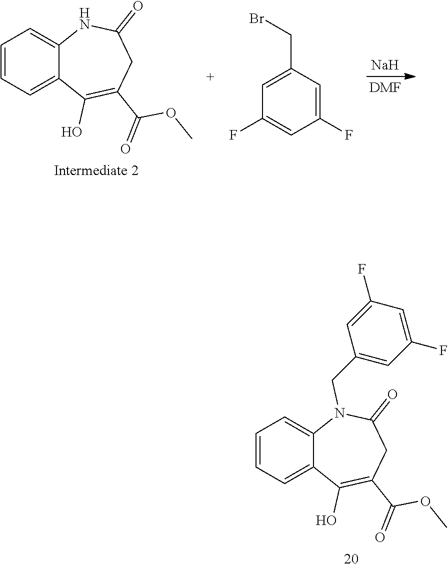

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-3,5-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 35 mg of the white solid compound with a yield of 95%, purity=92%.

TLC R_{f}=0.5 (PE:EA=2:1)

LCMS (m/z)=360.2 (M+H).

### Example 20. Methyl 1-(3,4-difluorobenzyl)-5-hydroxy-2-oxo-2,3-dihydro-1H-benzo[b]azepine-4-carboxylate (21)

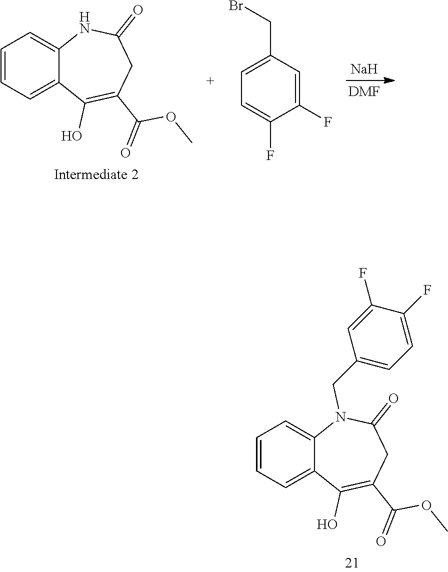

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-1,2-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 36 mg of the white solid compound with a yield of 98%, purity=92%.

TLC R_{f}=0.5 (PE:EA=2:1)

LCMS (m/z)=360.2 (M+H)

### Methyl 1-(2,3-difluorobenzyl)-5-hydroxy-2-oxo-2,3-dihydro-1H-benzo[b]azepine-4-carboxylate (22)

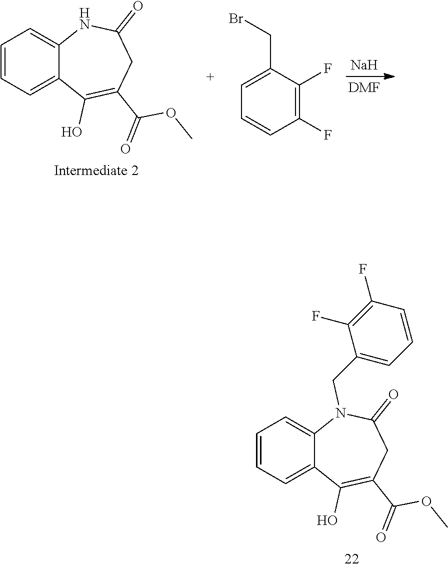

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2,3-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 35 mg of the white solid compound with a yield of 97%, purity=91%.

TLC R_{f}=0.5 (PE:EA=2:1)

LCMS (m/z)=360.2 (M+H).

### Methyl 1-(2,6-difluorobenzyl)-5-hydroxy-2-oxo-2,3-dihydro-1H-benzo[b]azepine-4-carboxylate (23)

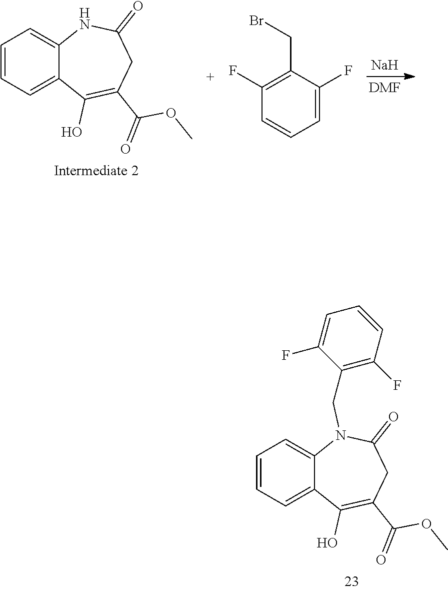

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-(bromomethyl)-1,3-difluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 38 mg of the white solid compound with a yield of 97%, purity=85%.

TLC R_{f}=0.5 (PE:EA=2:1)

LCMS (m/z)=360.2 (M+H).

### Example 24. Methyl 1-(2-cyanobenzyl)-5-hydroxy-2-oxo-2,3-dihydro-1H-benzo[b]azepine-4-carboxylate (24)

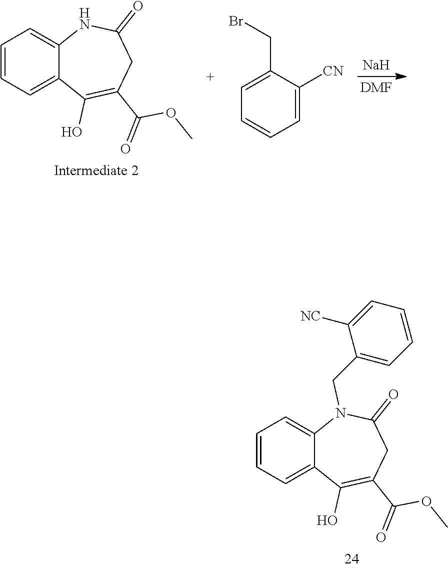

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-(bromomethyl) benzonitrile (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 32 mg of the white solid compound with a yield of 98%, purity=90%.

TLC R_{f}=0.5 (PE:EA=2:1)

LCMS (m/z)=349.2 (M+H).

### Example 25. Methyl 1-(3-cyanobenzyl)-5-hydroxy-2-oxo-2,3-dihydro-1H-benzo[b]azepine-4-carboxylate (25)

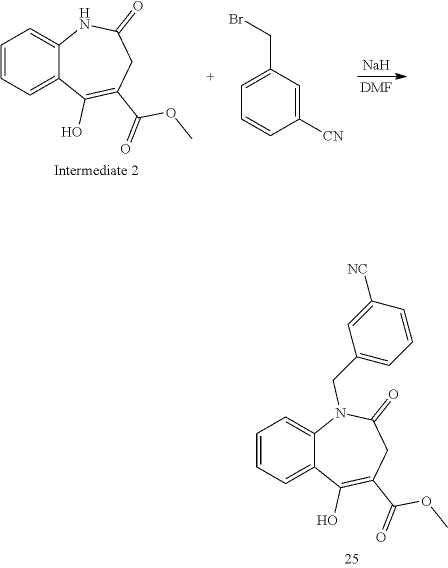

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 3-(bromomethyl) benzonitrile (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 39 mg of the white solid compound with a yield of 98%, purity=85%.

TLC R_{f}=0.5 (PE:EA=2:1)

LCMS (m/z)=349.2 (M+H).

### Example 26. Methyl 1-(4-cyanobenzyl)-5-hydroxy-2-oxo-2,3-dihydro-1H-benzo[b]azepine-4-carboxylate (26)

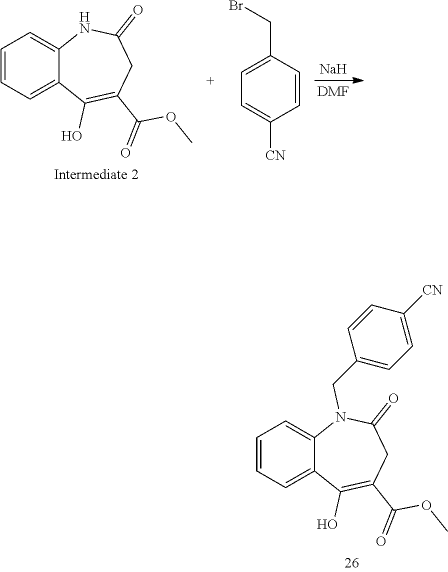

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl) benzonitrile (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 7 mg of the white solid compound with a yield of 15%, purity=60%.

TLC R_{f}=0.5 (PE:EA=2:1)

LCMS (m/z)=349.2 (M+H).

### Example 27. Methyl 5-hydroxy-1-(3-methoxybenzyl)-2-oxo-2,3-dihydro-1H-benzo[b]azepine-4-carboxylate (27)

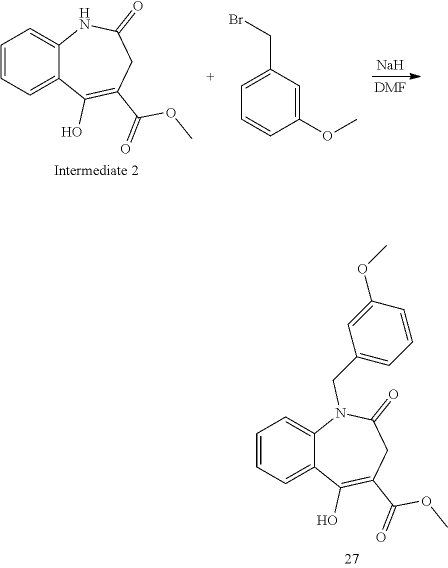

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-3-methoxybenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 6 mg of the white solid compound with a yield of 18%, purity=75%.

TLC R_{f}=0.5 (PE:EA=2:1)

LCMS (m/z)=354.2 (M+H).

### Example 28. Methyl 5-hydroxy-2-oxo-1-(3,4,5-trifluorobenzyl)-2,3-dihydro-1H-benzo[b]azepine-4-carboxylate (28)

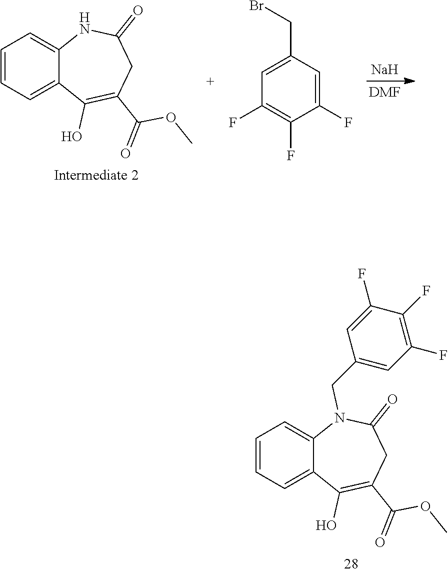

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 5-(bromomethyl)-1,2,3-trifluorobenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 38 mg of the white solid compound with a yield of 85%, purity=90%.

TLC R_{f}=0.5 (PE:EA=2:1)

LCMS (m/z)=378.2 (M+H).

### Example 29. Methyl 1-(2,4-dimethylbenzyl)-5-hydroxy-2-oxo-2,3-dihydro-1H-benzo[b]azepine-4-carboxylate (29)

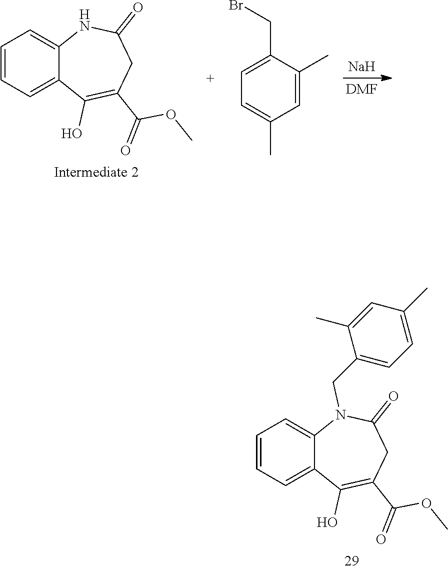

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2,4-dimethylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 37 mg of the white solid compound with a yield of 93%, purity=90%.

TLC R_{f}=0.5 (PE:EA=2:1)

LCMS (m/z)=352.3 (M+H).

### Example 30. Methyl 5-hydroxy-1-(4-isopropylbenzyl)-2-oxo-2,3-dihydro-1H-benzo[b]azepine-4-carboxylate (30)

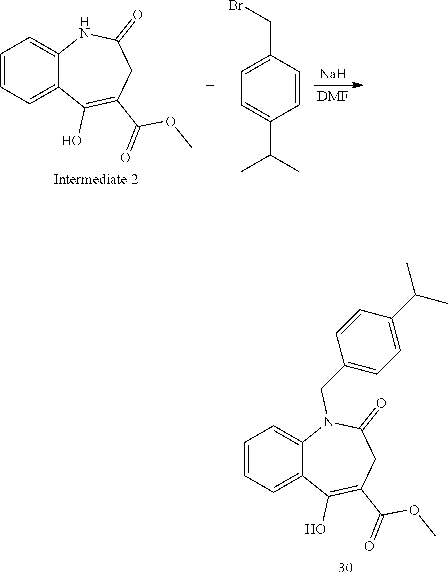

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-isopropylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 10 mg of the white solid compound with a yield of 30%, purity=70%.

TLC R_{f}=0.5 (PE:EA=2:1)

LCMS (m/z)=366.3 (M+H).

### Example 31. Methyl 1-(4-(tert-butyl) benzyl)-5-hydroxy-2-oxo-2,3-dihydro-1H-benzo[b]azepine-4-carboxylate (31)

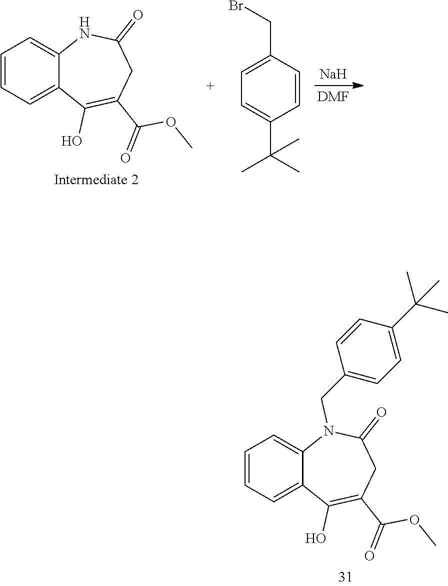

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-(tert-butyl) benzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 42 mg of the white solid compound with a yield of 90%, purity=80%.

TLC R_{f}=0.5 (PE:EA=2:1)

LCMS (m/z)=380.2 (M+H).

### Example 32. Methyl 5-hydroxy-2-oxo-1-(4-(trifluoromethoxy) benzyl)-2,3-dihydro-1H-benzo[b]azepine-4-carboxylate (32)

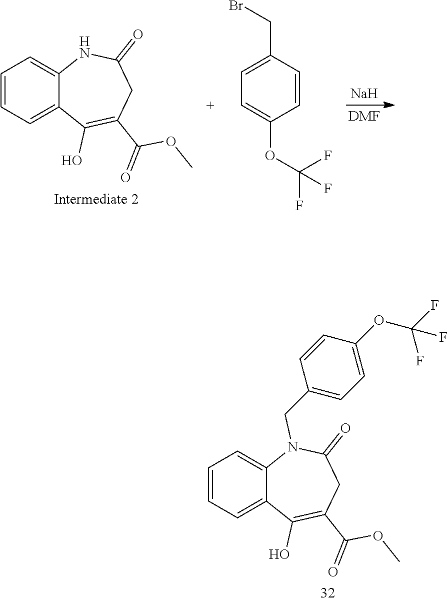

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-(trifluoromethoxy) benzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 50 mg of the white solid compound with a yield of 94%, purity=90%.

TLC R_{f}=0.5 (PE:EA=2:1)

LCMS (m/z)=408.2 (M+H).

### Example 33. Methyl 1-(4-(difluoromethoxy) benzyl)-5-hydroxy-2-oxo-2,3-dihydro-1H-benzo[b]azepine-4-carboxylate (33)

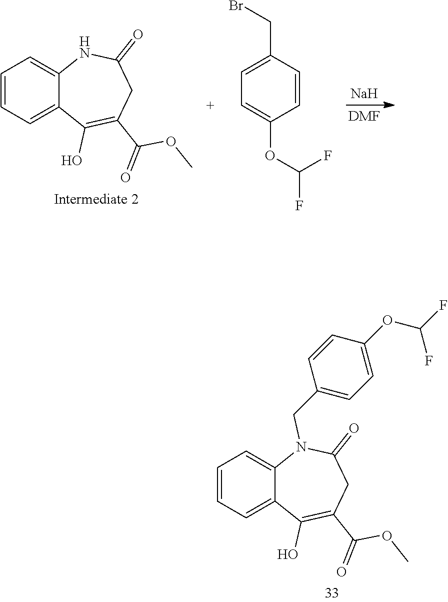

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-(difluoromethoxy) benzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 53 mg of the white solid compound with a yield of 90%, purity=97%.

TLC R_{f}=0.5 (PE:EA=2:1)

LCMS (m/z)=390.2 (M+H).

### Example 34. Methyl 5-hydroxy-1-(4-methoxybenzyl)-2-oxo-2,3-dihydro-1H-benzo[b]azepine-4-carboxylate (34)

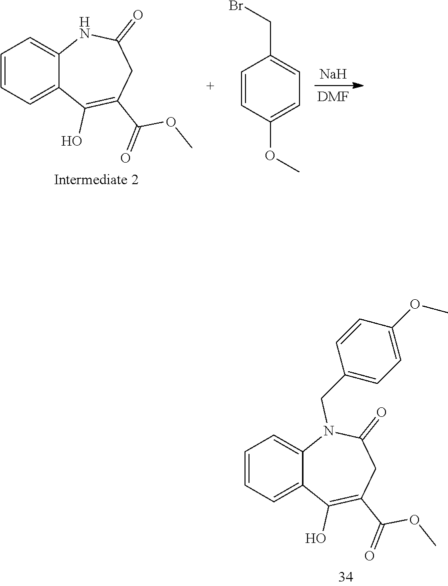

To a solution of methyl 5-hydroxy-2-oxo-2,3-dihydro-1H-benzo[b]azepine-4-carboxylate (Intermediate 2) (1943 mg, 8.33 mmol, 1 equiv) in DMF (54 mL) was added NaH 60% dispersion in mineral oil (1000 mg, 24.99 mmol, 3 equiv). To this solution was added 1-(bromomethyl)-4-methoxybenzene (10 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 820 mg of the white solid compound with a yield of 27.8%, purity=95%;

TLC R_{f}=0.4 (PE:EA=2:1)

LCMS (m/z)=354.60 (M+H)

¹H NMR (400 MHZ, DMSO) δ 12.27 (s, 1H), 7.78-7.68 (m, 1H), 7.59-7.48 (m, 2H), 7.29 (ddd, J=8.1, 6.4, 2.1 Hz, 1H), 6.93 (d, J=8.6 Hz, 2H), 6.77 (d, J=8.6 Hz, 2H), 5.22 (d, J=15.7 Hz, 1H), 4.87 (d, J=15.7 Hz, 1H), 3.85 (s, 3H), 3.66 (s, 3H), 3.48 (d, J=13.9 Hz, 1H), 2.62 (d, J=13.9 Hz, 1H); ¹³C NMR (100 MHz, DMSO) δ 170.33, 170.24, 165.63, 158.12, 140.62, 131.73, 129.21, 128.25, 127.97, 127.71, 125.02, 123.30, 113.76, 97.71, 54.91, 52.55, 49.21, 31.13.

### Example 35. Methyl 5-hydroxy-1-(2-methylbenzyl)-2-oxo-2,3-dihydro-1H-benzo[b]azepine-4-carboxylate (35)

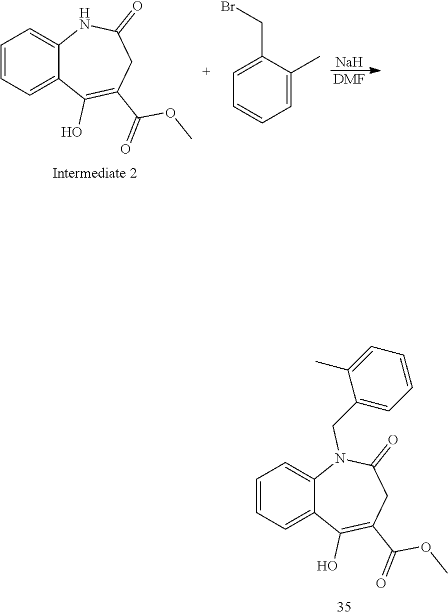

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-2-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 31 mg of the white solid compound with a yield of 97%, purity=93%.

TLC R_{f}=0.5 (PE:EA=2:1)

LCMS (m/z)=338.2 (M+H).

### Example 36. Methyl 1-(3,4-dimethylbenzyl)-5-hydroxy-2-oxo-2,3-dihydro-1H-benzo[b]azepine-4-carboxylate (36)

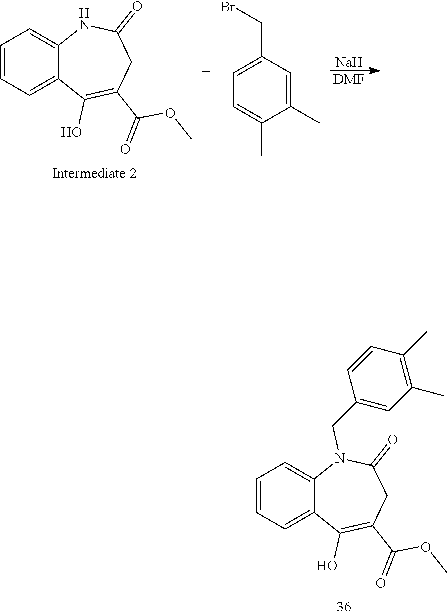

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-1,2-dimethylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 34 mg of the white solid compound with a yield of 97%, purity=90%.

TLC R_{f}=0.5 (PE:EA=2:1)

LCMS (m/z)=352.3 (M+H).

### Example 37. Methyl 1-(4-ethylbenzyl)-5-hydroxy-2-oxo-2,3-dihydro-1H-benzo[b]azepine-4-carboxylate (37)

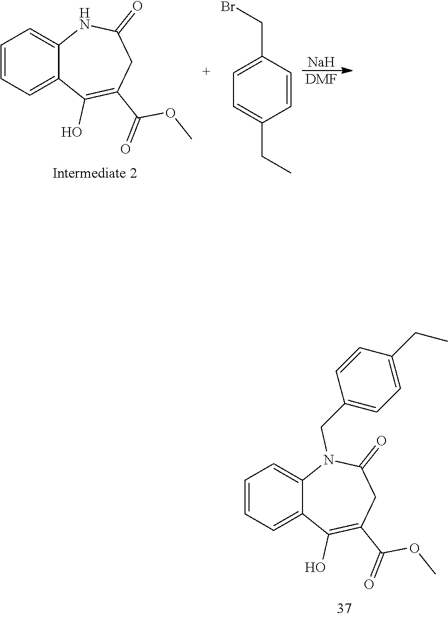

To a solution of methyl 5-hydroxy-2-oxo-2,3-dihydro-1H-benzo[b]azepine-4-carboxy late (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-ethylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 17 mg of the white solid compound with a yield of 67%, purity=87%.

TLC R_{f}=0.5 (PE:EA=2:1)

LCMS (m/z)=352.2 (M+H).

¹H NMR (400 MHZ, DMSO) δ 12.29 (s, 1H), 7.74 (d, J=7.8 Hz, 1H), 7.53 (d, J=3.6 Hz, 2H), 7.05 (t, J=11.2 Hz, 3H), 6.94 (d, J=7.9 Hz, 2H), 5.21 (d, J=16.0 Hz, 1H), 4.94 (d, J=16.0 Hz, 1H), 3.85 (s, 3H), 3.50 (d, J=13.9 Hz, 1H), 2.65 (d, J=13.8 Hz, 1H), 2.55-2.50 (m, 2H), 1.11 (t, J=7.6 Hz, 3H). ¹³C NMR (100 MHZ, DMSO) δ 170.36, 170.23, 165.64, 142.26, 140.76, 134.65, 131.80, 128.13, 127.75, 127.37, 126.50, 125.01, 123.10, 97.71, 52.55, 49.72, 31.11, 27.64, 15.38.

### Example 38. Methyl 5-hydroxy-1-(naphthalen-1-ylmethyl)-2-oxo-2,3-dihydro-1H-benzo[b]azepine-4-carboxylate (38)

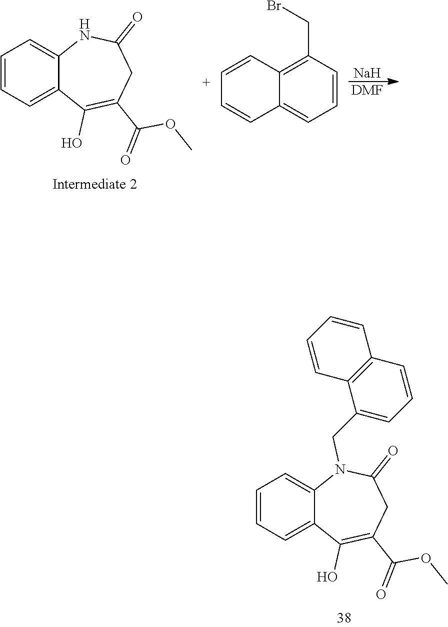

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl) naphthalene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 46 mg of the white solid compound with a yield of 91%, purity=90%.

TLC R_{f}=0.5 (PE:EA=2:1)

LCMS (m/z)=374.2 (M+H).

### Example 39. Methyl 5-hydroxy-2-oxo-1-(quinolin-8-ylmethyl)-2,3-dihydro-1H-benzo[b]azepine-4-carboxylate (39)

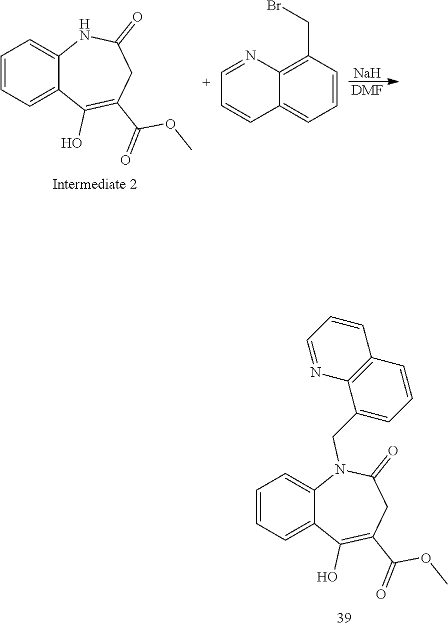

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 8-(bromomethyl) quinoline (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 16 mg of the white solid compound with a yield of 40%, purity=75%.

TLC R_{f}=0.5 (PE:EA=2:1)

LCMS (m/z)=375.2 (M+H).

### Example 40. Methyl 5-hydroxy-2-oxo-1-(4-(trifluoromethyl) benzyl)-2,3-dihydro-1H-benzo[b]azepine-4-carboxylate (40)

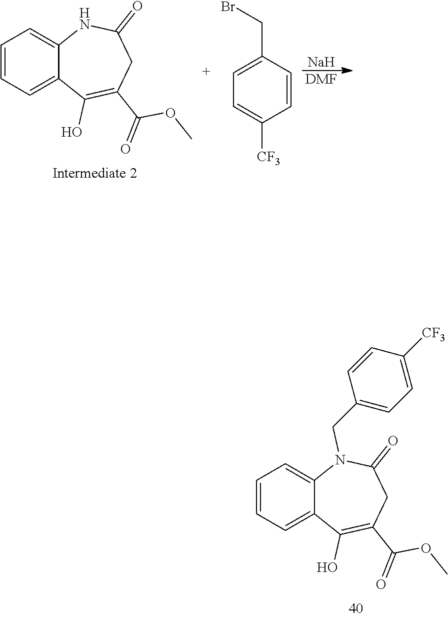

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-(trifluoromethyl) benzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 13 mg of the white solid compound with a yield of 33%, purity=70%.

TLC R_{f}=0.5 (PE:EA=2:1)

LCMS (m/z)=392.2 (M+H).

### Example 41. Methyl 1-(3-fluoro-4-methylbenzyl)-5-hydroxy-2-oxo-2,3-dihydro-1H-benzo[b]azepine-4-carboxylate (41)

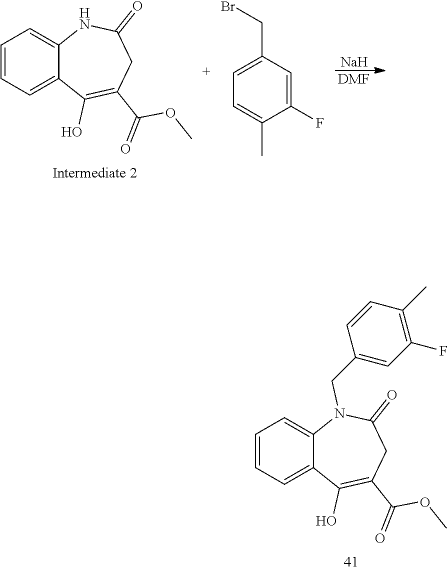

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-2-fluoro-1-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 39 mg of the white solid compound with a yield of 90%, purity=90%.

TLC R_{f}=0.5 (PE:EA=2:1)

LCMS (m/z)=356.2 (M+H).

¹H NMR (400 MHZ, DMSO) δ 12.28 (s, 1H), 7.74 (dd, J=7.9, 1.4 Hz, 1H), 7.60-7.43 (m, 2H), 7.36-7.24 (m, 1H), 7.13 (t, J=8.0 Hz, 1H), 6.84-6.61 (m, 2H), 5.20 (d, J=16.1 Hz, 1H), 4.94 (d, J=16.1 Hz, 1H), 3.85 (s, 3H), 3.49 (d, J=13.9 Hz, 1H), 2.66 (d, J=13.9 Hz, 1H), 2.13 (s, 3H).

### Example 42. Methyl 5-hydroxy-1-(4-(2-methoxy-2-oxoethyl) benzyl)-2-oxo-2,3-dihydro-1H-benzo[b]azepine-4-carboxylate (42)

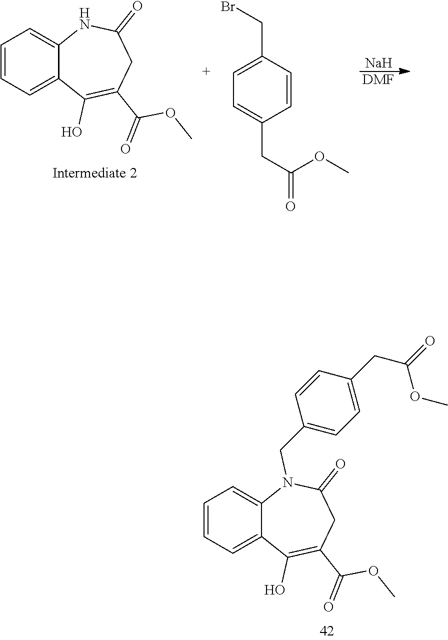

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added K₂CO₃ (20 mg, 0.3 mmol, 3 equiv). To this solution was added methyl 2-(4-(bromomethyl) phenyl) acetate (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 11 mg of the white solid compound with a yield of 40%, purity=96%.

TLC R_{f}=0.5 (PE:EA=1:1)

LCMS (m/z)=396.3 (M+H).

### Example 43. Methyl 1-(4-acetylbenzyl)-5-hydroxy-2-oxo-2,3-dihydro-1H-benzo[b]azepine-4-carboxylate (43)

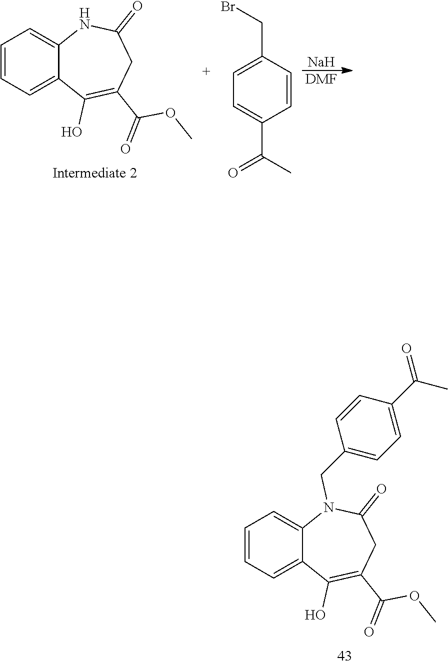

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(4-(bromomethyl) phenyl) ethan-1-one (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 5 mg of the white solid compound with a yield of 14%, purity=95%.

TLC R_{f}=0.3 (PE:EA=1:1)

LCMS (m/z)=366.2 (M+H).

### Example 44. Methyl 5-hydroxy-1-(4-(methylsulfonyl) benzyl)-2-oxo-2,3-dihydro-1H-benzo[b]azepine-4-carboxylate (44)

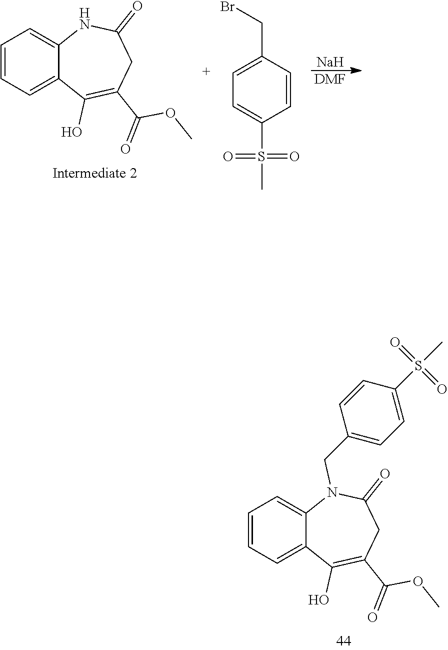

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-(methylsulfonyl) benzene (0.12 mmol, 1.2 equiv), and the reaction mixture was allowed to stir at rt for 4 h. The reaction progress was monitored by TLC until the complete disappearance of Intermediate 2. The resulting solution was poured into water (30 ml) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 15 mg of the white solid compound with a yield of 37%, purity=75%.

TLC R_{f}=0.2 (PE:EA=1:1)

LCMS (m/z)=402.2 (M+H).

### Example 45. Methyl 1-(3-chloro-4-methylbenzyl)-5-hydroxy-2-oxo-2,3-dihydro-1H-benzo[b]azepine-4-carboxylate (45)

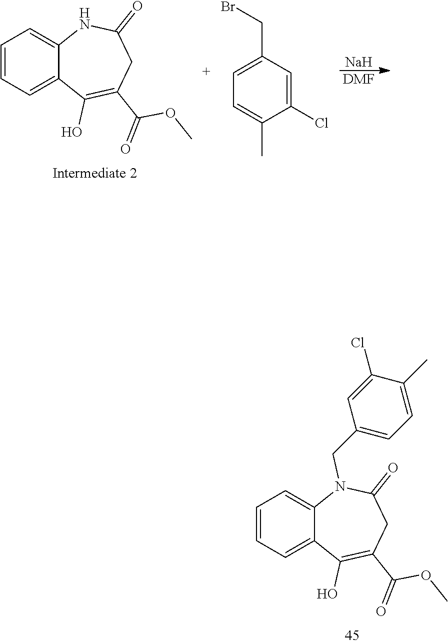

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-2-chloro-1-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 46 mg of the white solid compound with a yield of 90%, purity=94%.

TLC R_{f}=0.5 (PE:EA=2:1)

LCMS (m/z)=372.4 (M+H).

### Example 46. Methyl 1-(3-bromo-4-methylbenzyl)-5-hydroxy-2-oxo-2,3-dihydro-1H-benzo[b]azepine-4-carboxylate (46)

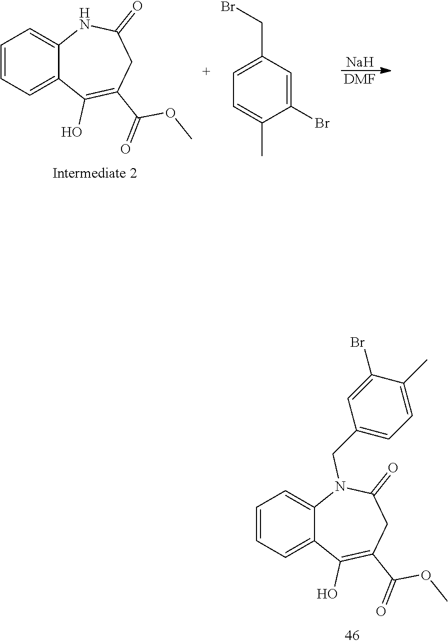

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 2-bromo-4-(bromomethyl)-1-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 ml) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 24 mg of the white solid compound with a yield of 70%, purity=93%.

TLC R_{f}=0.5 (PE:EA=2:1)

LCMS (m/z)=418.0 (M+H).

### Example 47. Methyl 5-hydroxy-1-(3-methoxy-4-methylbenzyl)-2-oxo-2,3-dihydro-1H-benzo[b]azepine-4-carboxylate (47)

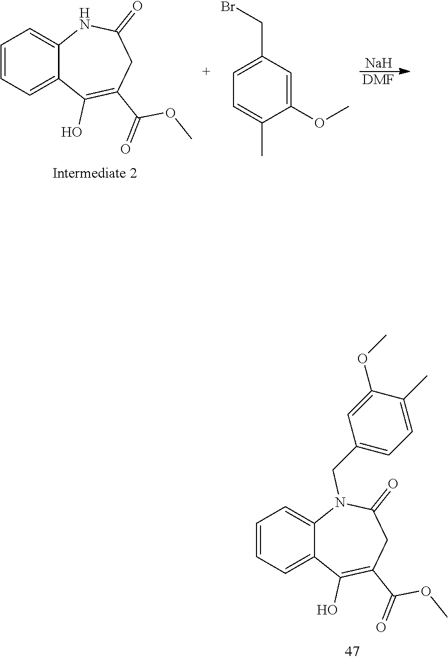

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-2-methoxy-1-methylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brie (1×40 mL), dried over anhydrous Na₂SO₄, and concentrated by rotary evaporation. Then, the resulting concentrate was purified on a silica column to get 7 mg of the white solid compound with a yield of 11%, purity=93%.

TLC R_{f}=0.5 (PE:EA=2:1)

LCMS (m/z)=368.60 (M+H).

### Example 48. Methyl 1-(3-fluoro-4-methoxybenzyl)-5-hydroxy-2-oxo-2,3-dihydro-1H-benzo[b]azepine-4-carboxylate (48)

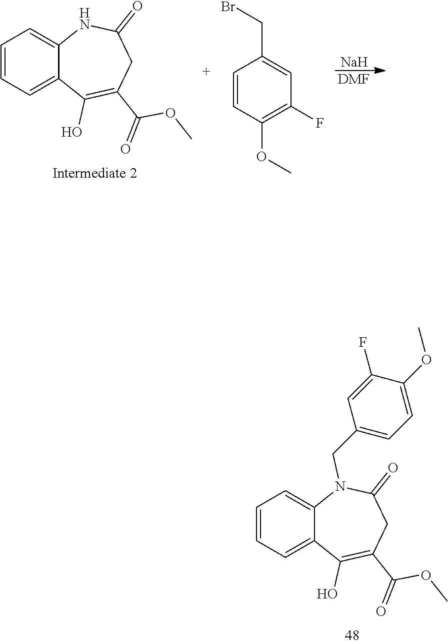

To a solution of methyl 5-hydroxy-2-oxo-2,3-dihydro-1H-benzo[b]azepine-4-carboxy late (Intermediate 2) (2300 mg, 10 mmol, 1 equiv) in DMF (60 mL) was added NaH 60% dispersion in mineral oil (1200 mg, 30 mmol, 3 equiv). To this solution was added 4-(bromomethyl)-2-fluoro-1-methoxybenzene (12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 820 mg of the white solid compound with a yield of 37.7%, purity=94%.

TLC R_{f}=0.4 (PE:EA=2:1)

LCMS (m/z)=372.40 (M+H).

¹H NMR (400 MHZ, DMSO) δ 12.28 (s, 1H), 7.73 (d, J=7.5 Hz, 1H), 7.60-7.47 (m, 2H), 7.30 (ddd, J=8.2, 6.3, 2.1 Hz, 1H), 7.01 (t, J=8.7 Hz, 1H), 6.90-6.71 (m, 2H), 5.19 (d, J=15.9 Hz, 1H), 4.89 (d, J=15.9 Hz, 1H), 3.85 (s, 3H), 3.75 (s, 3H), 3.49 (d, J=13.9 Hz, 1H), 2.65 (d, J=13.9 Hz, 1H). ¹³C NMR (100 MHZ, DMSO) δ 170.44, 170.22, 165.57, 152.39, 149.96, 145.98, 145.87, 140.50, 131.82, 130.28, 130.23, 128.22, 127.75, 125.15, 123.23, 122.90, 122.87, 114.38, 114.20, 113.69, 97.75, 55.83, 52.56, 48.97, 31.10.

### Example 49. Methyl 5-hydroxy-2-oxo-1-(4-propylbenzyl)-2,3-dihydro-1H-benzo[b]azepine-4-carboxylate (49)

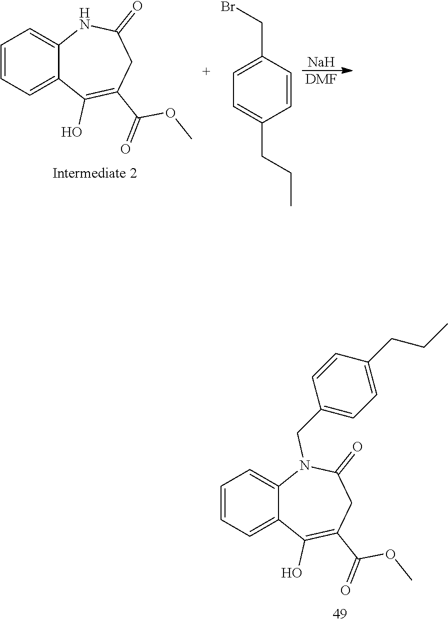

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-propylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 20 mg of the white solid compound with a yield of 75%, purity-70%.

TLC R_{f}=0.6 (PE:EA=2:1)

LCMS (m/z)=366.50 (M+H).

### Example 50. Methyl 1-(4-butylbenzyl)-5-hydroxy-2-oxo-2,3-dihydro-1H-benzo[b]azepine-4-carboxylate (50)

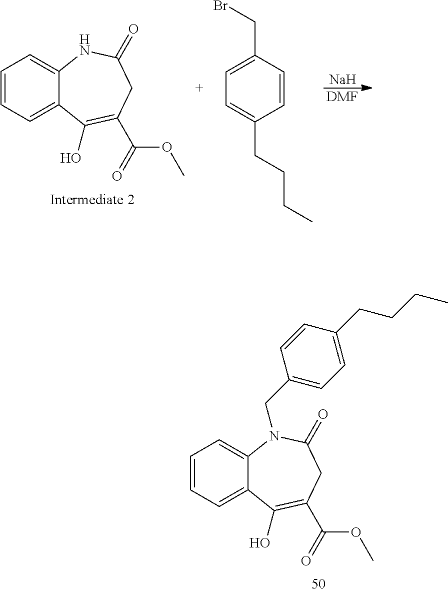

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 1-(bromomethyl)-4-butylbenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 ml) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 20 mg of the white solid compound with a yield of 70%, purity=72%.

TLC R_{f}=0.6 (PE:EA=2:1)

LCMS (m/z)=380.50 (M+H).

### Example 51. Methyl 1-(3,4-dimethoxybenzyl)-5-hydroxy-2-oxo-2,3-dihydro-1H-benzo[b]azepine-4-carboxylate (51)

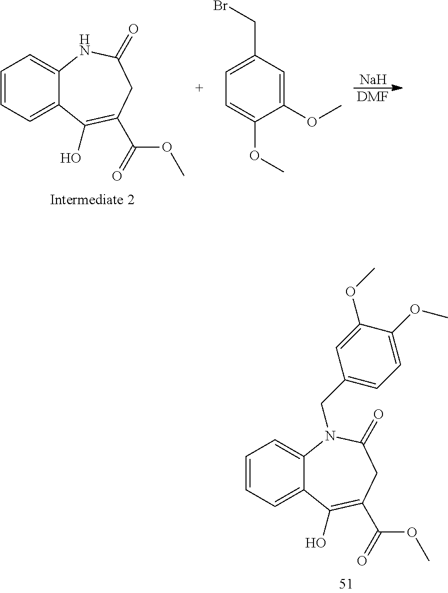

To a solution of methyl 5-hydroxy-2-oxo-2,3-dihydro-1H-benzo[b]azepine-4-carboxylate (Intermediate 2) (23 mg, 0.1 mmol, 1 equiv) in DMF (0.7 mL) was added NaH 60% dispersion in mineral oil (20 mg, 0.3 mmol, 3 equiv). To this solution was added 4-(bromomethyl)-1,2-dimethoxybenzene (0.12 mmol, 1.2 equiv), and the reaction mixture was allowed to stir at rt for 4 h. The reaction progress was monitored by TLC until the complete disappearance of Intermediate 2. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 12 mg of the white solid compound with a yield of 40%, purity=82%.

TLC R_{f}=0.3 (PE:EA=2:1)

LCMS (m/z)=384.00 (M+H).

### Example 52. Methyl 7-fluoro-1-(3-fluoro-4-methylbenzyl)-5-hydroxy-2-oxo-2,3-dihydro-1H-benzo[b]azepine-4-carboxylate (52)

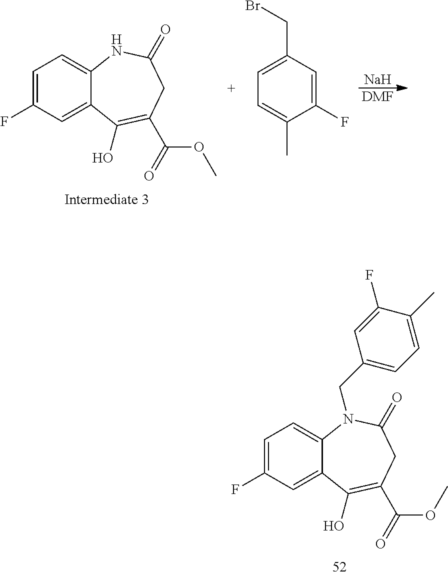

To a solution of methyl 7-fluoro-5-hydroxy-2-oxo-2,3-dihydro-1H-benzo[b]azepine-4-carboxylate (Intermediate 3) (70 mg, 0.3 mmol, 1 equiv) in DMF (0.2 mL) was added NaH 60% dispersion in mineral oil (40 mg, 0.9 mmol, 3 equiv). To this solution was added 4-(bromomethyl)-2-fluoro-1-methylbenzene (0.36 mmol, 1.2 equiv), and the reaction mixture was allowed to stir at rt for 4 h. The reaction progress was monitored by TLC until the complete disappearance of Intermediate 3. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 60 mg of the white solid compound with a yield of 54%, purity=98%.

TLC R_{f}=0.5 (PE:EA=2:1)

LCMS (m/z)=374.60 (M+H).

¹H NMR (400 MHZ, DMSO) δ 12.17 (s, 1H), 7.56 (dd, J=9.1, 4.9 Hz, 1H), 7.50-7.39 (m, 2H), 7.14 (t, J=8.0 Hz, 1H), 6.76 (t, J=8.4 Hz, 2H), 5.21 (d, J=16.1 Hz, 1H), 4.90 (d, J=16.1 Hz, 1H), 3.85 (s, 3H), 3.49 (d, J=14.0 Hz, 1H), 2.69 (d, J=13.9 Hz, 1H), 2.13 (s, 3H). ¹³C NMR (100 MHZ, DMSO) δ 170.35, 170.35, 169.99, 169.99, 164.08, 164.08, 161.71, 161.71, 159.57, 159.57, 159.29, 159.29, 157.15, 157.15, 137.38, 137.31, 137.06, 137.03, 131.63, 131.58, 130.10, 130.02, 125.79, 125.71, 122.81, 122.64, 122.41, 122.38, 119.13, 118.91, 113.72, 113.49, 113.31, 113.09, 98.43, 98.43, 52.65, 52.65, 49.35, 49.35, 31.04, 31.04, 13.73, 13.73, 13.70, 13.70.

### Example 53. Methyl 1-(3-fluoro-4-methylbenzyl)-5-hydroxy-2-oxo-2,3-dihydro-1H-pyrido[4,3-b] azepine-4-carboxylate (53)

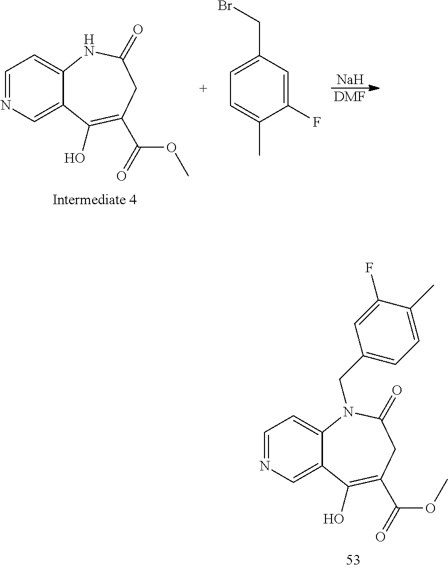

To a solution of methyl 5-hydroxy-2-oxo-2,3-dihydro-1H-pyrido[4,3-b] azepine-4-carboxylate (Intermediate 4) (70 mg, 0.3 mmol, 1 equiv) in DMF (0.2 mL) was added NaH 60% dispersion in mineral oil (40 mg, 0.9 mmol, 3 equiv). To this solution was added 4-(bromomethyl)-2-fluoro-1-methylbenzene (0.36 mmol, 1.2 equiv), and the reaction mixture was allowed to stir at rt for 4 h. The reaction progress was monitored by TLC until the complete disappearance of Intermediate 3. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 10 mg of the white solid compound with a yield of 9%, purity=87%.

TLC R_{f}=0.5 (EA)

LCMS (m/z)=357.60 (M+H).

### Example 54. Methyl 1-(3-fluoro-4-methylbenzyl)-5-hydroxy-7,8-dimethoxy-2-oxo-2,3-dihydro-1H-benzo[b]azepine-4-carboxylate (54)

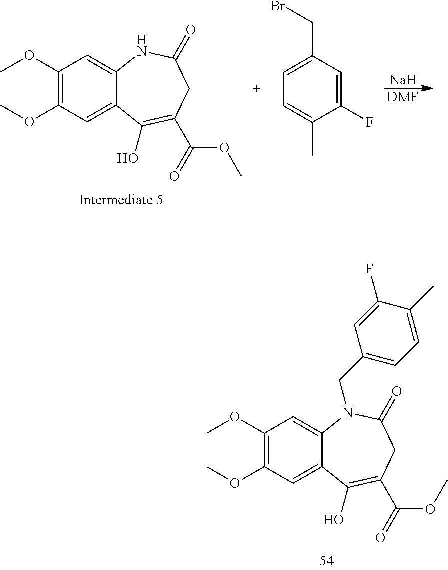

To a solution of methyl 5-hydroxy-7,8-dimethoxy-2-oxo-2,3-dihydro-1H-benzo[b]azepine-4-carboxylate (Intermediate 5) (146 mg, 0.5 mmol, 1 equiv) in DMF (1 mL) was added NaH 60% dispersion in mineral oil (60 mg, 1.5 mmol, 3 equiv). To this solution was added 4-(bromomethyl)-2-fluoro-1-methylbenzene (0.6 mmol, 1.2 equiv), and the reaction mixture was allowed to stir at rt for 4 h. The reaction progress was monitored by TLC until the complete disappearance of Intermediate 5. The resulting solution was poured into water (30 mL) and extracted with Ethyl acetate (3×30 mL). The combined organic layers were washed with brine (1×40 mL), dried over anhydrous Na₂SO₄, and concentrated by rotary evaporation. Then, the resulting concentrate was purified on a silica column to get 150 mg of the white solid compound with a yield of 72%, purity=98%.

TLC R_{f}=0.5 (PE:EA=2:1)

LCMS (m/z)=416.60 (M+H).

¹H NMR (400 MHZ, DMSO) δ 12.36 (s, 1H), 7.18-7.09 (m, 2H), 7.07 (s, 1H), 6.75 (d, J=9.4 Hz, 2H), 5.30 (d, J=15.8 Hz, 1H), 4.89 (d, J=15.8 Hz, 1H), 3.84 (s, 3H), 3.77 (d, J=2.1 Hz, 6H), 3.47 (d, J=13.7 Hz, 1H), 2.62 (d, J=13.7 Hz, 1H), 2.12 (s, 3H). ¹³C NMR (100 MHZ, DMSO) & 170.49, 170.26, 166.02, 161.64, 159.22, 151.24, 145.78, 137.77, 137.70, 134.73, 131.53, 131.48, 122.74, 122.67, 122.64, 122.57, 120.63, 113.45, 113.23, 108.66, 106.83, 96.24, 55.82, 55.49, 52.45, 49.02, 31.20, 13.74, 13.71.

### Intermediate 6. 1-(3-fluoro-4-methylbenzyl)-3,4-dihydro-1H-benzo[b]azepine-2,5-dione

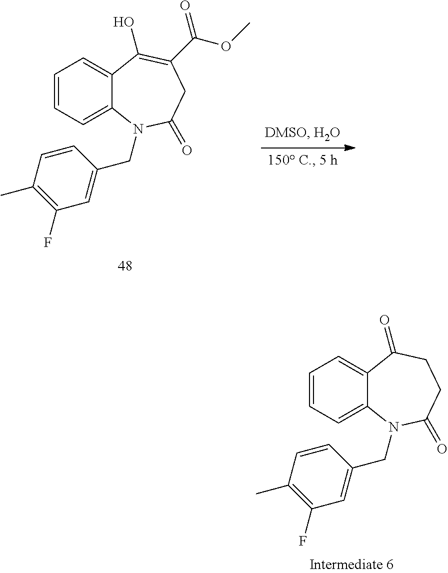

To a solution of methyl 1-(3-fluoro-4-methylbenzyl)-5-hydroxy-2-oxo-2,3-dihydro-1H-benzo[b]azepine-4-carboxylate 48 (800 mg, 2.254 mmol, 1 equiv) in DMSO (6.33 mL), was added H₂O (0.18 mL) under Nitrogen, and the reaction mixture was allowed to stir at 150° C. for 5 h. The reaction progress was monitored by TLC until the complete disappearance of compound 48. The reaction mixture was allowed to cool to rt, ice (11.30 mL) was added, and the mixture was allowed to stir 12 h. To the flask was added 1N HCl (11.30 mL) at 0° C. and the mixture allowed to stir for 3 h. The resulting precipitate was filtered. Then, the crude product was purified on a silica column (The height and diameter of the silica gel column is 10 and 2 cm, respectively; silica gel: 200-300 mesh, eluent: EA/PE=1/2, flow rate: 50 mL/min, collections at 11-14 min elution time contained the target compound) to get 580 mg (purity=80%) light yellow oil compound with a yield of 74%.

LCMS (FA) m/z=298.60 (M+H)

### Example 55. 1-(3-fluoro-4-methylbenzyl)-5-hydroxy-4-propionyl-1,3-dihydro-2H-benzo[b]azepin-2-one (55)

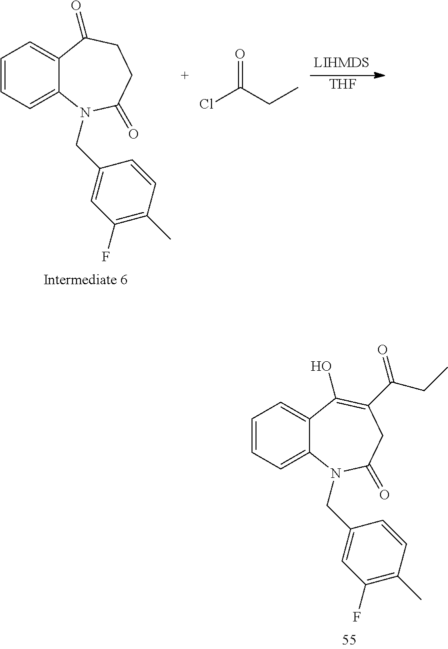

The starting ketone (Intermediate 6) (50 mg, 0.16 mmol, 1 equiv) was dissolved in THE, cooled to 0° C., and lithium hexamethyldisilzeide (LIHMDS, 1 M in THF) (320 μL, 0.32 mmol, 2 equiv) added dropwise via syringe. The reaction mixture was then stirred for approximately 30 minutes, after which time the corresponding acid chloride (18 μL, 0.192 mmol, 1.2 equiv) was added. The resulting mixture was stirred at 0° C. and then allowed slowly to room temperature overnight. The solution was quenched with saturated ammonium chloride and extracted with EA (3×10 mL). The organic solutions were combined, washed with H₂O (2×10 mL) and brine (1×20 ml), dried over anhydrous Na₂SO₄, filtered and concentrated by rotary evaporation to get the crude compound. Then, the resulting concentrate was purified by pre-TLC get 13 mg (purity=95%) light yellow solid compound with a yield of 23%.

TLC R_{f}=0.5 (PE:EA=2:1)

LCMS (FA) m/z=354.60 (M+H)

### Example 56. 4-acetyl-1-(3-fluoro-4-methylbenzyl)-5-hydroxy-1,3-dihydro-2H-benzo[b]azepin-2-one (56)

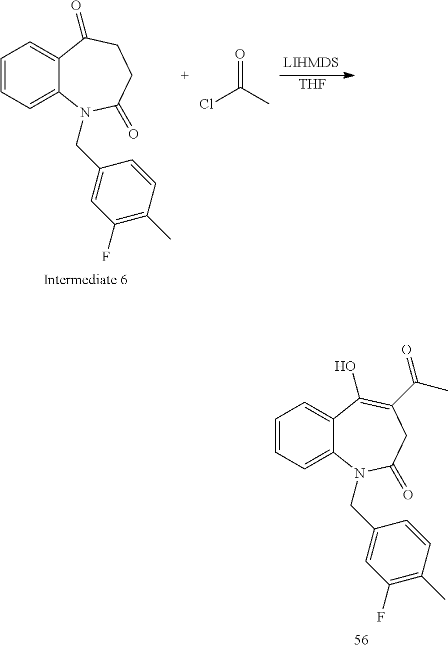

The starting ketone (Intermediate 6) (50 mg, 0.16 mmol, 1 equiv) was dissolved in THE, cooled to 0° C., and lithium hexamethyldisilzeide (LIHMDS, 1 M in THF) (320 μL, 0.32 mmol, 2 equiv) added dropwise via syringe. The reaction mixture was then stirred for approximately 30 minutes, after which time the corresponding acid chloride (18 μL, 0.192 mmol, 1.2 equiv) was added. The resulting mixture was stirred at 0° C.′ and then allowed slowly to room temperature overnight. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 4 mg (purity=99%) light yellow solid compound with a yield of 7%.

TLC R_{f}=0.5 (PE:EA=2:1)

LCMS (FA) m/z=340.60 (M+H)

### Example 57. 4-butyryl-1-(3-fluoro-4-methylbenzyl)-5-hydroxy-1,3-dihydro-2H-benzo[b]azepin-2-one (57)

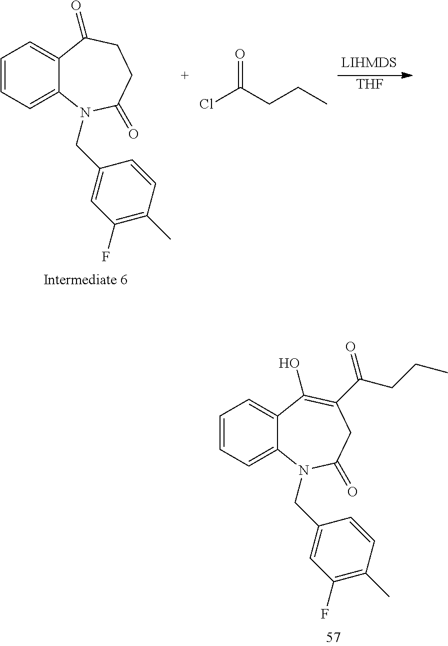

The starting ketone (Intermediate 6) (200 mg, 0.67 mmol, 1 equiv) was dissolved in THE, cooled to 0° C., and lithium bexamethyldisilzeide (LIHMDS, 1 M in THF) (1.34 mL, 1.34 mmol, 2 equiv) added dropwise via syringe. The reaction mixture was then stirred for approximately 30 minutes, after which time the corresponding acid chloride (84 μL, 0.8 mmol, 1.2 equiv) was added. The resulting mixture was stirred at 0° C. and then allowed slowly to room temperature overnight. The solution was quenched with saturated ammonium chloride and extracted with EA (3×10 mL). The organic solutions were combined, washed with H₂O (2×10 ml) and brine (1×20 mL), dried over anhydrous Na₂SO₄, filtered and concentrated by rotary evaporation to get the crude compound. Then, the resulting concentrate was purified by pre-TLC get 50 mg (purity=97%) light yellow solid compound with a yield of 20%.

TLC R_{f}=0.5 (PE:EA=2:1)

LCMS (FA) m/z=368.40 (M+H)

### Example 58. 4-(cyclopropanecarbonyl)-1-(3-fluoro-4-methylbenzyl)-5-hydroxy-1,3-dihydro-2H-benzo[b]azepin-2-one (58)

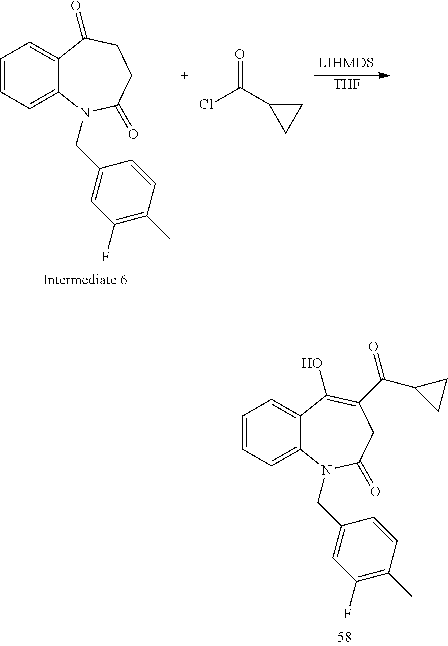 [SMILES: Cc1ccc(CN2C(=O)CCC(=O)c3ccccc32)cc1F ; O=C(Cl)C1CC1 ; Cc1ccc(CN2C(=O)CC(C(=O)C3CC3)=C(O)c3ccccc32)cc1F ; "THF"]

The starting ketone (Intermediate 6) (148 mg, 0.5 mmol, 1 equiv) was dissolved in THE, cooled to 0° C., and lithium hexamethyldisilzeide (LIHMDS, 1 M in THF) (1 mL, 1 mmol, 2 equiv) added dropwise via syringe. The reaction mixture was then stirred for approximately 30 minutes, after which time the corresponding acid chloride (54 μL, 0.6 mmol, 1.2 equiv) was added. The resulting mixture was stirred at 0° C. and then allowed slowly to room temperature overnight. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 4 mg (purity=95%) light yellow solid compound with a yield of 27%.

TLC R_{f}=0.5 (PE:EA=2:1)

LCMS (FA) m/z=366.70 (M+H)

¹H NMR (400 MHz, DMSO) δ 16.10 (s, 1H), 7.76 (dd, J=7.8, 1.2 Hz, 1H), 7.62-7.45 (m, 2H), 7.33 (t, J=7.3 Hz, 1H), 7.14 (t, J=8.0 Hz, 1H), 6.79 (t, J=7.6 Hz, 2H), 5.22 (d, J=16.1 Hz, 1H), 4.99 (d, J=16.1 Hz, 1H), 3.73 (d, J=14.1 Hz, 1H), 2.92 (d, J=14.2 Hz, 1H), 2.14 (s, 3H), 1.30-1.02 (m, 4H). ¹³C NMR (100 MHz, DMSO) δ 200.96, 172.43, 171.09, 162.23, 159.81, 141.19, 138.10, 138.03, 132.85, 132.06, 129.48, 129.07, 125.81, 123.55, 123.25, 123.08, 122.80, 113.72, 113.49, 107.65, 49.69, 32.58, 16.77, 14.23, 14.20, 13.10, 11.72.

### Example 59. 1-(3-fluoro-4-methylbenzyl)-5-hydroxy-4-isobutyryl-1,3-dihydro-2H-benzo[b]azepin-2-one (59)

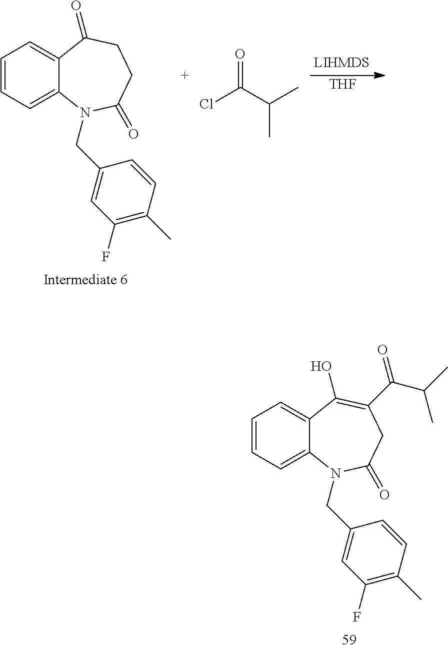

The starting ketone (Intermediate 6) (148 mg, 0.5 mmol, 1 equiv) was dissolved in THE, cooled to 0° C., and lithium bexamethyldisilzeide (LIHMDS, 1 M in THF) (1 mL, 1 mmol, 2 equiv) added dropwise via syringe. The reaction mixture was then stirred for approximately 30 minutes, after which time the corresponding acid chloride (63 μL, 0.6 mmol, 1.2 equiv) was added. The resulting mixture was stirred at 0° C. and then allowed slowly to room temperature overnight. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 12 mg (purity=90%) light yellow solid compound with a yield of 6%.

TLC R_{f}=0.5 (PE:EA=2:1)

LCMS (FA) m/z=368.60 (M+H)

### Example 60. 1-(3-fluoro-4-methylbenzyl)-5-hydroxy-4-pivaloyl-1,3-dihydro-2H-benzo[b]azepin-2-one (60)

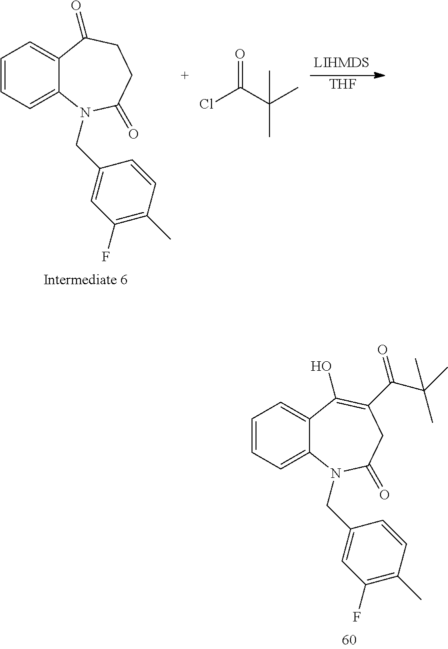

The starting ketone (Intermediate 6) (148 mg, 0.5 mmol, 1 equiv) was dissolved in THF, cooled to 0° C., and lithium hexamethyldisilzeide (LIHMDS, 1 M in THF) (1 mL, 1 mmol, 2 equiv) added dropwise via syringe. The reaction mixture was then stirred for approximately 30 minutes, after which time the corresponding acid chloride (73 μL, 0.6 mmol, 1.2 equiv) was added. The resulting mixture was stirred at 0° C. and then allowed slowly to room temperature overnight. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 10 mg (purity=90%) light yellow solid compound with a yield of 5%.

TLC R_{f}=0.5 (PE:EA=2:1)

LCMS (FA) m/z=382.40 (M+H)

### Example 61. 1-(3-fluoro-4-methylbenzyl)-5-hydroxy-4-(2-methoxyacetyl)-1,3-dihydro-2H-benzo[b]azepin-2-one (61)

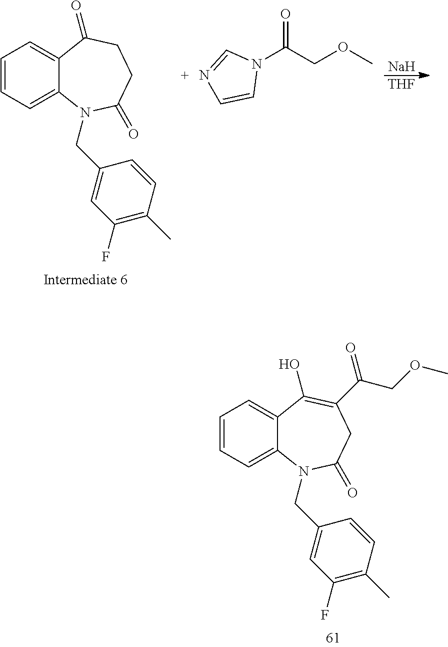

To a stirred solution of ketone (Intermediate 6) (136 mg, 0.458 mmol, 1 equiv) and 1-(1H-imidazol-1-yl)-2-methoxyethan-1-one (77 mg, 0.550 mmol, 1.2 equiv) in THF (2 mL), was added NaH 60% dispersion in mineral oil (110 mg, 4.58 mmol, 10 equiv) at ambient temperature under N₂ atmosphere. The reaction mixture was then stirred at r.t for 1 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by Pre-TLC to get 40 mg (purity=96%) light yellow solid compound with a yield of 24%.

TLC (EA:PE=1:2) Rf=0.3

LCMS (FA) m/z=370.50 (M+H)

¹H NMR (400 MHZ, DMSO) δ 14.54 (s, 1H), 7.73 (dd, J=7.8, 1.3 Hz, 1H), 7.55 (ddd, J=17.3, 12.1, 4.5 Hz, 2H), 7.36-7.28 (m, 1H), 7.13 (t, J=8.0 Hz, 1H), 6.76 (dd, J=8.8, 4.2 Hz, 2H), 5.23 (d, J=16.1 Hz, 1H), 4.93 (d, J=16.0 Hz, 1H), 4.63 (d, J=17.4 Hz, 1H), 4.41 (d, J=17.4 Hz, 1H), 3.42 (s, 1H), 3.37 (s, 3H), 2.70 (d, J=14.1 Hz, 1H), 2.12 (s, 3H).

### Example 62. 1-(3-fluoro-4-methylbenzyl)-5-hydroxy-4-(tetrahydrofuran-2-carbonyl)-1,3-dihydro-2H-benzo[b]azepin-2-one (62)

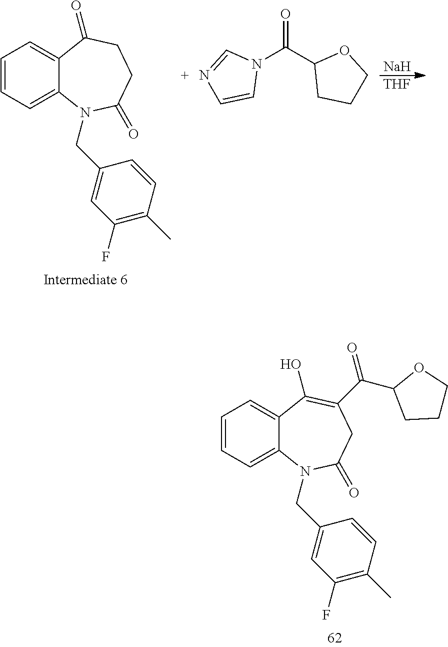

To a stirred solution of ketone (Intermediate 6) (100 mg, 0.337 mmol, 1 equiv) and (1H-imidazol-1-yl)(tetrahydrofuran-2-yl) methanone (67 mg, 0.404 mmol, 1.2 equiv) in THF (2 mL), was added NaH 60% dispersion in mineral oil (80 mg, 3.33 mmol, 10 equiv) at ambient temperature under N₂ atmosphere. The reaction mixture was then stirred at r.t for 1 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by Pre-TLC to get 11 mg (purity=99%) light yellow solid compound with a yield of 8%.

TLC (EA:PE=1:2) Rf=0.5

LCMS (FA) m/z=396.60 (M+H)

### Example 63. 1-(3-fluoro-4-methylbenzyl)-5-hydroxy-N-methyl-2-oxo-2,3-dihydro-1H-benzo[b]azepine-4-carboxamide (63)

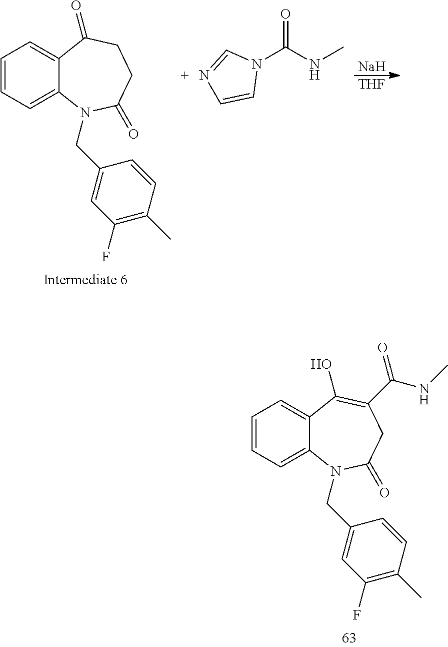

To a stirred solution of ketone (Intermediate 6) (118 mg, 0.4 mmol, 1 equiv) and N-methyl-1H-imidazole-1-carboxamide (150 mg, 1.2 mmol, 3 equiv) in THF (4 mL), was added NaH 60% dispersion in mineral oil (160 mg, 4 mmol, 10 equiv) at ambient temperature under N₂ atmosphere. The reaction mixture was then stirred at RT for 4 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 95 mg (purity=98%) light yellow solid compound with a yield of 65%.

TLC R_{f}=0.3 (PE:EA=2:1)

LCMS (FA) m z=355.50 (M+H)

¹H NMR (400 MHZ, DMSO) δ 15.25 (s, 1H), 8.41 (s, 1H), 7.71 (d, J=7.8 Hz, 1H), 7.48 (d, J=3.6 Hz, 2H), 7.33-7.22 (m, 1H), 7.14 (t, J=7.9 Hz, 1H), 6.83-6.72 (m, 2H), 5.22 (d, J=16.1 Hz, 1H), 4.95 (d, J=16.2 Hz, 1H), 3.53 (d, J=14.3 Hz, 1H), 2.77 (d, J=3.4 Hz, 3H), 2.60 (d, J=14.3 Hz, 1H), 2.14 (s, 3H). ¹³C NMR (100 MHz, DMSO) δ 170.76, 170.72, 164.50, 162.22, 159.80, 140.49, 138.22, 138.15, 132.06, 132.01, 131.40, 129.91, 128.13, 125.45, 123.15, 122.99, 122.75, 113.64, 113.42, 99.07, 49.62, 30.87, 26.64, 14.22, 14.19.

### Example 64. N-ethyl-1-(3-fluoro-4-methylbenzyl)-5-hydroxy-2-oxo-2,3-dihydro-1H-benzo[b]azepine-4-carboxamide (64)

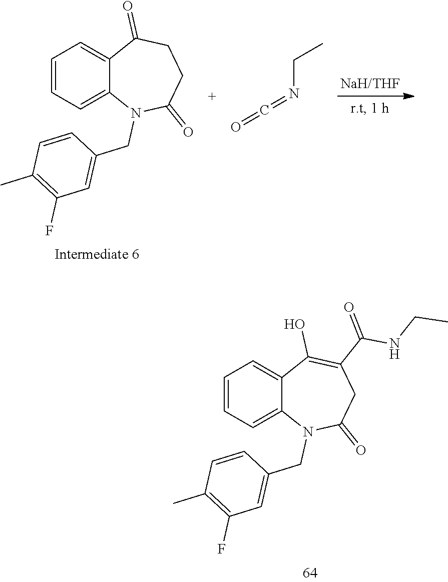

The starting ketone (Intermediate 6) (50 mg, 0.168 mmol, 1 equiv) was dissolved in THE, and NaH 60% dispersion in mineral oil (40 mg, 1.67 mmol, 10 equiv) added. The reaction mixture was then stirred for approximately 5 minutes, after which time the isocyanatoethane (17 μL, 0.211 mmol, 1.2 equiv) was added under Nitrogen, and the reaction mixture was allowed to stir at r.t for 1 h. The reaction progress was monitored by TLC until the complete disappearance of Intermediate 6. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 18 mg (purity=85%) light yellow solid compound with a yield of 29%.

TLC (EA:PE=1:2) Rf=0.45

LCMS (FA) m/z=369.50 (M+H)

### Example 65. 1-(3-fluoro-4-methylbenzyl)-5-hydroxy-N-isopropyl-2-oxo-2,3-dihydro-1H-benzo[b]azepine-4-carboxamide (65)

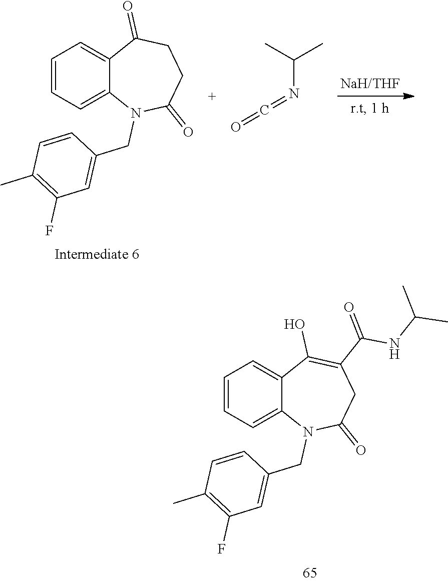

The starting ketone (Intermediate 6) (50 mg, 0.168 mmol, 1 equiv) was dissolved in THE, and NaH 60% dispersion in mineral oil (40 mg, 1.67 mmol, 10 equiv) added. The reaction mixture was then stirred for approximately 5 minutes, after which time the 2-isocyanatopropane (21 μL, 0.211 mmol, 1.2 equiv) was added under Nitrogen, and the reaction mixture was allowed to stir at r.t for 1 h. The reaction progress was monitored by TLC until the complete disappearance of compound Intermediate 6. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 33 mg (purity=98%) light yellow solid compound with a yield of 51%.

TLC (EA:PE=1:2) Rf=0.6

LCMS (FA) m/z=383.50 (M+H)

¹H NMR (400 MHZ, DMSO) δ 15.35 (s, 1H), 8.19 (s, 1H), 7.70 (d, J=7.6 Hz, 1H), 7.45 (s, 2H), 7.28-7.18 (m, 1H), 7.14 (t, J=7.9 Hz, 1H), 6.77 (d, J=8.6 Hz, 2H), 5.19 (d, J=16.1 Hz, 1H), 4.97 (d, J=16.2 Hz, 1H), 3.71 (d, J=14.2 Hz, 1H), 2.55 (s, 1H), 2.14 (s, 3H), 1.39-0.90 (m, 6H). ¹³C NMR (101 MHz, DMSO) δ 170.96, 169.46, 162.22, 159.81, 140.42, 138.25, 132.05, 132.00, 131.18, 128.19, 125.34, 123.12, 122.95, 122.72, 113.64, 113.41, 98.97, 49.57, 41.50, 30.64, 22.64, 22.56, 14.23, 14.20.

### Example 66. N-cyclohexyl-1-(3-fluoro-4-methylbenzyl)-5-hydroxy-2-oxo-2,3-dihydro-1H-benzo[b]azepine-4-carboxamide (66)

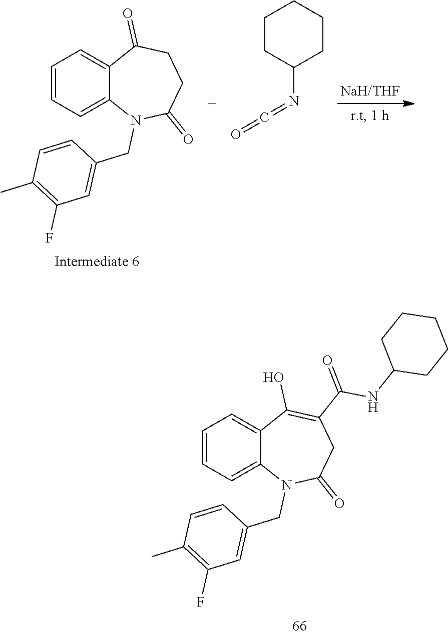 [SMILES: Cc1ccc(CN2C(=O)CCC(=O)c3ccccc32)cc1F ; O=C=NC1CCCCC1 ; Cc1ccc(CN2C(=O)CC(C(=O)NC3CCCCC3)=C(O)c3ccccc32)cc1F]

The starting ketone (Intermediate 6) (50 mg, 0.168 mmol, 1 equiv) was dissolved in THE, and NaH 60% dispersion in mineral oil (40 mg, 1.67 mmol, 10 equiv) added. The reaction mixture was then stirred for approximately 5 minutes, after which time the isocyanatocyclohexane (21 uL, 0.211 mmol, 1.2 equiv) was added under Nitrogen, and the reaction mixture was allowed to stir at r.t for 1 h. The reaction progress was monitored by TLC until the complete disappearance of compound Intermediate 6. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 33 mg (purity=98%) light yellow solid compound with a yield of 51%.

TLC (EA:PE=1:2) Rf=0.6

LCMS (FA) m/z=423.60 (M+H)

### Example 67. 1-(3-fluoro-4-methylbenzyl)-5-hydroxy-N-methyl-2-oxo-2,3-dihydro-1H-benzo[b]azepine-4-carbothioamide (67)

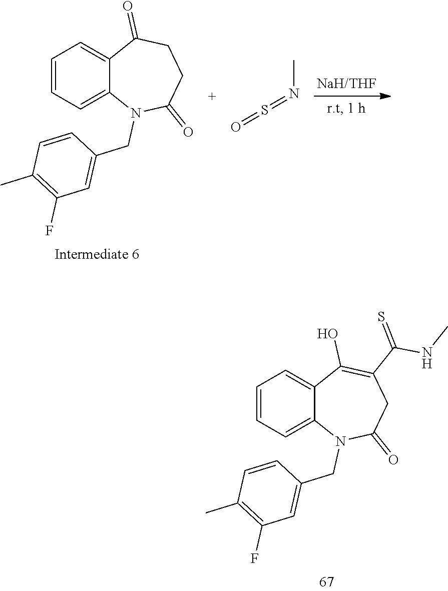

The starting ketone (Intermediate 6) (50 mg, 0.168 mmol, 1 equiv) was dissolved in THE, and NaH 60% dispersion in mineral oil (40 mg, 1.67 mmol, 10 equiv) added. The reaction mixture was then stirred for approximately 5 minutes, after which time the isothiocyanatomethane (30 mg, 0.411 mmol, 1.2 equiv) was added under Nitrogen, and the reaction mixture was allowed to stir at r.t for 1 h. The reaction progress was monitored by TLC until the complete disappearance of compound Intermediate 6. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by pre-TLC get 35 mg (purity=88%) light yellow solid compound with a yield of 28%.

TLC (MeOH:DCM=1:10) Rf=0.7

LCMS (FA) m/z=371.40 (M+H)

### Example 68. 1-(3-fluoro-4-methylbenzyl)-5-hydroxy-4-(2,2,2-trifluoroacetyl)-1,3-dihydro-2H-benzo[b]azepin-2-one (68)

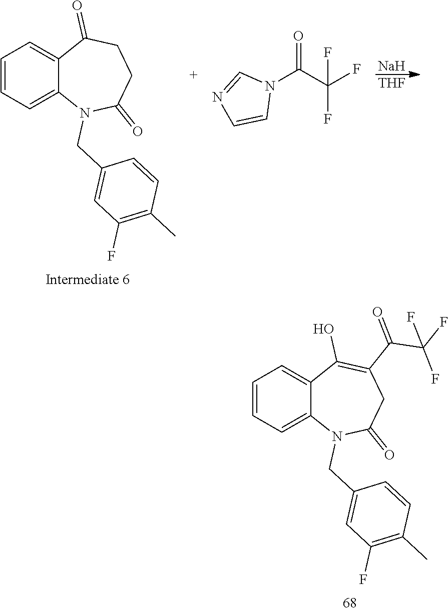

To a stirred solution of ketone (Intermediate 6) (200 mg, 0.673 mmol, 1 equiv) and 2,2,2-trifluoro-1-(1H-imidazol-1-yl)ethan-1-one (132 mg, 0.805 mmol, 1.2 equiv) in THF (4 mL), was added NaH 60% dispersion in mineral oil (160 mg, 6.67 mmol, 10 equiv) at ambient temperature under N₂ atmosphere. The reaction mixture was then stirred at rt for 1 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified by Pre-TLC to get 151 mg (purity=93%) light yellow solid compound with a yield of 57%.

TLC (EA:PE=1:2) Rf=0.3

LCMS (FA) m/z=394.50 (M+H)

¹H NMR (400 MHZ, DMSO) δ 13.54 (s, 1H), 7.73 (d, J=7.7 Hz, 1H), 7.69-7.52 (m, 2H), 7.35 (t, J=7.3 Hz, 1H), 7.13 (t, J=8.0 Hz, 1H), 6.80 (dd, J=8.1, 5.5 Hz, 2H), 5.29 (d, J=16.0 Hz, 1H), 4.93 (d, J=16.0 Hz, 1H), 3.48 (d, J=14.1 Hz, 1H), 2.88 (d, J=14.1 Hz, 1H), 2.13 (s, 3H). ¹³C NMR (100 MHz, DMSO) δ 176.84, 176.49, 173.46, 170.04, 161.76, 159.34, 140.94, 137.39, 137.32, 133.27, 131.59, 131.53, 129.05, 128.68, 125.32, 123.36, 122.83, 122.66, 122.44, 122.41, 121.17, 118.31, 115.44, 113.68, 113.29, 113.07, 104.56, 48.76, 31.35, 13.72, 13.69.

### Example 69. 1-(3-fluoro-4-methylbenzyl)-5-hydroxy-N,N-dimethyl-2-oxo-2,3-dihydro-1H-benzo[b]azepine-4-carboxamide (69)

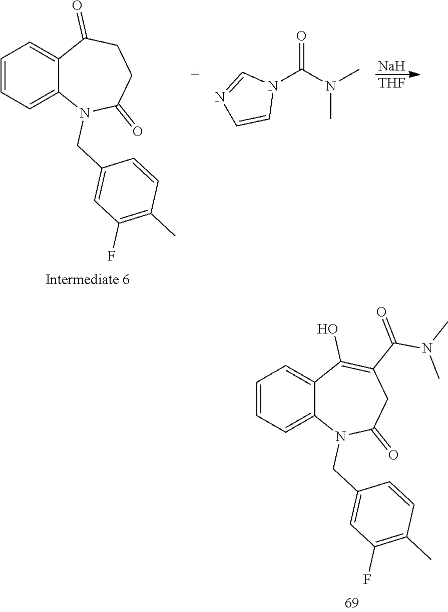

To a stirred solution of ketone (Intermediate 6) (297 mg, 1 mmol, 1 equiv) and N,N-dimethyl-1H-imidazole-1-carboxamide (417 mg, 3 mmol, 3 equiv) in THE (7 mL), was added NaH 60% dispersion in mineral oil (400 mg, 10 mmol, 10 equiv) at ambient temperature under N₂ atmosphere. The reaction mixture was then stirred at 60° C. for 0.5 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 57 mg (purity=99%) light yellow oil compound with a yield of 15%.

TLC R_{f}=0.3 (PE:EA=1:1)

LCMS (FA) m/z=369.50 (M+H)

### Example 70. 1-(cyclopropylmethyl)-5-hydroxy-N-methyl-2-oxo-2,3-dihydro-1H-benzo[b]azepine-4-carboxamide (70)

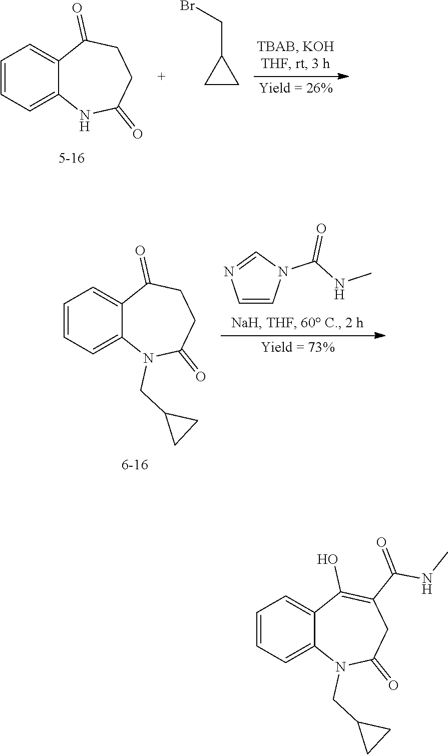

To a stirred solution of ketone (5-16) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added (bromomethyl)cyclopropane (162 mg, 1.2 mmol, 1.2 equiv) at ambient temperature. The reaction mixture was then stirred at RT for 3 h. The reaction progress was monitored by TLC until the complete disappearance of compound 5-16. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 59 mg (purity=99%) light yellow solid compound 6-16 with a yield of 26%.

TLC R_{f}=0.45 (PE:EA=1:1)

LCMS (m/z)=230.40 (M+H)

To a stirred solution of ketone (6-16) (59 mg, 0.258 mmol, 1 equiv) and N-methyl-1H-imidazole-1-carboxamide (97 mg, 0.776 mmol, 3 equiv) in THF (2 mL), was added NaH 60% dispersion in mineral oil (103 mg, 2.58 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 54 mg (purity=95%) white solid compound 70 with a yield of 73%.

TLC R_{f}=0.45 (PE:EA=1:1)

LCMS (m/z)=287.40 (M+H)

### Example 71. 5-hydroxy-N-methyl-2-oxo-1-(thiophen-3-ylmethyl)-2,3-dihydro-1H-benzo[b]azepine-4-carboxamide (71)

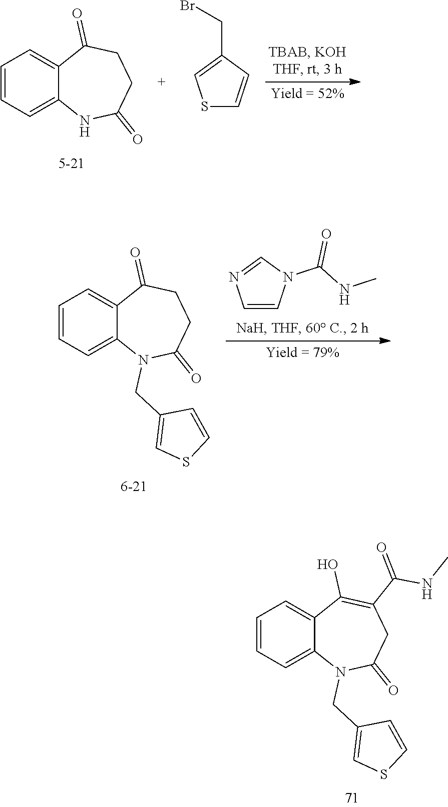

To a stirred solution of ketone (5-21) (175 mg, 1 mmol, 1 equiv), Tetrabutylammonium bromide (33 mg, 0.1 mmol, 0.1 equiv) and KOH (79 mg, 1.6 mmol, 1.4 equiv) in THF (4 mL), was added 3-(bromomethyl)thiophene (213 mg, 1.2 mmol, 1.2 equiv) at ambient temperature. The reaction mixture was then stirred at RT for 3 h. The reaction progress was monitored by TLC until the complete disappearance of compound 5-21. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 142 mg (purity=98%) light yellow solid compound 6-21 with a yield of 52%.

TLC R_{f}=0.5 (PE:EA=1:1)

LCMS (m/z)=272.50 (M+H)

To a stirred solution of ketone (6-21) (142 mg, 0.524 mmol, 1 equiv) and N-methyl-1H-imidazole-1-carboxamide (196 mg, 1.57 mmol, 3 equiv) in THE (3 mL), was added NaH 60% dispersion in mineral oil (210 mg, 5.24 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 137 mg (purity=90%) white solid compound 71 with a yield of 79%.

TLC R_{f}=0.45 (PE:EA=1:1)

LCMS (m/z)=329.60 (M+H)

### Example 72. 1-benzyl-5-hydroxy-N-methyl-2-oxo-2,3-dihydro-1H-benzo[b]azepine-4-carboxamide (72)

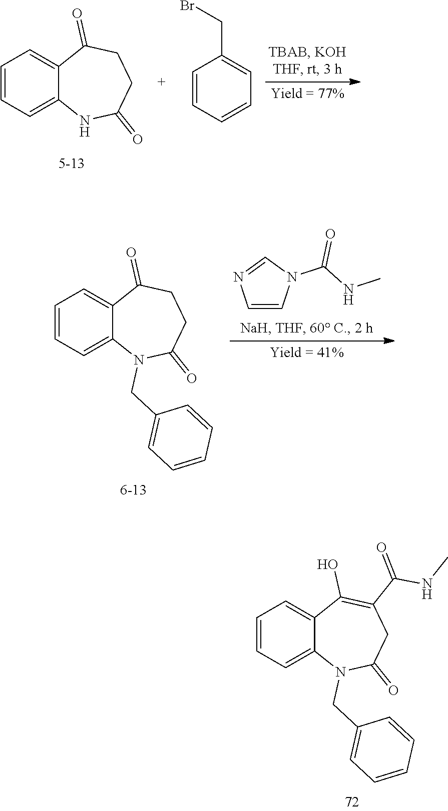

To a stirred solution of ketone (5-13) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added (bromomethyl)benzene (171 mg, 1.2 mmol, 1.2 equiv) at ambient temperature. The reaction mixture was then stirred at RT for 3 h. The reaction progress was monitored by TLC until the complete disappearance of compound 5-13. The solution was quenched with saturated ammonium chloride and extracted with EA (3×20 mL). The organic solutions were combined, washed with H₂O (2×20 ml) and brine (1×20 mL), dried over anhydrous Na₂SO₄, filtered and concentrated by rotary evaporation to get the crude compound. Then, the resulting concentrate was purified on a silica column to get 208 mg (purity=99%) light yellow solid compound 6-13 with a yield of 77%.

TLC R_{f}=0.45 (PE:EA=1:1)

LCMS (m/z)=266.40 (M+H)

To a stirred solution of ketone (6-13) (208 mg, 0.768 mmol, 1 equiv) and N-methyl-1H-imidazole-1-carboxamide (288 mg, 2.30 mmol, 3 equiv) in THF (4 mL), was added NaH 60% dispersion in mineral oil (307 mg, 7.68 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 102 mg (purity=95%) white solid compound 5-13 with a yield of 41%.

TLC R_{f}=0.45 (PE:EA=1:1)

LCMS (m/z)=323.40 (M+H)

### Example 73. 5-hydroxy-N-methyl-1-(naphthalen-2-ylmethyl)-2-oxo-2,3-dihydro-1H-benzo[b]azepine-4-carboxamide (73)

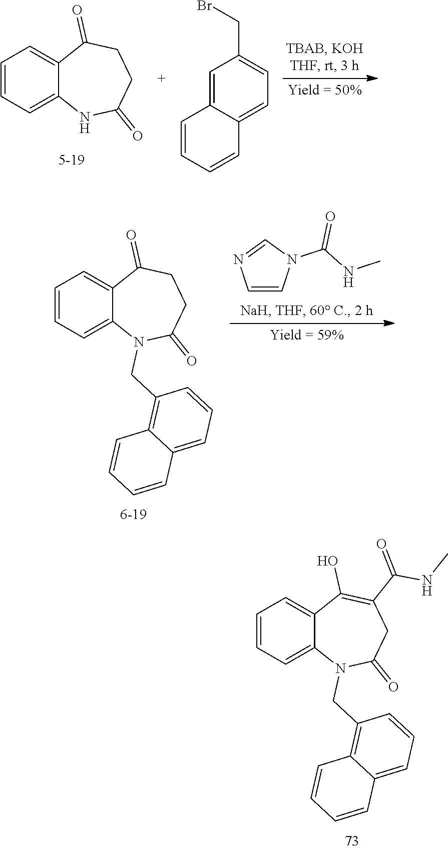

To a stirred solution of ketone (5-19) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 2-(bromomethyl)naphthalene (265 mg, 1.2 mmol, 1.2 equiv) at ambient temperature. The reaction mixture was then stirred at RT for 3 h. The reaction progress was monitored by TLC until the complete disappearance of compound 5-19. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 156 mg (purity=99%) light yellow solid compound 6-19 with a yield of 50%.

TLC R_{f}=0.5 (PE:EA=1:1)

LCMS (m/z)=316.40 (M+H)

To a stirred solution of ketone (6-19) (156 mg, 0.494 mmol, 1 equiv) and N-methyl-1H-imidazole-1-carboxamide (186 mg, 1.49 mmol, 3 equiv) in THF (3 mL), was added NaH 60% dispersion in mineral oil (173 mg, 4.94 mmol, 10 equiv) at ambient temperature under N₂ atmosphere. The reaction mixture was then stirred at 60° C. for 2 h. The solution was quenched with saturated ammonium chloride and extracted with EA (3×10 ml). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 109 mg (purity=98%) white solid compound 73 with a yield of 59%.

TLC R_{f}=0.5 (PE:EA=1:1)

LCMS (m/z)=373.50 (M+H)

### Example 74. 5-hydroxy-N-methyl-2-oxo-1-(pyridin-4-ylmethyl)-2,3-dihydro-1H-benzo[b]azepine-4-carboxamide (74)

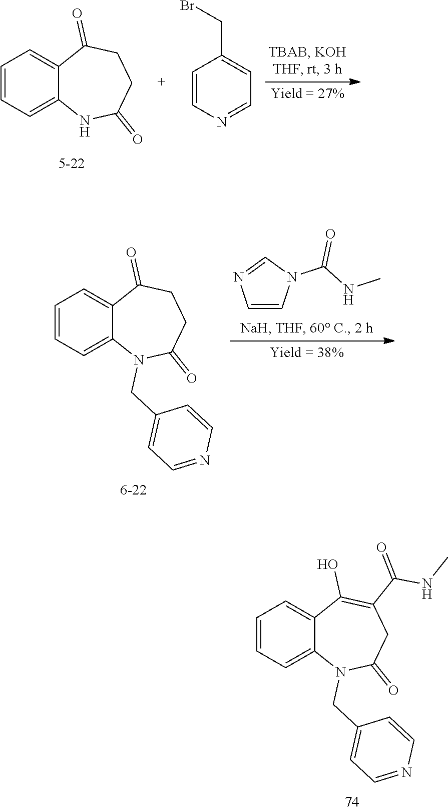

To a stirred solution of ketone (5-22) (175 mg, 1 mmol, 1 equiv), Tetrabutylammonium bromide (33 mg, 0.1 mmol, 0.1 equiv) and KOH (145 mg, 2.6 mmol, 2.6 equiv) in THF (4 mL), was added 4-(bromomethyl)pyridine (304 mg, 1.2 mmol, 1.2 equiv) at ambient temperature. The reaction mixture was then stirred at RT for 3 h. The reaction progress was monitored by TLC until the complete disappearance of compound 5-22. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 72 mg (purity=99%) light yellow solid compound 6-22 with a yield of 27%.

TLC R_{f}=0.45 (MeOH:DCM=1:10)

LCMS (m/z)=267.40 (M+H)

To a stirred solution of ketone (6-22) (72 mg, 0.271 mmol, 1 equiv) and N-methyl-1H-imidazole-1-carboxamide (108 mg, 0.864 mmol, 3 equiv) in THF (3 mL), was added NaH 60% dispersion in mineral oil (102 mg, 2.71 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 33 mg (purity=99%) white solid compound 74 with a yield of 38%.

TLC R_{f}=0.2 (MeOH:DCM=1:20)

LCMS (m/z)=324.50 (M+H)

### Example 75. 5-hydroxy-N-methyl-2-oxo-1-(pyridin-3-ylmethyl)-2,3-dihydro-1H-benzo[b]azepine-4-carboxamide (75)

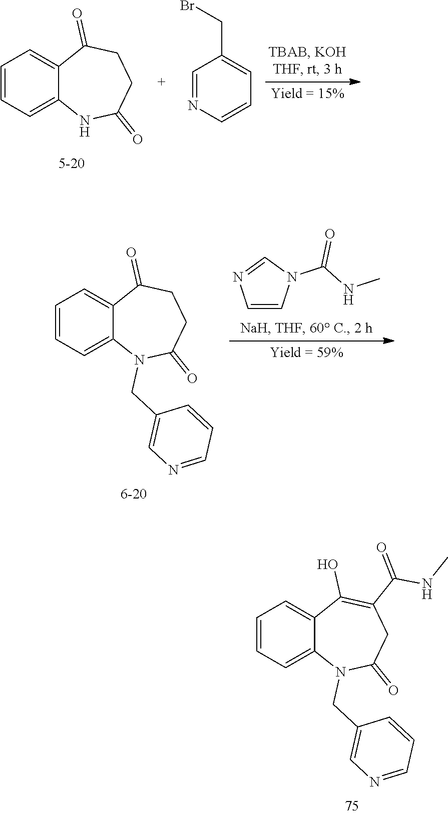

To a stirred solution of ketone (5-20) (175 mg, 1 mmol, 1 equiv), Tetrabutylammonium bromide (33 mg, 0.1 mmol, 0.1 equiv) and KOH (145 mg, 2.6 mmol, 2.6 equiv) in THE (4 mL), was added 3-(bromomethyl)pyridine (304 mg, 1.2 mmol, 1.2 equiv) at ambient temperature. The reaction mixture was then stirred at RT for 3 h. The reaction progress was monitored by TLC until the complete disappearance of compound 5-20. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 41 mg (purity=90%) light yellow solid compound 6-20 with a yield of 15%.

TLC R_{f}=0.25 (PE:EA=1:1)

LCMS (m/z)=267.40 (M+H)

To a stirred solution of ketone (6-20) (41 mg, 0.154 mmol, 1 equiv) and N-methyl-1H-imidazole-1-carboxamide (58 mg, 0.464 mmol, 3 equiv) in THF (1 mL), was added NaH 60% dispersion in mineral oil (62 mg, 1.54 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 15 mg (purity=98%) white solid compound 75 with a yield of 59%.

TLC R_{f}=0.45 (MeOH:DCM=1:10)

LCMS (m/z)=324.30 (M+H)

### Example 76. 1-(4-fluorobenzyl)-5-hydroxy-N-methyl-2-oxo-2,3-dihydro-1H-benzo[b]azepine-4-carboxamide (76)

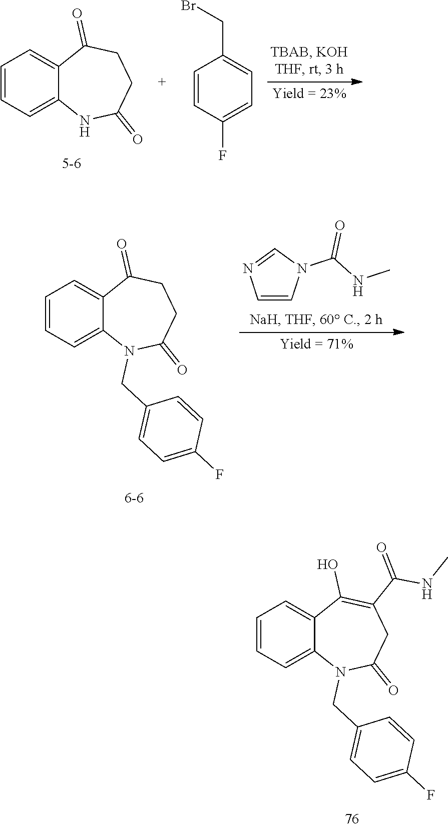

To a stirred solution of ketone (5-6) (200 mg, 1.14 mmol, 1 equiv), Tetrabutylammonium bromide (36 mg, 0.11 mmol, 0.1 equiv) and KOH (71 mg, 1.27 mmol, 1.1 equiv) in THE (4 mL), was added 1-(bromomethyl)-4-fluorobenzene (258 mg, 1.36 mmol, 1.2 equiv) at ambient temperature. The reaction mixture was then stirred at RT for 3 h. The reaction progress was monitored by TLC until the complete disappearance of compound 5-6. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 75 mg (purity=93%) light yellow solid compound 6-6 with a yield of 23%.

TLC R_{f}=0.3 (PE:EA=2:1)

LCMS (m/z)=284.40 (M+H)

To a stirred solution of ketone (6-6) (75 mg, 0.265 mmol, 1 equiv) and N-methyl-1H-imidazole-1-carboxamide (100 mg, 0.80 mmol, 3 equiv) in THF (1.5 mL), was added NaH 60% dispersion in mineral oil (106 mg, 2.65 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 47 mg (purity=99%) white solid compound 76 with a yield of 71%.

TLC R_{f}=0.5 (EA:PE=1:1)

LCMS (m/z)=341.50 (M+H)

### Example 77. 1-(3-fluorobenzyl)-5-hydroxy-N-methyl-2-oxo-2,3-dihydro-1H-benzo[b]azepine-4-carboxamide (77)

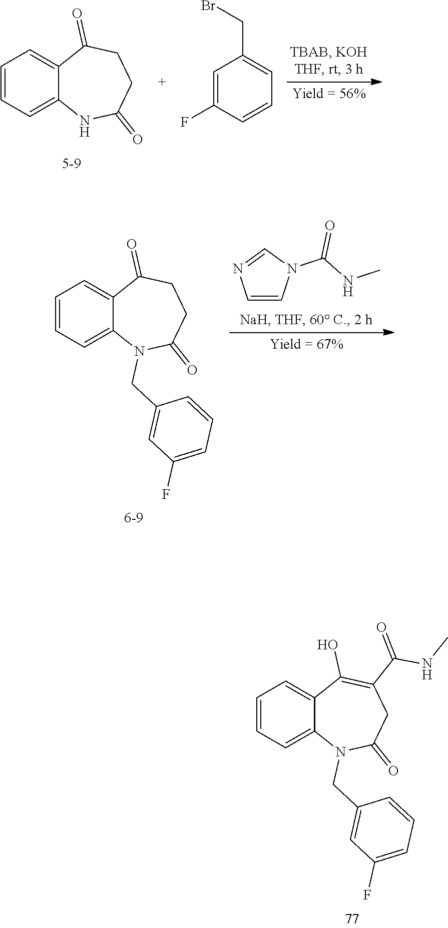

To a stirred solution of ketone (5-9) (150 mg, 0.857 mmol, 1 equiv), Tetrabutylammonium bromide (28 mg, 0.09 mmol, 0.1 equiv) and KOH (67 mg, 1.20 mmol, 1.4 equiv) in THF (3 mL), was added 1-(bromomethyl)-3-fluorobenzene (186 mg, 1.03 mmol, 1.2 equiv) at ambient temperature. The reaction mixture was then stirred at RT for 3 h. The reaction progress was monitored by TLC until the complete disappearance of compound 5-9. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 135 mg (purity=99%) light yellow solid compound 6-9 with a yield of 56%.

TLC R_{f}=0.55 (PE:EA=1:1)

LCMS (m/z)=284.60 (M+H)

To a stirred solution of ketone (6-9) (135 mg, 0.477 mmol, 1 equiv) and N-methyl-1H-imidazole-1-carboxamide (179 mg, 1.43 mmol, 3 equiv) in THF (3 mL), was added NaH 60% dispersion in mineral oil (191 mg, 4.77 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 109 mg (purity=94%) white solid compound 77 with a yield of 67%.

TLC R_{f}=0.3 (PE:EA=1:1)

LCMS (m/z)=341.50 (M+H)

### Example 78. 1-(2-fluorobenzyl)-5-hydroxy-N-methyl-2-oxo-2,3-dihydro-1H-benzo[b]azepine-4-carboxamide (78)

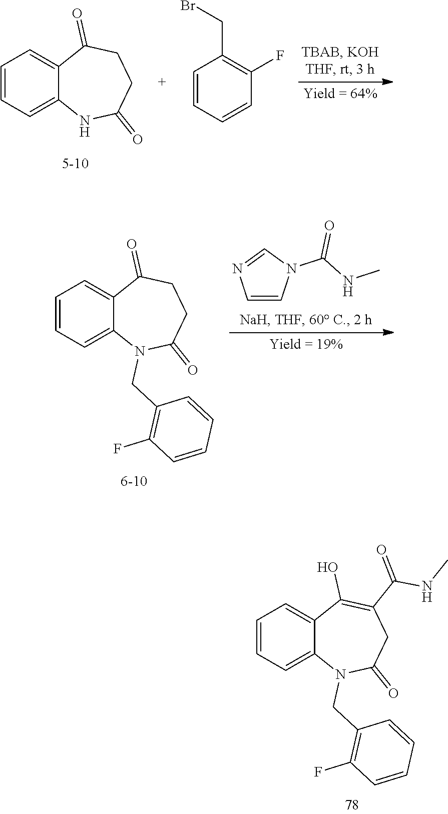

To a stirred solution of ketone (5-10) (150 mg, 0.857 mmol, 1 equiv), Tetrabutylammonium bromide (28 mg, 0.09 mmol, 0.1 equiv) and KOH (67 mg, 1.20 mmol, 1.4 equiv) in THE (3 mL), was added 1-(bromomethyl)-2-fluorobenzene (186 mg, 1.03 mmol, 1.2 equiv) at ambient temperature. The reaction mixture was then stirred at RT for 3 h. The reaction progress was monitored by TLC until the complete disappearance of compound 5-10. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 155 mg (purity=99%) light yellow solid compound 6-10 with a yield of 64%.

TLC R_{f}=0.55 (PE:EA=1:1)

LCMS (m/z)=284.40 (M+H)

To a stirred solution of ketone (6-10) (155 mg, 0.548 mmol, 1 equiv) and N-methyl-1H-imidazole-1-carboxamide (205 mg, 1.64 mmol, 3 equiv) in THF (3 mL), was added NaH 60% dispersion in mineral oil (219 mg, 5.48 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 36 mg (purity=91%) white solid compound 78 with a yield of 19%.

TLC R_{f}=0.3 (EA:PE=1:1)

LCMS (m/z)=341.50 (M+H)

### Example 79. 5-hydroxy-N-methyl-1-(4-methylbenzyl)-2-oxo-2,3-dihydro-1H-benzo[b]azepine-4-carboxamide (79)

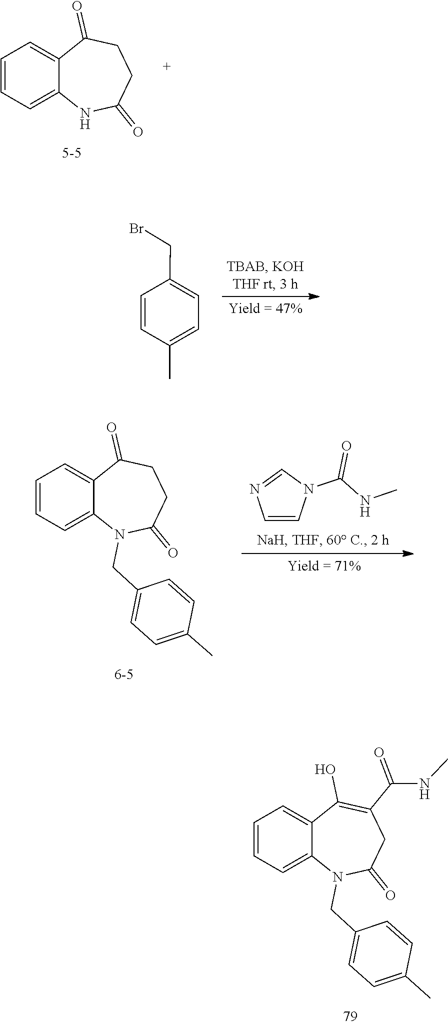

To a stirred solution of ketone (5-5) (200 mg, 1.14 mmol, 1 equiv), Tetrabutylammonium bromide (36 mg, 0.11 mmol, 0.1 equiv) and KOH (71 mg, 1.27 mmol, 1.1 equiv) in THF (4 mL), was added 1-(bromomethyl)-4-methylbenzene (252 mg, 1.36 mmol, 1.2 equiv) at ambient temperature. The reaction mixture was then stirred at RT for 3 h. The reaction progress was monitored by TLC until the complete disappearance of compound 5-5. The solution was quenched with saturated ammonium chloride and extracted with EA (3×20 ml). The organic solutions were combined, washed with H₂O) (2×20 ml) and brine (1×20 mL), dried over anhydrous Na₂SO₄, filtered and concentrated by rotary evaporation to get the crude compound. Then, the resulting concentrate was purified on a silica column to get 149 mg (purity=99%) light yellow solid compound 6-5 with a yield of 47%.

TLC R_{f}=0.4 (PE:EA=2:1)

LCMS (m/z)=280.40 (M+H)

To a stirred solution of ketone (6-5) (149 mg, 0.534 mmol, 1 equiv) and N-methyl-1H-imidazole-1-carboxamide (200 mg, 1.60 mmol, 3 equiv) in THF (3 mL), was added NaH 60% dispersion in mineral oil (212 mg, 5.34 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 127 mg (purity=99%) white solid compound 79 with a yield of 71%.

TLC R_{f}=0.5 (EA:PE=1:1)

LCMS (m/z)=337.40 (M+H)

### Example 80. 5-hydroxy-N-methyl-1-(3-methylbenzyl)-2-oxo-2,3-dihydro-1H-benzo[b]azepine-4-carboxamide (80)

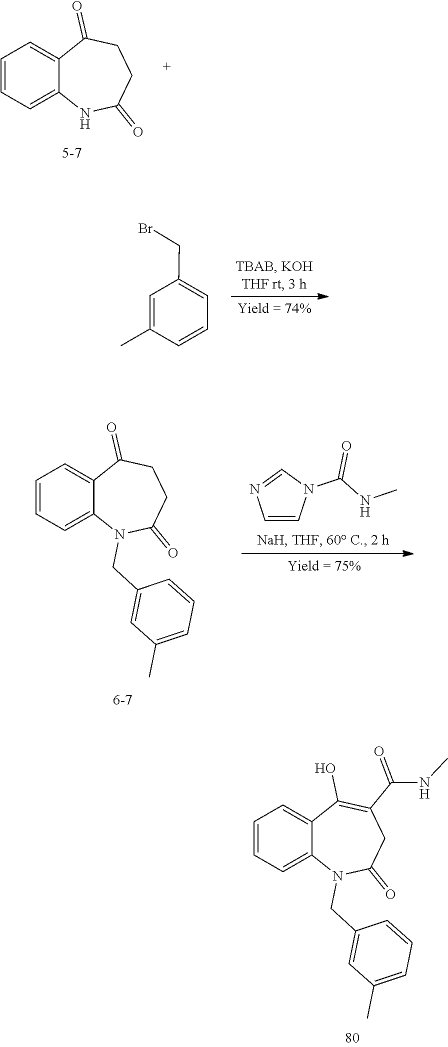

To a stirred solution of ketone (5-7) (200 mg, 1.14 mmol, 1 equiv), Tetrabutylammonium bromide (37 mg, 0.11 mmol, 0.1 equiv) and KOH (90 mg, 1.61 mmol, 1.4 equiv) in THF (4 mL), was added 1-(bromomethyl)-3-methylbenzene (252 mg, 1.36 mmol, 1.2 equiv) at ambient temperature. The reaction mixture was then stirred at RT for 3 h. The reaction progress was monitored by TLC until the complete disappearance of compound 5-7. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 237 mg (purity=93%) light yellow solid compound 6-7 with a yield of 74%.

TLC R_{f}=0.3 (PE:EA=2:1)

LCMS (m/z)=280.40 (M+H)

To a stirred solution of ketone (6-7) (237 mg, 0.849 mmol, 1 equiv) and N-methyl-1H-imidazole-1-carboxamide (319 mg, 2.55 mmol, 3 equiv) in THF (3 mL), was added NaH 60% dispersion in mineral oil (340 mg, 8.49 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 215 mg (purity=99%) white solid compound 80 with a yield of 75%.

TLC R_{f}=0.5 (EA:PE=1:1)

LCMS (m/z)=337.40 (M+H)

### Example 81. 5-hydroxy-N-methyl-1-(2-methylbenzyl)-2-oxo-2,3-dihydro-1H-benzo[b]azepine-4-carboxamide (81)

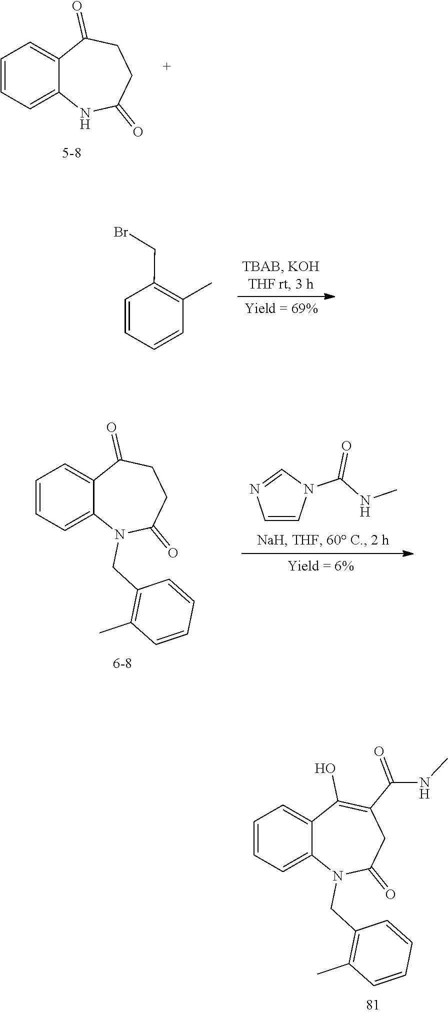

To a stirred solution of ketone (5-8) (200 mg, 1.14 mmol, 1 equiv), Tetrabutylammonium bromide (37 mg, 0.11 mmol, 0.1 equiv) and KOH (90 mg, 1.61 mmol, 1.4 equiv) in THE (4 mL), was added 1-(bromomethyl)-2-methylbenzene (252 mg, 1.36 mmol, 1.2 equiv) at ambient temperature. The reaction mixture was then stirred at RT for 3 h. The reaction progress was monitored by TLC until the complete disappearance of compound 5-8. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 220 mg (purity=85%) light yellow solid compound 6-8 with a yield of 69%.

TLC R_{f}=0.3 (PE:EA=2:1)

LCMS (m/z)=280.60 (M+H)

To a stirred solution of ketone (6-8) (220 mg, 0.789 mmol, 1 equiv) and N-methyl-1H-imidazole-1-carboxamide (296 mg, 2.37 mmol, 3 equiv) in THF (3 mL), was added NaH 60% dispersion in mineral oil (315 mg, 7.89 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 17 mg (purity=99%) white solid compound 81 with a yield of 6%.

TLC R_{f}=0.5 (EA:PE=1:1)

LCMS (m/z)=337.40 (M+H)

### Example 82. 1-(1-(4-fluorophenyl) ethyl)-3,4-dihydro-1H-benzo[b]azepine-2,5-dione (82)

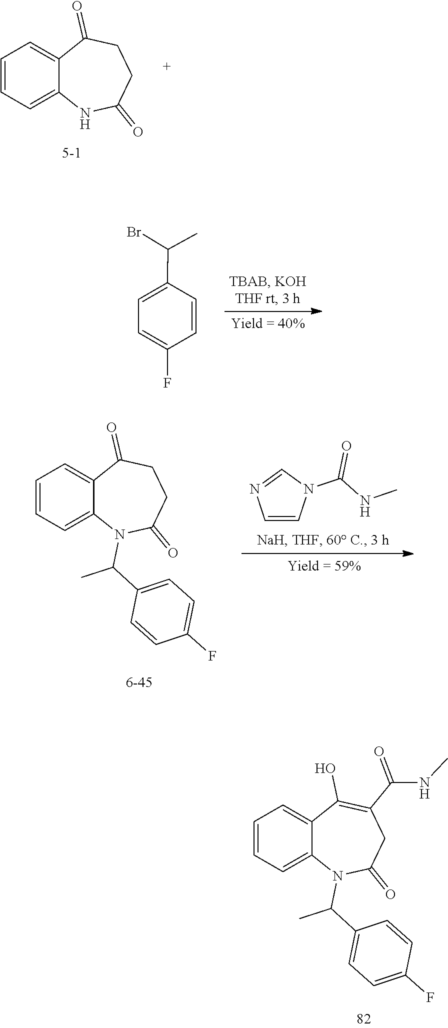

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(1-bromoethyl)-4-fluorobenzene (244 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 120 mg (purity=95%) light yellow solid compound 6-45 with a yield of 40%.

TLC R_{f}=0.75 (EA:PE=1:1)

LCMS (m/z)=298.40 (M+H)

To the solution of resulting compound (6-45) (120 mg, 0.404 mmol, 1 equiv) and N-methyl-1H-imidazole-1-carboxamide (152 mg, 1.216 mmol, 3 equiv) in THF (3 mL), was added NaH 60% dispersion in mineral oil (162 mg, 4.04 mmol, 10 equiv) at ambient temperature under N, atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 83 mg (purity=90%) white solid compound 82 with a yield of 59%.

TLC R_{f}=0.65 (EA:PE=1:1)

LCMS (m/z)=355.50 (M+H)

### Example 83. 1-(4-chlorobenzyl)-5-hydroxy-N-methyl-2-oxo-2,3-dihydro-1H-benzo[b]azepine-4-carboxamide (83)

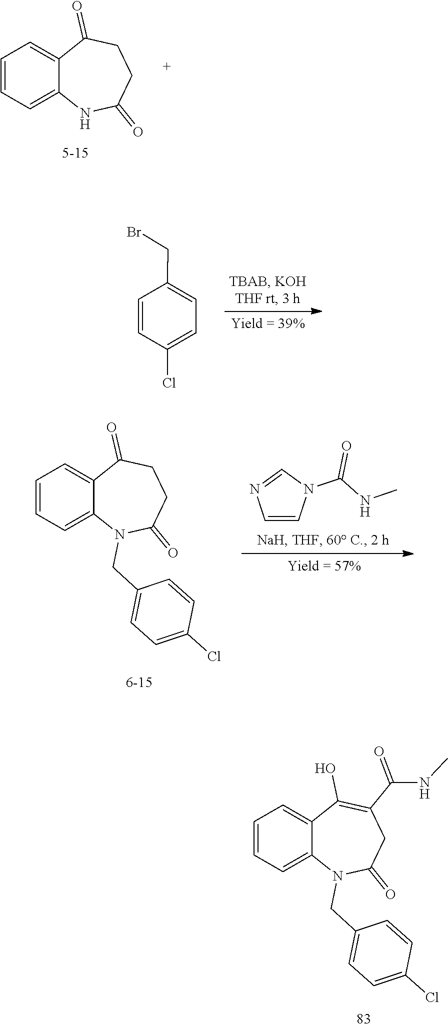

To a stirred solution of ketone (5-15) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 1-(bromomethyl)-4-chlorobenzene (171 mg, 1.2 mmol, 1.2 equiv) at ambient temperature. The reaction mixture was then stirred at RT for 3 h. The reaction progress was monitored by TLC until the complete disappearance of compound 5-15. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 117 mg (purity=98%) light yellow solid compound 6-15 with a yield of 39%.

TLC R_{f}=0.45 (PE:EA=1:1)

LCMS (m/z)=300.50 (M+H)

To a stirred solution of ketone (6-15) (110 mg, 0.33 mmol, 1 equiv) and N-methyl-1H-imidazole-1-carboxamide (124 mg, 0.99 mmol, 3 equiv) in THF (4 mL), was added NaH 60% dispersion in mineral oil (132 mg, 3.3 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 73 mg (purity=97%) white solid compound 83 with a yield of 57%.

TLC R_{f}=0.45 (PE:EA=1:1)

LCMS (m/z)=357.40 (M+H)

### Example 84. 5-hydroxy-N-methyl-2-oxo-1-(4-(trifluoromethyl)benzyl)-2,3-dihydro-1H-benzo[b]azepine-4-carboxamide (84)

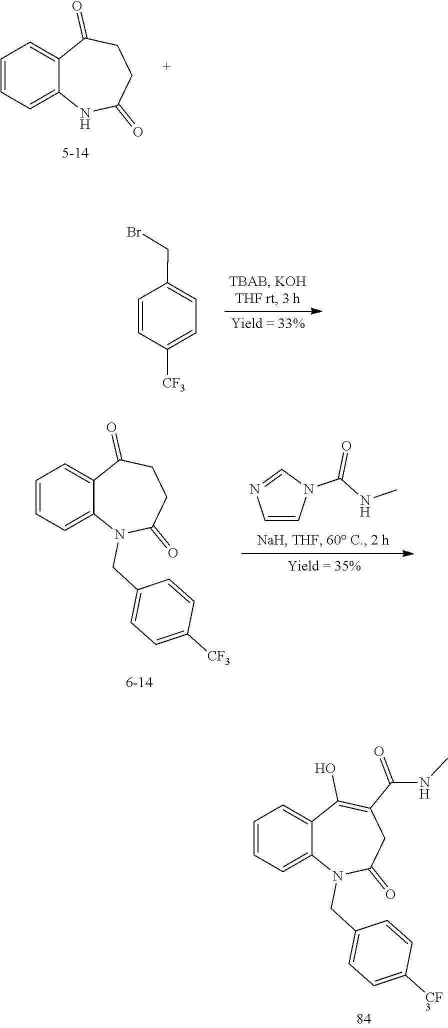

To a stirred solution of ketone (5-14) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-4-(trifluoromethyl)benzene (171 mg, 1.2 mmol, 1.2 equiv) at ambient temperature. The reaction mixture was then stirred at RT for 3 h. The reaction progress was monitored by TLC until the complete disappearance of compound 5-14. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 110 mg (purity=98%) light yellow solid compound 6-14 with a yield of 33%.

TLC R_{f}=0.45 (PE:EA=1:1)

LCMS (m/z)=334.50 (M+H)

To a stirred solution of ketone (6-14) (110 mg, 0.33 mmol, 1 equiv) and N-methyl-1H-imidazole-1-carboxamide (124 mg, 0.99 mmol, 3 equiv) in THE (4 mL), was added NaH 60% dispersion in mineral oil (132 mg, 3.3 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 45 mg (purity=99%) white solid compound 84 with a yield of 35%.

TLC R_{f}=0.45 (PE:EA=1:1)

LCMS (m/z)=391.50 (M+H)

### Example 85. 1-(4-cyanobenzyl)-5-hydroxy-N-methyl-2-oxo-2,3-dihydro-1H-benzo[b]azepine-4-carboxamide (85)

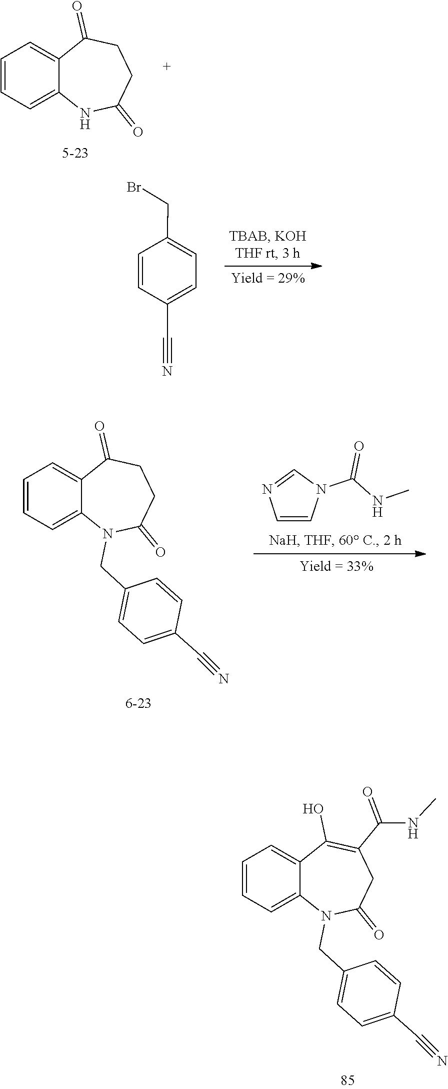

To a stirred solution of ketone (5-23) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 4-(bromomethyl)benzonitrile (235 mg, 1.2 mmol, 1.2 equiv) at ambient temperature. The reaction mixture was then stirred at RT for 3 h. The reaction progress was monitored by TLC until the complete disappearance of compound 5-23. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 175 mg (purity=48%) light yellow solid compound 6-23 with a yield of 29%.

TLC R_{f}=0.55 (EA:PE=1:1)

LCMS (m/z)=291.60 (M+H)

To a stirred solution of ketone (6-23) (175 mg, 0.603 mmol, 1 equiv) and N-methyl-1H-imidazole-1-carboxamide (226 mg, 1.808 mmol, 3 equiv) in THF (3 mL), was added NaH 60% dispersion in mineral oil (102 mg, 6.03 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 31 mg (purity=88%) white solid compound 85 with a yield of 33%.

TLC R_{f}=0.5 (EA:PE=1:1)

LCMS (m/z)=348.50 (M+H)

### Example 86. 1-(4-ethylbenzyl)-5-hydroxy-N-methyl-2-oxo-2,3-dihydro-1H-benzo[b]azepine-4-carboxamide (86)

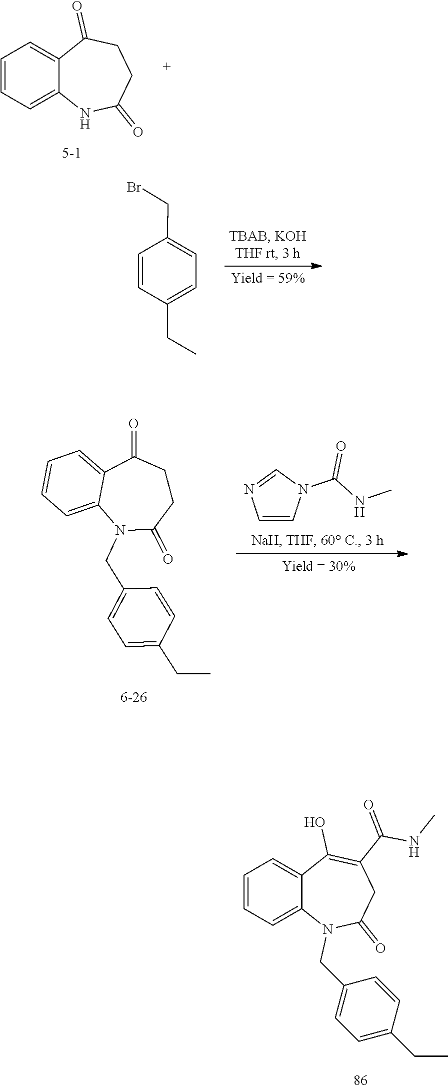

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-4-ethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 173 mg (purity=95%) light yellow solid compound 6-26 with a yield of 59%.

TLC R_{f}=0.6 (EA:PE=1:1)

LCMS (m/z)=294.40 (M+H)

To the solution of resulting compound 6-26 (173 mg, 0.590 mmol, 1 equiv) and N-methyl-1H-imidazole-1-carboxamide (221 mg, 1.768 mmol, 3 equiv) in THE (3 mL), was added NaH 60% dispersion in mineral oil (231 mg, 5.90 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 63 mg (purity=98%) white solid compound 86 with a yield of 30%.

TLC R_{f}=0.5 (EA:PE=1:1)

LCMS (m/z)=351.60 (M+H)

### Example 87. 5-hydroxy-1-(4-isopropylbenzyl)-N-methyl-2-oxo-2,3-dihydro-1H-benzo[b]azepine-4-carboxamide (87)

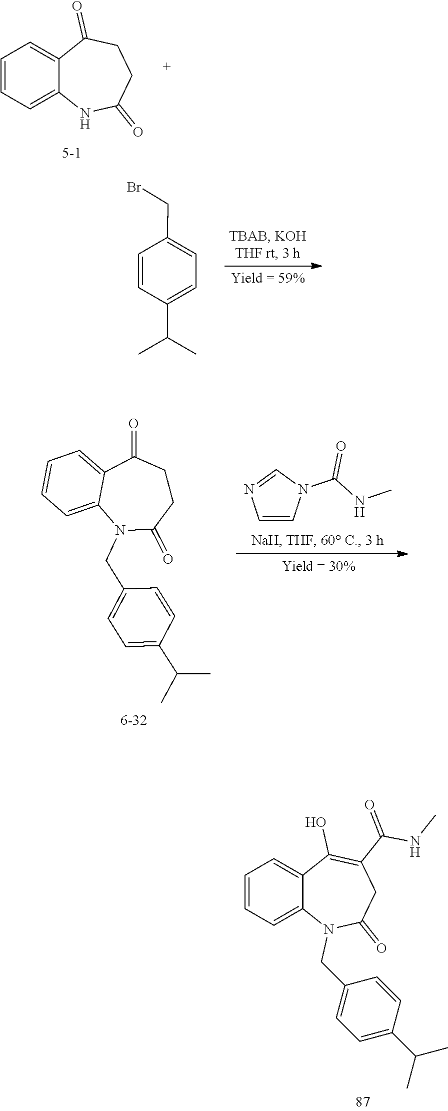

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-4-isopropylbenzene (256 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 181 mg (purity=99%) light yellow solid compound 6-32 with a yield of 59%.

TLC R_{f}=0.65 (EA:PE=1:1)

LCMS (m/z)=308.40 (M+H)

To the solution of resulting compound 6-32 (181 mg, 0.590 mmol, 1 equiv) and N-methyl-1H-imidazole-1-carboxamide (221 mg, 1.768 mmol, 3 equiv) in THF (4 mL), was added NaH 60% dispersion in mineral oil (236 mg, 5.90 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 65 mg (purity=64%) white solid compound 87 with a yield of 30%.

TLC R_{f}=0.6 (EA:PE=1:1)

LCMS (m/z)=365.40 (M+H)

### Example 88. 5-hydroxy-N-methyl-2-oxo-1-(4-pentylbenzyl)-2,3-dihydro-1H-benzo[b]azepine-4-carboxamide (88)

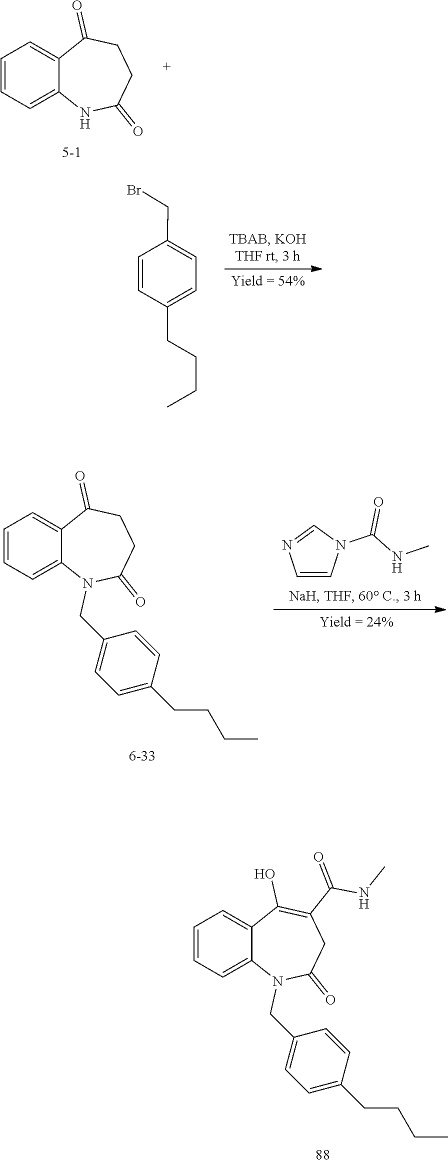

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 1-(bromomethyl)-4-butylbenzene (272 mg, 1.2 mmol, 1.2 equiv) at ambient temperature. The reaction mixture was then stirred at RT for 3b. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 151 mg (purity=99%) light yellow solid compound 6-33 with a yield of 54%.

TLC R_{f}=0.65 (EA:PE=1:1)

LCMS (m/z)=322.60 (M+H)

To the solution of resulting compound 6-33 (151 mg, 0.470 mmol, 1 equiv) and N-methyl-1H-imidazole-1-carboxamide (176 mg, 1.408 mmol, 3 equiv) in THF (3 mL), was added NaH 60% dispersion in mineral oil (188 mg, 4.70 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 43 mg (purity=65%) white solid compound 88 with a yield of 24%.

TLC R_{f}=0.6 (EA:PE=1:1)

LCMS (m/z)=378.60 (M+H)

### Example 89. 1-([1,1′-biphenyl]-4-ylmethyl)-5-hydroxy-N-methyl-2-oxo-2,3-dihydro-1H-benzo[b]azepine-4-carboxamide (89)

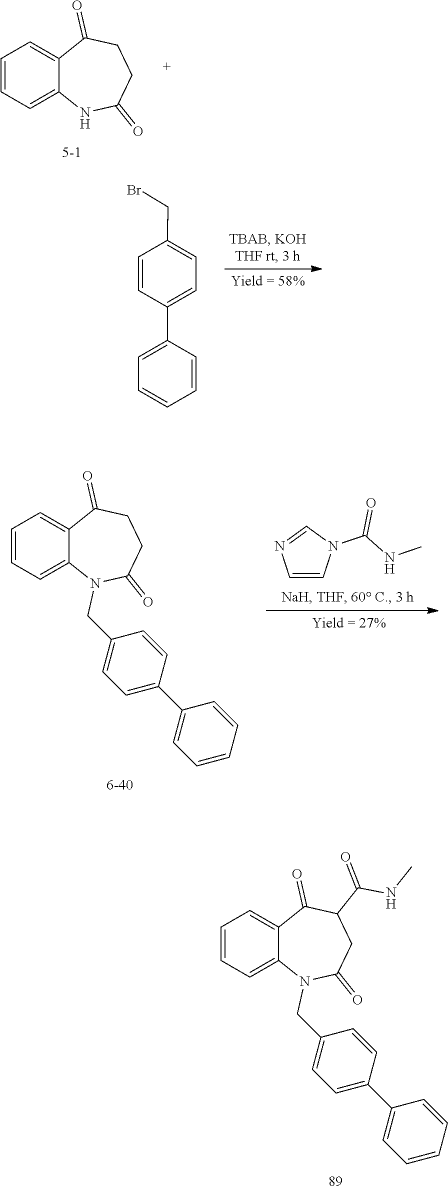

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 4-(bromomethyl)-1,1′-biphenyl (296 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 199 mg (purity=96%) light yellow solid compound 6-40 with a yield of 58%.

TLC R_{f}=0.65 (EA:PE=1:1)

LCMS (m/z)=342.50 (M+H)

To the solution of resulting compound 6-40 (199 mg, 0.584 mmol, 1 equiv) and N-methyl-1H-imidazole-1-carboxamide (219 mg, 1.752 mmol, 3 equiv) in THF (3 mL), was added NaH 60% dispersion in mineral oil (233 mg, 5.22 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 62 mg (purity=99%) white solid compound 89 with a yield of 27%.

TLC R_{f}=0.6 (EA:PE=1:1)

LCMS (m/z)=399.60 (M+H)

### Example 90. 1-(4-acetylbenzyl)-5-hydroxy-N-methyl-2-oxo-2,3-dihydro-1H-benzo[b]azepine-4-carboxamide (90)

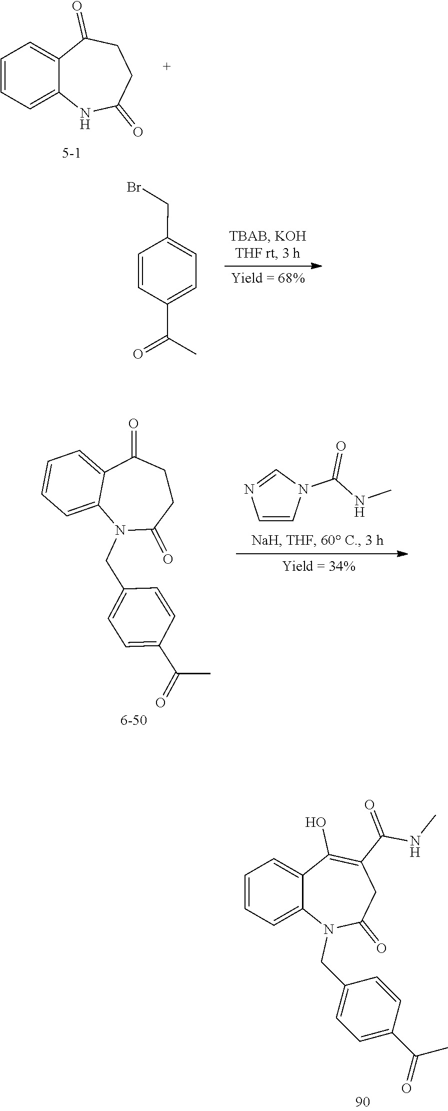

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 1-(4-(bromomethyl)phenyl)ethan-1-one (256 mg, 1.2 mmol, 1.2 equiv) at ambient temperature. The reaction mixture was then stirred at RT for 3 h. The reaction progress was monitored by TLC until the complete disappearance of compound 5-1

The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 208 mg (purity=59%) light yellow solid compound 6-50 with a yield of 68%.

TLC R_{f}=0.55 (EA:PE=1:1)

LCMS (m/z)=308.40 (M+H)

To the solution of resulting compound 6-50 (208 mg, 0.678 mmol, 1 equiv) and N-methyl-1H-imidazole-1-carboxamide (254 mg, 2.032 mmol, 3 equiv) in THF (3 mL), was added NaH 60% dispersion in mineral oil (271 mg, 6.78 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 ml), dried over anhydrous Na₂SO₄, filtered and concentrated by rotary evaporation to get the crude compound. Then, the resulting concentrate was purified on a silica column to get 84 mg (purity=99%) white solid compound 90 with a yield of 34%.

TLC R_{f}=0.7 (MeOH:DCM=1:10)

LCMS (m/z)=365.40 (M+H)

### Example 91. 5-hydroxy-1-(4-methoxybenzyl)-N-methyl-2-oxo-2,3-dihydro-1H-benzo[b]azepine-4-carboxamide (90)

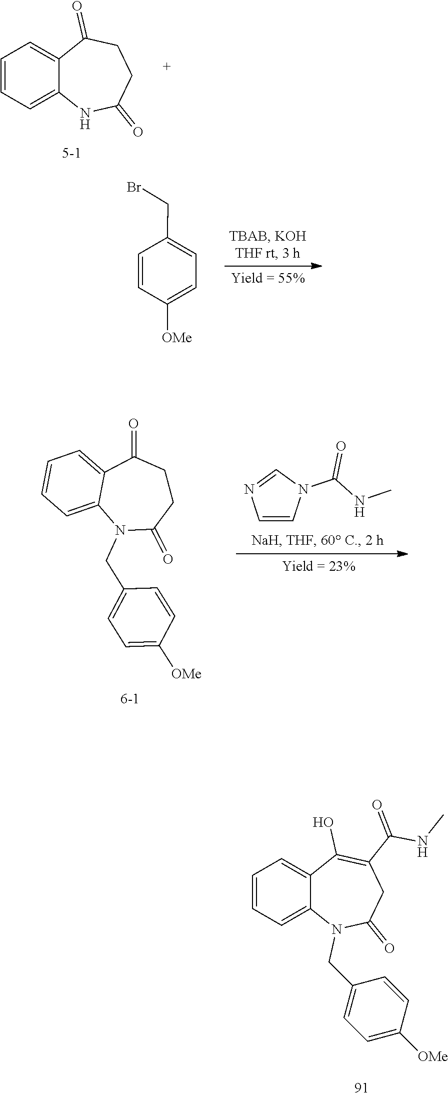

To a stirred solution of ketone (5-1) (175 mg, 1 mmol, 1 equiv), Tetrabutylammonium bromide TBAB (32 mg, 0.1 mmol, 0.1 equiv.) and KOH (78.4 mg, 1.4 mmol, 1.4 equiv) in THF (4 mL), was added 1-(bromomethyl)-4-methoxybenzene (200 mg, 1.1 mmol, 1.1 equiv.) at ambient temperature. The reaction mixture was then stirred at rt for 3 h. The reaction progress was monitored by TLC until the complete disappearance of compound 5-1. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 150 mg (purity=87%) of compound 6-1 in light yellow solid with a yield of 55%.

TLC R_{f}=0.4 (PE:EA=1:1);

LCMS (m/z)=296.50 (M+H)

To the solution of resulting compound (6-1) (70 mg, 0.23 mmol, 1 equiv) and N-methyl-1H-imidazole-1-carboxamide (86 mg, 0.69 mmol, 3 equiv) in THE (1.5 mL), was added NaH 60% dispersion in mineral oil (92 mg, 2.3 mmol, 10 equiv.) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 20 mg (purity=98%) of 90 (light yellow oil, Yield=23%)

TLC R_{f}=0.3 (PE:EA=1:1);

LCMS (m/z)=353.60 (M+H)

### Example 92. 5-hydroxy-1-(3-methoxybenzyl)-N-methyl-2-oxo-2,3-dihydro-1H-benzo[b]azepine-4-carboxamide (92)

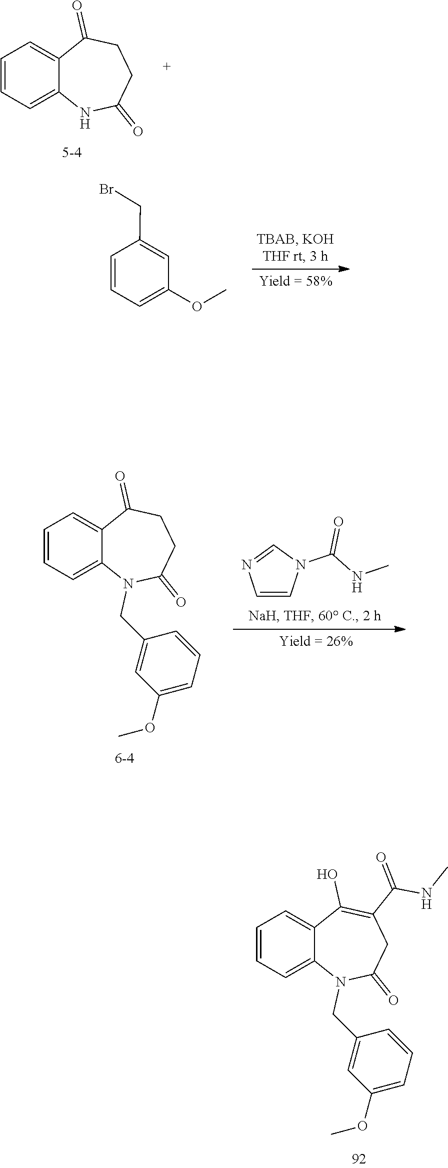

To a stirred solution of ketone (5-4) (200 mg, 1.14 mmol, 1 equiv), Tetrabutylammonium bromide (36 mg, 0.11 mmol, 0.1 equiv) and KOH (71 mg, 1.59 mmol, 1.4 equiv) in THE (4 mL), was added 1-(bromomethyl)-3-methoxybenzene (276 mg, 1.37 mmol, 1.2 equiv) at ambient temperature. The reaction mixture was then stirred at RT for 3 h. The reaction progress was monitored by TLC until the complete disappearance of compound 5-4. The solution was quenched with saturated ammonium chloride and extracted with EA (3×20 mL). The organic solutions were combined, washed with H₂O (2×20 mL) and brine (1×20 ml), dried over anhydrous Na₂SO₄, filtered and concentrated by rotary evaporation to get the crude compound. Then, the resulting concentrate was purified on a silica column to get 194 mg (purity=91%) light yellow solid compound 6-4 with a yield of 58%.

TLC R_{f}=0.3 (PE:EA=2:1)

LCMS (m/z)=296.50 (M+H)

To a stirred solution of ketone (6-4) (194 mg, 0.658 mmol, 1 equiv) and N-methyl-1H-imidazole-1-carboxamide (242 mg, 1.934 mmol, 3 equiv) in THF (4 mL), was added NaH 60% dispersion in mineral oil (258 mg, 6.58 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 61 mg (purity=96%) white solid compound 92 with a yield of 26%.

TLC R_{f}=0.45 (EA:PE=1:2)

LCMS (m/z)=352.23 (M+H)

### Example 93. 5-hydroxy-1-(2-methoxybenzyl)-N-methyl-2-oxo-2,3-dihydro-1H-benzo[b]azepine-4-carboxamide (93)

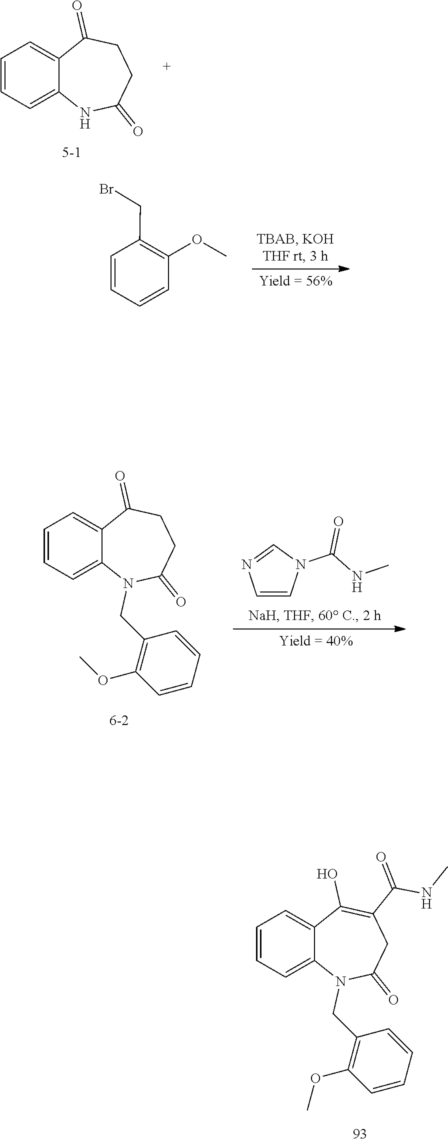

To a stirred solution of ketone (5-1) (175 mg, 1 mmol, 1 equiv), Tetrabutylammonium bromide (32 mg, 0.1 mmol, 0.1 equiv) and KOH (78.4 mg, 1.4 mmol, 1.4 equiv) in THF (4 mL), was added 1-(bromomethyl)-2-methoxybenzene (200 mg, 1.1 mmol, 1.1 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×10 mL). The organic solutions were combined, washed with (2×10 mL) and brine (1×20 mL), dried over anhydrous Na₂SO₄, filtered and concentrated by rotary evaporation to get the crude compound. Then, the resulting concentrate was purified on a silica column to get 157 mg (purity=65%) light yellow solid compound 6-2 with a yield of 56%.

TLC R_{f}=0.4 (PE:EA=1:1);

LCMS (m/z)=296.50 (M+H)

To the solution of resulting compound (6-2) (70 mg, 0.23 mmol, 1 equiv) and N-methyl-1H-imidazole-1-carboxamide (86 mg, 0.69 mmol, 3 equiv) in THF (1.5 mL), was added NaH 60% dispersion in mineral oil (92 mg, 2.3 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 45 mg (purity=84%) light yellow oil compound 93 with a yield of 40%.

TLC R_{f}=0.3 (PE:EA=1:1)

LCMS (m/z)=353.60 (M+H)

### Example 94. 1-(4-(difluoromethoxy)benzyl)-5-hydroxy-N-methyl-2-oxo-2,3-dihydro-1H-benzo[b]azepine-4-carboxamide (94)

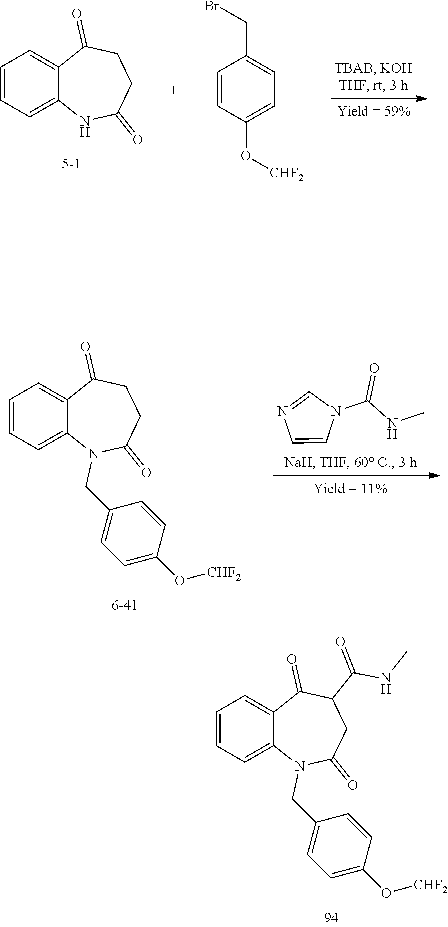

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 1-(bromomethyl)-4-(difluoromethoxy)benzene (284 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 205 mg (purity=88%) light yellow solid compound 6-41 with a yield of 59%

TLC R_{f}=0.65 (EA:PE=1:1)

LCMS (m/z)=332.40 (M+H)

To the solution of resulting compound 6-41 (205 mg, 0.587 mmol, 1 equiv) and N-methyl-1H-imidazole-1-carboxamide (220 mg, 1.760 mmol, 3 equiv) in THF (3 mL), was added NaH 60% dispersion in mineral oil (235 mg, 5.87 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 24 mg (purity=90%) white solid compound 94 with a yield of 11%.

TLC R_{f}=0.6 (EA:PE=1:1)

LCMS (m/z)=389.40 (M+H)

### Example 95. 1-(3-fluoro-4-methoxyphenyl)-5-hydroxy-N-methyl-2-oxo-2,3-dihydro-1H-benzo[b]azepine-4-carboxamide (95)

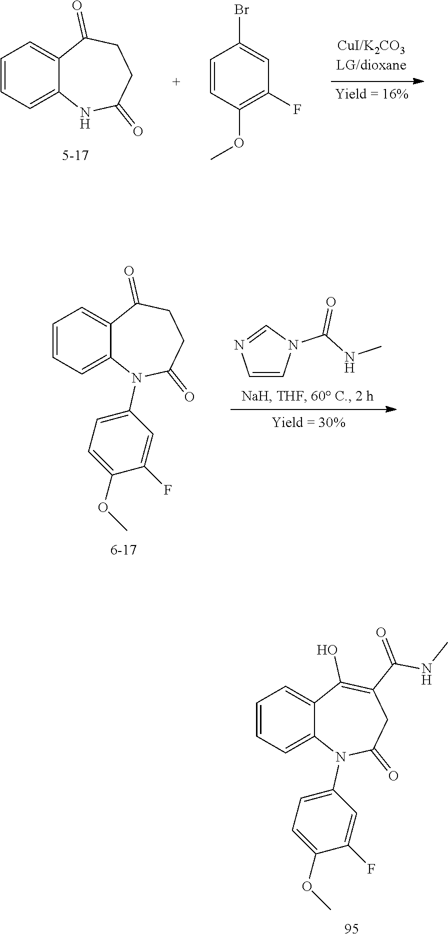

To a solution of ketone (5-17) (350 mg, 2 mmol) in 1,4-dioxane (10 mL) were added 4-bromo-2-fluoro-1-methoxybenzene (284 mg, 3.3 mmol), copper(I) iodide (190 mg, 1 mmol), potassium carbonate ($$2 mg, 4 mmol), and trans-N,N′-dimethylcyclohexane-1,2-diamine (114 mg, 0.8 mmol) under N₂. After being heated with stirring at reflux for 6 h, the reaction mixture was filtered through a pad of Celite. The filtrate was concentrated in vacuo. Then, the resulting concentrate was purified on a silica column to get 95 mg (purity=96%) light yellow solid compound 6-17 with a yield of 16%.

TLC R_{f}=0.45 (PE:EA=1:1)

LCMS (m/z)=300.50 (M+H)

To a stirred solution of ketone (6-17) (95 mg, 0.31 mmol, 1 equiv) and N-methyl-1H-imidazole-1-carboxamide (116 mg, 0.93 mmol, 3 equiv) in THF (2 mL), was added NaH 60% dispersion in mineral oil (124 mg, 3.1 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 33 mg (purity=98%) white solid compound 95 with a yield of 30%.

TLC R_{f}=0.45 (PE:EA=1:1)

LCMS (m/z)=357.40 (M+H)

### Example 96. 5-hydroxy-1-(4-methoxyphenethyl)-N-methyl-2-oxo-2,3-dihydro-1H-benzo[b]azepine-4-carboxamide (96)

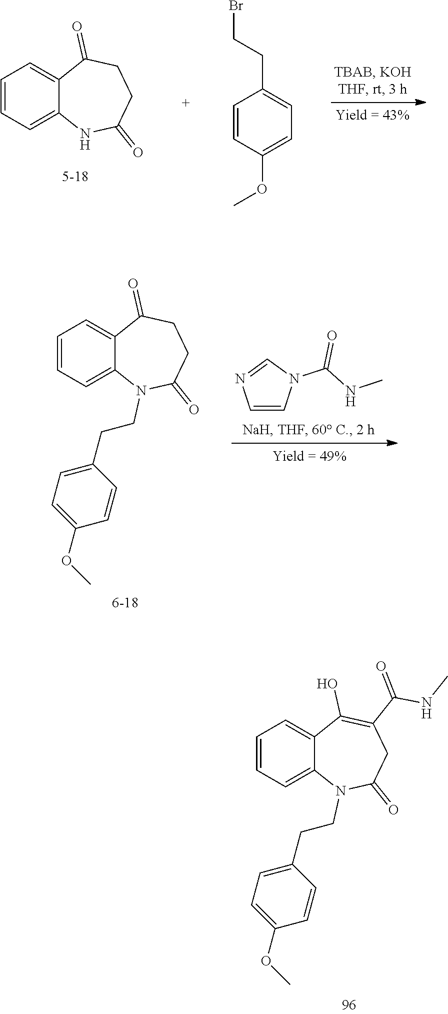

To a stirred solution of ketone (5-18) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added (bromomethyl)cyclopropane (162 mg, 1.2 mmol, 1.2 equiv) at ambient temperature. The reaction mixture was then stirred at RT for 3b. The reaction progress was monitored by TLC until the complete disappearance of compound 5-18. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 75 mg (purity=95%) light yellow solid compound 6-18 with a yield of 43%.

TLC R_{f}=0.45 (PE:EA=1:1)

LCMS (m/z)=310.70 (M+H)

To a stirred solution of ketone (6-18) (75 mg, 0.24 mmol, 1 equiv) and N-methyl-1H-imidazole-1-carboxamide (90 mg, 0.72 mmol, 3 equiv) in THF (2 mL), was added NaH 60% dispersion in mineral oil (96 mg, 2.4 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 43 mg (purity=99%) white solid compound 96 with a yield of 49%.

TLC R_{f}=0.45 (PE:EA=1:1)

LCMS (m/z)=367.40 (M+H)

### Example 97. 1-(3,5-dimethylbenzyl)-5-hydroxy-N-methyl-2-oxo-2,3-dihydro-1H-benzo[b]azepine-4-carboxamide (97)

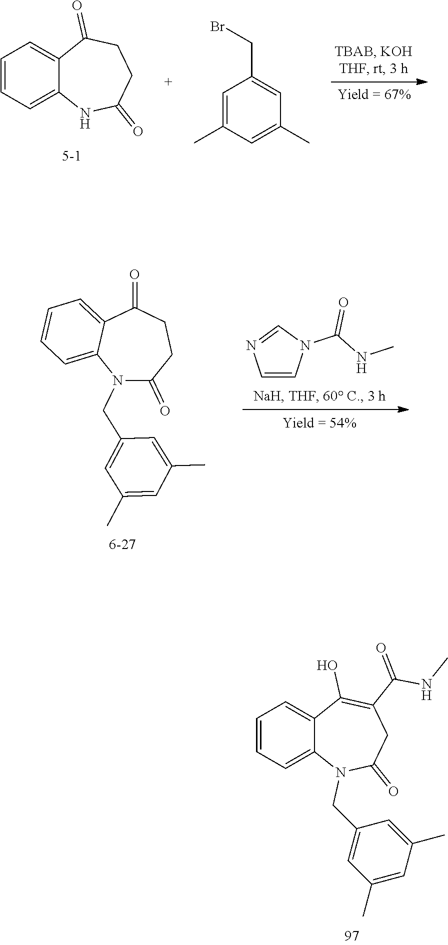

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-3,5-dimethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 195 mg (purity=95%) light yellow solid compound 6-27 with a yield of 67%

TLC R_{f}=0.6 (EA:PE=1:1)

LCMS (m/z)=294.20 (M+H)

To the solution of resulting compound 6-27 (195 mg, 0.666 mmol, 1 equiv) and N-methyl-1H-imidazole-1-carboxamide (250 mg, 2 mmol, 3 equiv) in THF (3 mL), was added NaH 60% dispersion in mineral oil (266 mg, 6.66 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 125 mg (purity=99%) white solid compound 97 with a yield of 54%.

TLC R_{f}=0.5 (EA:PE=1:1)

LCMS (m/z)=351.40 (M+H)

### Example 98. 1-(2,3-dimethylbenzyl)-5-hydroxy-N-methyl-2-oxo-2,3-dihydro-1H-benzo[b]azepine-4-carboxamide (98)

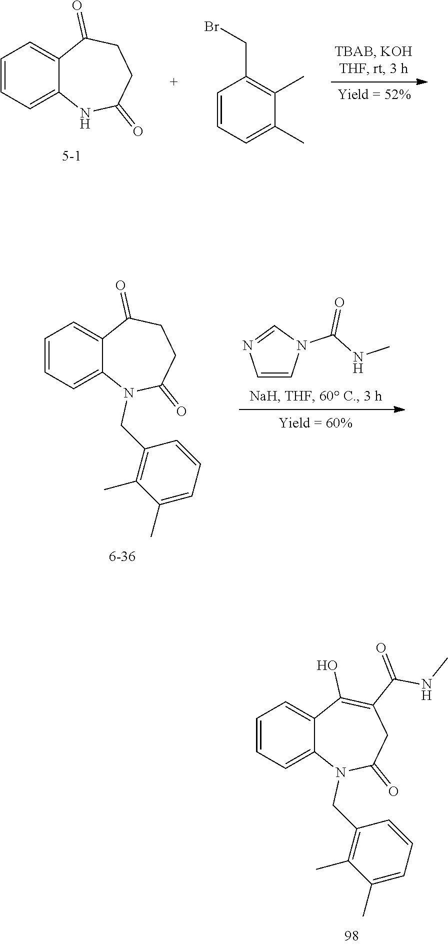

To the solution of resulting compound 5-1 (175 mg, 1 mmol, 1 equiv). Tetrabutylammonium bromide (33 mg, 0.1 mmol, 0.1 equiv) and KOH (79 mg, 1.4 mmol, 1.4 equiv) in THE (4 mL), was added 1-(bromomethyl)-2,3-dimethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 151 mg (purity=91%) light yellow solid compound 6-36 with a yield of 52%.

TLC R_{f}=0.65 (EA:PE=1:1)

LCMS (m/z)=294.40 (M+H)

To the solution of resulting compound 6-36 (151 mg, 0.515 mmol, 1 equiv) and N-methyl-1H-imidazole-1-carboxamide (193 mg, 1.544 mmol, 3 equiv) in THF (3 mL), was added NaH 60% dispersion in mineral oil (206 mg, 5.15 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 108 mg (purity=97%) white solid compound 98 with a yield of 60%.

TLC R_{f}=0.6 (EA:PE=1:1)

LCMS (m/z)=351.40 (M+H)

### Example 99. 1-(2,6-dimethylbenzyl)-5-hydroxy-N-methyl-2-oxo-2,3-dihydro-1H-benzo[b]azepine-4-carboxamide (99)

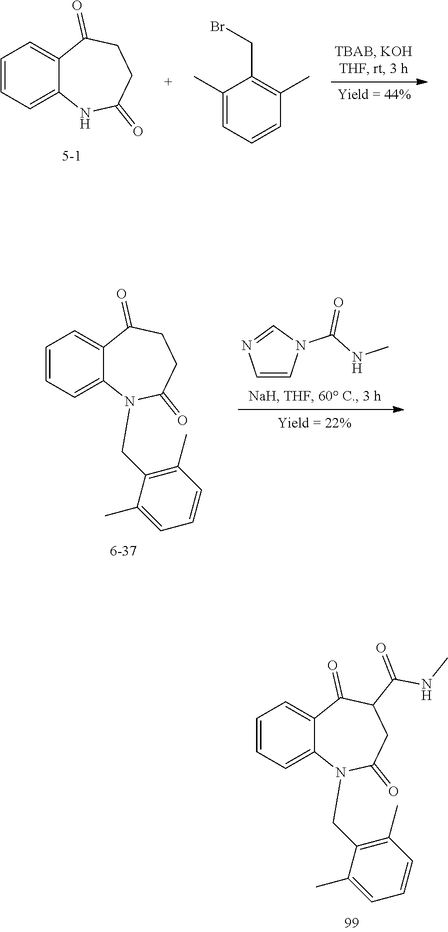

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 2-(bromomethyl)-1,3-dimethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 ml), dried over anhydrous Na₂SO₄, filtered and concentrated by rotary evaporation to get the crude compound. Then, the resulting concentrate was purified on a silica column to get 130 mg (purity=93%) light yellow solid compound 6-37 with a yield of 44%.

TLC R_{f}=0.65 (EA:PE=1:1)

LCMS (m/z)=294.40 (M+H)

To the solution of resulting compound 6-37 (130 mg, 0.444 mmol, 1 equiv) and N-methyl-1H-imidazole-1-carboxamide (166 mg, 1.328 mmol, 3 equiv) in THF (3 mL), was added NaH 60% dispersion in mineral oil (177 mg, 4.44 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 34 mg (purity=92%) white solid compound 99 with a yield of 22%.

TLC R_{f}=0.6 (EA:PE=1:1)

LCMS (m/z)=351.40 (M+H)

### Example 100. 1-(3,4-dimethylbenzyl)-5-hydroxy-N-methyl-2-oxo-2,3-dihydro-1H-benzo[b]azepine-4-carboxamide (100)

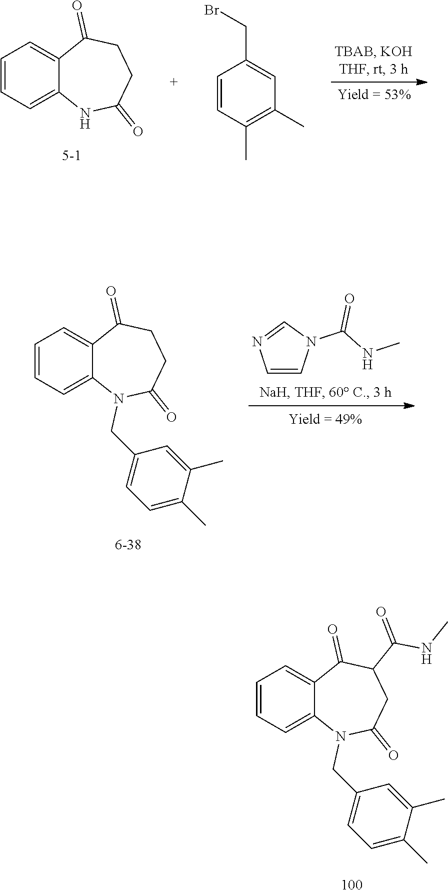

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 4-(bromomethyl)-1,2-dimethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 155 mg (purity=99%) light yellow solid compound 6-38 with a yield of 53%.

TLC R_{f}=0.65 (EA:PE=1:1)

LCMS (m/z)=294.60 (M+H)

To the solution of resulting compound 6-38 (155 mg, 0.529 mmol, 1 equiv) and N-methyl-1H-imidazole-1-carboxamide (198 mg, 1.584 mmol, 3 equiv) in THF (3 mL), was added NaH 60% dispersion in mineral oil (212 mg, 5.29 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 91 mg (purity=74%) white solid compound 100 with a yield of 49%.

TLC R_{f}=0.6 (EA:PE=1:1)

LCMS (m/z)=351.60 (M+H)

### Example 101. 1-(2,4-dimethylbenzyl)-5-hydroxy-N-methyl-2-oxo-2,3-dihydro-1H-benzo[b]azepine-4-carboxamide (101)

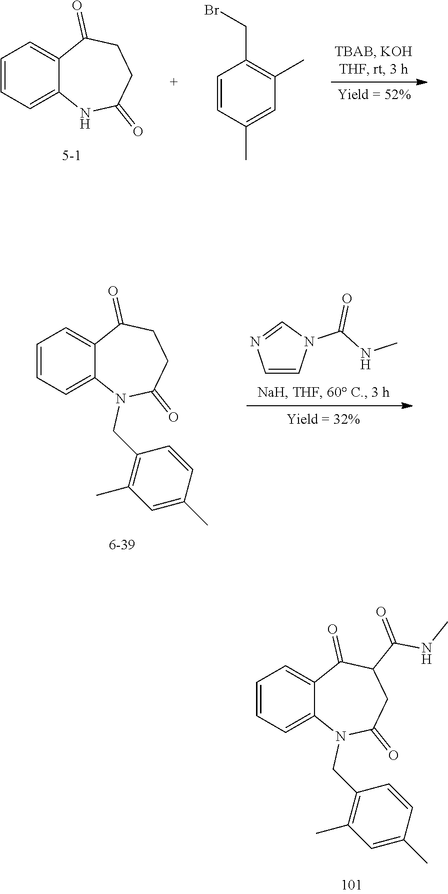

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 1-(bromomethyl)-2,4-dimethylbenzene (239 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 ml) and brine (1×20 mL), dried over anhydrous Na₂SO₄ filtered and concentrated by rotary evaporation to get the crude compound. Then, the resulting concentrate was purified on a silica column to get 153 mg (purity=96%) light yellow solid compound 6-39 with a yield of $2%.

TLC R_{f}=0.65 (EA:PE=1:1)

LCMS (m/z)=294.40 (M+H)

To the solution of resulting compound 6-39 (155 mg, 0.522 mmol, 1 equiv) and N-methyl-1H-imidazole-1-carboxamide (196 mg, 1.568 mmol, 3 equiv) in THF (3 mL), was added NaH 60% dispersion in mineral oil (209 mg, 5.22 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 59 mg (purity=92%) white solid compound 101 with a yield of 32%.

TLC R_{f}=0.6 (EA:PE=1:1)

LCMS (m/z)=351.40 (M+H)

### Example 102. 1-(3,4-difluorobenzyl)-5-hydroxy-N-methyl-2-oxo-2,3-dihydro-1H-benzo[b]azepine-4-carboxamide (102)

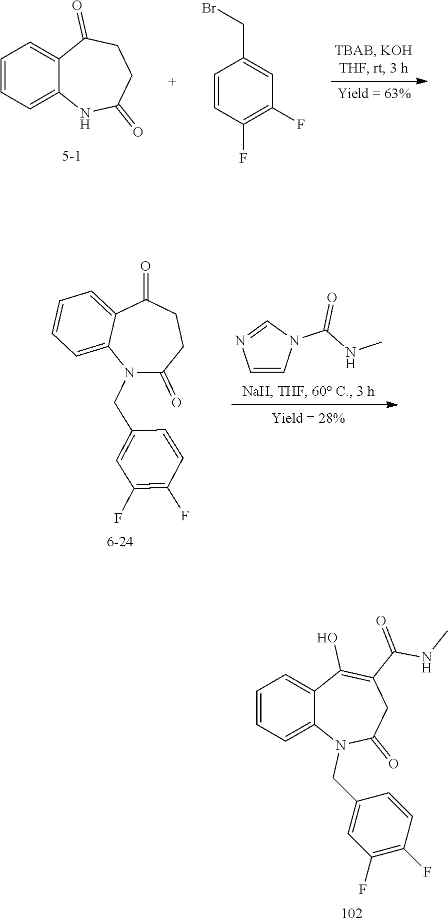

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 4-(bromomethyl)-1,2-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound.

Then, the resulting concentrate was purified on a silica column to get 190 mg (purity=94%) light yellow solid compound (6-24) with a yield of 63%

TLC R_{f}=0.45 (EA:PE=1:1)

LCMS (m/z)=302.40 (M+H)

To the solution of resulting compound 6-24 (190 mg, 0.631 mmol, 1 equiv) and N-methyl-1H-imidazole-1-carboxamide (237 mg, 1.896 mmol, 3 equiv) in THF (3 mL), was added NaH 60% dispersion in mineral oil (252 mg, 6.31 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 63 mg (purity=70%) white solid compound 102 with a yield of 28%.

TLC R_{f}=0.45 (EA:PE=1:1)

LCMS (m/z)=359.50 (M+H)

### Example 103. 1-(3,4-difluorobenzyl)-5-hydroxy-N-methyl-2-oxo-2,3-dihydro-1H-benzo[b]azepine-4-carboxamide (103)

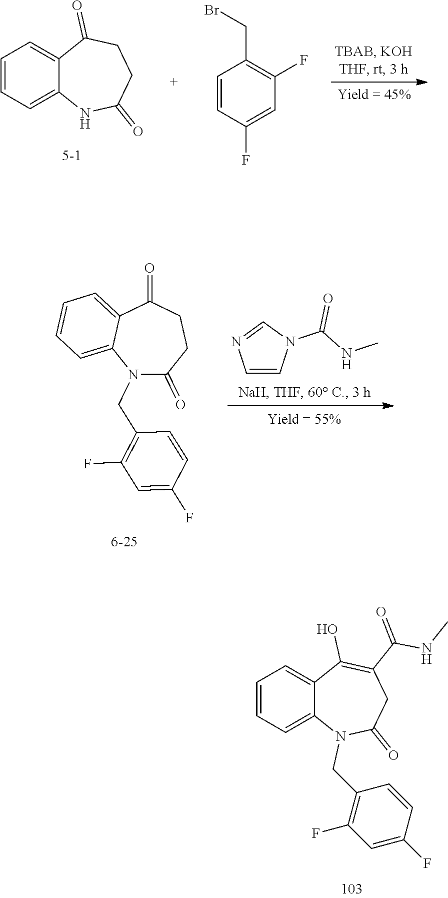

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-2,4-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 136 mg (purity=99%) light yellow solid compound 6-25 with a yield of 45%.

TLC R_{f}=0.45 (EA:PE=1:1)

LCMS (m/z)=302.40 (M+H)

To the solution of resulting compound 6-25 (136 mg, 0.452 mmol, 1 equiv) and N-methyl-1H-imidazole-1-carboxamide (169 mg, 1.352 mmol, 3 equiv) in THF (3 mL), was added NaH 60% dispersion in mineral oil (181 mg, 4.52 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 89 mg (purity=98%) white solid compound 103 with a yield of 55%.

TLC R_{f}=0.45 (EA:PE=1:1)

LCMS (m/z)=359.50 (M+H)

### Example 104. 1-(3,5-difluorobenzyl)-5-hydroxy-N-methyl-2-oxo-2,3-dihydro-1H

benzo[b]azepine-4-carboxamide (104)

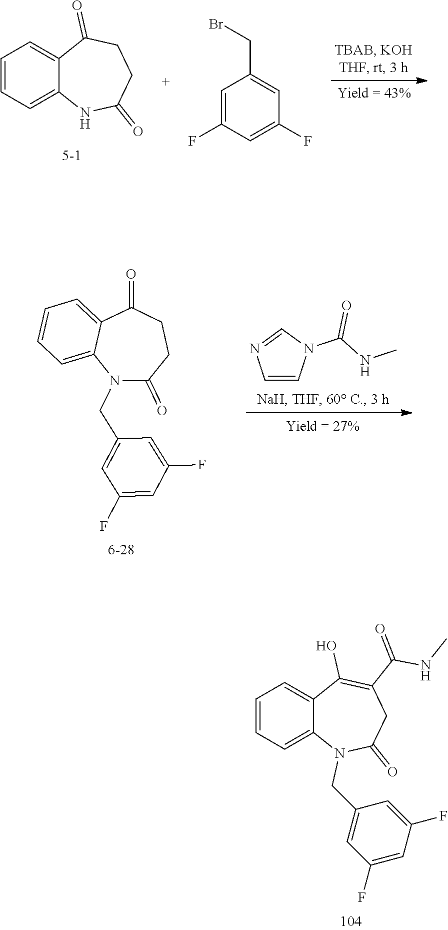

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-3,5-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 129 mg (purity=99%) light yellow solid compound 6-28 with a yield of 43%.

TLC R_{f}=0.45 (EA:PE=1:1)

LCMS (m/z)=302.40 (M+H)

To the solution of resulting compound 6-28 (129 mg, 0.429 mmol, 1 equiv) and N-methyl-1H-imidazole-1-carboxamide (161 mg, 1.288 mmol, 3 equiv) in THF (3 mL), was added NaH 60% dispersion in mineral oil (171 mg, 4.29 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with HO (2×10 mL) and brine (1×20 mL), dried over anhydrous Na₂SO₄, filtered and concentrated by rotary evaporation to get the crude compound. Then, the resulting concentrate was purified on a silica column to get 42 mg (purity=97%) white solid compound 104 with a yield of 27%.

TLC R_{f}=0.45 (EA:PE=1:1)

LCMS (m/z)=359.50 (M+H)

### Example 105. 5-hydroxy-N-methyl-2-oxo-1-(3,4,5-trifluorobenzyl)-2,3-dihydro-1H-benzo[b]azepine-4-carboxamide (105)

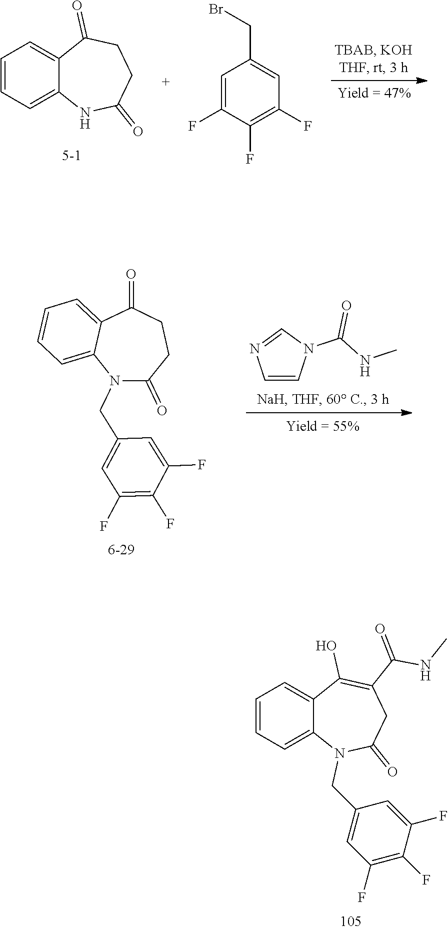

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 5-(bromomethyl)-1,2,3-trifluorobenzene (270 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 151 mg (purity=99%) light yellow solid compound 6-29 with a yield of 47%.

TLC R_{f}=0.65 (EA:PE=1:1)

LCMS (m/z)=320.30 (M+H)

To the solution of resulting compound 6-29 (151 mg, 0.433 mmol, 1 equiv) and N-methyl-1H-imidazole-1-carboxamide (178 mg, 1.424 mmol, 3 equiv) in THF (3 mL), was added NaH 60% dispersion in mineral oil (189 mg, 4.33 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 97 mg (purity=93%) white solid compound 105 with a yield of 55%.

TLC R_{f}=0.5 (EA:PE=1:1)

LCMS (m/z)=376.80 (M+H)

### Example 106. 1-(2,5-difluorobenzyl)-5-hydroxy-N-methyl-2-oxo-2,3-dihydro-1H-benzo[b]azepine-4-carboxamide (106)

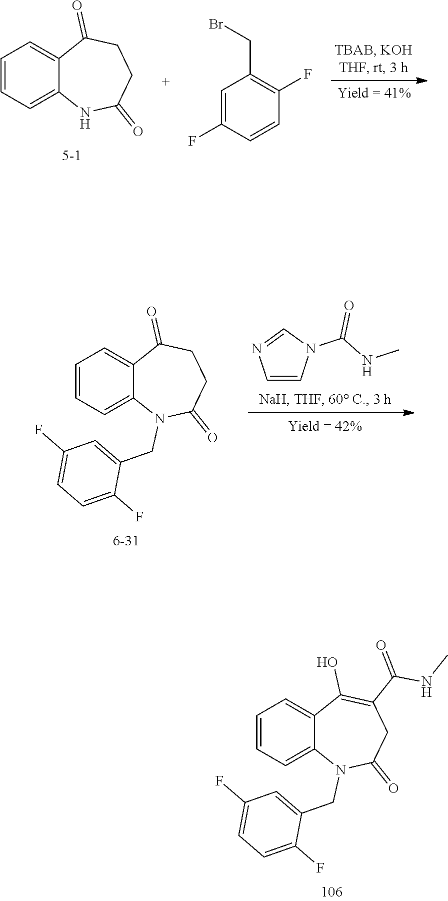

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 2-(bromomethyl)-1,4-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 124 mg (purity=99%) light yellow solid compound 6-31 with a yield of 41%.

TLC R_{f}=0.6 (EA:PE=1:1)

LCMS (m/z)=302. 40 (M+H)

To the solution of resulting compound 6-31 (124 mg, 0.412 mmol, 1 equiv) and N-methyl-1H-imidazole-1-carboxamide (154 mg, 1.232 mmol, 3 equiv) in THF (3 mL), was added NaH 60% dispersion in mineral oil (165 mg, 4.12 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 61 mg (purity=97%) white solid compound 106 with a yield of 42%.

TLC R_{f}=0.6 (EA:PE=1:1)

LCMS (m/z)=359.50 (M+H)

### Example 107. 1-(2,3-difluorobenzyl)-5-hydroxy-N-methyl-2-oxo-2,3-dihydro-1H-benzo[b]azepine-4-carboxamide (107)

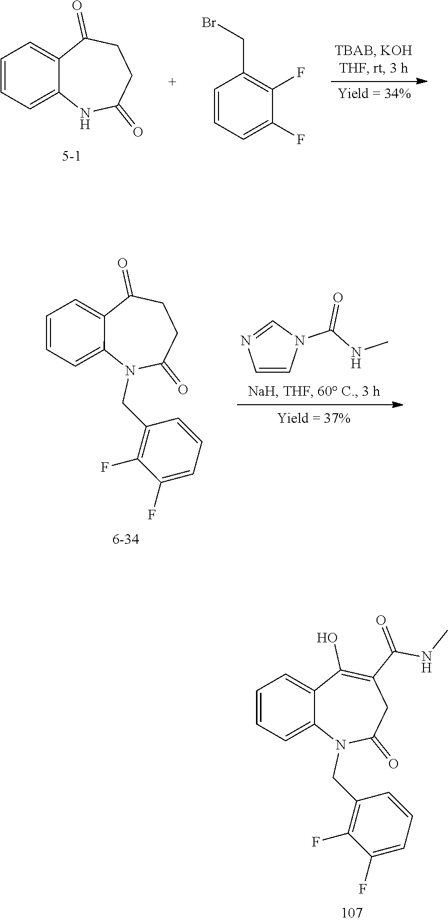

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-2,3-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 102 mg (purity=99%) light yellow solid compound 6-34 with a yield of 34%.

TLC R_{f}=0.6 (EA:PE=1:1)

LCMS (m/z)=302.40 (M+H)

To the solution of resulting compound 6-34 (102 mg, 0.339 mmol, 1 equiv) and N-methyl-1H-imidazole-1-carboxamide (127 mg, 1.016 mmol, 3 equiv) in THF (3 mL), was added NaH 60% dispersion in mineral oil (136 mg, 3.39 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 45 mg (purity=99%) white solid compound 107 with a yield of 37%.

TLC R_{f}=0.45 (EA:PE=1:1)

LCMS (m/z)=359.30 (M+H)

### Example 108. 1-(2,6-difluorobenzyl)-5-hydroxy-N-methyl-2-oxo-2,3-dihydro-1H-benzo[b]azepine-4-carboxamide (108)

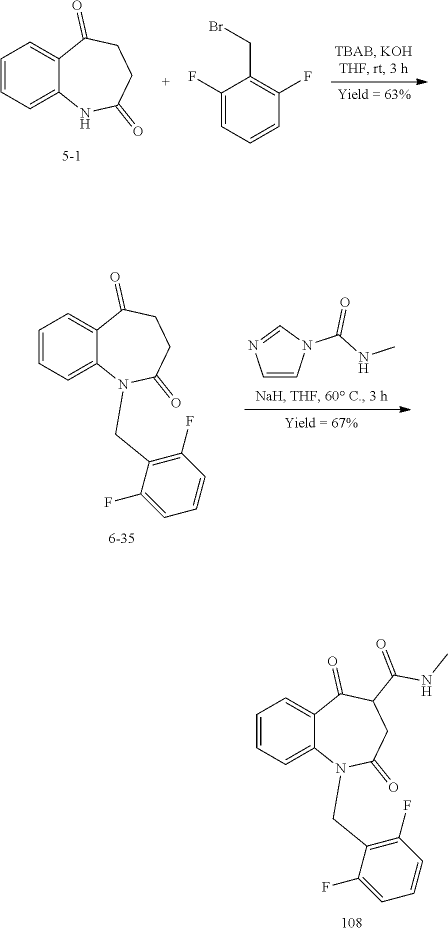

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 2-(bromomethyl)-1,3-difluorobenzene (248 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 191 mg (purity=98%) light yellow solid compound 6-35 with a yield of 63%.

TLC R_{f}=0.6 (EA:PE=1:1)

LCMS (m/z)=302.40 (M+H)

To the solution of resulting compound 6-35 (191 mg, 0.635 mmol, 1 equiv) and N-methyl-1H-imidazole-1-carboxamide (238 mg, 1.889 mmol, 3 equiv) in THF (3 mL), was added NaH 60% dispersion in mineral oil (254 mg, 6.35 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 152 mg (purity=99%) white solid compound 108 with a yield of 67%.

TLC R_{f}=0.5 (EA:PE=1:1)

LCMS (m/z)=359.50 (M+H)

### Example 109. 1-(4-fluoro-3-methylbenzyl)-5-hydroxy-N-methyl-2-oxo-2,3-dihydro-1H-benzo[b]azepine-4-carboxamide (109)

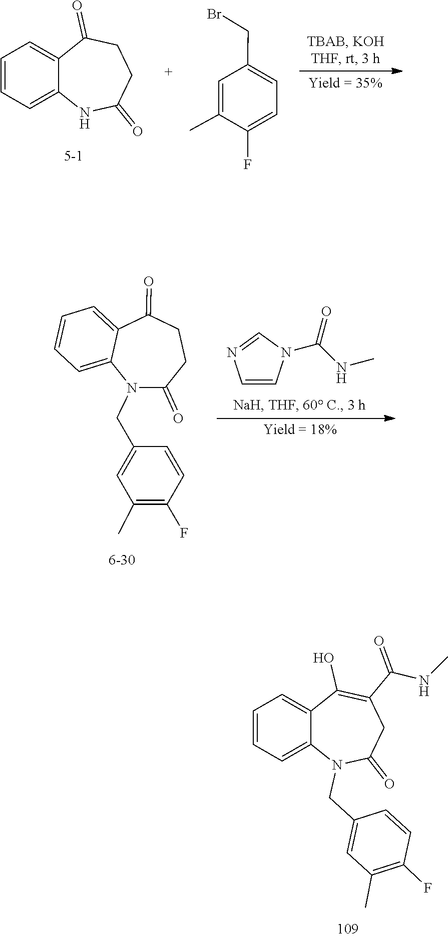

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 4-(bromomethyl)-1-fluoro-2-methylbenzene (241 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 104 mg (purity=99%) light yellow solid compound 6-30 with a yield of 35%.

TLC R_{f}=0.6 (EA:PE=1:1)

LCMS (m/z)=298.40 (M+H)

To the solution of resulting compound 6-30 (104 mg, 0.350 mmol, 1 equiv) and N-methyl-1H-imidazole-1-carboxamide (131 mg, 1.048 mmol, 3 equiv) in THF (2 mL), was added NaH 60% dispersion in mineral oil (140 mg, 3.50 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 23 mg (purity=99%) white solid compound 109 with a yield of 18%.

TLC R_{f}=0.5 (EA:PE=1:1)

LCMS (m/z)=355.30 (M+H)

### Example 111. 1-(3-fluoro-4-methoxybenzyl)-5-hydroxy-N-methyl-2-oxo-2,3-dihydro-1H-benzo[b]azepine-4-carboxamide (111)

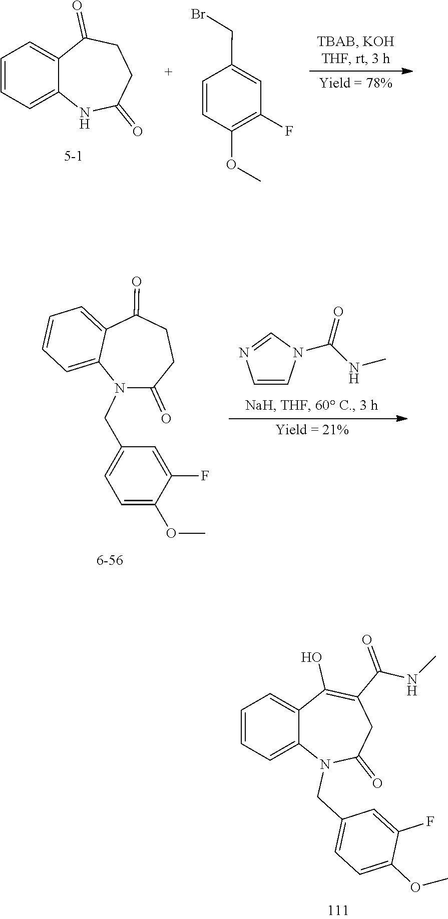

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 4-(bromomethyl)-2-fluoro-1-methoxybenzene (263 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 244 mg (purity=99%) light yellow solid compound 6-56 with a yield of 78%.

TLC R_{f}=0.6 (PE:EA=2:1)

LCMS (m/z)=314.40 (M+H)

To the solution of resulting compound 6-56 (244 mg, 0.780 mmol, 1 equiv) and NaH (315 mg, 7.80 mmol, 10 equiv) in THF (4 mL), was added N-methyl-1H-imidazole-1-carboxamide (119 mg, 0.952 mmol, 1.2 equiv) under Nitrogen, and the reaction mixture was allowed to stir at r.t for 4 h. The organic solution was separated and the aqueous solution was extracted with EA (3×40 mL). The organic solutions were combined, washed with H₂O (2×40 mL) and brine (1×100 mL), dried over anhydrous Na₂SO₄, filtered and concentrated by rotary evaporation to get the crude compound. Then, the resulting concentrate was purified by Pre-TLC to get 60 mg (purity=96%) white solid compound with a yield of 21%.

TLC R_{f}=0.6 (EA:PE=1:2)

LCMS (m/z)=371.60 (M+H)

### Example 112. 1-(4-fluoro-3-methoxybenzyl)-5-hydroxy-N-methyl-2-oxo-2,3-dihydro-1H-benzo[b]azepine-4-carboxamide (112)

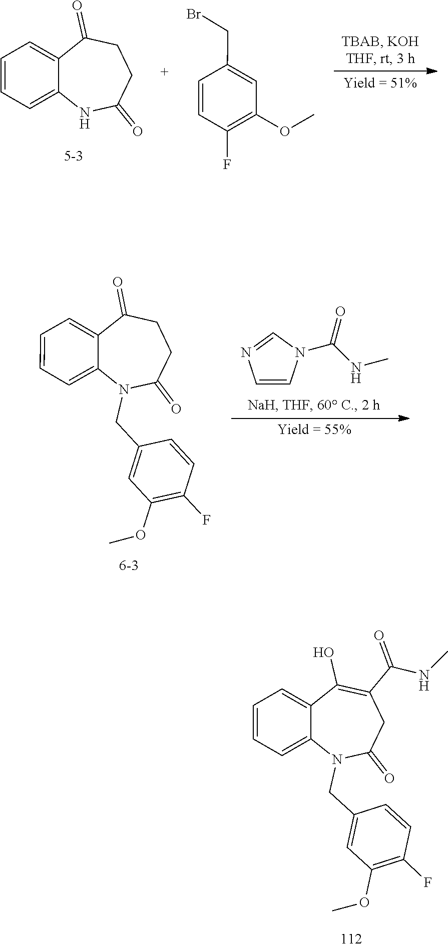

To a stirred solution of ketone (5-3) (350 mg, 2 mmol, 1 equiv), Tetrabutylammonium bromide (64 mg, 0.2 mmol, 0.1 equiv) and KOH (157 mg, 2.8 mmol, 1.4 equiv) in THF (8 mL), was added 4-(bromomethyl)-1-fluoro-2-methoxybenzene (482 mg, 2.2 mmol, 1.1 equiv) at ambient temperature. The reaction mixture was then stirred at RT for 3 h. The reaction progress was monitored by TLC until the complete disappearance of compound 5-3. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 320 mg (purity=98%) light yellow solid compound 6-3 with a yield of 51%.

TLC R_{f}=0.3 (PE:EA=1:1)

LCMS (m/z)=314.50 (M+H)

¹H NMR (400 MHZ, DMSO) δ 7.59-7.50 (m, 1H), 7.46 (d, J=8.1 Hz, 1H), 7.43-7.37 (m, 1H), 7.26 (t, J=7.4 Hz, 1H), 7.04 (dd, J=11.5, 8.3 Hz, 1H), 6.83-6.75 (m, 1H), 6.70-6.62 (m, 1H), 5.07 (s, 2H), 3.71 (s, 3H), 3.09-2.97 (m, 2H), 2.84-2.72 (m, 2H). ¹³C NMR (100 MHz, DMSO) δ 203.80, 172.16, 152.11, 149.69, 147.49, 147.38, 140.68, 134.73, 134.55, 134.51, 133.40, 129.29, 126.65, 124.09, 119.97, 119.90, 116.13, 115.95, 112.61, 56.05, 49.35, 41.66, 31.20.

To a stirred solution of ketone (6-3) (200 mg, 0.63 mmol, 1 equiv) and N-methyl-1H-imidazole-1-carboxamide (239 mg, 1.91 mmol, 3 equiv) in THF (3 mL), was added NaH 60% dispersion in mineral oil (252 mg, 6.3 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 130 mg (purity=96%) light yellow oil compound 112 with a yield of 55%.

TLC R_{f}=0.3 (PE:EA=1:1)

LCMS (m/z)=371.60 (M+H)

¹H NMR (400 MHZ, DMSO) δ 15.28 (s, 1H), 8.43 (d, J=3.9 Hz, 1H), 7.70 (d, J=7.2 Hz, 1H), 7.58-7.43 (m, 2H), 7.27 (t, J=7.3 Hz, 1H), 7.05 (dd, J=11.4, 8.3 Hz, 1H), 6.72-6.54 (m, 2H), 5.34 (d, J=16.0 Hz, 1H), 4.90 (d, J=16.0 Hz, 1H), 3.67 (s, 3H), 3.54 (d, J=14.3 Hz, 1H), 2.77 (d, J=3.8 Hz, 3H), 2.60 (d, J=14.3 Hz, 1H). ¹³C NMR (100 MHz, DMSO) δ 170.78, 164.50, 151.97, 149.56, 147.46, 147.36, 140.37, 134.74, 134.71, 131.41, 130.11, 128.06, 125.55, 123.55, 119.27, 119.20, 116.15, 115.97, 111.72, 99.15, 55.79, 49.59, 30.87, 26.65.

### Example 113. 1-(2-fluoro-3-methoxybenzyl)-5-hydroxy-N-methyl-2-oxo-2,3-dihydro-1H-benzo[b]azepine-4-carboxamide (113)

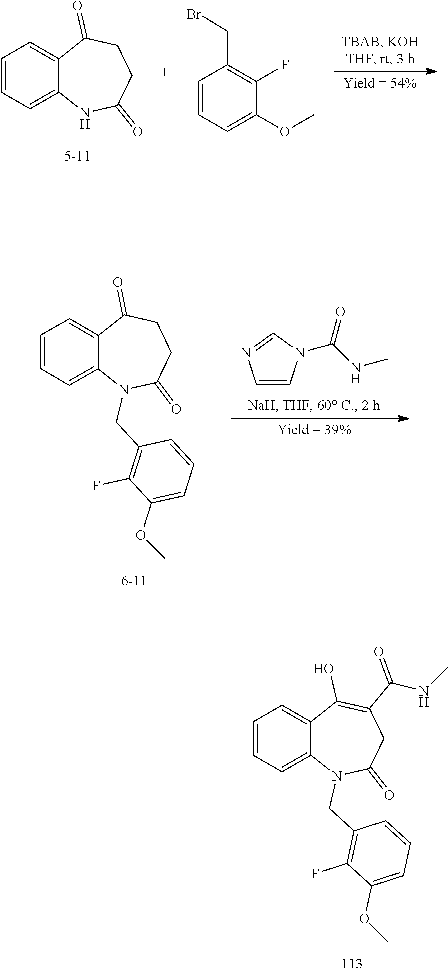

To a stirred solution of ketone (5-11) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-2-fluoro-3-methoxybenzene (263 mg, 1.2 mmol, 1.2 equiv) at ambient temperature. The reaction mixture was then stirred at RT for 3 h. The reaction progress was monitored by TLC until the complete disappearance of compound 5-11. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 168 mg (purity=99%) light yellow solid compound 6-11 with a yield of 54%.

TLC R_{f}=0.45 (PE:EA=1:1)

LCMS (m/z)=314.50 (M+H)

To a stirred solution of ketone (6-11) (168 mg, 0.537 mmol, 1 equiv) and N-methyl-1H-imidazole-1-carboxamide (201 mg, 1.61 mmol, 3 equiv) in THF (3 mL), was added NaH 60% dispersion in mineral oil (214 mg, 5.37 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 78 mg (purity=92%) white solid compound 113 with a yield of 39%.

TLC R_{f}=0.35 (PE:EA=1:1)

LCMS (m/z)=371.40 (M+H)

### Example 114. 1-(5-fluoro-2-methoxybenzyl)-5-hydroxy-N-methyl-2-oxo-2,3-dihydro-1H-benzo[b]azepine-4-carboxamide (114)

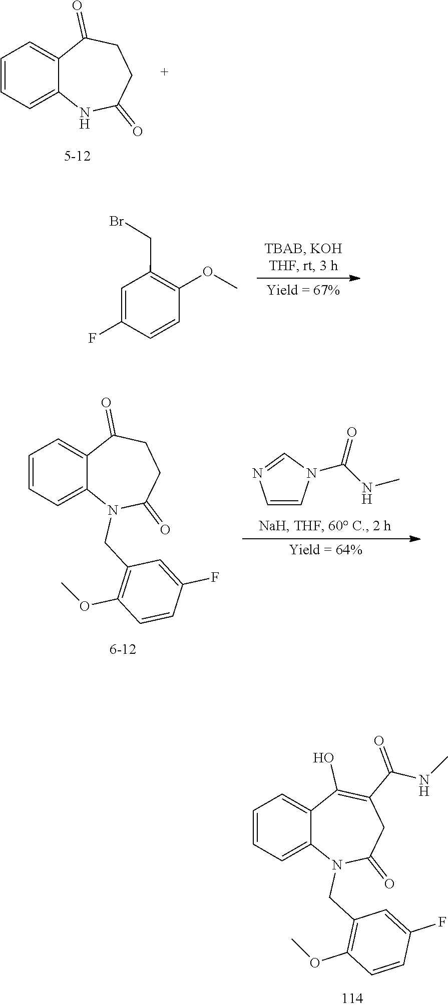

To a stirred solution of ketone (5-12) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 1-(bromomethyl)-5-fluoro-2-methoxy-3-methylbenzene (263 mg, 1.2 mmol, 1.2 equiv) at ambient temperature. The reaction mixture was then stirred at RT for 3 h. The reaction progress was monitored by TLC until the complete disappearance of compound 5-12. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 209 mg (purity=97%) light yellow solid compound 6-12 with a yield of 67%.

TLC R_{f}=0.45 (PE:EA=1:1)

LCMS (m/z)=314.50 (M+H)

To a stirred solution of ketone (6-12) (209 mg, 0.666 mmol, 1 equiv) and N-methyl-1H-imidazole-1-carboxamide (250 mg, 2 mmol, 3 equiv) in THF (4 mL), was added NaH 60% dispersion in mineral oil (214 mg, 6.66 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 158 mg (purity=95%) white solid compound 114 with a yield of 64%.

TLC R_{f}=0.35 (PE:EA=1:1)

LCMS (m/z)=371.40 (M+H)

### Example 115. 1-(5-fluoro-2-methylbenzyl)-5-hydroxy-N-methyl-2-oxo-2,3-dihydro-1H-benzo[b]azepine-4-carboxamide (115)

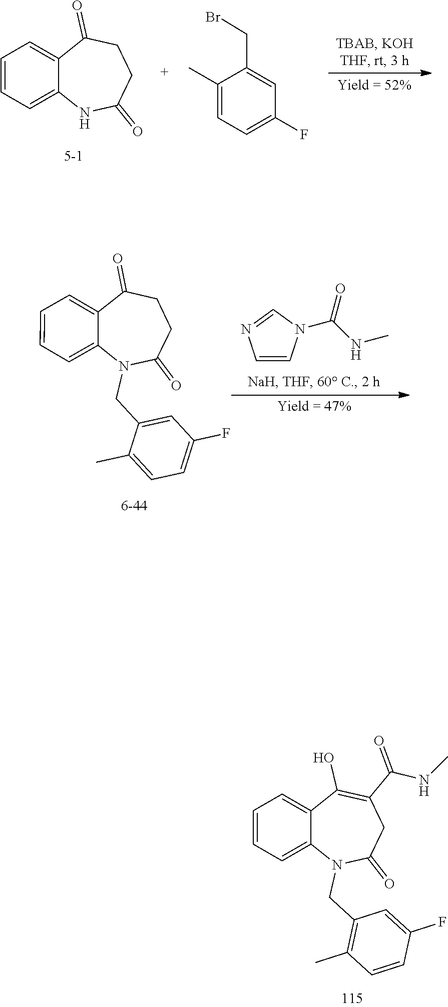

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 2-(bromomethyl)-4-fluoro-1-methylbenzene (244 mg, 1.2 mmol, 1.2 equiv) at ambient temperature. The reaction mixture was then stirred at RT for 3 h. The reaction progress was monitored by TLC until the complete disappearance of compound (5-1). The solution was quenched with saturated ammonium chloride and extracted with EA (3×20 mL). The organic solutions were combined, washed with H₂O (2×20 mL) and brine (1×20 ml), dried over anhydrous Na₂SO₄, filtered and concentrated by rotary evaporation to get the crude compound. Then, the resulting concentrate was purified on a silica column to get 153 mg (purity=93%) light yellow solid compound 6-44 with a yield of $2%.

TLC R_{f}=0.6 (EA:PE=1:1)

LCMS (m/z)=298.40 (M+H)

To the solution of resulting compound 6-44 (153 mg, 0.515 mmol, 1 equiv) and N-methyl-1H-imidazole-1-carboxamide (193 mg, 1.544 mmol, 3 equiv) in THF (3 mL), was added NaH 60% dispersion in mineral oil (206 mg, 5.15 mmol, 10 equiv) at ambient temperature under N, atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 85 mg (purity=98%) white solid compound 115 with a yield of 47%.

TLC R_{f}=0.5 (EA:PE=1:1)

LCMS (m/z)=355.50 (M+H)

### Example 116. 1-(3-chloro-4-fluorobenzyl)-5-hydroxy-N-methyl-2-oxo-2,3-dihydro-1H-benzo[b]azepine-4-carboxamide (116)

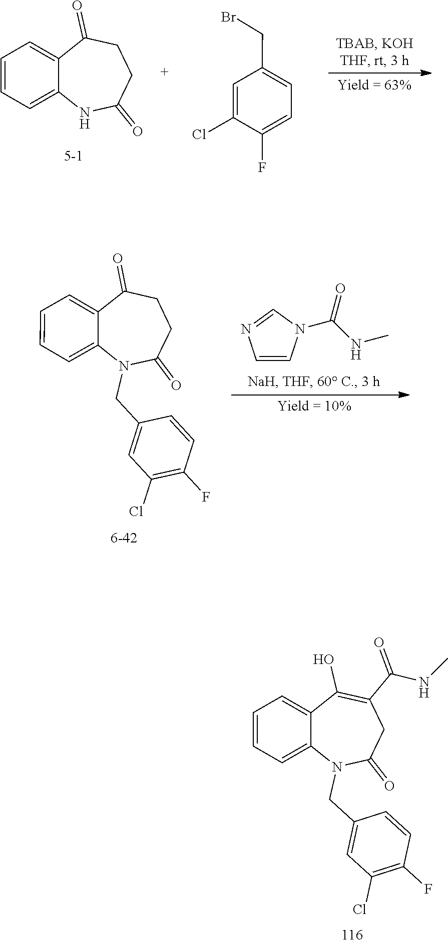

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 4-(bromomethyl)-2-chloro-1-Fluorobenzene (268 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 201 mg (purity=99%) light yellow solid compound 6-42 with a yield of 63%.

TLC R_{f}=0.7 (EA:PE=1:1)

LCMS (m/z)=318.40 (M+H)

To the solution of resulting compound 6-42 (201 mg, 0.634 mmol, 1 equiv) and N-methyl-1H-imidazole-1-carboxamide (235 mg, 1.880 mmol, 3 equiv) in THF (3 mL), was added NaH 60% dispersion in mineral oil (254 mg, 6.34 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 24 mg (purity=95%) white solid compound 116 with a yield of 10%.

TLC R_{f}=0.4 (EA:PE=1:1)

LCMS (m/z)=375.10 (M+H)

### Example 117. 1-(3-cyano-4-fluorobenzyl)-5-hydroxy-N-methyl-2-oxo-2,3-dihydro-1H-benzo[b]azepine-4-carboxamide (117)

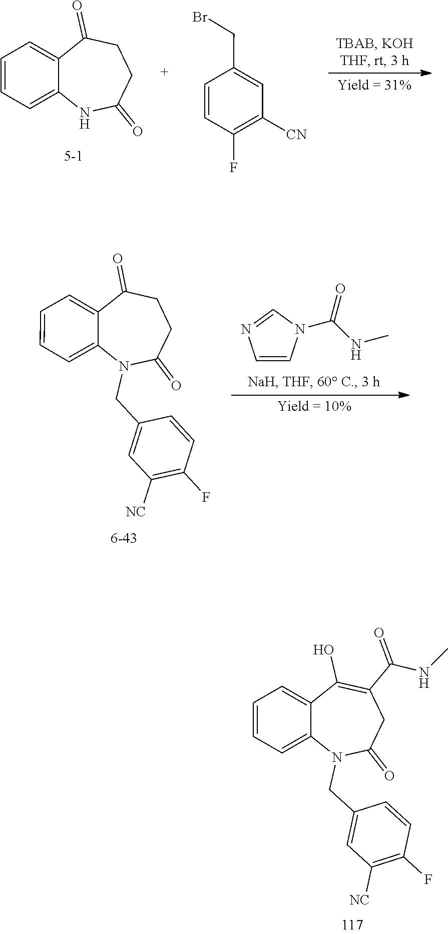

To the solution of resulting compound 5-1 (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was added 5-(bromomethyl)-2-fluorobenzonitrile (257 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 96 mg (purity=99%) light yellow solid compound 6-43 with a yield of 31%.

TLC R_{f}=0.7 (EA:PE=1:1)

LCMS (m/z)=309.40 (M+H)

To the solution of resulting compound 6-43 (96 mg, 0.312 mmol, 1 equiv) and N-methyl-1H-imidazole-1-carboxamide (117 mg, 0.936 mmol, 3 equiv) in THF (3 mL), was added NaH 60% dispersion in mineral oil (125 mg, 31.2 mmol, 10 equiv) at ambient temperature under N, atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 1.5 mg (purity=65%) white solid compound 117 with a yield of 1.3%.

TLC R_{f}=0.4 (EA:PE=1:1)

LCMS (m/z)=366.30 (M+H)

### Example 118. 1-(4-fluoro-3-(trifluoromethyl) benzyl)-5-hydroxy-N-methyl-2-oxo-2,3-dihydro-1H-benzo[b]azepine-4-carboxamide (118)

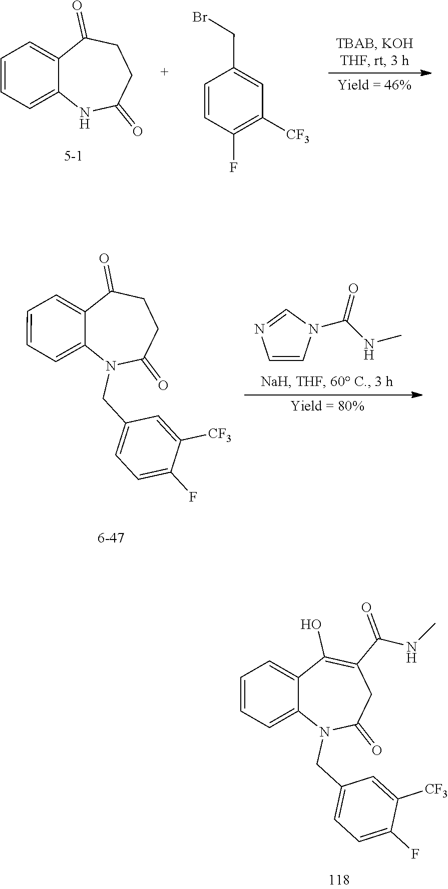

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 4-(bromomethyl)-1-fluoro-2-(trifluoromethyl)benzene (304 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 160 mg (purity=99%) light yellow solid compound 6-47 with a yield of 46%.

TLC R_{f}=0.7 (EA:PE=1:1)

LCMS (m/z)=352.50 (M+H)

To the solution of resulting compound 6-47 (160 mg, 0.456 mmol, 1 equiv) and N-methyl-1H-imidazole-1-carboxamide (171 mg, 1.368 mmol, 3 equiv) in THF (3 mL), was added NaH 60% dispersion in mineral oil (182 mg, 45.6 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 132 mg (purity=99%) white solid compound 118 with a yield of 80%.

TLC R_{f}=0.65 (EA:PE=1:1)

LCMS (m/z)=359.80 (M+H)

### Example 119. 1-(3-chloro-4-methylbenzyl)-5-hydroxy-N-methyl-2-oxo-2,3-dihydro-1H-benzo[b]azepine-4-carboxamide (119)

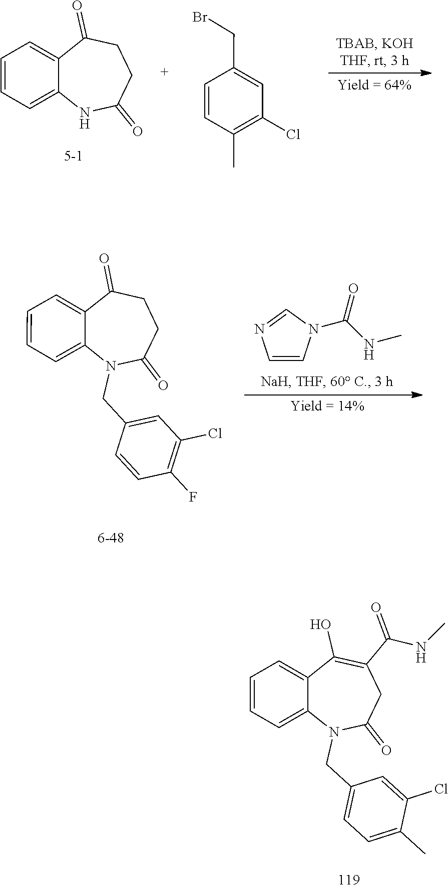

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 4-(bromomethyl)-2-chloro-1-methylbenzene (263 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 202 mg (purity=99%) light yellow solid compound 6-48 with a yield of 64%.

TLC R_{f}=0.6 (EA:PE=1:1)

LCMS (m/z)=314.50 (M+H)

To the solution of resulting compound 6-48 (202 mg, 0.642 mmol, 1 equiv) and N-methyl-1H-imidazole-1-carboxamide (242 mg, 1.936 mmol, 3 equiv) in THF (3 mL), was added NaH 60% dispersion in mineral oil (258 mg, 64.2 mmol, 10 equiv) at ambient temperature under N, atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 34 mg (purity=99%) white solid compound 119 with a yield of 14%.

TLC R_{f}=0.5 (EA:PE=1:1)

LCMS (m/z)=371.60 (M+H)

### Example 120. 1-(3-chloro-4-methylbenzyl)-5-hydroxy-N-methyl-2-oxo-2,3-dihydro-1H-benzo[b]azepine-4-carboxamide (120)

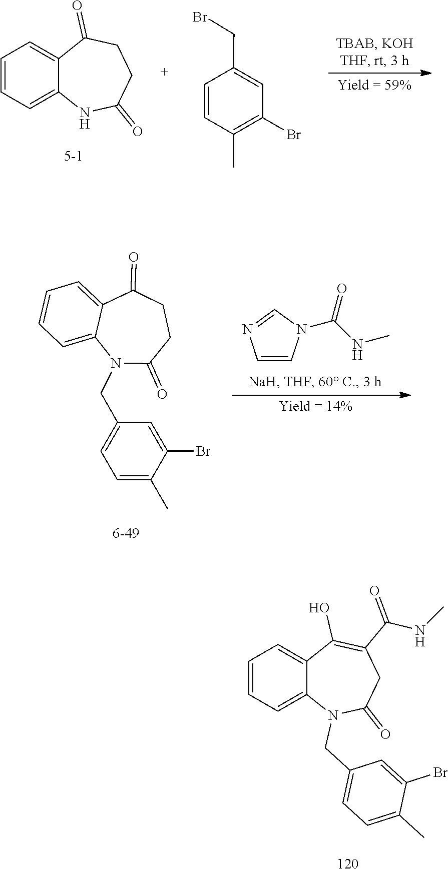

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 2-bromo-4-(bromomethyl)-1-methylbenzene (316 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 213 mg (purity=99%) light yellow solid compound 6-49 with a yield of 59%.

TLC R_{f}=0.55 (EA:PE=1:1)

LCMS (m/z)=359.80 (M+H)

To the solution of resulting compound 6-49 (213 mg, 0.595 mmol, 1 equiv) and N-methyl-1H-imidazole-1-carboxamide (223 mg, 1.784 mmol, 3 equiv) in THF (3 mL), was added NaH 60% dispersion in mineral oil (238 mg, 59.5 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 74 mg (purity=99%) white solid compound 120 with a yield of 14%.

TLC R_{f}=0.5 (EA:PE=1:1)

LCMS (m/z)=416.90 (M+H)

### Example 121. 1-(4-fluoro-3,5-dimethylbenzyl)-5-hydroxy-N-methyl-2-oxo-2,3-dihydro-1H-benzo[b]azepine-4-carboxamide (121)

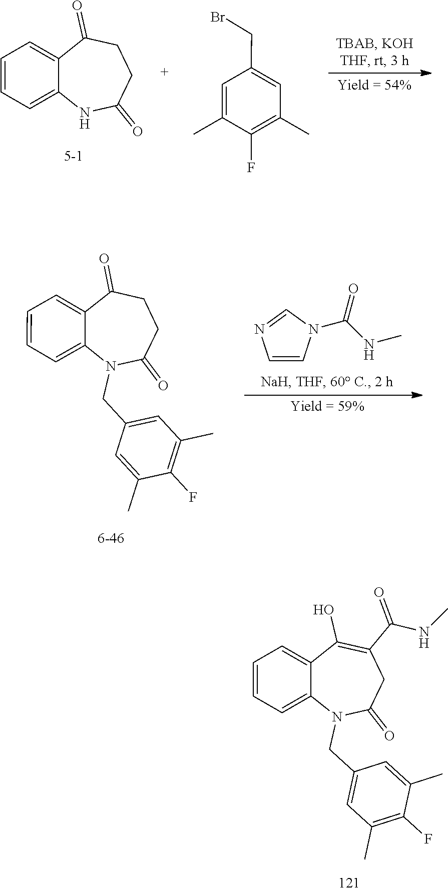

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was added 5-(bromomethyl)-2-fluoro-1,3-dimethylbenzene (260 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 169 mg (purity=99%) light yellow solid compound 6-46 with a yield of 54%.

TLC R_{f}=0.7 (EA:PE=1:1)

LCMS (m/z)=312.40 (M+H)

To the solution of resulting compound 6-46 (169 mg, 0.543 mmol, 1 equiv) and N-methyl-1H-imidazole-1-carboxamide (204 mg, 1.632 mmol, 3 equiv) in THF (3 mL), was added NaH 60% dispersion in mineral oil (217 mg, 54.3 mmol, 10 equiv) at ambient temperature under N, atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 57 mg (purity=97%) white solid compound 121 with a yield of 59%.

TLC R_{f}=0.65 (EA:PE=1:1)

LCMS (m/z)=369.50 (M+H)

### Example 122. 1-(3,5-difluoro-4-methoxybenzyl)-5-hydroxy-N-methyl-2-oxo-2,3-dihydro-1H-benzo[b]azepine-4-carboxamide (122)

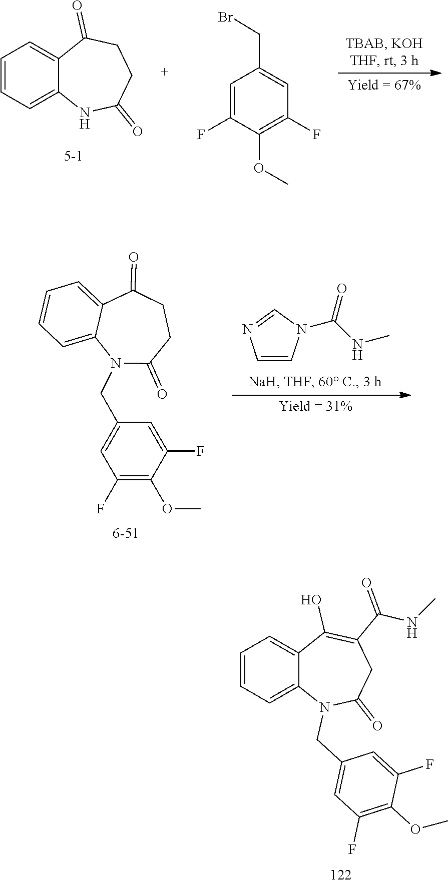

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was 5-(bromomethyl)-1,3-difluoro-2-methoxybenzene (275 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 223 mg (purity=99%) light yellow solid compound 6-51 with a yield of 67%.

TLC R_{f}=0.55 (EA:PE=1:1)

LCMS (m/z)=332.60 (M+H)

To the solution of resulting compound 6-51 (233 mg, 0.674 mmol, 1 equiv) and N-methyl-1H-imidazole-1-carboxamide (253 mg, 2.024 mmol, 3 equiv) in THF (3 mL), was added NaH 60% dispersion in mineral oil (269 mg, 6.74 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 80 mg (purity=99%) white solid compound 122 with a yield of 31%.

TLC R_{f}=0.5 (EA:PE=1:1)

LCMS (m/z)=389.40 (M+H)

### Example 123. 5-hydroxy-1-(4-methoxy-3-(trifluoromethyl) benzyl)-N-methyl-2-oxo-2,3-dihydro-1H-benzo[b]azepine-4-carboxamide (123)

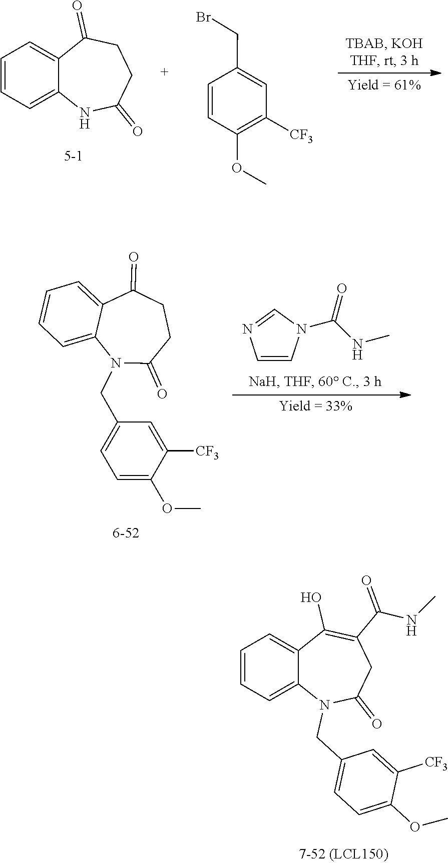

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 4-(bromomethyl)-1-methoxy-2-(trifluoromethyl) benzene (333 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 223 mg (purity=75%) light yellow solid compound 6-52 with a yield of 61%.

TLC R_{f}=0.55 (EA:PE=1:1)

LCMS (m/z)=364.60 (M+H)

To the solution of resulting compound 6-52 (233 mg, 0.614 mmol, 1 equiv) and N-methyl-1H-imidazole-1-carboxamide (232 mg, 1.856 mmol, 3 equiv) in THF (3 mL), was added NaH 60% dispersion in mineral oil (245 mg, 6.14 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 84 mg (purity=99%) white solid compound 123 with a yield of 33%.

TLC R_{f}=0.5 (EA:PE=1:1)

LCMS (m/z)=421.60 (M+H)

### Example 124. 1-(4-chloro-3-fluorobenzyl)-5-hydroxy-N-methyl-2-oxo-2,3-dihydro-1H-benzo[b]azepine-4-carboxamide (124)

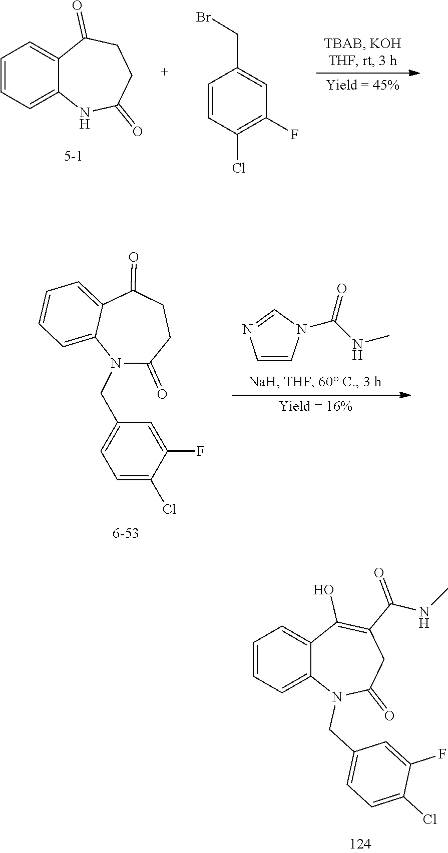

To a stirred solution of ketone (5-1) (175 mg, 1 mmol, 1 equiv). Tetrabutylammonium bromide (33 mg, 0.1 mmol, 0.1 equiv) and KOH (79 mg, 1.4 mmol, 1.4 equiv) in THE (4 mL), was 4-(bromomethyl)-1-chloro-2-fluorobenzene (268 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 142 mg (purity=99%) light yellow solid compound 6-53 with a yield of 45%.

TLC R_{f}=0.5 (EA:PE=1:1)

LCMS (m/z)=318.38 (M+H)

To the solution of resulting compound 6-53 (142 mg, 0.448 mmol, 1 equiv) and N-methyl-1H-imidazole-1-carboxamide (168 mg, 1.344 mmol, 3 equiv) in THF (3 mL), was added NaH 60% dispersion in mineral oil (179 mg, 4.48 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 27 mg (purity=98%) white solid compound 124 with a yield of 16%.

TLC R_{f}=0.5 (EA:PE=1:1)

LCMS (m/z)=375.20 (M+H)

### Example 125. 1-(4-bromo-3-fluorobenzyl)-5-hydroxy-N-methyl-2-oxo-2,3-dihydro-1H-benzo[b]azepine-4-carboxamide (125)

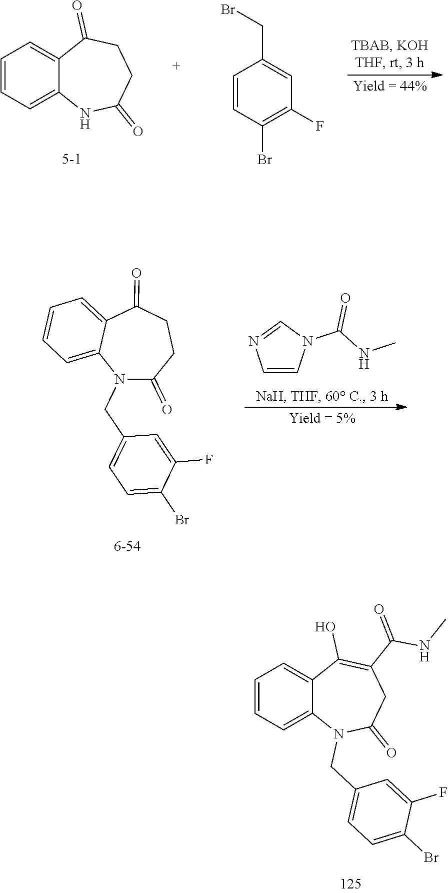

To a stirred solution of ketone (5-1) (175 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 1-bromo-4-(bromomethyl)-2-fluorobenzene (268 mg, 1.2 mmol, 1.2 equiv) at ambient temperature. The reaction mixture was then stirred at RT for 3 h. The reaction progress was monitored by TLC until the complete disappearance of compound 5-1. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 160 mg (purity=99%) light yellow solid compound 6-54 with a yield of 44%

TLC R_{f}=0.5 (EA:PE=1:1)

LCMS (m/z)=362.48 (M+H)

To the solution of resulting compound 6-54 (160 mg, 0.443 mmol, 1 equiv) and N-methyl-1H-imidazole-1-carboxamide (166 mg, 1.328 mmol, 3 equiv) in THE (3 mL), was added NaH 60% dispersion in mineral oil (177 mg, 4.43 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 9 mg (purity=92%) white solid compound 125 with a yield of 5%.

TLC R_{f}=0.5 (EA:PE=1:1)

LCMS (m/z)=421.10 (M+H)

### Example 127. 1-(3-fluoro-4-methylbenzyl)-5-hydroxy-8-methoxy-N-methyl-2-oxo-2,3-dihydro-1H-benzo[b]azepine-4-carboxamide (127)

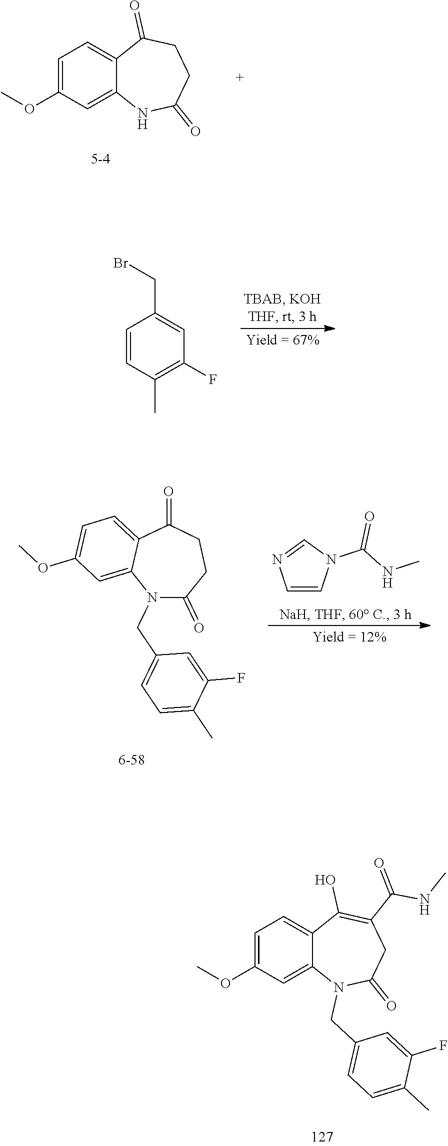

To a stirred solution of ketone (5-4) (205 mg, 1 mmol, 1 equiv), Tetrabutylammonium bromide (33 mg, 0.1 mmol, 0.1 equiv) and KOH (79 mg, 1.4 mmol, 1.4 equiv) in THE (4 mL), was 4-(bromomethyl)-2-fluoro-1-methylbenzene (244 mg, 1.2 mmol, 1.2 equiv) at ambient temperature. The reaction mixture was then stirred at RT for 3 h. The reaction progress was monitored by TLC until the complete disappearance of compound 5-4. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 219 mg (purity=99%) light yellow solid compound 6-58 with a yield of 67%

TLC R_{f}=0.5 (EA:PE=1:1)

LCMS (m/z)=328.50 (M+H)

To the solution of resulting compound 6-58 (219 mg, 0.670 mmol, 1 equiv) and N-methyl-1H-imidazole-1-carboxamide (251 mg, 2.01 mmol, 3 equiv) in THF (3 mL), was added NaH 60% dispersion in mineral oil (268 mg, 6.70 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 31 mg (purity=99%) white solid compound 127 with a yield of 12%.

TLC R_{f}=0.5 (EA:PE=1:1)

LCMS (m/z)=385.60 (M+H)

### Example 128. 8-ethoxy-1-(3-fluoro-4-methylbenzyl)-5-hydroxy-N-methyl-2-oxo-2,3-dihydro-1H-benzo[b]azepine-4-carboxamide (128)

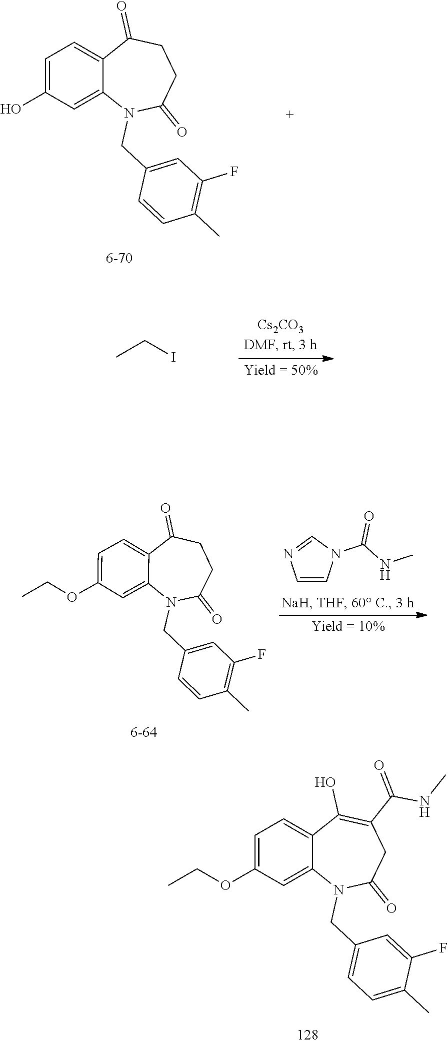

To a stirred solution of ketone 6-70 (100 mg, 0.319 mmol, 1 equiv), Cs2CO3 (208 mg, 0.638 mmol, 2 equiv) in DMF (3 mL), was added iodoethane (27 mg, 0.351 mmol, 1.1 equiv) at 0° C. The reaction mixture was then stirred at RT for 16 h. The reaction progress was monitored by TLC until the complete disappearance of 6-70. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 40 mg (purity=99%) light yellow solid compound 6-64 with a yield of 50%.

TLC R_{f}=0.7 (DCM:MeOH=40:1)

LCMS (m/z)=342.50 (M+H)

To the solution of resulting compound 6-64 (40 mg, 0.117 mmol, 1 equiv) and N-methyl-1H-imidazole-1-carboxamide (44 mg, 0.351 mmol, 3 equiv) in THF (3 mL), was added NaH 60% dispersion in mineral oil (147 mg, 1.17 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 7 mg (purity=95%) white solid compound 128 with a yield of 12%.

TLC R_{f}=0.3 (EA:PE=1:1)

LCMS (m/z)=399.80 (M+H)

### Example 129. 8-(difluoromethoxy)-1-(3-fluoro-4-methylbenzyl)-5-hydroxy-N-methyl-2-oxo-2,3-dihydro-1H-benzo[b]azepine-4-carboxamide (129)

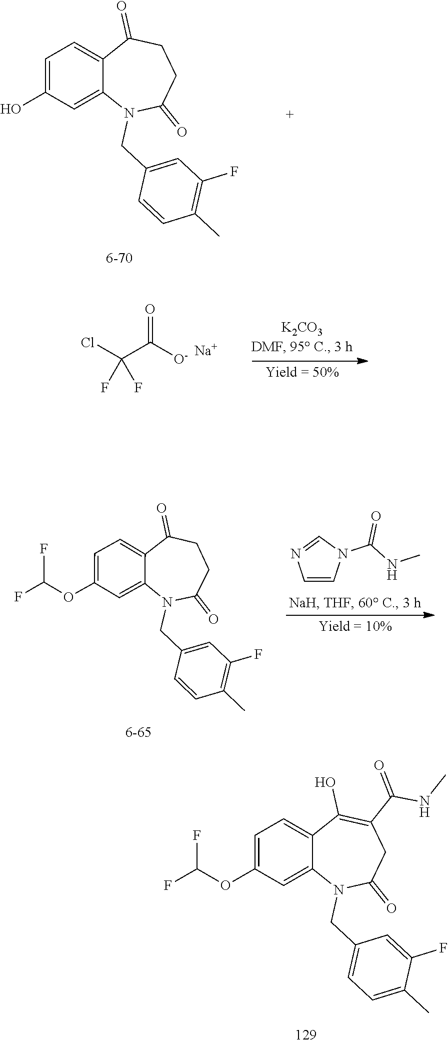

A solution of sodium chlorodifluoroacetate (152 mg, 1 mmol, 2 equiv) and ketone (6-70) (156 mg, 0.5 mmol, 1 equiv) in DMF (0.3 mL) was added slowly to a suspension of K₂CO₃ (104 mg, 0.75 mmol, 5 equiv) in DMF (0.4 mL) at 95° C. for 3 h. When the addition was complete, the suspension was stirred for a further 15 min and then cooled to 15° C. H₂O (0.3 L) was added and the resulting mixture was extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 50 mg (purity=97%) light yellow solid compound 6-65 with a yield of 50%.

TLC R_{f}=0.4 (EA:PE=2:1)

LCMS (m/z)=364.40 (M+H)

To the solution of resulting compound 6-65 (50 mg, 0.137 mmol, 1 equiv) and N-methyl-1H-imidazole-1-carboxamide (44 mg, 0.351 mmol, 3 equiv) in THF (3 mL), was added NaH 60% dispersion in mineral oil (147 mg, 1.17 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 32 mg (purity=99%) white solid compound 129 with a yield of 52%.

TLC R_{f}=0.3 (EA:PE=1:1)

LCMS (m/z)=421.20 (M+H)

### Example 130. 1-(4-fluoro-3-methylbenzyl)-5-hydroxy-N,8-dimethyl-2-oxo-2,3-dihydro-1H-benzo[b]azepine-4-carboxamide (130)

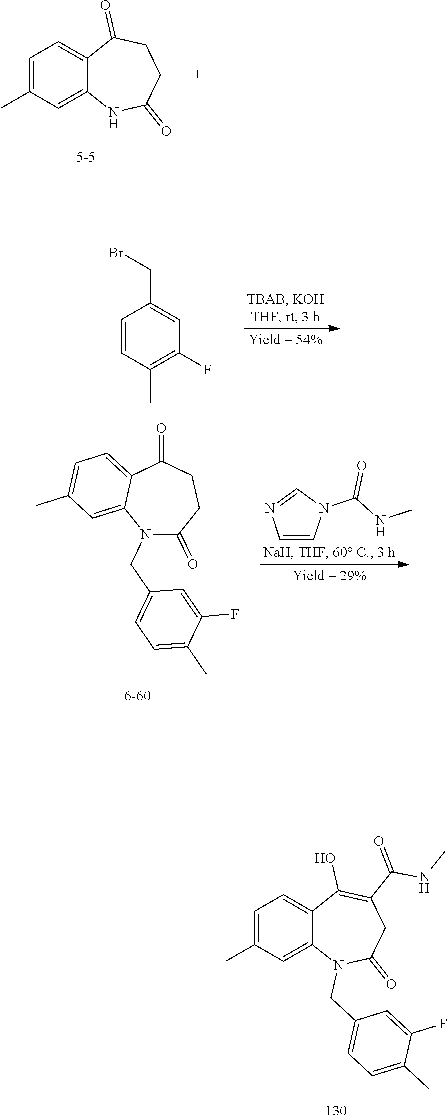

To a stirred solution of ketone (5-5) (189 mg, 1 mmol, 1 equiv), Tetrabutylammonium bromide (32 mg, 0.1 mmol, 0.1 equiv) and KOH (67 mg, 1.4 mmol, 1.4 equiv) in THF (4 mL), was added 4-(bromomethyl)-2-fluoro-1-methylbenzene (153 mg, 1.1 mmol, 1.2 equiv) at ambient temperature. The reaction mixture was then stirred at RT for 3 h. The reaction progress was monitored by TLC until the complete disappearance of compound 5-5. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 170 mg (purity=99%) light yellow solid compound 6-60 with a yield of 54%.

TLC R_{f}=0.5 (PE:EA=1:1)

LCMS (m/z)=312.40 (M+H)

To the solution of resulting compound 6-60 (170 mg, 0.54 mmol, 1 equiv) and N-methyl-1H-imidazole-1-carboxamide (205 mg, 1.64 mmol, 6 equiv) in THF (2 mL), was added NaH 60% dispersion in mineral oil (110 mg, 2.74 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 62 mg (purity=98%) white solid compound 130 with a yield of 29%.

TLC R_{f}=0.45 (EA:PE=1:1)

LCMS (m/z)=369.60 (M+H)

### Example 131. 1-(4-fluoro-3-methylbenzyl)-5-hydroxy-N-methyl-2-oxo-8-(trifluoromethyl)-2,3-dihydro-1H-benzo[b]azepine-4-carboxamide (131)

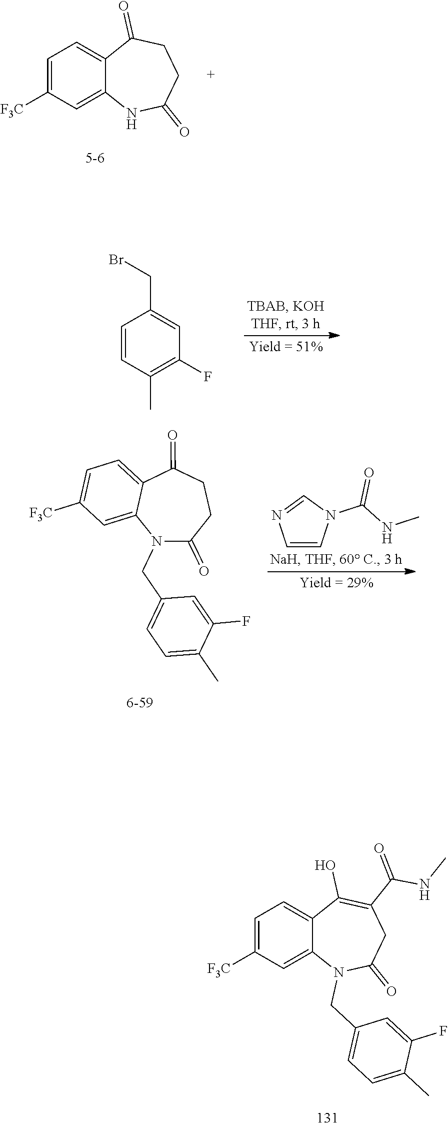

To a stirred solution of ketone (5-6) (500 mg, 2.06 mmol, 1 equiv), Tetrabutylammonium bromide (16 mg, 0.205 mmol, 0.1 equiv) and KOH (162 mg, 2.89 mmol, 1.4 equiv) in THF (10 mL), was added 4-(bromomethyl)-2-fluoro-1-methylbenzene (501 mg, 2.469 mmol, 1.2 equiv) at ambient temperature. The reaction mixture was then stirred at RT for 3 h. The reaction progress was monitored by TLC until the complete disappearance of compound 5-6. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 371 mg (purity=99%) light yellow solid compound 6-59 with a yield of 51%.

TLC R_{f}=0.5 (PE:EA=1:1)

LCMS (m/z)=366.30 (M+H)

To the solution of resulting compound 6-59 (100 mg, 0.274 mmol, 1 equiv) and N-methyl-1H-imidazole-1-carboxamide (205 mg, 1.64 mmol, 6 equiv) in THF (2 mL), was added NaH 60% dispersion in mineral oil (110 mg, 2.74 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 34 mg (purity=93%) white solid compound 131 with a yield of 29%.

TLC R_{f}=0.45 (EA:PE=1:1)

LCMS (m/z)=423.40 (M+H)

### Example 132. 8-bromo-1-(4-fluoro-3-methylbenzyl)-5-hydroxy-N-methyl-2-oxo-2,3-dihydro-1H-benzo[b]azepine-4-carboxamide (132)

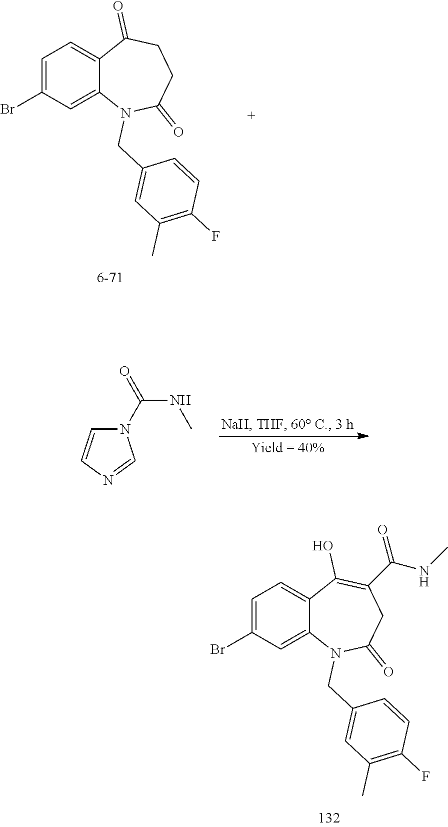

To a stirred solution of ketone (6-71) (100 mg, 0.266 mmol, 1 equiv) and N-methyl-1H-imidazole-1-carboxamide (100 mg, 0.8 mmol, 3 equiv) in THE (2 mL), was added NaH 60% dispersion in mineral oil (105 mg, 2.66 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 46 mg (purity=90%) white solid compound 132 with a yield of 40%.

TLC R_{f}=0.45 (EA:PE=1:2)

LCMS (m/z)=433.70 (M+H)

### Example 133. 8-((3-fluoro-4-methoxybenzyl) oxy)-1-(3-fluoro-4-methylbenzyl)-5-hydroxy-N-methyl-2-oxo-2,3-dihydro-1H-benzo[b]azepine-4-carboxamide (133)

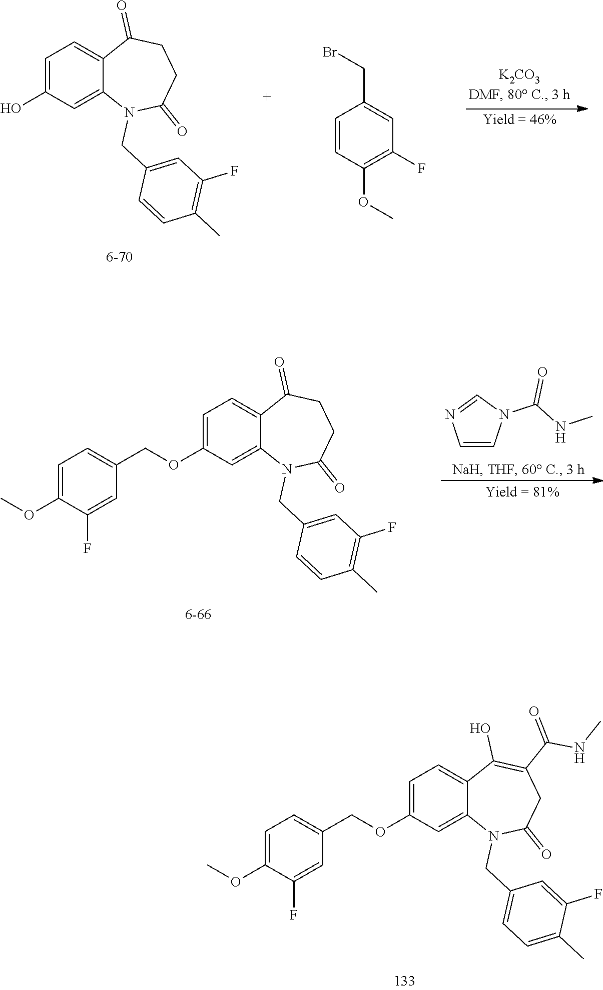

To a stirred solution of ketone (6-70) (174 mg, 0.61 mmol, 1 equiv), K₂CO₃ (168 mg, 1.22 mmol, 2 equiv) in DMF (30 mL), was added 4-(bromomethyl)-2-fluoro-1-methoxybenzene (160 mg, 0.73 mmol, 1.2 equiv) at ambient temperature. The reaction mixture was then stirred at 80° C. for 3 h. The reaction progress was monitored by TLC until the complete disappearance of compound 6-70. The solution was quenched with saturated ammonium chloride and extracted with EA (3×20 mL). The organic solutions were combined, washed with H₂O) (2×20 mL) and brine (1×20 mL), dried over anhydrous Na₂SO₄, filtered and concentrated by rotary evaporation to get the crude compound. Then, the resulting concentrate was purified on a silica column to get 126 mg (purity=99%) light yellow solid compound 6-66 with a yield of 46%.

TLC R_{f}=0.35 (PE:EA=1:2)

LCMS (m/z)=452.10 (M+H)

To the solution of resulting compound 6-66 (126 mg, 0.4 mmol, 1 equiv) and N-methyl-1H-imidazole-1-carboxamide (500 mg, 4 mmol, 10 equiv) in THF (3 mL), was added NaH 60% dispersion in mineral oil (96 mg, 4 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 189.7 mg (purity=96%) white solid compound 133 with a yield of 81%.

TLC R_{f}=0.4 (EA:PE=1:2)

LCMS (m/z)=509.40 (M+H)

### Example 134. 1-(3-fluoro-4-methylbenzyl)-5-hydroxy-N-methyl-2-oxo-8-(thiophen-3-ylmethoxy)-2,3-dihydro-1H-benzo[b]azepine-4-carboxamide (134)

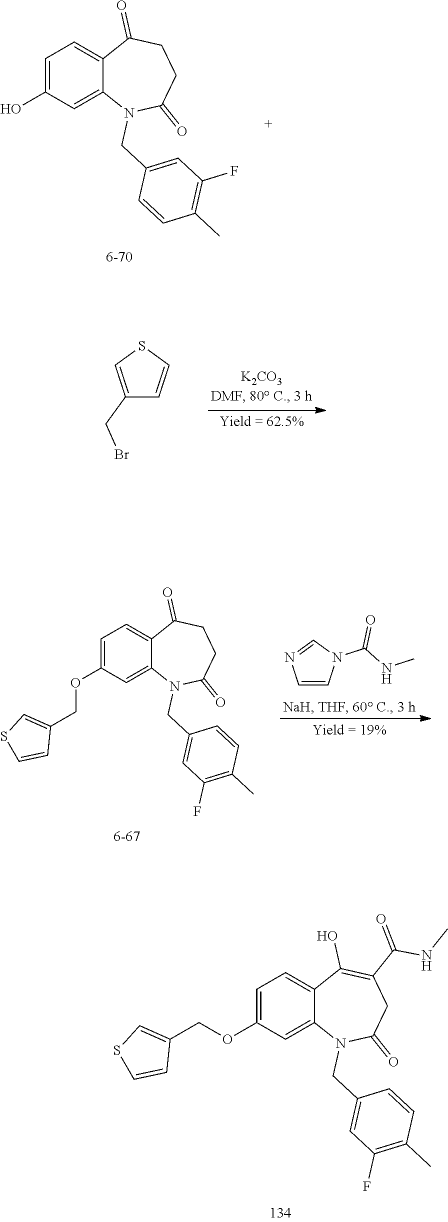

To a stirred solution of ketone (6-70) (150 mg, 0.48 mmol, 1 equiv), K₂CO₃ (132.5 mg, 0.96 mmol, 2 equiv) in DMF (1 mL), was added 3-(bromomethyl) thiophene (64.5 mg, 0.58 mmol, 1.2 equiv), K₂CO₃ (132.5 mg, 0.96 mmol, 2 equiv) at ambient temperature. The reaction mixture was then stirred at 80° C. for 3 h. The reaction progress was monitored by TLC until the complete disappearance of compound 6-70. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 18 mg (purity=99%) white solid compound 6-67 with a yield of 62.5%.

TLC R_{f}=0.2 (EA:PE=1:2)

LCMS (m/z)=410.4 (M+H)

To the solution of resulting compound 6-67 (120 mg, 0.3 mmol, 1 equiv) and N-methyl-1H-imidazole-1-carboxamide (562.5 mg, 4.5 mmol, 15 equiv) in TMF (3 mL), was added NaH 60% dispersion in mineral oil (72 mg, 3 mmol, 10 equiv) at ambient temperature under N₂ atmosphere. The reaction mixture was then stirred at room temperature r.t for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 26.7 mg (purity=96%) white solid compound 134 with a yield of 19%.

TLC R_{f}=0.5 (DCM:MeOH=10:1)

LCMS (m/z)=467.30 (M+H)

### Example 135. 7-fluoro-1-(3-fluoro-4-methylbenzyl)-5-hydroxy-N-methyl-2-oxo-2,3-dihydro-1H-benzo[b]azepine-4-carboxamide (135)

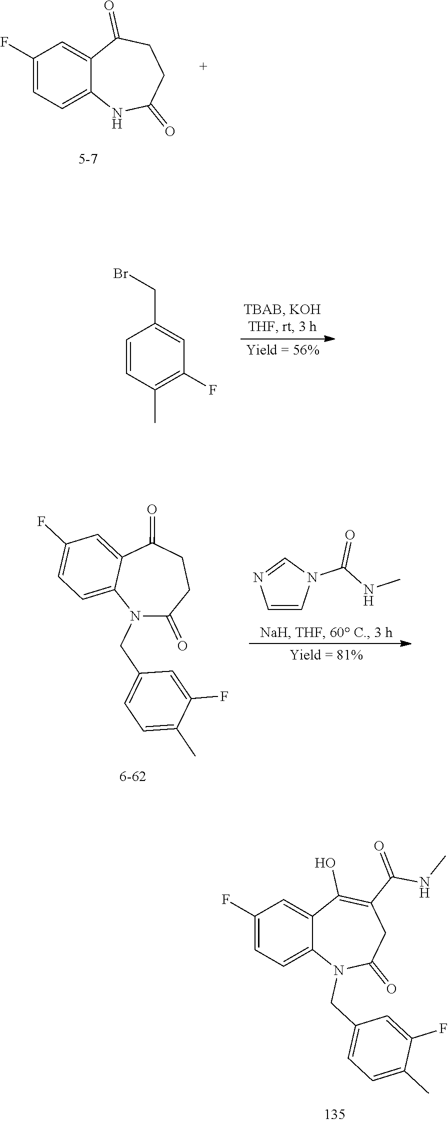

To a stirred solution of ketone (5-7) (500 mg, 2.59 mmol, 1 equiv), Tetrabutylammonium bromide (83 mg, 0.26 mmol, 0.1 equiv) and KOH (203 mg, 3.63 mmol, 1.4 equiv) in THF (10 mL), was added 4-(bromomethyl)-2-fluoro-1-methylbenzene (0.43 mL, 3.11 mmol, 1.2 equiv) at ambient temperature. The reaction mixture was then stirred at RT for 3 h. The reaction progress was monitored by TLC until the complete disappearance of compound 5-7. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 454.9 mg (purity=99%) of the light yellow oil compound 6-62 with a yield of 56%.

TLC R_{f}=0.6 (EA:PE=2:1)

LCMS (m/z)=316.40 (M+H)

To the solution of resulting compound 6-62 (200 mg, 0.64 mmol, 1 equiv) and N-methyl-1H-imidazole-1-carboxamide (1.2 g, 9.6 mmol, 15 equiv) in THF (4 mL), was added NaH 60% dispersion in mineral oil (153.6 mg, 6.4 mmol, 10 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 189.7 mg (purity=96%) white solid compound 135 with a yield of 81%.

TLC R_{f}=0.33 (EA:PE=1:2)

LCMS (m/z)=373.50 (M+H)

### Example 136. 1-(3-fluoro-4-methylbenzyl)-5-hydroxy-N,7-dimethyl-2-oxo-2,3-dihydro-1H-benzo[b]azepine-4-carboxamide (136)

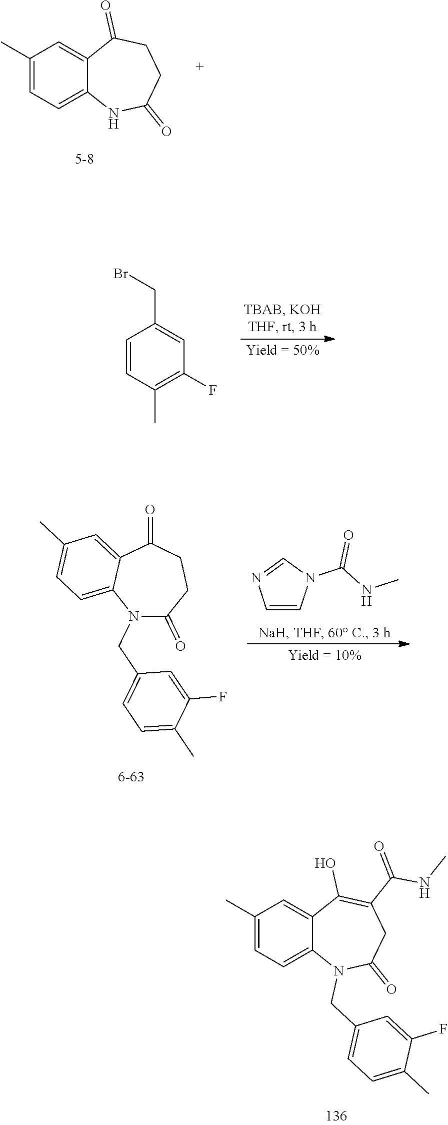

To a stirred solution of ketone (5-8) (500 mg, 2.59 mmol, 1 equiv), Tetrabutylammonium bromide (83 mg, 0.26 mmol, 0.1 equiv) and KOH (203 mg, 3.63 mmol, 1.4 equiv) in THF (10 mL), was added 4-(bromomethyl)-2-fluoro-1-methylbenzene (0.43 mL, 3.11 mmol, 1.2 equiv) at ambient temperature. The reaction mixture was then stirred at RT for 3 h. The reaction progress was monitored by TLC until the complete disappearance of compound 5-8. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 408 mg (purity=99%) of the light yellow oil compound 6-63 with a yield of 50%.

TLC R_{f}=0.6 (EA:PE=1:1)

LCMS (m/z)=316.40 (M+H)

To the solution of resulting compound 6-63 (155 mg, 0.5 mmol, 1 equiv) and N-methyl-1H-imidazole-1-carboxamide (625 mg, S mmol, 10 equiv) in THF (4 mL), was added NaH 60% dispersion in mineral oil (120 mg, 5 mmol, 110 equiv) at ambient temperature under N₂ atmosphere. The reaction mixture was then stirred at 60° C. for 2 h. The solution was quenched with saturated ammonium chloride and extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 18 mg (purity=94%) white sold compound 136 with a yield of 10%.

TLC R_{f}=0.5 (EA:PE=1:2)

LCMS (m/z)=369.10 (M+H)

### Example 137. 1-(3-fluoro-4-methylbenzyl)-5-hydroxy-7-methoxy-N-methyl-2-oxo-2,3-dihydro-1H-benzo[b]azepine-4-carboxamide (137)

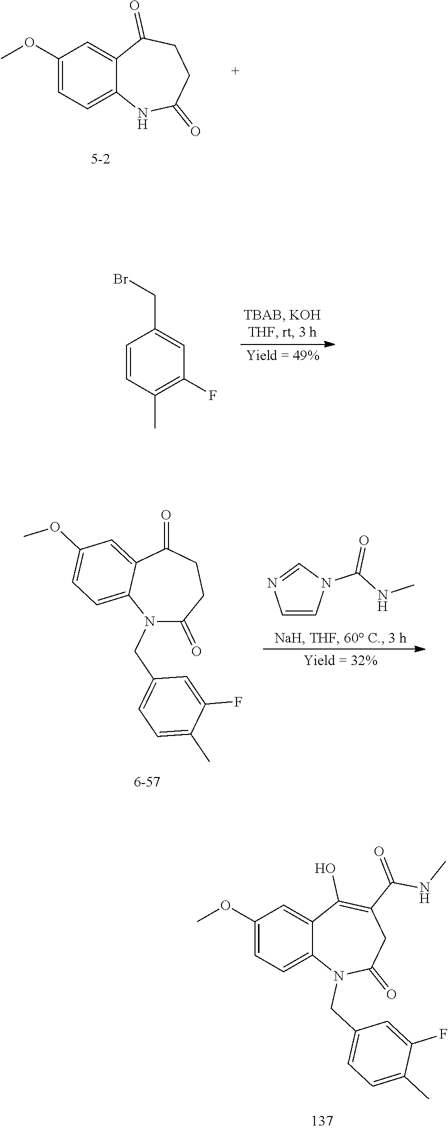

To a stirred solution of ketone (5-2) (205 mg, 1 mmol, 1 equiv), Tetrabutylammonium bromide (33 mg, 0.1 mmol, 0.1 equiv) and KOH (79 mg, 1.4 mmol, 1.4 equiv) in THF (4 mL), was 4-(bromomethyl)-2-fluoro-1-methylbenzene (244 mg, 1.2 mmol, 1.2 equiv) at ambient temperature. The reaction mixture was then stirred at RT for 3 h. The reaction progress was monitored by TLC until the complete disappearance of compound 5-2. The solution was quenched with saturated ammonium chloride and extracted with EA (3×20 mL). The organic solutions were combined, washed with H₂O (2×20 mL) and brine (1×20 mL), dried over anhydrous Na₂SO₄, filtered and concentrated by rotary evaporation to get the crude compound. Then, the resulting concentrate was purified on a silica column to get 160 mg (purity=99%) light yellow solid compound 6-57 with a yield of 49%.

TLC R_{f}=0.5 (EA:PE=1:1)

LCMS (m/z)=327.80 (M+H)

To the solution of resulting compound 6-57 (160 mg, 0.489 mmol, 1 equiv) and N-methyl-1H-imidazole-1-carboxamide (183 mg, 1.464 mmol, 3 equiv) in THF (3 mL), was added NaH 60% dispersion in mineral oil (196 mg, 4.89 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 60 mg (purity=99%) white solid compound 137 with a yield of 32%.

TLC R_{f}=0.5 (EA:PE=1:1)

LCMS (m/z)=385.60 (M+H)

### Example 138. 1-(3-fluoro-4-methylbenzyl)-5-hydroxy-N-methyl-8-(1-methyl-1H-pyrazol-4-yl)-2-oxo-2,3-dihydro-1H-benzo[b]azepine-4-carboxamide (138)

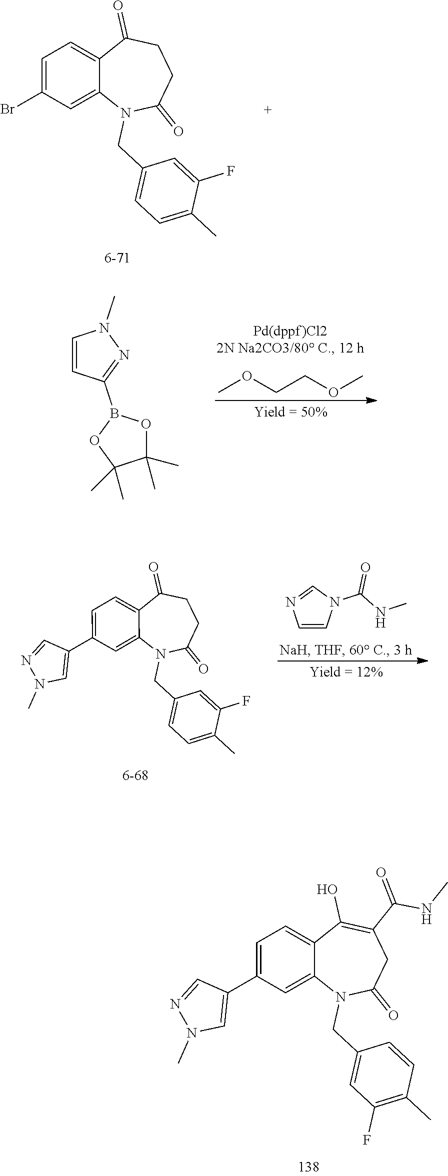

6-71 (187 mg, 0.5 mmol, 1 equiv) and 1-methyl-3-(4,4,5,5-tetramethyl-1,3,2-dioxaborolan-2-yl)-1H-pyrazole (208 mg, 1 mmol, 2 equiv) were placed in a round-bottom flask, followed by DME and 2N Na₂CO3 aqueous solution. The solution was degassed before addition of Pd(dppf)Cl₂ (92 mg, 0.125 mmol, 0.25 equiv) in one portion, the solution was degassed again and heated at 80° C. for 12 h. the solution was extracted with EA (3×10 mL). The organic solutions were combined, washed with H₂O) (2×10 mL) and brine (1×20 mL), dried over anhydrous Na₂SO₄, filtered and concentrated by rotary evaporation to get the crude compound. Then, the resulting concentrate was purified on a silica column to get 140 mg (purity=99%) white solid compound 6-68 with a yield of 50%.

TLC R_{f}=0.4 (DCM:EA=10:1)

LCMS (m/z)=378.40 (M+H)

To the solution of resulting compound 6-68 (140 mg, 0.37 mmol, 1 equiv) and N-methyl-1H-imidazole-1-carboxamide (44 mg, 0.351 mmol, 3 equiv) in THF (3 mL), was added NaH 60% dispersion in mineral oil (147 mg, 1.17 mmol, 10 equiv) at ambient temperature under N₂ atmosphere. The reaction mixture was then stirred at 60° C. for 3 h. The solution was quenched with saturated ammonium chloride and extracted with EA (3×10 mL). The organic solutions were combined, washed with H₂O (2×10 mL) and brine (1×20 mL), dried over anhydrous Na₂SO₄, filtered and concentrated by rotary evaporation to get the crude compound. Then, the resulting concentrate was purified on a silica column to get 18 mg (purity=92%) white solid compound with a yield of 12%.

TLC R_{f}=0.3 (EA:PE=1:1)

LCMS (m/z)=435.40 (M+H)

### Example 139. +-1-(3-fluoro-4-methylbenzyl)-8-isocyano-N-methyl-2,5-dioxo-2,3,4,5-tetrahydro-1H-benzo[b]azepine-4-carboxamide (139)

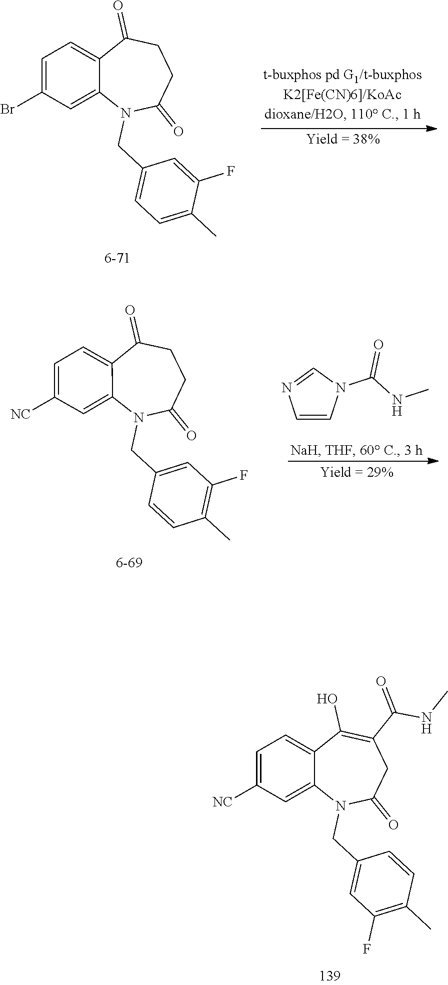

To a solution of 6-71 (188 mg, 0.5 mmol, 1 equiv) in 1,4-dioxane (3.5 mL) were added potassium acetate (13 mg, 0.125 mmol, 0.25 equiv), potassium hexacyanoferrate (II) trihydrate (83 mg, 0.25 mmol, 0.5 equiv), t-BuXPhos palladium(II) phenethylamine chloride (17 mg, 0.025 mmol, 0.05 equiv), 2-di-tert-butylphosphino-2′,4′,6′-triisopropylbiphenyl (11 mg, 0.025 mmol, 0.05 equiv), and water (3.5 mL). After being heated with stirring at 110° C. for 1 h, the reaction mixture was diluted with EtOAc. After removal of the catalyst by filtration, the filtrate was washed with water and brine. The organic layer was dried over Na₂SO₄ and concentrated in vacuo. The residue was purified by column chromatography to get 62 mg (purity=95%) yellow solid compound 6-69 with a yield of 38%.

TLC R_{f}=0.3 (EA:PE=1:2)

LCMS (m/z)=323.20 (M+H)

To the solution of resulting compound 6-69 (62 mg, 0.19 mmol, 1 equiv) and N-methyl-1H-imidazole-1-carboxamide (237.5 mg, 1.9 mmol, 10 equiv) in DMF (1 mL), was added NaH 60% dispersion in mineral oil (45.6 mg, 1.9 mmol, 10 equiv) at ambient temperature under N₂ atmosphere. The reaction mixture was then stirred at room temperature r.t for 0.5 h. The solution was quenched with saturated ammonium chloride and extracted with EA (3×10 mL). The organic solutions were combined, washed with H₂O (2×10 mL) and brine (1×20 mL), dried over anhydrous Na₂SO₁, filtered and concentrated by rotary evaporation to get the crude compound. Then, the resulting concentrate was purified on a silica column to get 21.6 mg (purity=92%) white solid compound 139 with a yield of 29%.

TLC R_{f}=0.45 (PE:EA=2:1)

LCMS (m/z)=380.00 (M+H)

### Example 141. 4-(cyclopropanecarbonyl)-1-(3-fluoro-4-methylbenzyl)-5-hydroxy-8-methoxy-1,3-dihydro-2H-benzo[b]azepin-2-one (141)

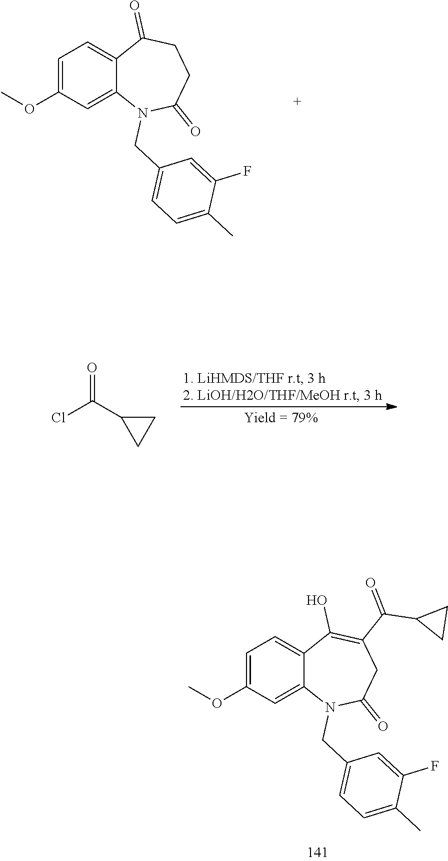 [SMILES: COc1ccc2c(c1)N(Cc1ccc(C)c(F)c1)C(=O)CCC2=O ; O=C(Cl)C1CC1 ; COc1ccc2c(c1)N(Cc1ccc(C)c(F)c1)C(=O)CC(C(=O)C1CC1)=C2O]

To a stirred solution of ketone (100 mg, 0.306 mmol, 1 equiv) and LiHMDS (0.61 mL, 0.612 mmol, 2 equiv) in THF (2 mL) for 20 min, was added cyclopropanecarbonyl chloride (42 μL mg, 0.459 mmol, 1.5 equiv) at 0° C. under N₂ atmosphere. The reaction mixture was then stirred at r.t for 3 h. The solution was quenched with saturated ammonium chloride and extracted with EA (3×10 mL). The organic solutions were combined, washed with H₂O (2×10 ml) and brine (1×20 mL), dried over anhydrous Na₂SO₄, filtered and concentrated by rotary evaporation to get the crude compound. To the crude compound added LiOH·H₂O (51 mg, 1.2 mmol, 4 equiv) and MeOH/THF/H₂O (0.5 mL/0.5 mL/0.5 mL), then stir at r.t for 3 h. The solution was quenched with saturated ammonium chloride and extracted with EA (3×10 mL). The organic solutions were combined, washed with 1 mol/L HCl (2×10 mL) and brine (1×20 mL), dried over anhydrous Na₂SO₄, filtered and concentrated by rotary evaporation to get the crude compound. Then, the resulting concentrate was purified on a silica column to get 95 mg (purity=99%) white solid compound with a yield of 79%.

TLC R_{f}=0.85 (EA:PE=1:1)

LCMS (m/z)=396.60 (M+H)

### Example 142. In Vitro Assays

### Cell Culture and Treatment

Human cancer cell lines were obtained from ATCC and cultured in DMEM (Gibco, Cleveland, TN, USA) supplemented with 5% fetal bovine serum (Gibco), penicillin (100 U/mL)-streptomycin (100 μg/mL) (Gibco, Cat No. 15140-122), 2 mM L-glutamine (Gibco, 200 mM solution, Cat. No. 25030081), and 1 mM sodium pyruvate (Gibco, 100 mM solution, Cat. No. 11360070) at 37° C. in a humidified incubator that was maintained at 5% CO₂.

### Phenotypic Screening Assays and MTT Assays

Screening was conducted as described before (Li et al. 2019). Briefly, RPE-Neo or RPE-MYC or RPE-MYC expressing an EGFP-Histone 2B fusion protein were passaged as batches of 96-well plates, 18-24 hours before exposure to the chemical compounds of the present disclosure at concentrations from 20 nM to 20 μM. At 24, 48 or 72 hours after initiation of treatment, cells were analyzed for either an arrest in mitosis or a change in DNA content by GE IN-Cell Analyzer 2000. MTT assays of cellular proliferation were performed as previously described (Li et al. 2019).

### Immunofluorescent Staining

The immunofluorescence staining procedure has been previously described (Yang et al. 2010). Briefly, cells were cultured on coverslips in a 6-well plate, fixed with 4% paraformaldehyde, and then permeabilized with 0.3% Triton X-100 in PBS. After incubation with a primary antibody, cells were detected with TRITC-conjugated or FITC-conjugated secondary antibodies that were purchased from Jackson ImmunoResearch. After immunostaining, cells were mounted on microscope slides with DAPI-containing Vectashield mounting solution (Vector Laboratories, USA) and fluorescence was detected with an EVOS Auto FL fluorescence microscope (ThermoFisher, USA)

Antibodies Used in this Study

### Primary Antibodies:

Histone H3 (phospho-Ser10), Mouse Monoclonal Antibody, Upstate, Cat #05-806, 1:1000
Phospho-Aurora A (Thr288)(C39D8), Rabbit mAb, Cell Signalling, #3079, 1:1000
Aurora B Antibody, Rabbit, Absin, abs1460-100 ug, lot #AH22, 1:50 dilution
CREST antibody, Thermofisher, Cat. #PA5-30500, 1:200 dilution
Mouse Anti-β-tubulin, Sigma, Cat. #F 2043, clone TUB 2.1 1:50 dilution

### Secondary Antibodies:

Rhodamine (TRITC) AffiniPure Goat Anti-mouse IgG (H+L), Proteintech, SA00007-1, 1:100
Rhodamine (TRITC) AffiniPure Goat Anti-Rabbit IgG (H+L), Jackson, 111-025-003, 1:100
Fluorescein (FITC)-conjugated Affinipure Goat Anti-Rabbit IgG (H+L), Jackson, 111-095-144, 1:100
Rhodamine Red™-X-conjugated AffiniPure Donkey Anti-Human IgG (H+L), Jackson, 709-295-149, 1:100

### Crystal Violet Assay

Cells were fixed in 4% PFA, stained with 0.1% crystal violet (Aladdin, 548-62-9). Bound crystal violet was eluted with 95% ethanol and the absorbance of eluate was measured at 570 nm using a Microplate reader (BioTek ELX808iu).

### Soft Agar Assay

The assay was performed in 6-well plates with two layers of agar. For the first, 0.75% agar in DMEM medium was melted in a microwave oven and poured to form a bottom layer. Once solidified, 10-100K cells in 1 ml of DMEM containing 0.35% agar was added to form the top layer, which was later covered with 0.5 ml DMEM. Cell culture medium was changed once every two days until colonies were ready to photograph

The results of these assays are summarized in Table 2. As set forth in table 2 below, an values of greater than or equal to 1 nM and less than or equal to 1.0 μM is marked “A”; a value greater than 1.00 μM and less than or equal to 10.0 μM is marked “B”; a value greater than 10.0 μM and less than or equal to 30.0 μM is marked “C”; and a value greater than 30.0 μM and less than 1000 μM is marked “D.”

TABLE 2

| | | Min. | | |
|---|---|---|---|---|
| | | Effective | | |
| | Mitotic | conc. for | Cell | Proliferative |
| | Arrest | Polyploidy | Death | arrest |
| Code | (≥10%) | (≥10%) | (>30%) | (<50%) |
| 1 | D | D | D | — |
| 5 | D | D | D | |
| 6 | D | D | D | — |
| 7 | A | A | A | — |
| 8 | — | — | — | D |
| 9 | D | D | — | — |
| 10 | D | D | — | — |
| 11 | — | — | — | D |
| 12 | — | — | D | — |
| 14 | B | B | B | — |
| 16 | — | — | D | — |
| 17 | B | B | B | — |
| 18 | D | D | D | — |
| 19 | D | D | D | — |
| 20 | D | D | D | — |
| 22 | — | — | D | — |
| 26 | — | — | — | D |
| 27 | C | C | — | — |
| 28 | — | — | D | — |
| 29 | B | B | B | — |
| 30 | C | C | C | — |
| 32 | — | — | — | D |
| 33 | B | B | B | — |
| 34 | A | A | A | — |
| 35 | D | D | D | — |
| 36 | C | C | C | — |
| 37 | A | A | A | — |
| 38 | D | D | D | D |
| 39 | — | — | — | — |
| 40 | B | B | B | — |
| 41 | A | A | A | — |
| 42 | — | — | — | D |
| 45 | B | B | B | — |
| 46 | B | B | B | — |
| 48 | A | A | A | — |
| 49 | C | C | C | — |
| 50 | C | C | C | — |
| 51 | D | D | D | — |
| 52 | A | A | A | — |
| 53 | B | B | B | — |
| 54 | A | A | A | — |
| 55 | A | A | | |
| 56 | B | B | | |
| 57 | A | B | B | — |
| 58 | A | A | A | — |
| 59 | A | A | A | — |
| 60 | B | B | B | — |
| 61 | — | — | — | — |
| 62 | B | B | B | — |
| 63 | B | B | B | — |
| 64 | B | B | B | — |
| 65 | B | B | B | — |
| 66 | C | C | C | — |
| 67 | C | C | C | — |
| 68 | C | C | C | — |
| 69 | — | — | — | — |
| 70 | D | D | D | D |
| 71 | D | D | D | D |
| 72 | D | D | D | D |
| 73 | B | B | B | B |
| 74 | D | D | D | D |
| 75 | D | D | D | D |
| 76 | B | B | B | B |
| 77 | D | D | D | D |
| 78 | C | C | C | C |
| 79 | B | B | B | B |
| 80 | B | B | B | B |
| 81 | D | D | D | D |
| 82 | D | D | D | D |
| 83 | C | C | C | C |
| 84 | C | D | D | D |
| 85 | D | D | D | D |
| 86 | B | B | B | B |
| 87 | D | D | D | D |
| 88 | D | D | D | D |
| 89 | D | D | D | D |
| 90 | D | D | D | D |
| 91 | B | C | C | C |
| 92 | C | C | C | C |
| 93 | C | C | C | C |
| 94 | D | D | D | D |
| 95 | B | C | C | C |
| 96 | D | D | D | D |
| 97 | B | B | B | B |
| 98 | C | C | D | D |
| 99 | D | D | D | D |
| 100 | B | B | B | B |
| 101 | B | B | B | B |
| 102 | D | D | D | D |
| 103 | D | D | D | D |
| 104 | D | D | D | D |
| 105 | D | D | D | D |
| 106 | D | D | D | D |
| 107 | D | D | D | D |
| 108 | D | D | D | D |
| 109 | C | C | D | D |
| 110 | B | B | B | B |
| 111 | B | B | B | B |
| 112 | B | B | B | B |
| 113 | C | C | C | C |
| 114 | B | B | B | B |
| 115 | C | D | D | D |
| 116 | D | D | D | D |
| 117 | D | D | D | D |
| 118 | D | D | D | D |
| 119 | C | C | C | C |
| 120 | C | C | C | C |
| 121 | C | C | C | C |
| 122 | D | D | D | D |
| 123 | D | D | D | D |
| 124 | B | B | B | B |
| 125 | B | B | B | B |
| 126 | B | B | B | B |
| 127 | A | A | A | A |
| 128 | B | B | B | B |
| 129 | A | A | A | A |
| 130 | A | A | A | A |
| 131 | B | B | B | B |
| 132 | B | B | B | B |
| 133 | B | B | B | B |
| 134 | B | B | B | B |
| 135 | B | B | B | B |
| 136 | B | B | B | B |
| 137 | B | B | B | B |
| 138 | B | B | B | B |
| 139 | B | B | B | B |
| 140 | A | A | A | A |
| 141 | A | A | A | A |

### Example 143. Xenograft Studies

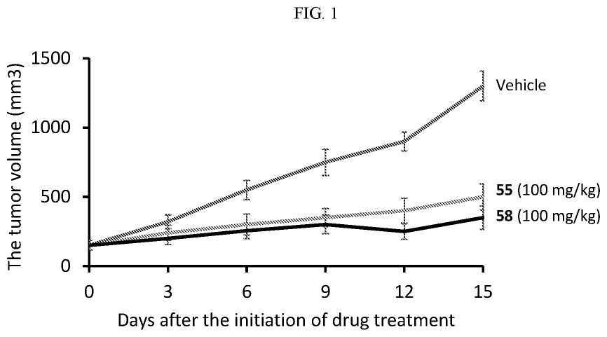

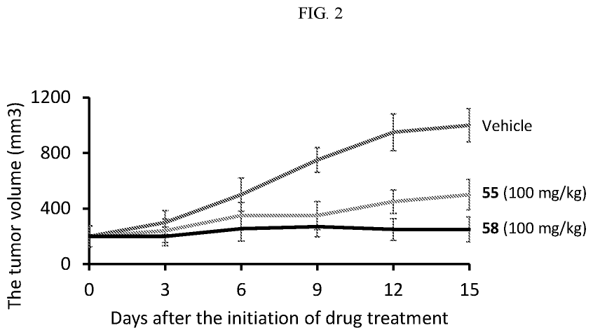

Xenografts were initiated in immunocompromised (Nu/Nu) mice with the human lung adenocarcinoma cell line NCI-H23 (FIG. 1) and the human mammary tumor derived cell line MDA-MB-231 (FIG. 2). Five million cells were injected subcutaneously into mice and treatment was initiated when the average tumor volumes reached 150-200 mm³ (n=8/groups). The indicated compounds were administered through oral gavage twice a day for five days followed by a resting period of 2 days. Day 0 on the X-axis indicates the day that treatment was initiated. For these experiments, 55 and 58 were first dissolved in DMSO and then diluted 1:10 into a mixture containing 70% PEG300 and 30% PBS, pH7.4. 100 ml of drug solution was administered with each dose.

### REFERENCES

Li et al, 2019
Purification, identification, and characterization of two benzophenanthridine alkaloids from Corydalis longicalcarata rhizomes with anti-mitotic and polyploidy-inducing activities Jinhua Li, Ziqi Yan, Hongmei Li, Qiong Shi, Linfang Huang, Thaddeus D. Allen, Dun Yang, Jing Zhang, bioRxiv 821124; doi: https://doi.org/10.1101/821124
Yang et al. 2010
Therapeutic potential of a synthetic lethal interaction between the MYC proto-oncogene and inhibition of aurora-B kinase
Dun Yang, Hong Liu, Andrei Goga, Suwon Kim, Mariia Yuneva, and J. Michael Bishop PNAS Aug. 3, 2010 107 (31) 13836-13841; https://doi.org/10.1073/pnas.1008366107

1. A compound of formula (I): 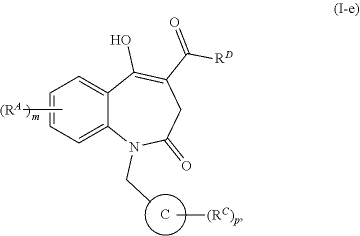
or a pharmaceutically acceptable salt thereof,
wherein
L is —CH₂—;
Ring C is optionally substituted phenyl;
each R^{A} is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OR¹, —SR¹, —N(R¹)₂, —C(O)OR¹, C(O)N(R¹)₂, —N(H)C(O)R¹, and —N(H)C(O)N(R¹)₂; or
each R^{C} is independently selected from the group consisting of halogen, —CN, optionally substituted C₁-C₆ aliphatic, optionally substituted phenyl, —OR³, —S(O)₂R³, —C(O)R³, and —C(O)OR³;
R^{D} is selected from the group consisting of optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —(CH₂)₀₋₃OR², —SR², and —N(R²)₂;
each R¹ is independently selected from the group consisting of hydrogen, —(CH₂)₁₋₃R², optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R¹ is optionally substituted with 1-6 instances of R⁴;
each R² is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R² is optionally substituted with 1-6 instances of R⁴;
each R³ is independently selected from the group consisting of hydrogen, optionally substituted C₁-C₆ aliphatic, optionally substituted phenyl, optionally substituted 5-6-membered heteroaryl, optionally substituted 3-7 membered carbocyclyl, and optionally substituted 3-7 membered heterocyclyl, wherein R³ is optionally substituted with 1-6 instances of R⁴;
each R⁴ is independently selected from the group consisting of halogen, —CN, optionally substituted C₁-C₆ aliphatic, optionally substituted phenyl, optionally substituted 5-6-membered heteroaryl, optionally substituted 3-7 membered carbocyclyl, optionally substituted 3-7 membered heterocyclyl, —OH, —O(C₁-C₃ alkyl), —O(C₁-C₃ haloalkyl), —SH, —S(C₁-C₃ alkyl), —S(C₁-C₃ haloalkyl), —C(O)OH, and —C(O)O(C₁-C₃ alkyl); m is 0, 1, 2, 3, or 4;
p is 0, 1, 2, 3, 4, or 5.

2-6. (canceled)

7. The compound of claim 1, wherein R^{C} is selected from halogen, optionally substituted C₁-C₆ aliphatic, —COOR³, —CN, and —OR³.

8. The compound of claim 7, wherein p is 2.

9. The compound of claim 8, wherein one instance of R^{C} is halogen and the other is optionally substituted C₁-C₆ aliphatic or —OR³.

10. The compound of claim 9, wherein R^{C} is selected from —F, -Me, and —OMe.

11. The compound of claim 1, wherein p is 2 and one instance of R^{C} is fluoro and the other is -Me or —OMe.

12. The compound of claim 1, wherein L and Ring C are taken together to form a group selected from

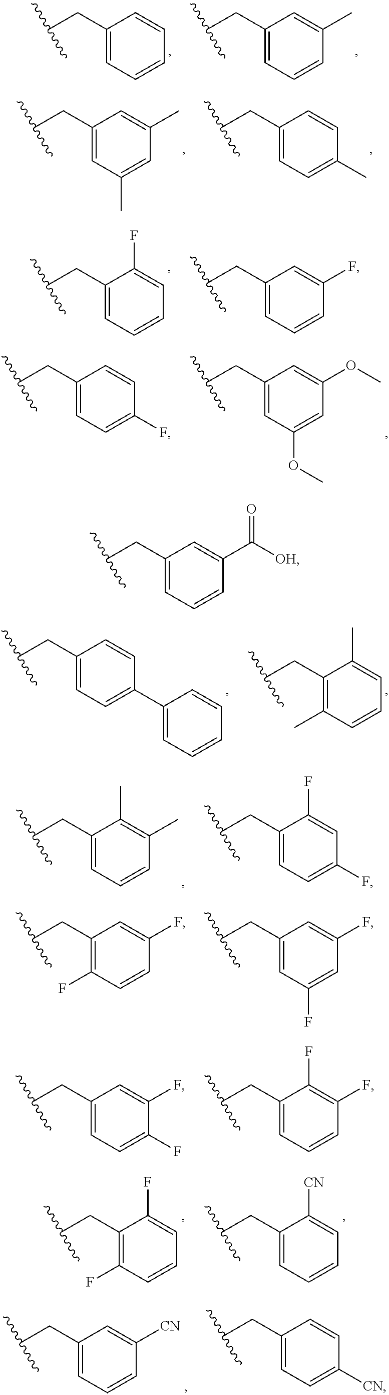

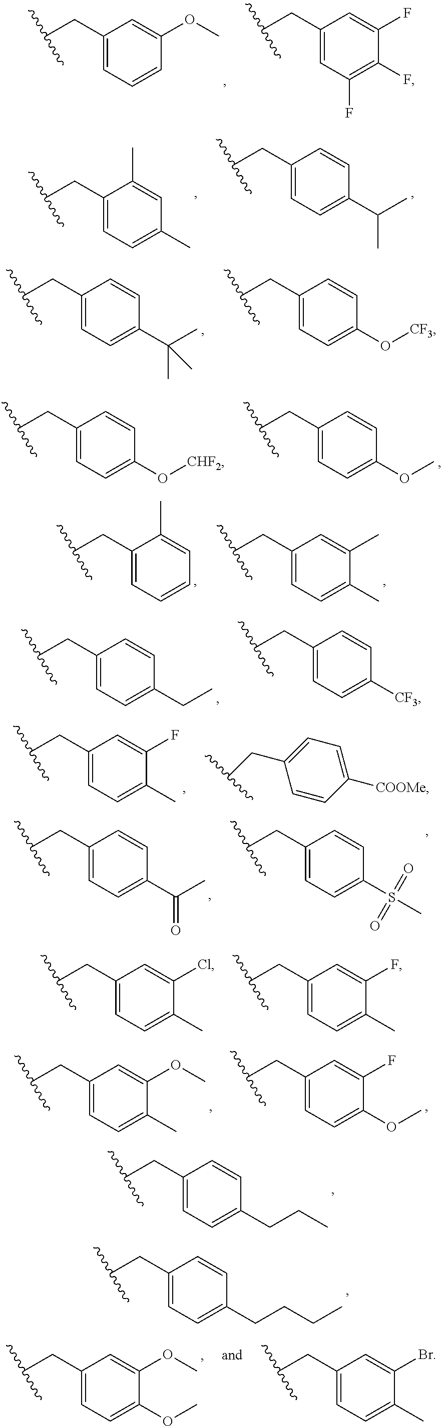

13. The compound of claim 1, wherein R^{D} is selected from optionally substituted C₁-C₃ alphatic and OR².

14. The compound of claim 13, wherein R² is optionally substituted C₁-C₆ aliphatic.

15. The compound of claim 1, wherein R² is optionally substituted C₁-C₃ alkyl.

16. The compound of claim 1, wherein R^{D} is selected from the group consisting of optionally substituted methyl, optionally substituted ethyl, and —OMe.

17. The compound of claim 1, wherein m is 0.

18. The compound of claim 1, wherein the compound is selected from the group consisting of

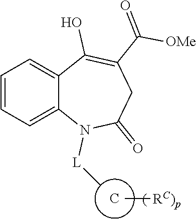

| Compound No. | -L-Ring C |
|---|---|
| 1 | 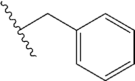 [SMILES: *Cc1ccccc1] |
| 5 | 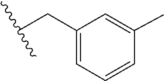 [SMILES: *Cc1cccc(C)c1] |
| 6 | 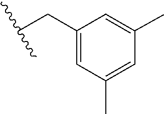 [SMILES: *Cc1cc(C)cc(C)c1] |
| 7 | 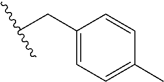 [SMILES: *Cc1ccc(C)cc1] |
| 8 | 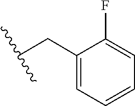 [SMILES: *Cc1ccccc1F] |
| 9 | 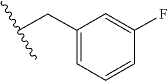 [SMILES: *Cc1cccc(F)c1] |
| 10 | 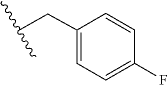 [SMILES: *Cc1ccc(F)cc1] |
| 11 | 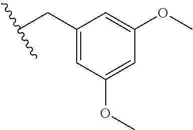 [SMILES: *Cc1cc(OC)cc(OC)c1] |
| 13 | 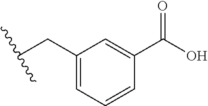 [SMILES: *Cc1cccc(C(=O)O)c1] |
| 15 | 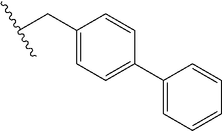 [SMILES: *Cc1ccc(-c2ccccc2)cc1] |
| 16 | 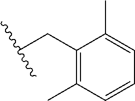 [SMILES: *Cc1c(C)cccc1C] |
| 17 | 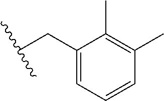 [SMILES: *Cc1cccc(C)c1C] |
| 18 | 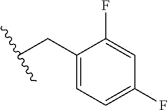 [SMILES: *Cc1ccc(F)cc1F] |
| 19 | 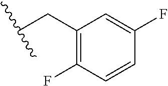 [SMILES: *Cc1cc(F)ccc1F] |
| 20 | 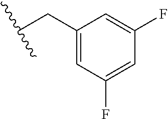 [SMILES: *Cc1cc(F)cc(F)c1] |
| 21 | 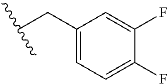 [SMILES: *Cc1ccc(F)c(F)c1] |
| 22 | 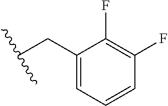 [SMILES: *Cc1cccc(F)c1F] |
| 23 | 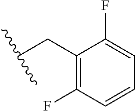 [SMILES: *Cc1c(F)cccc1F] |
| 24 | 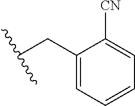 [SMILES: *Cc1ccccc1C#N] |
| 25 | 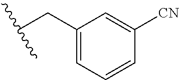 [SMILES: *Cc1cccc(C#N)c1] |
| 26 | 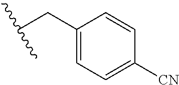 [SMILES: *Cc1ccc(C#N)cc1] |
| 27 | 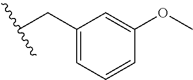 [SMILES: *Cc1cccc(OC)c1] |
| 28 | 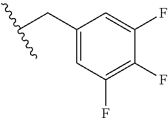 [SMILES: *Cc1cc(F)c(F)c(F)c1] |
| 29 | 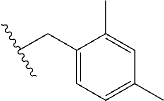 [SMILES: *Cc1ccc(C)cc1C] |
| 30 | 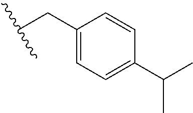 [SMILES: *Cc1ccc(C(C)C)cc1] |
| 31 | 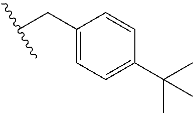 [SMILES: *Cc1ccc(C(C)(C)C)cc1] |
| 32 | 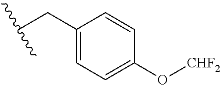 [SMILES: *Cc1ccc(OC(F)F)cc1] |
| 33 | 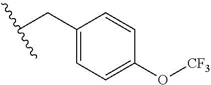 [SMILES: *Cc1ccc(OC(F)(F)F)cc1] |
| 34 | 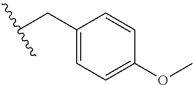 [SMILES: *Cc1ccc(OC)cc1] |
| 35 | 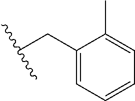 [SMILES: *Cc1ccccc1C] |
| 36 | 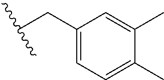 [SMILES: *Cc1ccc(C)c(C)c1] |
| 37 | 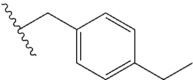 [SMILES: *Cc1ccc(CC)cc1] |
| 40 | 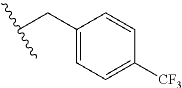 [SMILES: *Cc1ccc(C(F)(F)F)cc1] |
| 41 | 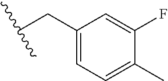 [SMILES: *Cc1ccc(C)c(F)c1] |
| 42 | 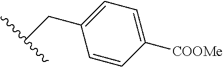 [SMILES: *Cc1ccc(C(=O)OC)cc1] |
| 43 | 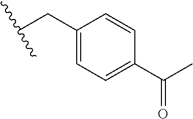 [SMILES: *Cc1ccc(C(C)=O)cc1] |
| 44 | 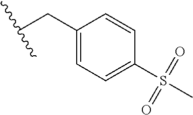 [SMILES: *Cc1ccc(S(C)(=O)=O)cc1] |
| 45 | 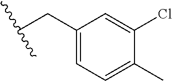 [SMILES: *Cc1ccc(C)c(Cl)c1] |
| 46 | 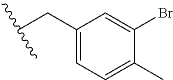 [SMILES: *Cc1ccc(C)c(Br)c1] |
| 47 | 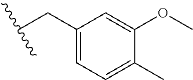 [SMILES: *Cc1ccc(C)c(OC)c1] |
| 48 | 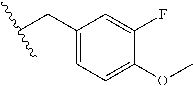 [SMILES: *Cc1ccc(OC)c(F)c1] |
| 49 | 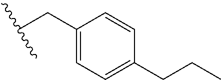 [SMILES: *Cc1ccc(CCC)cc1] |
| 50 | 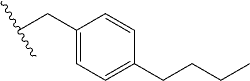 [SMILES: *Cc1ccc(CCCC)cc1] |
| 51 | 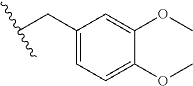 [SMILES: *Cc1ccc(OC)c(OC)c1] |
| Compound No. | Structure |
|---|---|
| 52 | 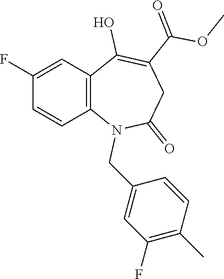 [SMILES: COC(=O)C1=C(O)c2cc(F)ccc2N(Cc2ccc(C)c(F)c2)C(=O)C1] |
| 54 | 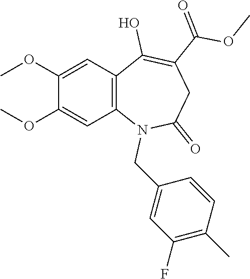 |
| 55 | 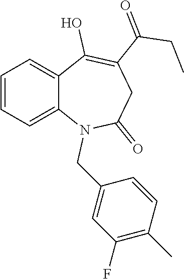 [SMILES: CCC(=O)C1=C(O)c2ccccc2N(Cc2ccc(C)c(F)c2)C(=O)C1] |
| 56 | 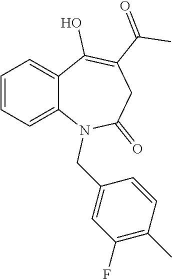 [SMILES: CC(=O)C1=C(O)c2ccccc2N(Cc2ccc(C)c(F)c2)C(=O)C1] |
| 57 | 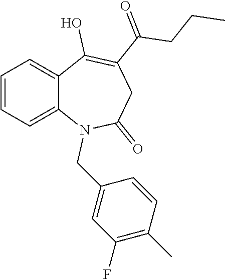 [SMILES: CCCC(=O)C1=C(O)c2ccccc2N(Cc2ccc(C)c(F)c2)C(=O)C1] |
| 58 | 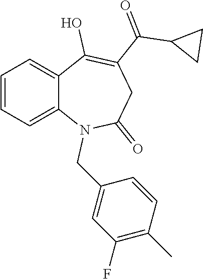 [SMILES: Cc1ccc(CN2C(=O)CC(C(=O)C3CC3)=C(O)c3ccccc32)cc1F] |
| 59 | 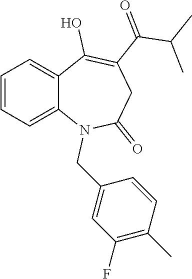 [SMILES: Cc1ccc(CN2C(=O)CC(C(=O)C(C)C)=C(O)c3ccccc32)cc1F] |
| 60 | 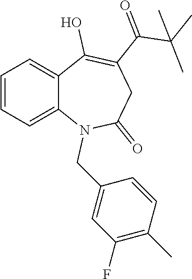 [SMILES: Cc1ccc(CN2C(=O)CC(C(=O)C(C)(C)C)=C(O)c3ccccc32)cc1F] |
| 61 | 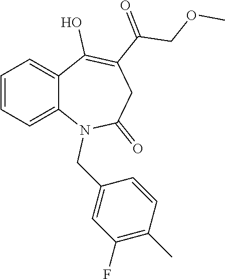 [SMILES: COCC(=O)C1=C(O)c2ccccc2N(Cc2ccc(C)c(F)c2)C(=O)C1] |
| 62 | 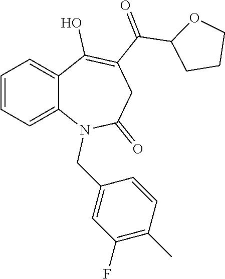 [SMILES: Cc1ccc(CN2C(=O)CC(C(=O)C3CCCO3)=C(O)c3ccccc32)cc1F] |
| 63 | 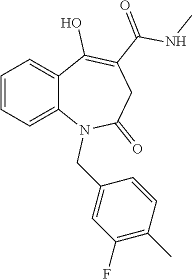 [SMILES: CNC(=O)C1=C(O)c2ccccc2N(Cc2ccc(C)c(F)c2)C(=O)C1] |
| 64 | 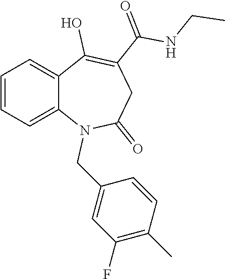 [SMILES: CCNC(=O)C1=C(O)c2ccccc2N(Cc2ccc(C)c(F)c2)C(=O)C1] |
| 65 | 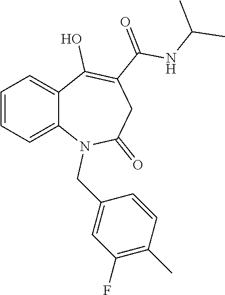 [SMILES: Cc1ccc(CN2C(=O)CC(C(=O)NC(C)C)=C(O)c3ccccc32)cc1F] |
| 66 | 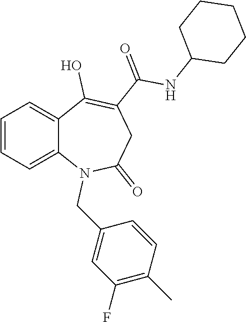 [SMILES: Cc1ccc(CN2C(=O)CC(C(=O)NC3CCCCC3)=C(O)c3ccccc32)cc1F] |
| 68 | 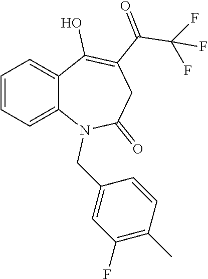 [SMILES: Cc1ccc(CN2C(=O)CC(C(=O)C(F)(F)F)=C(O)c3ccccc32)cc1F] |
| 69 | 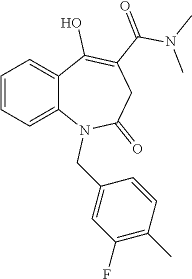 [SMILES: Cc1ccc(CN2C(=O)CC(C(=O)N(C)C)=C(O)c3ccccc32)cc1F] |

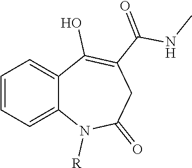
| No. | R = |
|---|---|
| 72 | 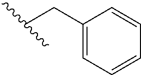 [SMILES: *Cc1ccccc1] |
| 76 | 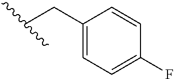 [SMILES: *Cc1ccc(F)cc1] |
| 77 | 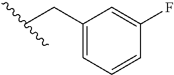 [SMILES: *Cc1cccc(F)c1] |
| 78 | 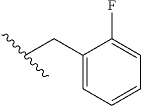 [SMILES: *Cc1ccccc1F] |
| 79 | 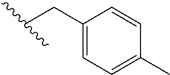 [SMILES: *Cc1ccc(C)cc1] |
| 80 | 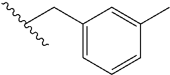 [SMILES: *Cc1cccc(C)c1] |
| 81 | 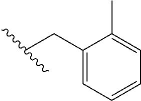 [SMILES: *Cc1ccccc1C] |
| 83 | 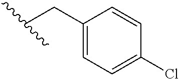 [SMILES: *Cc1ccc(Cl)cc1] |
| 84 | 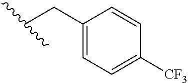 [SMILES: *Cc1ccc(C(F)(F)F)cc1] |
| 85 | 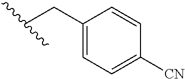 [SMILES: *Cc1ccc(C#N)cc1] |
| 86 | 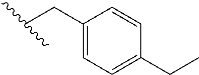 [SMILES: *Cc1ccc(CC)cc1] |
| 87 | 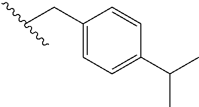 [SMILES: *Cc1ccc(C(C)C)cc1] |
| 88 | 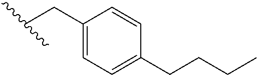 [SMILES: *Cc1ccc(CCCC)cc1] |
| 89 | 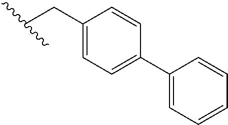 [SMILES: *Cc1ccc(-c2ccccc2)cc1] |
| 90 | 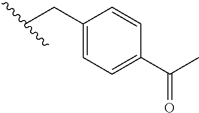 [SMILES: *Cc1ccc(C(C)=O)cc1] |
| 91 | 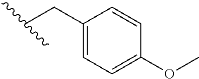 [SMILES: *Cc1ccc(OC)cc1] |
| 92 | 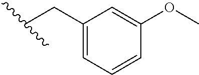 [SMILES: *Cc1cccc(OC)c1] |
| 93 | 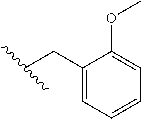 [SMILES: *Cc1ccccc1OC] |
| 94 | 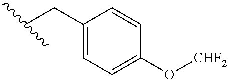 [SMILES: *Cc1ccc(OC(F)F)cc1] |
| 95 | 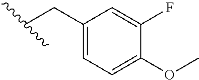 [SMILES: *Cc1ccc(OC)c(F)c1] |
| 96 | 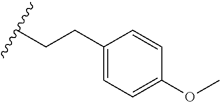 [SMILES: *CCc1ccc(OC)cc1] |
| 97 | 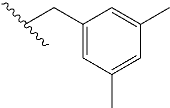 [SMILES: *Cc1cc(C)cc(C)c1] |
| 98 | 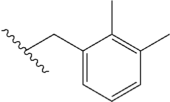 [SMILES: *Cc1cccc(C)c1C] |
| 99 | 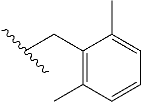 [SMILES: *Cc1c(C)cccc1C] |
| 100 | 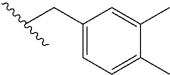 [SMILES: *Cc1ccc(C)c(C)c1] |
| 101 | 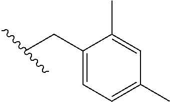 [SMILES: *Cc1ccc(C)cc1C] |
| 102 | 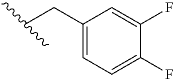 [SMILES: *Cc1ccc(F)c(F)c1] |
| 103 | 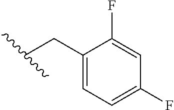 [SMILES: *Cc1ccc(F)cc1F] |
| 104 | 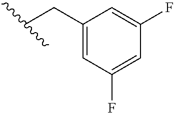 [SMILES: *Cc1cc(F)cc(F)c1] |
| 105 | 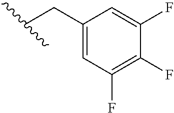 [SMILES: *Cc1cc(F)c(F)c(F)c1] |
| 106 | 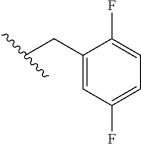 [SMILES: *Cc1cc(F)ccc1F] |
| 107 | 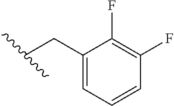 [SMILES: *Cc1cccc(F)c1F] |
| 108 | 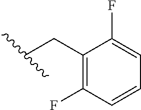 [SMILES: *Cc1c(F)cccc1F] |
| 109 | 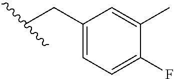 [SMILES: *Cc1ccc(F)c(C)c1] |
| 110 | 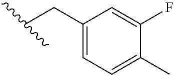 [SMILES: *Cc1ccc(C)c(F)c1] |
| 111 | 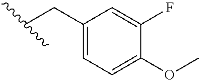 [SMILES: *Cc1ccc(OC)c(F)c1] |
| 112 | 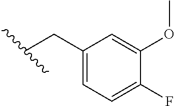 [SMILES: *Cc1ccc(F)c(OC)c1] |
| 113 | 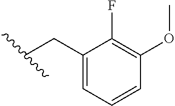 [SMILES: *Cc1cccc(OC)c1F] |
| 114 | 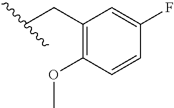 [SMILES: *Cc1cc(F)ccc1OC] |
| 115 | 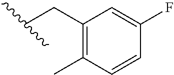 [SMILES: *Cc1cc(F)ccc1C] |
| 116 | 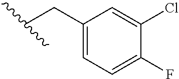 [SMILES: *Cc1ccc(F)c(Cl)c1] |
| 117 | 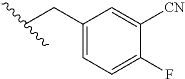 [SMILES: *Cc1ccc(F)c(C#N)c1] |
| 118 | 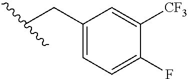 [SMILES: *Cc1ccc(F)c(C(F)(F)F)c1] |
| 119 | 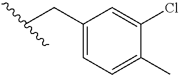 [SMILES: *Cc1ccc(C)c(Cl)c1] |
| 120 | 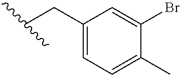 [SMILES: *Cc1ccc(C)c(Br)c1] |
| 121 | 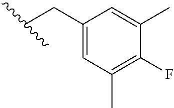 [SMILES: *Cc1cc(C)c(F)c(C)c1] |
| 122 | 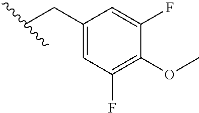 [SMILES: *Cc1cc(F)c(OC)c(F)c1] |
| 123 | 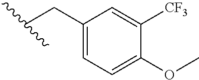 [SMILES: *Cc1ccc(OC)c(C(F)(F)F)c1] |
| 124 | 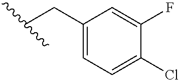 [SMILES: *Cc1ccc(Cl)c(F)c1] |
| 125 | 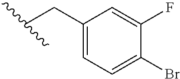 [SMILES: *Cc1ccc(Br)c(F)c1] |

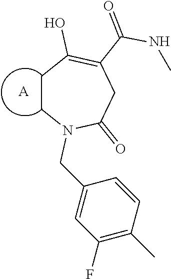
| No. | A = |
|---|---|
| 126 | 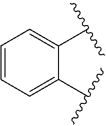 [SMILES: *c1ccccc1*] |
| 127 | 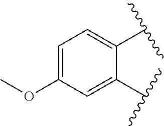 [SMILES: *c1ccc(OC)cc1*] |
| 128 | 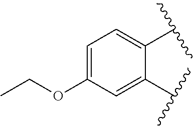 [SMILES: *c1ccc(OCC)cc1*] |
| 129 | 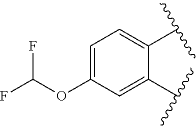 [SMILES: *c1ccc(OC(F)F)cc1*] |
| 130 | 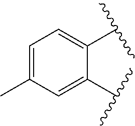 [SMILES: *c1ccc(C)cc1*] |
| 131 | 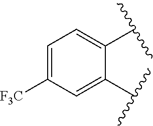 [SMILES: *c1ccc(C(F)(F)F)cc1*] |
| 132 | 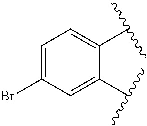 [SMILES: *c1ccc(Br)cc1*] |
| 133 | 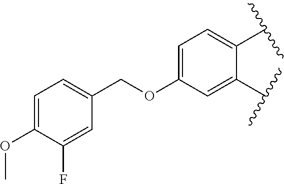 [SMILES: *c1ccc(OCc2ccc(OC)c(F)c2)cc1*] |
| 134 | 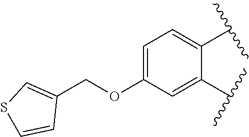 [SMILES: *c1ccc(OCc2ccsc2)cc1*] |
| 135 | 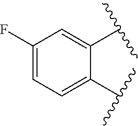 [SMILES: *c1ccc(F)cc1*] |
| 136 | 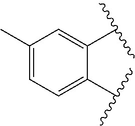 [SMILES: *c1ccc(C)cc1*] |
| 137 | 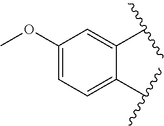 [SMILES: *c1ccc(OC)cc1*] |
| 138 | 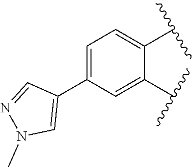 [SMILES: *c1ccc(-c2cnn(C)c2)cc1*] |
| 139 | 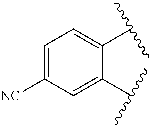 [SMILES: *c1ccc(C#N)cc1*] |
| 140 | 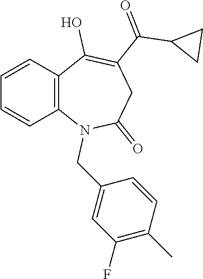 |
| 141 | 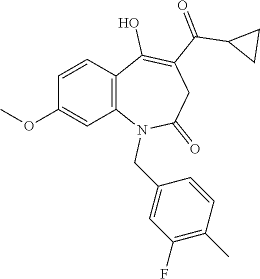 |

or a pharmaceutically acceptable salt thereof.

19. A pharmaceutical composition comprising a compound of claim 1 and a pharmaceutically acceptable excipient.

20. A method of treat cancer comprising administering to a patient in need thereof the claim 1.
